(19) United States
(12) Patent Application Publication
(10) Pub. No.: US 2019/0112618 A1
(43) Pub. Date: Apr. 18, 2019

(54) METHODS FOR CREATING BOTH MALE AND FEMALE STERILE PLANTS AND RESTORATION OF FERTILITY

(71) Applicant: UWM Research Foundation, Inc., Milwaukee, WI (US)

(72) Inventors: Dazhong Zhao, Whitefish Bay, WI (US); Jian Huang, Milwaukee, WI (US)

(21) Appl. No.: 15/748,939

(22) PCT Filed: Jul. 29, 2016
(86) PCT No.: PCT/US2016/044830

§ 371 Date: Jan. 30, 2018

Related U.S. Application Data
(60) Provisional application No. 62/198,979, filed on Jul. 30, 2015.

(51) Int. Cl. C12N 15/82; C12N 15/113

(52) U.S. Cl. CPC C12N 15/8287; C12N 15/113; C12N 15/8218

(57) ABSTRACT

Disclosed herein are compositions and methods for creating sterile plants by genetically ablating microspore and megaspore mother cells. Also disclosed herein are methods of restoring fertility of sterile male and female plants.

### CROSS REFERENCE TO RELATED APPLICATIONS

This application claims priority to U.S. Provisional Application No. 62/198,979, filed Jul. 30, 2015, which is incorporated herein by reference in its entirety.

### TECHNICAL FIELD

The present invention relates to compositions and methods for creating sterile plants by genetically ablating microspore and megaspore mother cells.

### BACKGROUND

Genetically modified (GM) plants, including GM trees, turf grasses, biofuel and forage crops, and ornamentals, improve commercially important traits, such as biomass and biofuel production, digestibility, bioremediation, ornamental value, and tolerance to stresses. However, commercial uses of GM plants are severely limited by stringent government regulations due to concerns over potential ecological effects of transgene flow and floral-modified plantations. Transgene flow from GM plants to non-GM plants and wild populations is mainly mediated by dispersal of pollen and seeds. Early studies found that the pollen-mediated gene flow from GM Roundup Ready creeping bentgrass (a turfgrass) occurred within 2 to 21 km. The non-GM rabbit food grass could pollinate the GM creeping bentgrass to produce transgenic intergeneric hybrid offspring, suggesting that the transgene escape can also be mediated by the female part of GM plants. Long distance pollen-mediated gene flow occurred between weed beets as far as 9.6 km and the resulting interfield gene flow is unavoidable. Pollen migration from poplars often goes beyond 10 km, indicating that similar issues happened in GM trees. Moreover, gene flow from GM crops to native populations was detected in maize, soybean, wheat, and canola. To overcome regulatory hurdles to field research and, ultimately, commercial uses of GM plants, a practical solution is to create sterile plants by ablating floral organs/tissues using toxic genes under control of specific promoters or by altering flowering time and floral organs via manipulating genes critical for flower development.

Strategies on making male sterility have been employed to prevent the pollen-mediated transgene flow. This strategy has also been applied to asexually propagated GM perennial grasses and trees. In addition, manipulating genes regulating flowering time, floral meristem identify, floral organ identity, and floral organ establishment is used to abolish plant fertility. Although male sterility has been successfully achieved via different approaches in various plant species, it cannot completely prevent transgene flow. Seed development in male sterile GM plants can be rescued by the long-distance transfer of pollen from non-GM plants. The same is also true for female sterile GM plants which disperse pollen to non-GM or male sterile GM plants. Thus, completely abolishing male and female fertility is the only fail-safe way to prevent transgene flow. Moreover, existing strategies for creating male sterility, female sterility, or both lead to loss or alterations of entire flowers or floral organs, which may cause potential ecological effects on biodiversity of species associated with flowers, such as insects. In addition, genetically engineered ornamental plants that do not produce flowers or exhibit floral organ alterations reduce their ornamental value. The remaining toxicity of BARNASE in non-target organs due to unspecific basal activities of employed promoters inhibits plant survival and growth. In addition, the male fertility restoring system BARNASE-BARSTAR has been used to restore the male fertility via suppressing the BARNASE enzyme activity by its protein inhibitor BARSTAR. Seed production of BARNASE-created male sterile plants is restored by introducing BARSTAR, a BARNASE inhibitor. However, the BARNASE:BARSTAR protein complex may cause potential health risk and no restoration system has been tested to restore female fertility.

Biotechnologies for engineering sterility without altering either growth or floral structure are needed to prevent dispersal of transgenes and to reduce concerns regarding ecological impacts from genetically modified (GM) plants, such as GM trees, turf grasses, biofuel and forage crops, and ornamentals. There is a need to generate sterility in both male and female reproductive organs without affecting plant growth or altering flower structure. In addition, a system to restore both male and female fertility is needed to directly down-regulate the expression of BARNASE.

### SUMMARY

The present invention is also directed to an isolated polynucleotide construct comprising a first polynucleotide and a second polynucleotide, the first polynucleotide comprising a SOLO-DANCERS (SDS) gene or fragment thereof, the second polynucleotide comprising a Barnase gene or fragment thereof, wherein the SDS gene comprises the SDS promoter. The present invention is directed to a vector comprising said isolated polynucleotide construct. The present invention is directed to a plant cell comprising said vector. The present invention is directed to a plant comprising said plant cell.

The present invention is also directed to a composition for generating a complete male sterile and female sterile transgenic plant. The composition comprises said isolated polynucleotide construct. The present invention is directed to a vector comprising said composition. The present invention is directed to a plant cell comprising said vector or said composition. The present invention is directed to a plant comprising said plant cell.

The present invention is also directed to a method for generating a complete male sterile and female sterile plant. The method comprises introducing into a target plant said isolated polynucleotide construct to generate a transgenic plant. The present invention is directed to a transgenic plant produced by said method.

The present invention is also directed to a method for ablating microspore and megaspore mother cells in a plant. The method comprises introducing into a target plant said isolated polynucleotide construct to generate a transgenic plant, wherein the microspore and megaspore mother cells are ablated.

The present invention is also directed to a method for restoring fertility in a male sterile and female sterile transgenic plant. The method comprises (a) introducing into a target plant said composition to generate a transgenic plant; (b) introducing into the transgenic plant generated in (a) said isolated polynucleotide construct to generate a double transgenic plant; and (c) inducing the expression of the amiRNA, thereby restoring fertility in a complete male sterile and female sterile transgenic sterile plant.

### BRIEF DESCRIPTION OF THE DRAWINGS

FIGS. 1A-ID show schematic diagrams of constructs. FIG. 1A shows the SDS::BARNASE construct. FIG. 1B shows the SDS::GUS construct. FIG. 1C shows the SDS::SDS-GFP construct. FIG. 1D shows the SDS::SDS-BARNASE construct. LB and RB, the T-DNA left and right border, respectively; BAR, the gene conferring resistance to the herbicide Basta; SDS::, the 1.5-kb promoter of the SDS gene; BARNASE, the bacterial ribonuclease; KAN, the kanamycin resistance gene; GUS, the gene encoding β-glucuronidase; GFP, the gene encoding green fluorescent protein; HPT, the hygromycin phosphotransferase gene; and SDS::SDS, the SDS genomic fragment containing a 1.5-kb promoter followed by a DNA fragment consisting of seven exons and six introns.

FIGS. 2A-2I show that the SDS::BARNASE Arabidopsis plants were abnormal in growth and development. FIGS. 2A-2C show that compared to wild type (FIG. 2A), three-week old SDS::BARNASE (FIGS. 2B and 2C) show plants produced less rosette leaves with irregular shape. Bars=0.5 cm. FIGS. 2D-2G show six-week old wild-type (WT, FIG. 2D) and SDS::BARNASE plants showing fertile but dwarf (FIG. 2E), dwarf and sterile (FIG. 2F), and no inflorescence (FIG. 2G) phenotypes. Bars=1 cm. FIG. 2H shows six-week old SDS::BARNASE plants were significantly shorter than the wild type. FIG. 2I shows the rosette leaf number of SDS::BARNASE adult plants was significantly reduced. “n” indicates the number of examined plants. Stars indicate significant difference (P<0.01).

FIGS. 3A-3F show that the entire SDS gene but not the SDS 1.5-kb promoter confers the SDS meiocyte-specific expression. FIGS. 3A-3D show GUS staining of SDS::GUS plants showing GUS signals in cotyledons, true leaves, and shoot apical meristem of a young seedling (FIG. 3A), as well as in carpels and stigmas of young buds (FIGS. 3B-3D). FIG. 3E shows a confocal image from an SDS::SDS-GFP stage-5 anther showing the GFP signal (green color) only in microspore mother cells (arrows). Red and yellow colors showing merged autofluorescences. FIG. 3F shows a confocal image from an SDS::SDS-GFP stage 2-IV ovule showing the GFP signal only in the megaspore mother cell (arrow). Bars=0.1 cm (FIGS. 3A and 3B), 0.5 mm (FIGS. 3C and 3D), 50 μm (FIG. 3E), and 10 μm (FIG. 3F).

FIGS. 4A-4H show that the SDS::SDS-BARNASE Arabidopsis plants showed normal growth and development. FIGS. 4A and 4B show three-week old WT (FIG. 4A) and SDS::SDS-BARNASE (FIG. 4B) plants. Bars=0.5 cm. FIGS. 4C and 4D show five-week old WT (FIG. 4C) and SDS::SDS-BARNASE (FIG. 4D) inflorescences. Bars=0.5 cm. FIGS. 4E and 4F show six-week old WT (FIG. 4E) and SDS::SDS-BARNASE (FIG. 4F) plants. Bars=1 cm. FIG. 4G shows no difference in average height between six-week old WT and SDS::SDS-BARNASE plants. FIG. 4H shows similar rosette leaf numbers indicating no difference in flowering time between WT and SDS::SDS-BARNASE plants. “n” in FIGS. 4G and 4H indicates the number of examined plants.

FIGS. 5A-5J show that the SDS::SDS-BARNASE Arabidopsis plants were completely both male and female sterile. FIGS. 5A-5C show primary branches showing normal siliques in wild type (FIG. 5A) and short siliques indicating no developing seeds in SDS::SDS-BARNASE plants without (FIG. 5B) and with (FIG. 5C) pollination. Bars=1 cm. FIGS. 5D and 5E show side view of mature flowers (One sepal was removed, respectively) showing the SDS::SDS-BARNASE flower (FIG. 5E) is similar to the wild type (FIG. 5D) except short filaments. Pollen grains released from WT anthers (FIG. 5D, inset), while no pollen grains from SDS::SDS-BARNASE anthers (FIG. 5E, inset). Bars=0.5 mm. (FIGS. 5F and 5G) Pollen staining showing the WT anther full of viable pollen grains (FIG. 5F), but no pollen grains from the SDS::SDS-BARNASE anther (FIG. 5G). Bars=30 μm. FIGS. 5H-5J show dissected individual siliques from primary inflorescences (positions 1-9) were long in wild type (FIG. 5H), but short in SDS::SDS-BARNASE plants (FIG. 5I, without pollination; FIG. 5J, pollinated with WT pollen). Bars=1 cm.

FIGS. 6A-6F show that the formation of male gametes was arrested in SDS::SDS-BARNASE Arabidopsis plants. FIGS. 6A-6C show WT anthers showing microsporocytes (microspore mother cells) and surrounding tapetal cells at stage 5 (FIG. 6A), tetrads and tapetal cells at stage 7 (FIG. 6B), and developing pollen grains at stage 9 (FIG. 6C). FIGS. 6D-6F show SDS::SDS-BARNASE anthers showing degenerating microsporocytes and precociously vacuolated tapetal cells at stage 5 (FIG. 6D), dead microsporocytes and tapetal cells at stage 7 (FIG. 6E), and a nearly empty anther lobe at stage 9 (only one dead pollen, FIG. 6F). M, microsporocytes (microspore mother cells); DP, developing pollen; T, tapetal cell; and Tds, tetrads.

FIGS. 7A-7F show that the formation of female gamete was arrested in SDS::SDS-BARNASE Arabidopsis plants. FIGS. 7A-7C show WT ovules showing two separated nuclei (arrows) at the FG3 stage (FIG. 7A), four nuclei (arrows) at the FG4 stage (FIG. 7B), and the central cell, the egg cell, and synergid cells in a mature embryo sac (white dots outlined) at the FG6 stage (FIG. 7C). FIGS. 7D-7F show SDS::SDS-BARNASE ovules showing one small nucleus (arrow) at both FG3 (FIG. 7D) and FG4 (FIG. 7E) stages and a small empty embryo sac (white dots outlined) at the FG6 stage (FIG. 7F). Bars=10 μm. cc, central cell; ec, egg cell; and syn, synergid cells.

FIG. 8 shows the expressions of tapetal cell as well as microspore and megaspore mother cell marker genes. Real-time qRT-PCR showing decreased expressions of tapetal cell marker genes A9 and ATA7 as well as microspore and megaspore mother cell marker genes DMC1 and SW11. Stars indicate significant difference (P<0.01).

FIGS. 9A-9F show that the SDS::SDS-BARNASE tobacco plants showed normal growth and development. FIG. 9A shows forty-day old tobacco WT and SDS::SDS-BARNASE plants. Bar=5 cm. FIGS. 9B and 9C show Sixty-day old WT (FIG. 9B) and SDS::SDS-BARNASE (FIG. 9C) plants. Bars=10 cm. FIG. 9D shows no difference in average height between WT and SDS::SDS-BARNASE adult plants. FIGS. 9E and 9F show flower size, color, and structure remained the same in WT and SDS::SDS-BARNASE plants. Bars=1 cm.

FIGS. 10A-10H show that the SDS::SDS-BARNASE tobacco plants were completely both male and female sterile. FIGS. 10A-10C show large fruits from the WT plant (FIG. 10A) and small fruits from SDS::SDS-BARNASE plants without (FIG. 10B) and with (FIG. 10C) manual pollination with WT pollen grains. Bars=1 cm. FIG. 10D shows the weight of seeds per self-pollinated and manually pollinated fruit (n=5), respectively. Numbers indicate examined independent transgenic lines. FIG. 10E shows WT viable pollen grains in red color. FIGS. 10F-10H show no (FIG. 10F), all dead (FIG. 10G) and a few viable (FIG. 10H) pollen grains in SDS::SDS-BARNASE plants. Numbers indicate examined independent transgenic lines. Bars=100 μm.

FIGS. 11A-11C show schematic diagrams of constructs. FIG. 11A shows a schematic diagram of the SDS::BARNASE construct. BARSTAR, the BARNASE inhibitor gene; KanR, the kanamycin resistance gene; LB, the T-DNA left border; BAR, the BASTA resistance gene; SDS::, the SDS 1.5-Kb promoter region; BARNASE, the bacterial ribonuclease; and RB, the T-DNA right border. FIG. 11B shows a schematic diagram of the SDS::SDS-BARNASE construct. SDS::SDS, the SDS genomic fragment containing a 1.5-Kb promoter region followed by a DNA fragment containing 7 exons and 6 introns; other components are the same as that of SDS::BARNASE. FIG. 11C shows a schematic diagram of the ER::amiR-BARNASE construct. ER, estrogen receptor; amiR-BARNASE, sequence for generating an artificial microRNA targeting BARNASE.

FIG. 12A-12M show the creation of complete male and female sterility in Arabidopsis by SDS::SDS-BARNASE and restoration of fertility by ER::amiR-BARNASE. FIGS. 12A-1F shows the side view of mature flowers (FIGS. 12A-12C) and pollen staining of mature anthers (FIGS. 12D-12F) showing plenty of pollen grains from wild type (FIGS. 12A and 12D), no pollen grains from SDS::SDS-BARNASE plants (FIGS. 12B and 12E), and some pollen grains from ER::amiR-BARNASE/SDS::SDS-BARNASE plants after estradiol induction (FIGS. 12C and 12F). One sepal was removed from each flower. FIGS. 12G-12J shows main branches showing normal siliques in wild type (FIG. 12G), short siliques indicating no developing seeds in SDS::SDS-BARNASE plants without (FIG. 12H) and with (FIG. 12I) pollination, and elongated siliques (arrows) in the ER::amiR-BARNASE SDS::SDS-BARNASE plant treated with estradiol for 7 days (FIG. 12J). FIG. 12K shows real-time qRT-PCR showing expression changes of BARNASE before and after estradiol induction from three examined ER::amiR-BARNASE/SDS::SDS-BARNASE lines. Stars indicate significant difference (P<0.01). FIG. 12L shows six-week old wild-type plants. FIG. 12M shows sterile six-week old FR::amiR-BARNASE/SDS::SDS-BARNASE offspring plants from induced seeds. Bars=0.5 mm (FIG. 12A), 20 μm (FIG. 12D), 1 cm (FIG. 12G), and 5 cm (FIG. 12L), FIGS. 12A-12C, FIGS. 12D-12F, FIGS. 12G-12J, and FIGS. 12L and 12M have the same magnifications.

FIGS. 13A-13D show that SDS::SDS-BARNASE Arabidopsis plants are female sterile and the estradiol induction partially rescues fertilities of ER::amiRBARNASE/SDS::SDS-BARNASE plants. FIGS. 13A-13C (same as FIGS. 5H-5J) show dissected individual siliques from primary inflorescences (positions 1-9) were long in wild type (FIG. 13H), but short in SDS::SDS-BARNASE plants (FIG. 13I, without pollination; FIG. 13J, pollinated with WT pollen). FIG. 13D shows the estradiol induction partially rescues fertilities of ER::amiRBARNASE/SDS::SDS-BARNASE plants.

FIG. 14 shows a comparison of SDS gene structure. Twenty one SDS orthologs in dicots, monocots, and chlorophyta were analyzed by searching PIECE (Plant Intron Exon Comparison and Evolution database; http://wheat.pw.usda.gov/piece/). The Exalign viewer of PIECE shows SDS gene structures (exons, introns, and protein domains) and the relationship of exons in examined SDS orthologous genes. The exon-intron gene structure links to the species phylogeny. Color lines indicate different exon comparison results. The names of species and gene IDs are: Aquilegia coerulea (AcoGoldSmith_v1.023056m; SEQ ID NO: 1); Arabidopsis lyrata (Aly_471662; SEQ ID NO:2); Arabidopsis thaliana (AT1G14750.1; SEQ ID NO:3); Brachypodium distachyon (Bradi1g69380.1; SEQ ID NO:4); Carica papaya (evm.model.supercontig_2.165; SEQ ID NO:5); Citrus clementine (clementine0.9_028383m; SEQ ID NO:6); Citrus sinensis (orange1.1g045573m; SEQ ID NO:7); Cucumis sativus (Cucsa.174110.1; SEQ ID NO:8); Eucalyptus grandis (Egrandis_v1_0.039610m; SEQ ID NO:9); Glycine max (Glyma02g09500.1; SEQ ID NO: 10); Manihot esculenta (cassava4.1_033727m; SEQ ID NO:11); Mimulus guttatus (mgv1a024744m; SEQ ID NO:12); Oryza sativa (LOC_Os03g12414.1; SEQ ID NO:13); Populus trichocarpa (POPTR_0010s11430.1; SEQ ID NO: 14); Prunus persica (ppa026778m; SEQ ID NO: 15); Ricinus communis (29968.m000642; SEQ ID NO: 16); Setaria italica (Si039334m; SEQ ID NO: 17); Sorghum bicolor (Sb01g042340.1; SEQ ID NO: 18); Vitis vinifera (GSVIVT01011625001; SEQ ID NO:19); Volvox carteri (Vca_96988; SEQ ID NO:20); Zea mays (GRMZM2G344416_T01; SEQ ID NO:21).

FIGS. 15A-15B show conserved regulatory motifs in introns of SDS genes. FIG. 15A shows MEME (Multiple Em for Motif Elicitation) suite motif sequence logos showing 5 regulatory motifs in introns of SDS genes: Motif 1 (SEQ ID NO:22); Motif 2 (SEQ ID NO: 23); Motif 3 (SEQ ID NO:24); Motif 4 (SEQ ID NO:25); and Motif 5 (SEQ ID NO:26). Introns from 18 SDS orthologous genes were extracted and joined to a single sequence. Conserved regulatory motifs were analyzed by the MEME suite (http://meme-suite.org/). FIG. 15B shows locations of motifs in intron sequences. Black lines indicate joint intron sequences. Colored bars showing sizes and positions of motifs. Motif 5 (the orange bar) is present in all dicots and monocots. Motifs 1-4 are mainly found in monocots. Numbers before the slash indicate the order number of intron containing the motif 5, and numbers after the slash indicate the total number of introns. Me, Manihot esculenta; Rc, Ricinus communis; Pt, Populus trichocarpa; Gm, Glycine max; Pp, Prunus persica; At, Arabidopsis thaliana; Al, Arabidopsis lyrata; Cp, Carica papaya; Cs, Citrus sinensis; Cc, Citrus clementina; Eg, Eucalyptus grandis; Vv, Vitis vinifera; Mg, Mimulus guttatus; Ac, Aquilegia coerulea; Sb, Sorghum bicolor; Zm, Zea mays; Si, Selaria italic; Os, Oryza sativa; Bd, Brachxpodium distachyon.

FIGS. 16A-16O show SDS::SDS-BARNASE results in completely bisexual sterility in Arabidopsis and tobacco plants. FIG. 16A-16C shows wild type Arabidopsis plants show red pollen in anther (FIG. 16A) and normal seed production (FIGS. 16B and 16C). FIGS. 16D-16F shows sterile Arabidopsis plants show no pollen (FIG. 16D) or seed production (FIGS. 16E and 16F). FIGS. 16G-16I shows fertility restored Arabidopsis plants show partially rescued red pollen (FIG. 16G) and seed production (FIGS. 16G and 16I). FIGS. 16J-16L shows wild type tobacco plants show normal pollen (FIG. 16J) and seed production (FIGS. 16K and 16L). FIGS. 16M-16O shows sterile tobacco plants show no pollen (FIG. 16M) or seed production (FIGS. 16N and 16O).

FIG. 17 shows conserved SDS gene structure in grasses.

FIGS. 18A-18D shows schematic diagrams of constructs. FIG. 18A shows the ablation construct previously used in dicot plants. FIG. 18B shows the ablation construct for generating bisexually sterile B. distachyon. FIG. 18C shows constructs for generating male sterile B. distachyon. Arrow heads indicate positions of regulatory motif1 (M1), M1, M3 and M4. FIG. 18D shows the ethanol-inducible amiR-BARNASE fertility restoration construct that contains the inducible and fertility ablation unit.

### DETAILED DESCRIPTION

The present invention provides a method for creating complete male and female sterility in plants, such as Arabidopsis (Arabidopsis thaliana), tobacco (Nicotiana tabacum), Brachypodium, and alfalfa. The disclosed methods provides an efficient strategy to specifically ablate microspore and megaspore mother cells using the SOLO DANCERS (SDS) and BARNASE fusion gene, which results in complete sterility in both male and female reproductive organs, but does not affect plant growth or development, including the production of all flower organs.

The present invention also relates to a fertility restoring system via inducible expression of an artificial microRNA targeting BARNASE. The fertility restoring system can restore fertility to male and female plants and can be used for plant hybrid breeding. The disclosed methods of restoring fertility suppresses the BARSTAR enzyme activity by directly down-regulating the expression of BARNASE, thus providing a new tool to restore the fertility of BARNASE-induced sterile plants.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Chemically-inducible promoters” or “chemically-regulated promoters” as used interchangeably herein refer to a class of promoters that are modulated by chemical compounds that either turn off or turn on gene transcription. The chemicals that influence promoter activity are not typically naturally present in the organism where expression of the transgene is sought; are not toxic, affect only the expression of the gene of interest; are easy to apply or removal; and induce a clearly detectable expression pattern of either high or very low gene expression for their optimal use as modulators of gene expression.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual plant or animal cell to which the nucleic acid is administered. The coding sequence may be codon optimize.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

As used herein, a “control plant” is a plant that is substantially equivalent to a test plant or modified plant in all parameters with the exception of the test parameters. For example, when referring to a plant into which a polynucleotide according to the present invention has been introduced, in certain embodiments, a control plant is an equivalent plant into which no such polynucleotide has been introduced. In certain embodiments, a control plant is an equivalent plant into which a control polynucleotide has been introduced. In such instances, the control polynucleotide is one that is expected to result in little or no phenotypic effect on the plant.

“Endogenous gene” as used herein refers to a gene that originates from within the plant or plant cell. An endogenous gene is native to the plant or plant cell, which is in its normal genomic and chromatin context, and which is not heterologous to the plant or plant cell.

A “functional homolog,” “functional equivalent,” or “functional fragment” of a polypeptide of the present invention is a polypeptide that is homologous to the specified polypeptide but has one or more amino acid differences from the specified polypeptide. A functional fragment or equivalent of a polypeptide retains at least some, if not all, of the activity of the specified polypeptide.

A “fusion protein” as used herein refers to an artificially made or recombinant molecule that comprises two or more protein sequences that are not naturally found within the same protein. The fusion protein may include non-proteinaceous elements as well as proteinaceous elements.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Genetically modified” or “GM” as used interchangeably herein refers to an organism or crop containing genetic material that has been artificially altered so as to produce a desired characteristic.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

Optimal alignment of sequences for comparison may be conducted by methods commonly known in the art, for example by the search for similarity method described by Pearson and Lipman 1988, Proc. Natl. Acad. Sci. USA 85: 2444-2448, by computerized implementations of algorithms such as GAP, BESTFIT, BLAST, FASTA, and TFASTA in the Wisconsin Genetics Software Package, Genetics Computer Group (GCG), Madison, Wis., or by inspection. In a preferred embodiment, protein and nucleic acid sequence identities are evaluated using the Basic Local Alignment Search Tool (“BLAST”), which is well known in the art (Karlin and Altschul, Proc. Natl. Acad. Sci. USA 87: 2267-2268 (1990); Altschul et al., Nucl. Acids Res. 25: 3389-3402 (1997)), the disclosures of which are incorporated by reference in their entireties. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990). The BLAST programs can be used with the default parameters or with modified parameters provided by the user.

The terms “isolated,” “purified” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated nucleic acid of the present invention is separated from open reading frames that flank the desired gene and encode proteins other than the desired protein. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

The specificity of single-stranded DNA to hybridize complementary fragments is determined by the “stringency” of the reaction conditions (Sambrook et al., Molecular Cloning and Laboratory Manual, Second Ed., Cold Spring Harbor (1989)). Hybridization stringency increases as the propensity to form DNA duplexes decreases. In nucleic acid hybridization reactions, the stringency can be chosen to favor specific hybridizations (high stringency), which can be used to identify, for example, full-length clones from a library. Less-specific hybridizations (low stringency) can be used to identify related, but not exact (homologous, but not identical), DNA molecules or segments.

DNA duplexes are stabilized by: (1) the number of complementary base pairs; (2) the type of base pairs; (3) salt concentration (ionic strength) of the reaction mixture; (4) the temperature of the reaction; and (5) the presence of certain organic solvents, such as formamide, which decrease DNA duplex stability. In general, the longer the probe, the higher the temperature required for proper annealing. A common approach is to vary the temperature; higher relative temperatures result in more stringent reaction conditions.

To hybridize under “stringent conditions” describes hybridization protocols in which nucleotide sequences at least 60% homologous to each other remain hybridized. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. Since the target sequences are generally present at excess, at Tm, 50% of the probes are occupied at equilibrium.

“Stringent hybridization conditions” are conditions that enable a probe, primer, or oligonucleotide to hybridize only to its target sequence. Stringent conditions are sequence-dependent and will differ. Stringent conditions comprise: (1) low ionic strength and high temperature washes, for example 15 mM sodium chloride, 1.5 mM sodium citrate, 0.1% sodium dodecyl sulfate, at 50° C.; (2) a denaturing agent during hybridization, e.g. 50% (v/v) formamide, 0.1% bovine serum albumin, 0.1% Ficoll, 0.1% polyvinylpyrrolidone, 50 mM sodium phosphate buffer (750 mM sodium chloride, 75 mM sodium citrate; pH 6.5), at 42° C.; or (3) 50% formamide. Washes typically also comprise 5×SSC (0.75 M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a wash at 42° C. in 0.2×SSC (sodium chloride/sodium citrate) and 50% formamide at 55° C., followed by a high-stringency wash consisting of 0.1×SSC containing EDTA at 55° C. Preferably, the conditions are such that sequences at least about 65%, 70%, 75%, 85%, 90%, 95%, 98%, or 99% homologous to each other typically remain hybridized to each other. These conditions are presented as examples and are not meant to be limiting.

“Moderately stringent conditions” use washing solutions and hybridization conditions that are less stringent, such that a polynucleotide will hybridize to the entire, fragments, derivatives, or analogs of the target sequence. One example comprises hybridization in 6×SSC, 5×Denhardt's solution, 0.5% SDS and 100 μg/ml denatured salmon sperm DNA at 55° C., followed by one or more washes in 1×SSC, 0.1% SDS at 37° C. The temperature, ionic strength, etc., can be adjusted to accommodate experimental factors such as probe length. Other moderate stringency conditions have been described (Ausubel et al., Current Protocols in Molecular Biology, Volumes 1-3, John Wiley & Sons, Inc., Hoboken, N.J. (1993); Kriegler, Gene Transfer and Expression: A Laboratory Manual, Stockton Press, New York, N.Y. (1990); Perbal, A Practical Guide to Molecular Cloning, 2nd edition, John Wiley & Sons, New York, N.Y. (1988)).

“Low stringent conditions” use washing solutions and hybridization conditions that are less stringent than those for moderate stringency, such that a polynucleotide will hybridize to the entire, fragments, derivatives, or analogs of the target sequence. A nonlimiting example of low stringency hybridization conditions includes hybridization in 35% formamide, 5×SSC, 50 mM Tris HCl (pH 7.5), 5 mM EDTA, 0.02% PVP, 0.02% Ficoll, 0.2% BSA, 100 μg/ml denatured salmon sperm DNA, 10% (wt/vol) dextran sulfate at 40° C., followed by one or more washes in 2×SSC, 25 mM Tris HCl (pH 7.4), 5 mM EDTA, and 0.1% SDS at 50° C. Other conditions of low stringency, such as those for cross-species hybridizations, are well-described (Ausubel et al., 1993; Kriegler, 1990).

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

As used herein, the term “plant” includes reference to whole plants, plant organs (e.g., leaves, stems, roots, etc.), seeds, plant cells, and progeny of same. Parts of transgenic plants comprise, for example, plant cells, protoplasts, tissues, callus, embryos as well as flowers, ovules, stems, fruits, leaves, roots originating in transgenic plants or their progeny previously transformed with a DNA. As used herein, the term “plant cell” includes, without limitation, protoplasts and cells of seeds, suspension cultures, embryos, meristematic regions, callus tissue, leaves, roots, shoots, gametophytes, sporophytes, pollen, and microspores.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

The term “substantial identity” of polynucleotide sequences means that a polynucleotide comprises a sequence that has at least 25% sequence identity compared to a reference sequence as determined using the programs described herein; preferably BLAST using standard parameters, as described. Alternatively, percent identity can be any integer from 25% to 100%. More preferred embodiments include polynucleotide sequences that have at least about: 25%, 30%, 35%, 40%, 45%, 50%, 55%, 60%, 65%, 700/%, 75%, 80%, 85%, 86%/0, 87%, 88%, 89%, 90%, 91%, 92%, 93%, 94%, 95%, 96%, 97%, 98% or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein of the present invention include nucleic acid sequences that have substantial identity to the nucleic acid sequences that encode the polypeptides of the present invention. Polynucleotides encoding a polypeptide comprising an amino acid sequence that has at least about: 40%, 45%, 50%, 55%, 60%, 65%, 70%, 75%, 80%, 85%, 86%, 87%, 88%, 89%, 90%, 91%/0, 92%, 93%, 94%, 95%, 96%, 97%, 98%, or 99% sequence identity compared to a reference polypeptide sequence are also preferred.

The term “substantial identity” of amino acid sequences (and of polypeptides having these amino acid sequences) normally means sequence identity of at least 40% compared to a reference sequence as determined using the programs described herein; preferably BLAST using standard parameters, as described. Preferred percent identity of amino acids can be any integer from 40% to 100%. More preferred embodiments include amino acid sequences that have at least about: 40%, 45%, 50%, 55%, 60%, 65%/0, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine. Accordingly, polypeptides or proteins, encoded by the polynucleotides of the present invention, include amino acid sequences that have substantial identity to the amino acid sequences of the polypeptides, encoded by the polynucleotides of the present invention, which are compositions for generating male sterility and female sterility and/or compositions for restoring fertility in the male sterile and female sterile plants.

“Target plant” as used herein refers to a plant or tree that will be transformed with recombinant genetic material not normally found in plants or trees of this type and which will be introduced into the plant in question (or into progenitors of the plant) by human manipulation.

“Transgene” as used herein refers to a gene or genetic material containing a gene sequence that has been isolated from one organism, such as one plant or plant cell, and is introduced into a different organism, such as a different plant or plant cell. This non-native segment of DNA may retain the ability to produce RNA or protein in the transgenic organism, such as the transgenic plant, or it may alter the normal function of the transgenic organism's genetic code. The introduction of a transgene has the potential to change the phenotype of an organism, such as a plant.

“Transgenic plant” as used herein refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid. For example, the vector may encode a composition for generating male sterility and female sterility and/or composition for restoring fertility in the male sterile and female sterile plants, as disclosed herein. Alternatively, the vector may comprise a polynucleotide sequence encoding a composition for generating male sterility and female sterility and/or composition for restoring fertility in the male sterile and female sterile plants, as disclosed herein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

### 2. COMPOSITIONS FOR GENERATING MALE STERILITY AND FEMALE STERILITY

Provided herein are compositions for generating male sterility and female sterility in plants. The SOLO-DANCERS (SDS)::SDS-BARNASE system can be used to generate both male and female sterile plants without affecting growth or flower structure. The SDS::SDS-BARNASE system includes an isolated polynucleotide construct that encodes a SDS-BARNASE fusion protein. The isolated polynucleotide construct includes a first polynucleotide and a second polynucleotide that are operably linked to a SDS promoter. The first polynucleotide includes a SOLO-DANCERS (SDS) gene or fragment thereof. The second polynucleotide includes a Barnase gene or fragment thereof. The SDS gene includes the SDS promoter.

a. SOLO-DANCERS (SDS) Gene

The SOLO-DANCERS (SDS) gene encodes a meiosis specific cyclin that is involved in homolog interaction during meiotic prophase I in Arabidopsis. With normal growth and development, the sds mutant is male and female sterile due to the meiosis defect. The SDS protein is exclusively present in pollen mother cells in anthers and megaspore mother cells in ovules. The SDS-BARNASE fusion protein does not create any toxicity in other cells or tissues. RNA in situ hybridization analysis shows that SDS is specifically expressed in micro- and megaspore mother cells (or male and female meiocytes); however, as disclosed herein, the SDS promoter does not achieve the exclusive expression of GUS or BARNASE in either micro- or megaspore mother cells. Conversely, the SDS genomic fragment containing the promoter, introns and exons does achieve the exclusive expression of GUS or BARNASE in either micro- or megaspore mother cells. Regulatory motifs in SDS introns may contribute to its specific spatial and temporal expression. Intron dependent spatial expression has been revealed in different genes in various species.

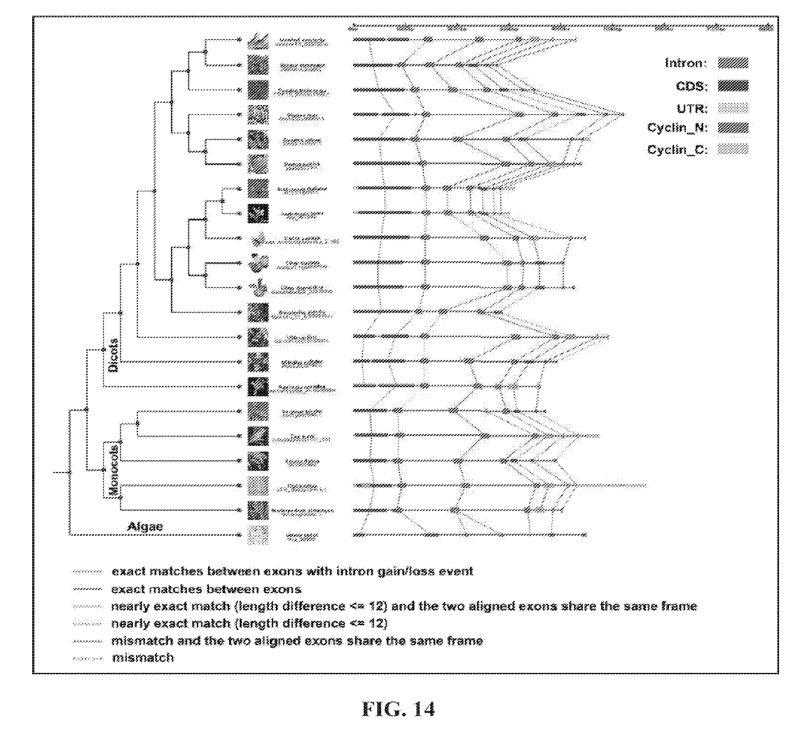

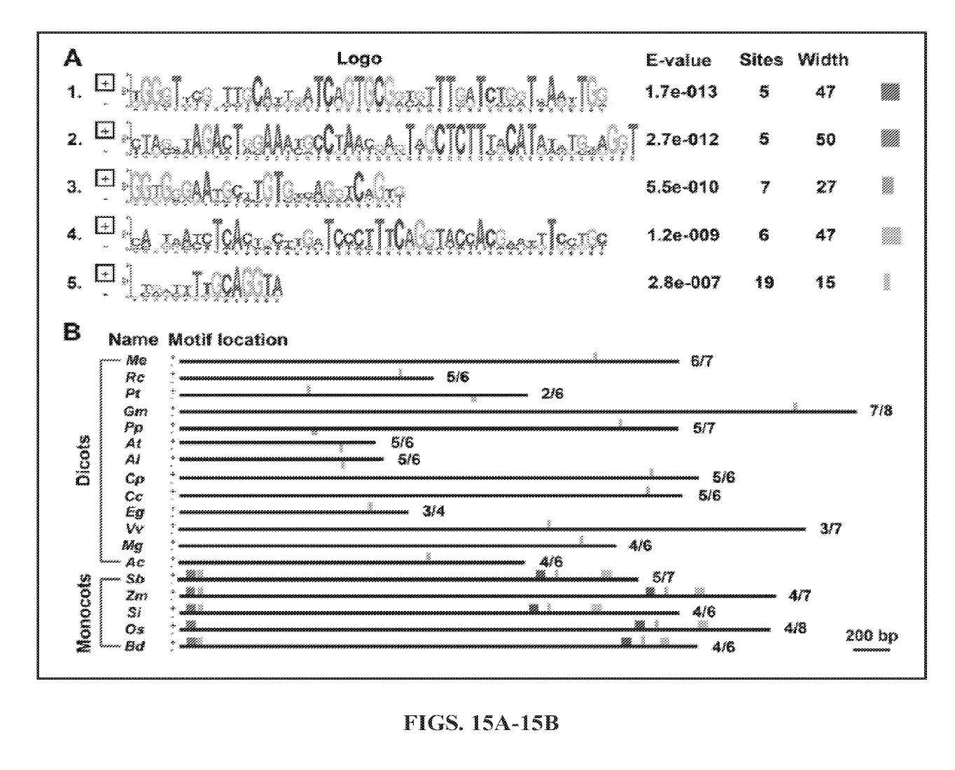

SDS, existing in both dicots and monocots, is distantly related to other cyclins, thus represents a unique type of (SDS-type) cyclin. Analysis of 21 SDS orthologs using PIECE (Plant Intron and Exon Comparative and Evolution; http://wheat.pw.usda.gov/piece/) shows that the length and numbers of exons in SDS genes are similar in higher plants, especially in the Cyclin N domain that spans 3 most conserved exons (see FIG. 14). The length of SDS introns among dicots is different, whereas the gene structure of SDS in monocots is conserved. 5 novel regulatory motifs were identified in SDS introns via the MEME (Multiple Em for Motif Elicitation) suite (http://meme-suite.org/tools/meme) (FIG. 15A). Among them, the motif 5 is present in all examined dicots and monocots, while the motif 1 is unique in monocots (FIG. 15B). The motif 5, which is found in all examined plants, can play an important role in the specific expression of SDS gene.

In some embodiments, the SDS gene can be the SDS gene from Arabidopsis (Arabidopsis thaliana), Purple false brome (Brachypodium distachyon), Brachypodium sylvaticum, Rice (Oryza saliva), False brome (Brachypodium stacei), Switchgrass (Panicum virgatum), Aquilegia coerulea. Arabidopsis lyrata, Carica papaya, Citrus clementine, Citrus sinensis, Turnip mustard (Brassica rapa), Barrel medic (Medicago truncatula), Soybean (Glycine max), Cucumber (Cucumis sativus), Potato (Solanum lycopersicum), Maize (Zea mays), Manihot esculenta, Mimulus guttatus, Hall's panicgrass (Panicum hallii), Foxtail millet (Setaria italica), Sorghum (Sorghum bicolor), Green foxtail (Setaria viridis), Poplar (Populus trichocarpa), Rose gum (Eucalyptus grandis), Ricinus communis, Vitis vinifera, Volvox carteri, or Cherry (Prunus persica).

In some embodiments, the SDS::SDS-BARNASE system includes a synthetic promoter that confers strong and specific SDS expression in micro and megaspore mother cells. The synthetic promoter can be used to produce absolute male and female sterility in various plants. In some embodiments, the synthetic promoter is the SDS promoter from the SDS gene from Arabidopsis (Arabidopsis thaliana), Purple false brome (Brachypodium distachyon), Brachypodium sylvaticum. Rice (Oryza sativa), False brome (Brachypodium stacei), Switchgrass (Panicum virgatum), Aquilegia coerulea, Arabidopsis lyrata, Carica papaya, Citrus clementine, Citrus sinensis, Turnip mustard (Brassica rapa), Barrel medic (Medicago truncatula), Soybean (Glycine max), Cucumber (Cucumis sativus), Potato (Solanum lycopersicum), Maize (Zea mays), Manihot esculenta, Mimulus guttatus, Hall's panicgrass (Panicum hallii), Foxtail millet (Setaria italica), Sorghum (Sorghum bicolor), Green foxtail (Setaria viridis), Poplar (Populus trichocarpa), Rose gum (Eucalyptus grandis), Ricinus communis, Vitis vinifera, Volvox carteri, or Cherry (Prunus persica). The synthetic promoter can be used with one or more regulatory introns. The one or more regulatory introns can include one or more of motifs 1-5.

In some embodiments, the SDS gene includes at least one regulatory intron. For example, the isolated SDS gene can include between 1 and 5 regulatory introns, between 2 and 5 regulatory introns, between 3 and 5 regulatory introns, between 4 and 5 regulatory introns, between 1 and 4 regulatory introns, between 2 and 4 regulatory introns, between 3 and 4 regulatory introns, between 1 and 3 regulatory introns, between 2 and 3 regulatory introns, or between 1 and 2 regulatory introns. In some embodiments, the SDS gene includes at least 1 regulatory intron, at least 2 regulatory introns, at least 3 regulatory introns, at least 4 regulatory introns, or at least 5 regulatory introns. In some embodiments, the SDS gene can include between 1 and 5 motifs, between 2 and 5 motifs, between 3 and 5 motifs, between 4 and 5 motifs, between 1 and 4 motifs, between 2 and 4 motifs, between 3 and 4 motifs, between 1 and 3 motifs, between 2 and 3 motifs, or between 1 and 2 motifs. In some embodiments, the SDS gene includes at least 1 motif, at least 2 motifs, at least 3 motifs, at least 4 motifs, or at least 5 motifs. In some embodiments, the regulatory intron includes a polynucleotide sequence of any one of SEQ ID NO: 22-26 or 47-51. In some embodiments, the motif includes a polynucleotide sequence of any one of SEQ ID NO: 22-26 or 47-51. In some embodiments, the SDS gene includes a polynucleotide sequence of any one of SEQ ID NO: 1-21 or 29-46.

b. BARNASE Gene

The barnase protein (also referred to as “Barnase”) is an RNase that has 110 amino acid residues and hydrolyzes RNA. Barnase originates from Bacillus amyloliquefaciens. When expressed in cells, this enzyme inhibits the functions of the cells as a result of its potent RNase activity and thus causes cell death in many cases. By using this characteristic, it is therefore expected that the function of the specific site can be selectively controlled by expressing the barnase gene in a specific site of a plant. In some embodiments, the barnase gene includes the polynucleotide sequence of SEQ ID NO: 27.

### 3. COMPOSITIONS FOR RESTORING FERTILITY

Provided herein are compositions for restoring fertility in the male sterile and female sterile plants that already includes a first isolated polynucleotide construct, as described above. The compositions for restoring fertility involves an artificial microRNA system that inhibits BARNASE expression to restore plant fertility. To restore fertility to both male and female sterile plants, the artificial microRNA system, such as the ER::amiR-BARNASE system, induces the expression of an artificial microRNA (amiRNA) to post-transcriptionally suppress the expression of BARNASE. Instead of inhibiting the BARNASE activity by BARSTAR at the protein level, the amiR-BARNASE system, under the control of an inducible promoter, such as the estradiol inducible promoter, suppresses the expression of BARNASE at the post-transcriptional level, which consequently decreases the accumulation of BARNASE protein. Not only does the inducible treatment, such as estradiol treatment, restore fertility of male sterile and female sterile plants, such as SDS::SDS-BARNASE ER::amiR-BARNASE double transgenic plants, but also the offspring of these plants are completely sterile. The amiR-BARNASE system, such as the ER::amiR-BARNASE system, can be used as an alternative approach to conveniently and efficiently restore fertility of BARNASE-induced sterile plants.

The compositions for restoring fertility include a second isolated polynucleotide construct. The second isolated polynucleotide construct includes an inducible promoter operably linked to an artificial microRNA (amiRNA) targeted to the Barnase gene or fragment thereof. The fertility of the plant is restored by inducing the expression of the amiRNA. In some embodiments, the plant becomes male fertile and female fertile after the induction of amiRNA. In some embodiments, the second isolated polynucleotide construct includes estradiol (ER)::amirBARNASE. In some embodiments, the amiRNA includes a polynucleotide sequence of SEQ ID NO: 28.

In some embodiments, the isolated polynucleotide construction that encodes the SDS-BARNASE fusion protein and the second isolated polynucleotide are encoded on the same vector. In some embodiments, the isolated polynucleotide construction that encodes the SDS-BARNASE fusion protein and the second isolated polynucleotide are encoded on separate vectors.

a. Inducible Promoter

An “inducible” promoter is one which is capable of directing a level of transcription of an operably linked nucleic acid sequence in the presence of a stimulus or environmental stress (e.g., heat shock, irradiation, chemicals, etc.), wherein the level of the transcription is different from that in the absence of the stimulus. In some embodiments, the inducible promoter is a promoter that induced by a chemical, such as estradiol, dexamethasone, methoxyfenozide, and ethanol, or heat shock. In some embodiments, the inducible promoter is an estradiol-inducible, glucocorticoid-inducible, tetracycline-inducible, pristamycin-inducible, pathogen-inducible, steroid-inducible, such as glucocorticoid-inducible, estrogen-inducible, metal-inducible, such as copper-inducible, herbicide safener-inducible, alcohol-inducible, such as an ethanol-inducible, iso-propyl β-D-1-thiogalactopyranoside-inducible, pathogen-inducible, or ecdysone-inducible promoter. In some embodiments, the inducible promoter is an estradiol inducible promoter, an ethanol inducible promoter, a dexamethasone inducible promoter, a methoxyfenozide inducible promoter or a temperature inducible promoter. In some embodiments, the inducible promoter is induced by environmental factors such as water or salt stress, anaerobiosis, temperature, such as cold- and heat-inducible, illumination, and wounding. In some embodiments, the inducible promoter is a heat shock inducible promoter or a heat inducible promoter. Examples of inducible promoters are described in U.S. Patent Publication No. 20130042371, which are incorporated by reference herein in its entirely.

In some embodiments, the inducible promoter is induced or activated by a chemical. In some embodiments, the chemical is applied to the transgenic plant by a foliar spray or root drenching. In some embodiments, the chemical is applied to the transgenic plant by dipping the reproductive organs of the plant in the chemical or solution containing said chemical. In some embodiments, the reproductive organ is an inflorescence.

### 4. METHODS OF GENERATING TRANSGENIC PLANTS WITH MALE STERILITY AND FEMALE STERILITY

The present invention is directed to a method for generating a complete male sterile and female sterile plant using the SDS::SDS-BARNASE system. The method includes introducing into a target plant an isolated polynucleotide construct containing the SOLO-DANCERS (SDS) gene or fragment thereof, and the Barnase gene or fragment thereof, as described above to generate a transgenic plant that is male sterile and female sterile. In some embodiments, the SDS gene is an endogenous gene of target plant. In some embodiments, the SDS gene is a transgene to the target plant.

### 5. METHODS OF RESTORING FERTILITY IN MALE STERILE AND FEMALE STERILE PLANTS

The present invention is directed to methods of restoring fertility in a male sterile and female sterile transgenic plant, as described above. The methods of restoring fertility can be used for plant hybrid breeding. The method includes introducing into a target plant a second isolated polynucleotide construct that includes an inducible promoter operably linked to an artificial microRNA (amiRNA) targeted to the Barnase gene or fragment thereof, thereby generating a transgenic plant, introducing into the generated transgenic plant an isolated polynucleotide construct that includes a first polynucleotide and a second polynucleotide, the first polynucleotide comprising a SOLO-DANCERS (SDS) gene or fragment thereof, the second polynucleotide comprising a Barnase gene or fragment thereof, wherein the SDS gene comprises the SDS promoter, as described above, thereby generating a double transgenic plant; and inducing the expression of the amiRNA, thereby restoring fertility in a complete male sterile and female transgenic sterile plant. In some embodiments, the transgenic plant becomes male fertile and female fertile after the induction of amiRNA.

In some embodiments, the expression of the amiRNA is induced when the transgenic plant is flowering. In some embodiments, the method restores at least about 20%, at least about 30% at least about 40%, at least about 50%, at least about 60% at least about 70%, at least about 80%, at least about 80%, at least about 90%, or at least about 100% fertility.

### 6. METHODS OF ABLATING MICROSPORE AND MEGASPORE MOTHER CELLS

The present invention is directed to a method of genetically ablating pollen and megaspore mother cells. Megaspore and pollen mother cells are two small groups of reproductive cells, which are differentiated after all floral organs are established. Ablating pollen and megaspore mother cells only leads to elimination of male and female gametes, but it does not affect differentiation of any other somatic cells and flower development. The method includes introducing into a target plant an isolated polynucleotide construct containing the SOLO-DANCERS (SDS) gene or fragment thereof, and the Barnase gene or fragment thereof, as described above to generate a transgenic plant wherein the microspore and megaspore mother cells are ablated. In some embodiments, the SDS gene is an endogenous gene of target plant. In some embodiments, the SDS gene is a transgene to the target plant.

### 7. TARGET PLANT

The methods described herein can be used to provide a valuable resource for wood production, biofuels, bioremediation, and many other applications. The methods can be used to produce transgenic trees, such as poplar, eucalypts, and pines, grasses for biofuels, such as miscanthus and switchgrass, wood production, bioremediation, such as with turf grasses and forage crops, ornamental plants to avoid fruit production (e.g. ornamental cherry or crabapple trees), or invasive and ornamental plants. Male and female sterilized invasive plants by our method can be planted for multiple purposes, such as forestry and horticulture.

The target plant to be transformed to produce the transgenic plant may be any plant species, including non-vascular plants and vascular plants. The non-vascular plant may include a bryophyte, such as Physcomitrella patens. The vascular plants may include pteridophyte, such as Selaginella martensii, angiosperms, and gymnosperms. The angiosperms may include a monocot plant or a dicot plant. The plant may be a crop plant, such as a cereal, a fruit, a legume, or a root crop, ornamental plants, or a non-food crop, such as cotton, hemp (Cannabis sativa), flax or linseed (Linum usitatissimum), oilseed rape or high erucic acid rape (Brassica napus), balsam poplar (Populus balsamifera), tobacco (Nicotiana tabacum), and switchgrass (e.g., Panicum virgatum).

In some embodiments, the target plant is a gymnosperm or angiosperm. In some embodiments, the plant is a grass, tree, or ornamental plant. Suitable plant species include, without limitation, corn (Zea mays), soybean (Glycine max), Brassica sp. (e.g., Arabidopsis thaliana, Brassica nalnpus, B. rapa, and B. juncea), alfalfa (Medicago sativa), rice (Oryza sativa), rye (Secale cereale), sorghum (Sorghum bicolor, Sorghum vulgare), millet (e.g., pearl millet (Penunisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), tobacco (Nicotiana tabacum), potato (Solanum tuberosum), peanuts (Arachis hypogaea), pea (Pisum sativum), cotton (Gossypium barbadense, Gossypium hirsutum), sweet potato (Ipomoea batatus), cassaya (Manihot esculenta), coffee (Cofea spp.), coconut (Cocos nucifera), pineapple (Ananas comosus), citrus trees (Citrus spp.), cocoa (Theobroma cacao), grape (Vitis vinifera), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats (Avena sativa), barley (Hordeum vulgare), vegetables, ornamentals, and conifers.

Vegetables include, without limitation, tomatoes (Lycopersicon esculentum), lettuce (e.g., Lactuca saliva), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). In some embodiments, the target plant is Arabidopsis, tobacco, alfalfa, soybean, maize, rice, Brachypodium, switchgrass, Miscanthus, poplars, cherry, or Eucalyptus.

a. Grasses

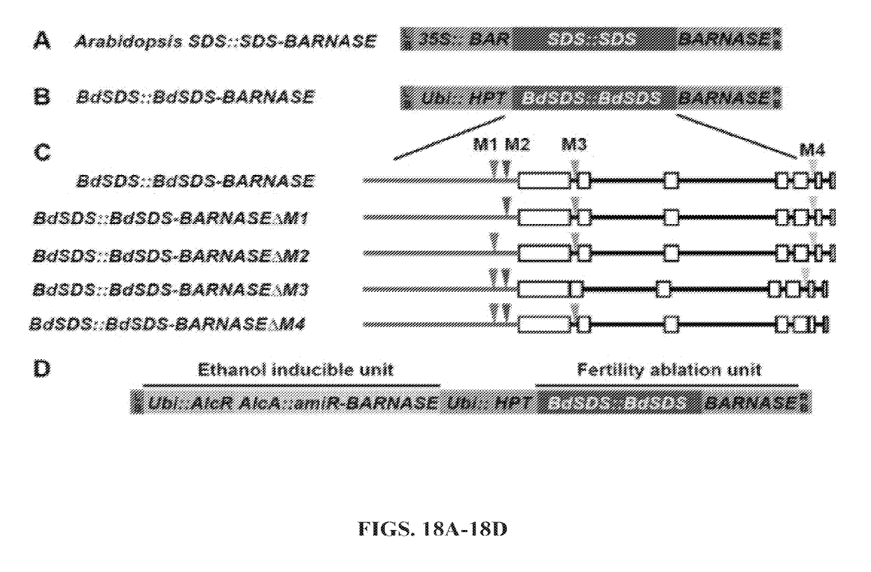

The grass family of monocotyledonous flowering plants (monocots) is the most important plant family for human and the environment where we live. Besides traditional uses of grasses, many grass species can provide a large and sustainable cellulosic biomass feedstock. Recently, switchgrass was selected as a biomass feedstock for renewable bioenergy by the U.S. Department of Energy (DOE) Bioenergy Feedstock Development Program since its broad adaption, high yield, and minimal agricultural inputs. Genetically modified (GM) switchgrass has been made to improve biomass and biofuel production, but the approval for commercial uses of GM plants is subject to complicated and stringent government regulations due to economic, politic or social concerns over potential ecological effects of transgene flow. Completely abolishing both male and female (bisexual) fertility is the only fail-safe way to prevent transgene flow; however, approaches to generating both bisexual sterility are limited. The gene structure of SDS in monocots is more conserved than that in dicots. In grass plants, two conserved regulatory motifs in the promoter region and the other two in introns may be possibly important for the SDS specific expression (see FIGS. 17 and 18A-18D).

b. Ornamental Plants

Ornamental plants are plants that are grown for decorative purposes in gardens and landscapes, as houseplants, and for cut flowers. For ornamental trees, such as cherries and plums, fruit setting affects flower numbers and quality. Moreover, fruits often make the garden messy. The methods disclosed herein can be used to generate ornamental trees that produce attractive flowers but no fruits.

### 8. CONSTRUCTS AND PLASMIDS

The genetic constructs may comprise a nucleic acid sequence that encodes the compositions for generating male sterility and female sterility and/or compositions for restoring fertility in the male sterile and female sterile plants, disclosed herein. The genetic construct, such as a plasmid, may comprise a nucleic acid that encodes the compositions for generating male sterility and female sterility and/or compositions for restoring fertility in the male sterile and female sterile plants. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant cauliflower mosaic virus, recombinant tobacco mosaic virus, and recombinant potato virus X-based vectors. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer an initiation codon, a stop codon, or a polyadenylation signal.

In certain embodiments, the polynucleotides to be introduced into the plant are operably linked to a promoter sequence and may be provided as a construct. As used herein, a polynucleotide is “operably linked” when it is placed into a functional relationship with a second polynucleotide sequence. For instance, a promoter is operably linked to a coding sequence if the promoter is connected to the coding sequence such that it may effect transcription of the coding sequence. In various embodiments, the polynucleotides may be operably linked to at least one, at least two, at least three, at least four, at least five, or at least ten promoters.

The nucleic acid sequences may make up a genetic construct that may be a vector. The vector may be capable of expressing the compositions for generating male sterility and female sterility and/or compositions for restoring fertility in the male sterile and female sterile plants in the cell of a plant. The vector may be recombinant. The vector may comprise heterologous nucleic acid encoding the compositions for generating male sterility and female sterility and/or compositions for restoring fertility in the male sterile and female sterile plants. The vector may be a plasmid. The vector may be useful for transfecting cells with nucleic acid encoding the compositions for generating male sterility and female sterility and/or compositions for restoring fertility in the male sterile and female sterile plants, after which the transformed host cell is cultured and maintained under conditions wherein expression of the compositions for generating male sterility and female sterility and/or compositions for restoring fertility in the male sterile and female sterile plants takes or can take place.

Coding sequences may be optimized for stability and high levels of expression. In some instances, codons are selected to reduce secondary structure formation of the RNA such as that formed due to intramolecular bonding.

The vector may comprise heterologous nucleic acid encoding the compositions for generating male sterility and female sterility and/or compositions for restoring fertility in the male sterile and female sterile plants and may further comprise an initiation codon, which may be upstream of the compositions for generating male sterility and female sterility and/or compositions for restoring fertility in the male sterile and female sterile plants coding sequence and a stop codon, which may be downstream of the compositions for generating male sterility and female sterility and/or compositions for restoring fertility in the male sterile and female sterile plants coding sequence. The initiation and termination codon may be in frame with the compositions for generating male sterility and female sterility and/or compositions for restoring fertility in the male sterile and female sterile plants coding sequence. The vector may also comprise a promoter that is operably linked to the compositions for generating male sterility and female sterility and/or compositions for restoring fertility in the male sterile and female sterile plants coding sequence. The promoter that is operably linked to the compositions for generating male sterility and female sterility and/or compositions for restoring fertility in the male sterile and female sterile plants coding sequence may be not natively associated with the polynucleotide encoding the compositions for generating male sterility and female sterility and/or compositions for restoring fertility in the male sterile and female sterile plants. Promoters useful in the practice of the present invention include, but are not limited to, constitutive, inducible, temporally-regulated, developmentally regulated, chemically regulated, tissue-preferred and tissue-specific promoters. Suitably, the promoter causes sufficient expression in the plant to produce the phenotypes described herein. Suitable promoters include, without limitation, the 35S promoter of the cauliflower mosaic virus, ubiquitin, tCUP cryptic constitutive promoter, the Rsyn7 promoter, pathogen-inducible promoters, the maize In2-2 promoter, the tobacco PR-1a promoter, glucocorticoid-inducible promoters, and tetracycline-inducible and tetracycline-repressible promoters.

The vector may also comprise a polyadenylation signal, which may be downstream of the compositions for generating male sterility and female sterility and/or compositions for restoring fertility in the male sterile and female sterile plants coding sequence. The vector may also comprise an enhancer upstream of the compositions for generating male sterility and female sterility and/or compositions for restoring fertility in the male sterile and female sterile plants coding sequence. The enhancer may be necessary for DNA expression. The vector may also comprise a plant origin of replication in order to maintain the vector extrachromosomally and produce multiple copies of the vector in a cell. The vector may also comprise a regulatory sequence, which may be well suited for gene expression in a plant cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., 1989, which is incorporated fully by reference. In some embodiments the vector may comprise the nucleic acid sequence encoding the compositions for generating male sterility and female sterility and/or compositions for restoring fertility in the male sterile and female sterile plants.

### 9. PLANT TRANSFORMATION

The compositions for generating male sterility and female sterility and/or compositions for restoring fertility in the male sterile and female sterile plants of the present invention may be introduced into a plant cell to produce a transgenic plant. As used herein, “introduced into a plant” with respect to polynucleotides encompasses the delivery of a polynucleotide into a plant, plant tissue, or plant cell using any suitable polynucleotide delivery method. Methods suitable for introducing polynucleotides into a plant useful in the practice of the present invention include, but are not limited to, freeze-thaw method, microparticle bombardment, direct DNA uptake, whisker-mediated transformation, electroporation, sonication, microinjection, plant virus-mediated, and Agrobacterium-mediated transfer to the plant. Any suitable Agrobacterium strain, vector, or vector system for transforming the plant may be employed according to the present invention. In certain embodiments, the polynucleotide is introduced using at least one of stable transformation methods, transient transformation methods, or virus-mediated methods.

By “stable transformation” is intended that the nucleotide construct introduced into a plant integrates into the genome of the plant and is capable of being inherited by progeny thereof. By “transient transformation” is intended that a nucleotide construct introduced into a plant does not integrate into the genome of the plant.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al., Biotechniques 4:320-334 (1986)), electroporation (Riggs et al., Proc. Natl. Acad. Sci. USA 83:5602-5606 (1986)), Agrobacterium-mediated transformation (U.S. Pat. Nos. 5,981,840 and 5,563,055), direct gene transfer (Paszkowski et al., EMBO J. 3:2717-2722 (1984)), and ballistic particle acceleration (see, for example, U.S. Pat. Nos. 4,945,050; 5,879,918; 5,886,244; 5,932,782; Tomes et al., in Plant Cell, Tissue, and Organ Culture: Fundamental Methods, ed. Gamborg and Phillips (Springer-Verlag, Berlin) (1995); and McCabe et al., Biotechnology 6:923-926 (1988)). Also see Weissinger et al., Ann. Rev. Genet. 22:421-477 (1988); Sanford et al., Particulate Science and Technology 5:27-37 (1987) (onion); Christou et al., Plant Physiol. 87:671-674 (1988) (soybean); McCabe et al., Bio/Technology 6:923-926 (1988) (soybean); Finer and McMullen, In Vitro Cell Dev. Biol. 27P:175-182 (1991) (soybean); Singh et al., Theor. Appl. Genet. 96:319-324 (1998) (soybean); Datta et al., Biotechnology 8:736-740 (1990) (rice); Klein et al., Proc. Natl. Acad. Sci. USA 85:4305-4309 (1988) (maize); Klein et al., Biotechnology 6:559-563 (1988) (maize); U.S. Pat. Nos. 5,240,855; 5,322,783 and 5,324,646; Klein et al., Plant Physiol. 91:440-444 (1988) (maize); Fromm et al., Biotechnology 8:833-839 (1990) (maize); Hooykaas-Van Slogteren et al., Nature (London) 311:763-764 (1984); U.S. Pat. No. 5,736,369 (cereals); Bytebier et al., Proc. Natl. Acad. Sci. USA 84:5345-5349 (1987) (Liliaceae); De Wet et al., in The Experimental Manipulation of Ovule Tissues, ed. Chapman et al., (Longman, N.Y.), pp. 197-209 (1985) (pollen); Kaeppler et al., Plant Cell Reports 9:415-418 (1990) and Kaeppler et al., Theor. Appl. Genet. 84:560-566 (1992) (whisker-mediated transformation); D'Halluin et al., Plant Cell 4:1495-1505 (1992) (electroporation); Li et al., Plant Cell Reports 12:250-255 (1993) and Christou and Ford, Annals of Botany 75:407-413 (1995) (rice); Osjoda et al., Nature Biotechnology 14:745-750 (1996) (maize via Agrobacterium tumefaciens); all of which are herein incorporated by reference in their entireties.

In some embodiments, a plant may be regenerated or grown from the plant, plant tissue or plant cell. Any suitable methods for regenerating or growing a plant from a plant cell or plant tissue may be used, such as, without limitation, tissue culture or regeneration from protoplasts. Suitably, plants may be regenerated by growing transformed plant cells on callus induction media, shoot induction media and/or root induction media. See, for example, McCormick et al., Plant Cell Reports 5:81-84 (1986). These plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. Thus as used herein, “transformed seeds” refers to seeds that contain the nucleotide construct stably integrated into the plant genome.

The present invention has multiple aspects, illustrated by the following non-limiting examples.

### 10. EXAMPLES

The foregoing may be better understood by reference to the following examples, which are presented for purposes of illustration and are not intended to limit the scope of the invention.

### Example 1

### Methods and Materials

Plants and Growth Condition.

Arabidopsis thaliana Landsberg erecta (Ler) and tobacco (Nicotiana tabacum Petit Havana SRI) were used. Plants were grown in Metro-Mix 360 soil (Sun-Gro Horticulture) in a growth chamber under a 16-hour light/8-hour dark photoperiod regime at 22° C. and 50% of humidity.

Generation of Constructs and Transgenic Plants.

PCR reactions (see all primers in Table 1) were performed using Phusion High-Fidelity DNA Polymerase (New England Biolabs).

TABLE 1
Primers

| | | | Enzyme | | SEQ |
|---|---|---|---|---|---|
| Primer | Primer | | digestion | | ID |
| ID | name | Purpose | site | Sequence (5′ to 3′) | NO: |
| zp1283 | SDS | pENTR-SDS | Kpn I | CACCGGTACCCCATCATTCTC | 52 |
| | promoter 5′ | | | GTCTCTCTCGCAC | |
| zp1284 | SDS | pENTR-SDS | BsrGI | CAGTGTACATTTTTCTCCGTA | 53 |
| | promoter 3′ | | | CGAAAGCTTGAAAC | |
| zp1823 | mGFP5er 5, | pEarleyGate303- | XhoI | CCGCTCGAGGCAGGCTTTATG | 54 |
| | | mGFP5er | | AAGAC | |
| zp1824 | mGFP5er 3′ | pEarleyGate303- | XbaI | GCTCTAGAGCGGCCGCCGATC | 55 |
| | | mGFP5er | | TAGTAAC | |
| zp1768 | BARSTAR 5′ | pCR2.1- | NsiI | CCAATGCATTGGCGTATAACA | 56 |
| | | BARSTAR | | TAG | |
| zp1769 | BARSTAR 3′ | pCR2.1- | NsiI | CCAATGCATATGGCAGCGCTG | 57 |
| | | BARSTAR | | GCA | |
| zp1770 | XhoI 5′ | pEarleyGate303- | BglII | GAAGATCTGGATCCGGCTTAC | 58 |
| | | BARSTAR(XhoI) | | | |
| zp1771 | XhoI 3′ | pEarleyGate303- | XbaI, | GCTCTAGACTCGAGCTGTTCC | 59 |
| | | BARSTAR(XhoI) | XhoI | ACC | |
| zp1772 | BARNASE 5′ | pEarleyGate303- | XhoI | CCGCTCGAGTACGCTGTGAGG | 60 |
| | | BARSTAR- | | ATCTGTG | |
| | | BARNASE | | | |
| zp1773 | BARNASE3′ | BARSTAR- | XbaI | GCTCTAGAAGGATATCCTGAT | 61 |
| | | BARNASE | | CCGTTGAC | |
| zp2163 | SW11 5′ | Real-time PCR | | GGAGGAAGACATGGGATGGC | 62 |
| zp2164 | SW11 3′ | Real-time PCR | | CCCTTGTTCACCACCTTCACTT | 63 |
| | | | | C | |
| zp2165 | DMC1 5′ | Real-time PCR | | GGAGAACTCGCAGACCGCC | 64 |
| zp2166 | DMC1 3′ | Real-time PCR | | CCACCTGGGTCAGCTATGAC | 65 |
| zp1196 | A9 5′ | Real-time PCR | | ATGGTATCTCTAAAGTCCCTT | 66 |
| | | | | G | |
| zp1197 | A9 3′ | Real-time PCR | | CCAAATCCTCGGAACTGAATG | 67 |
| zp851 | ATA7 5′ | Real-time PCR | | CGTCTCCAGGATCGAGGAAT | 68 |
| zp852 | ATA7 3′ | Real-time PCR | | GGAGATGGGAAAGCTGAGAG | 69 |
| zp853 | ACTIN2 5′ | Real-time PCR | | GTTGGGATGAACCAGAAGGA | 70 |
| zp854 | ACTIN2 3′ | Real-time PCR | | GAGGAGCCTCGGTAAGAAGA | 71 |

The SDS promoter was amplified and cloned into the pENTR/D-TOPO vector (Invitrogen) to generate pENTR-SDS. The 1.5 kb promoter of the SDS gene (upstream of the SDS coding region and the 3′ non-coding region of the SDS adjacent gene) was amplified and cloned into the pENTR/D-TOPO vector (Invitrogen). The SDS genomic fragment from the promoter region to the last exon was introduced into the pENTR/D-TOPO vector to generate pENTR-SDS::SDS. The SDS genomic fragment from the beginning of the 1.5 kb promoter region to the last exon was introduced in the pENTR/D-TOPE vector. The mGFP5er was amplified from the pBIN Gal4-mGFP5er vector and cloned into the pEarleyGate303 binary vector (Earley et al., 2006, Plant J 45: 616-629) using the BamHI and SacI sites to generate pEarleyGate303-mGFP5er. The BARSTAR gene was amplified from the pABGCZ vector that contains BARSTAR and BARNASE(H102E) genes (Zhang et al., 2012, Plant Physiol 159: 1319-1334), then it was cloned into the pCR2.1 vector (Invitrogen) to generate pCR2.1-BARSTAR. BARSTAR was introduced from pCR2.1-BARS TAR into the pEarleyGate303 vector at the Nsi site to generate pEarleyGate303-BARSTAR. An XhoI site was introduced between BglII and XbaI sites right after attR2 to generate pEarleyGate303-BARSTAR(XhoI). The BARNASE fragment that was amplified from pABGCZ was cloned into pEarleyGate303-BARSTAR(XhoI) using the XhoI and XbaI sites to generate pEarleyGate303-BARSTAR-BARNASE. The gene for generating artificial microRNAs targeting to BARNASE was designed, as described previously (Schwab et al., 2006, Plant Cell 18: 1121-1133; Ossowski et al., 2008, Plant J 53: 674-690). The amiR-BARNASE fragment was amplified and cloned into pRS300 vector, which contains miR319a precursor sequence in pBSK (Schwab et al., 2006, Plant Cell 18: 1121-1133). Then, the amiR-BARNASE fragment was introduced into the estradiol (ER) inducible vector (Zuo et al., 2000, Plant J 24: 265-273) at the XhoI and SpeI sites to generate ER::amiR-BARNASE. Using the Gateway LR recombinase II enzyme mix (Invitrogen), SDS::GUS, SDS::GFP, SDS::BARNASE, SDS::SDS-GUS, SDS::SDS-GFP, and SDS::SDS-BARNASE binary vectors were generated between pENTR-SDS and pENTR-SDS::SDS as well as pGBW3, pEarleyGate303-mGFP5er, and pEarleyGate303-BARSTAR-BARNASE. Then these vectors and ER::amiR-BARNASE were transformed into the Agrobacterium strain GV3101.

The floral dip method was used to generate transgenic Arabidopsis (Clough and Bent, 1998, Plant J 16:735-743). Transformants of SDS::GUS and SDS::SDS-GUS were screened on 50 μg/mL of kanamycin and 25 μg/mL of hygromycin. Transformants of SDS::GFP, SDS::SDS-GFP, SDS::BARNASE, and SDS::SDS-BARNASE were screened on 1% of Basta (PlantMedia). Transformants of ER::amiR-BARNASE was screened on 25 μg/mL of hygromycin. Tobacco transformation was performed. Briefly, leaf discs were inoculated with the Agrobacterium strain GV3101 containing the SDS::SDS-BARNASE binary vector and cultured for 1 day in the dark, followed by 2 days under light. Then, leaf discs were screened on shoot and root selection medium containing 4% of Basta. The regenerated plants were transferred into soil and sprayed with 4% of Basta solution one week later. The surviving plants were used for further analyses.

Pollen Staining and Anther Semi-Thin Sections.

To access pollen viability, Alexander pollen staining was carried as described previously (Zhao et al., 2002, Genes Dev 16: 2021-2031). Mature anthers of tobacco were collected and analyzed using the same method. Pollen grains were released from anthers before imaging. Semi-thin sectioning was performed as described in our previous studies (Zhao et al., 2002, Genes Dev 16: 2021-2031; Jia et al., 2008, PNAS 105:2220-2225).

Estradiol Induction of ER::amiR-BARNASE.

Induction [2 μmol/L estradiol (Sigma) and 0.02% Silwet L-77] and mock (without estradiol) solutions were dropped or sprayed to main inflorescences in the morning, respectively. Seven day induction resulted in fertility restoration under our growth chamber condition.

GUS Staining Assay.

Histochemical GUS staining assay was performed. Tissues were collected and fixed for 1 h in 90% acetone at −20° C. After washing tissues in washing buffer [0.1 M phosphate (pH 7.0), 10 mM EDTA, and 2 mM K₃Fe(CN)₆] twice for 5 min under the vacuum, the drained tissues were transferred into the GUS staining buffer [0.1 M phosphate (pH 7.0), 10 mM EDTA, 1 mM K3Fe(CN)₆, 1 mM K₄Fe(CN)₆.3H₂O, and 1 mg/ml X-GLUC)] and incubated overnight at 37° C. GUS-stained tissues were then fixed in a 3:1 mixture of ethanol and acetic acid. Tissues were mounted onto the glass slides for observation.

Real-Time qRT-PCR.

Inflorescences of wild-type, SDS::SDS-BARNASE and ER::amiR-BARNASE SDS::SDSBARNASE plants were collected for RNA isolation using the RNeasy Plant Mini Kit (Qiagen). RNA quantification was determined with a NanoDrop 2000c (Thermo Scientific). RNA reverse transcription was performed using the QuantiTect Reverse Transcription Kit (Qiagen). Real-time PCR (DNA Engine Opticon 2 system) and data analysis were performed as previously described (Liu et al., 2010, Plant J. 62, 416-428) to evaluate expression of BARNASE, DMC1, SW11, A9, and ATA7 (Table 1). The ACTIN2 gene was used as an internal control. Three independent biological repeats were carried out.

Microscopy. Pollen Staining Samples:

GUS staining was observed with an Olympus SZX7 microscope. Semi-thin sections were observed with an Olympus BX51 microscope. Images were obtained with an Olympus DP 70 digital camera. For confocal microscopy analysis, anthers and ovules were dissected and mounted in water. GFP signal was observed with a Leica TCS SP2 laser scanning confocal microscope using a 63x/1.4 water immersion objective lens. The 488-nm laser line was used to excite GFP and the emission capture PMT was set at 505-530 nm. The 488-nm laser line was used to excite GFP and it also induced chlorophyll autofluorescence. The PMT gain settings was held at 650. GFP and chlorophyll autofluorescence were detected at 505-530 nm and 644-719 nm, respectively.

### Example 2

### BARNASE Driven by the SDS Promoter Caused Defects in Growth and Reproduction

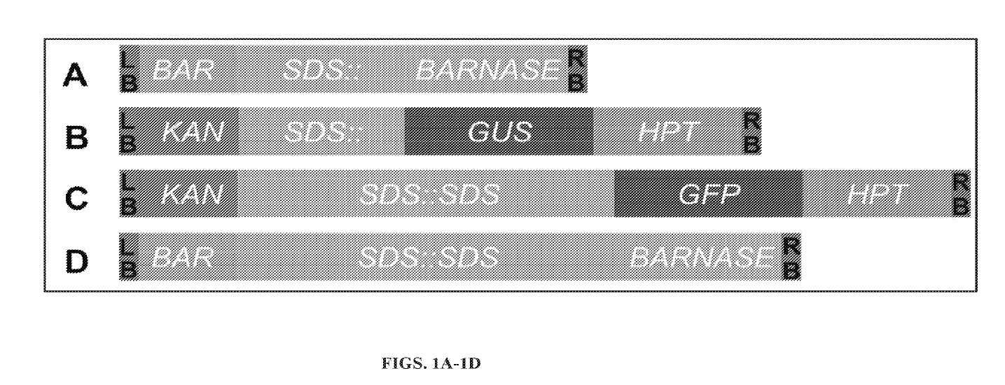

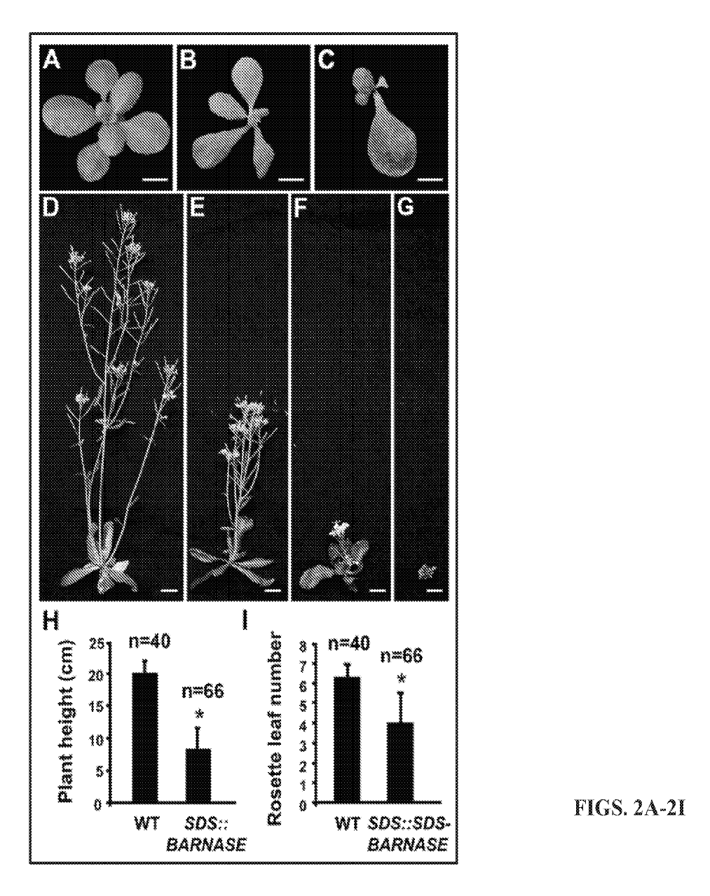

In Arabidopsis, the SDS gene, which encodes a meiosis-specific cyclin, is exclusively expressed in microspore mother cells (male meiocytes) in anthers and megaspore mother cells (female meiocytes) in ovules. To create completely both male and female sterile plants without altering flower structure, the SDS::BARNASE construct was generated using the 1.5-kb promoter of the SDS gene and a modified BARNASE (Zhang et al., 2012) to genetically ablate microspore and megaspore mother cells in Arabidopsis (FIG. 1A). Among 66 examined SDS::BARNASE transgenic plants, none of them showed the specific phenotype in sterility. Instead, compared with the wild-type (FIG. 2A), SDS::BARNASE young plants were defective in vegetative growth, indicated by abnormal shape and numbers of rosette leaves (FIGS. 2B and 2C). Different from the WT adult plant (FIG. 2D), SDS::BARNASE adult plants also exhibited various abnormal phenotypes, such as dwarf and fertile (FIG. 2E), dwarf and sterile (FIG. 2F), and even no inflorescence (FIG. 2G). The height of mature SDS::BARNASE plants was significantly reduced (FIG. 2H). Moreover, SDS::BARNASE plants produced significantly fewer rosette leaves than that of wild-type (FIG. 2I). Various defects of SDS::BARNASE plants in growth and development suggest that the 1.5-kb promoter of the SDS gene failed to drive the specific expression of BARNASE in microspore and megaspore mother cells.

### Example 3

### 1.5 kb Upstream Region of the SDS Gene Did not Confer its Meiocyte-Specific Expression

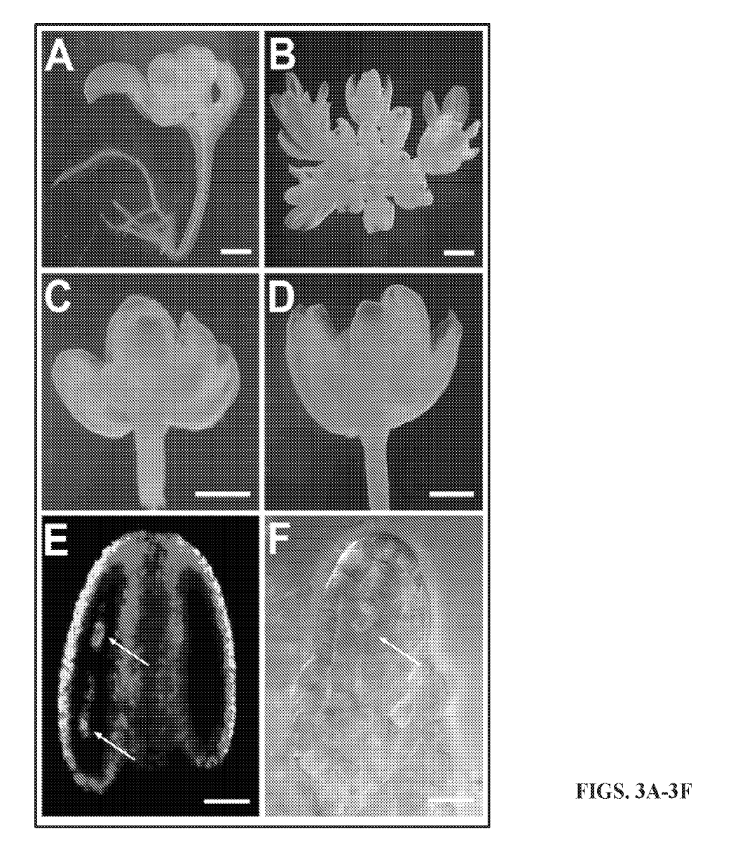

Genetic ablation relies on the specificity of employed promoters. To examine why BARNASE under the control of the 1.5-kb SDS promoter did not achieve specific ablation effects on microspore and megaspore mother cells, SDS::GUS plants were generated to test the transcriptional activity of the 1.5-kb promoter (FIG. 1B). Among 25 examined SDS::GUS transgenic plants, GUS signals were detected in cotyledons, true leaves, and shoot apical meristem of young seedlings (FIG. 3A), as well as in carpels and stigmas of young buds (FIGS. 3B-3D). Thus, the results suggest that the 1.5-kb promote of the SDS gene was not sufficient for conferring its meiocyte-specific expression, which resulted in abnormal plant growth and development when it drove the expression of BARNASE.

### Example 4

### SDS::SDS-BARNASE Causes Complete Male and Female Sterility but does not Affect Plant Growth and Development

The possible existence of regulatory elements in SDS introns may contribute to the SDS meiocyte-specific expression. To achieve the specific expression of SDS in microspore and megaspore mother cells, SDS::SDS-GFP constructs were generated by fusing the SDS genomic fragment, containing the 1.5-kb promoter, seven exons and six introns, with the GFP gene (FIG. 1C). In examined 18 SDS::SDS-GFP transgenic plants, the GFP signal was not detected during the seedling stage and later in the vegetative growth stage. We, however, observed GFP signals only in microspore mother cells in anthers (FIG. 3E) and megaspore mother cell in ovule during the reproductive stage (FIG. 3F). Therefore, our results indicate that the entire SDS gene led to the meiocyte-specific expression of the SDS protein.

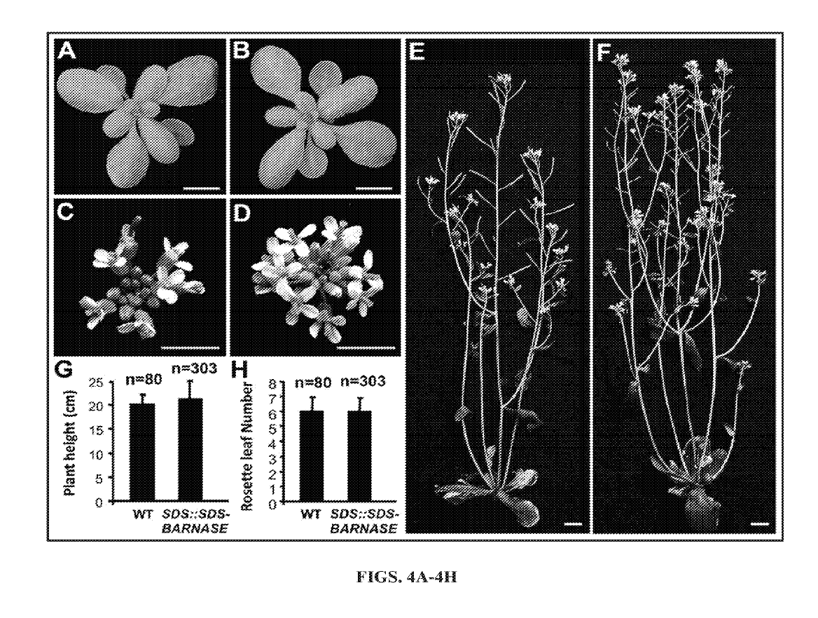

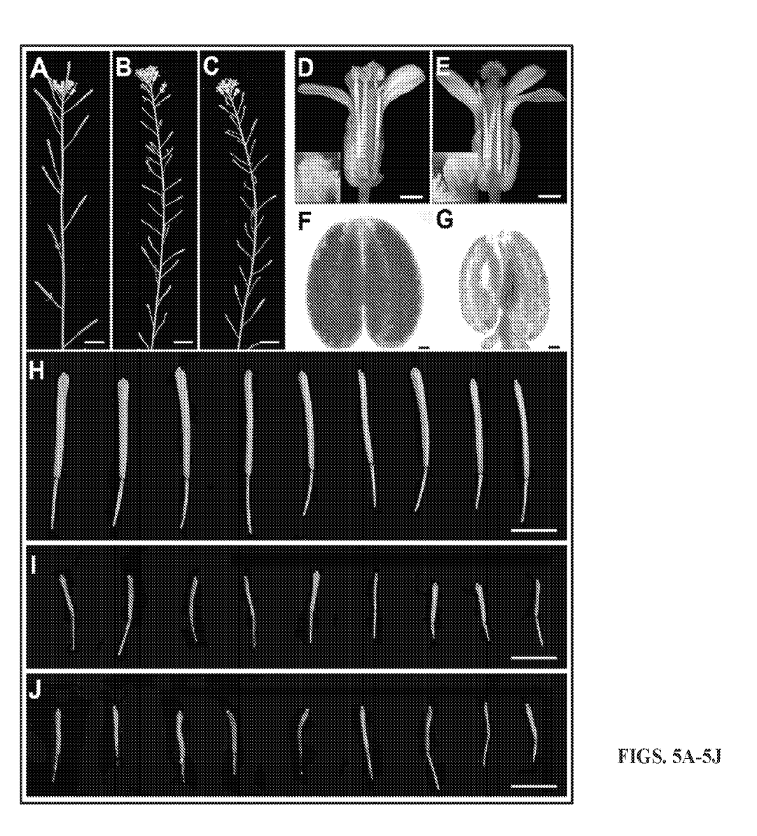

To generate complete both male and female sterility by specifically ablating microspore and megaspore mother cells, the SDS::SDS-BARNASE construct was made by fusing the SDS entire gene with the BARNASE gene (FIG. 1D). We performed three transformations, resulting in 97, 80, and 126 SDS::SDS-BARNASE transgenic plants, respectively. All independent transgenic plants were sterile. We first evaluated the effects of SDS::SDS-BARNASE on growth and development. SDS::SDS-BARNASE transgenic plants produced rosette leaves with the same number, size, and shape as that of WT plants (FIGS. 4A, 4B). No morphological changes were observed in SDS::SDS-BARNASE inflorescences and flowers (FIGS. 4C, 4D). Moreover, mature SDS::SDS-BARNASE plants had a similar height to the wild-type (FIGS. 4E-4G). The flowering time of SDS::SDS-BARNASE plants was not affected either, because the same rosette leaf numbers as the wild-type were produced when flowering (FIG. 4H). To further investigate sterility of SDS::SDS-BARNASE transgenic plants, we analyzed both male and female fertilities. Compared with the wild-type (FIGS. 5A, 5H), SDS::SDS-BARNASE plants produced short siliques (FIGS. 5B, 5I). Except short filaments, SDS::SDS-BARNASE plants formed flowers that were the same as the wild-type, indicated by four sepals, four petals, six stamens, and two carpels (FIGS. 5D, 5E). In the WT flower, pollen grains were released from anthers that reached the stigma (FIG. 5D), whereas in the SDS::SDS-BARNASE flower, no pollen grains were observed on the anther surface and anthers did not reach the stigma (FIG. 5E). Furthermore, different from the WT anther (FIG. 5F), the SDS::SDS-BARNASE anther did not produce pollen grains (FIG. 5G), indicating that SDS::SDS-BARNASE plants were male sterile. Because pollination using the WT pollen did not rescue the fertility (FIGS. 5C, 5J), SDS::SDS-BARNASE plants were female sterile too. Thus, using SDS::SDS-BARNASE, we efficiently created completely both male and female sterile Arabidopsis plants that had normal vegetative and reproductive growth and development, including the formation of all flower organs.

### Example 5

### SDS::SDS-BARNASE Inhibited Both Male and Female Gamete Formation

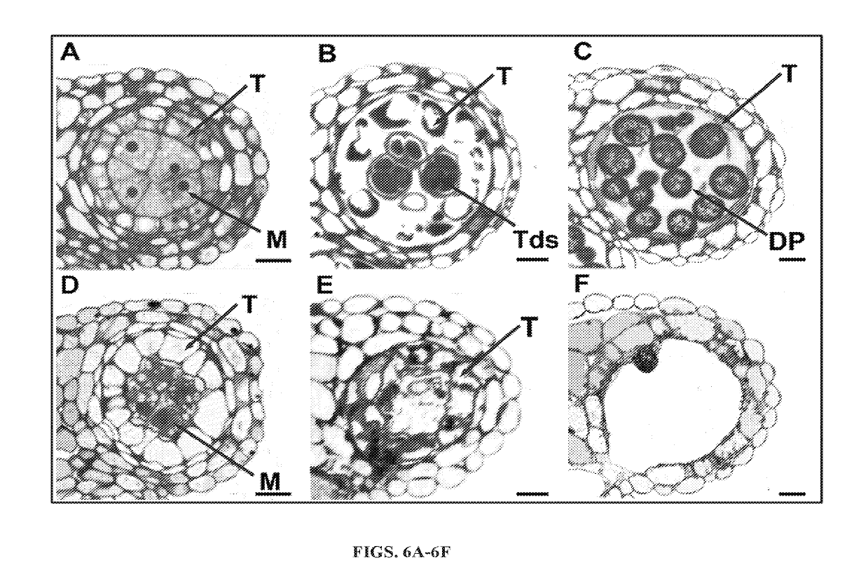

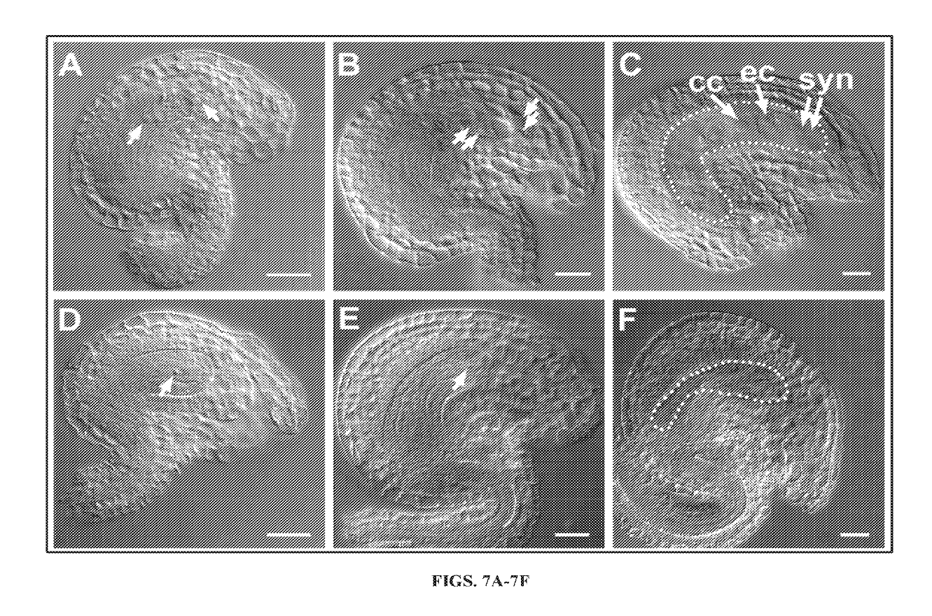

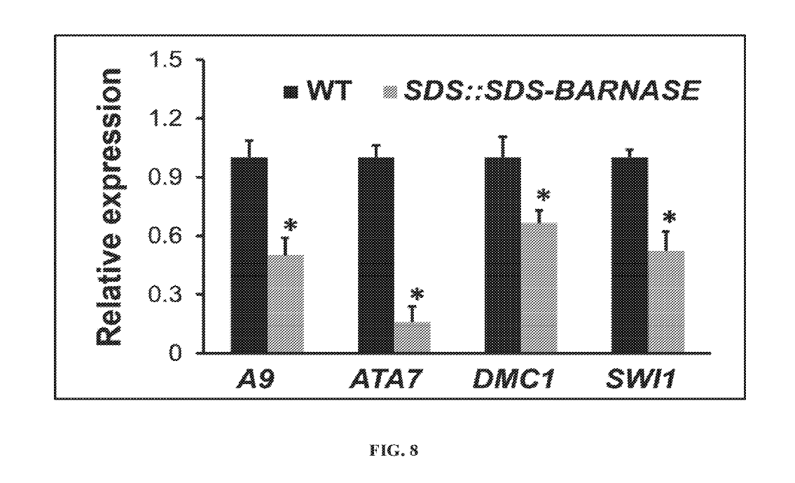

To further understand ablation effects on microspore and megaspore mother cells, we did semi-thin sectioning of anthers and whole-mount squashes of ovules. At stage 5, when compared with the WT anthers (FIG. 6A), the SDS::SDS-BARNASE anther showed vacuolated microsporocytes (microspore mother cells) and tapetal cells (FIG. 6D), indicating the degeneration of both cells. At stage 7 in the WT anther, successful male meiosis resulted in the formation of tetrads (FIG. 6B), whereas in the SDS::SDS-BARNASE anther, tetrads, and tapetal cells were collapsed (FIG. 6E). At stage 9, the WT anther contains developing pollen grains (FIG. 6C), but the SDS::SDS-BARNASE anther lacked developing microspore s (FIG. 6F). In embryo sacs of WT ovules, two nuclei at stage FG3 (FIG. 7A) and four nuclei at stage FG4 (FIG. 7B) were observed; however, in SDS::SDS-BARNASE embryo sacs, only a single nucleus was produced (FIGS. 7D, 7E). At stage FG6, the WT embryo sac showed the central cell, the egg cell, and synergid cells (FIG. 7C), but the SDS::SDS-BARNASE embryo sac is empty (FIG. 7F). Furthermore, our results showed that expressions of tapetal cell marker genes A9 and ATA7 as well as microspore and megaspore mother cell marker genes DMC1 and SWI1 were significantly decreased in SDS::SDS-BARNASE buds in comparison to the wild-type (FIG. 8). In summary, the specific expression of the SDS-BARNASE toxic fusion protein in microspore and megaspore mother cells efficiently impaired the production of both male and female gametes, which led to absolute both male and female sterility, but did not affect flower organ formation or plant growth and development.

### Example 6

### Combination of an Inducible System and Artificial MicroRNA Technology Restores Fertilities to SDS::SDS-BARNASE Plants

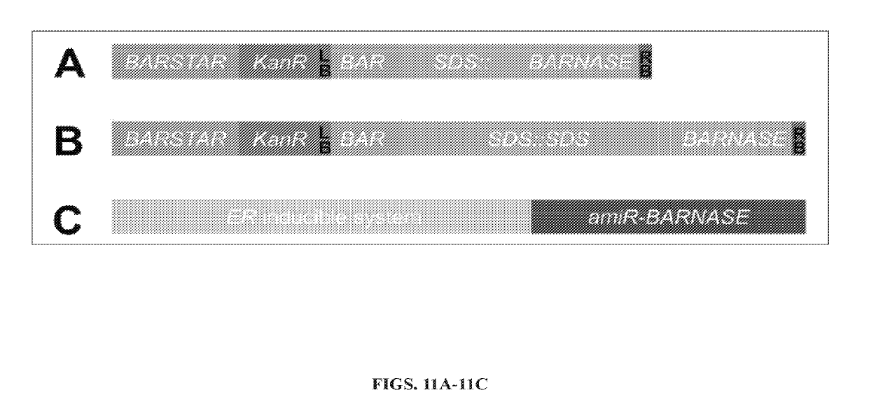

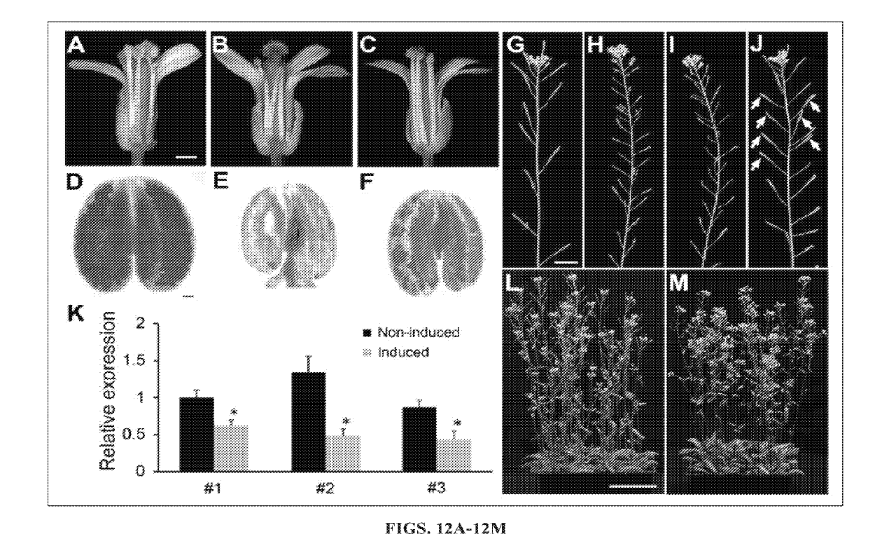

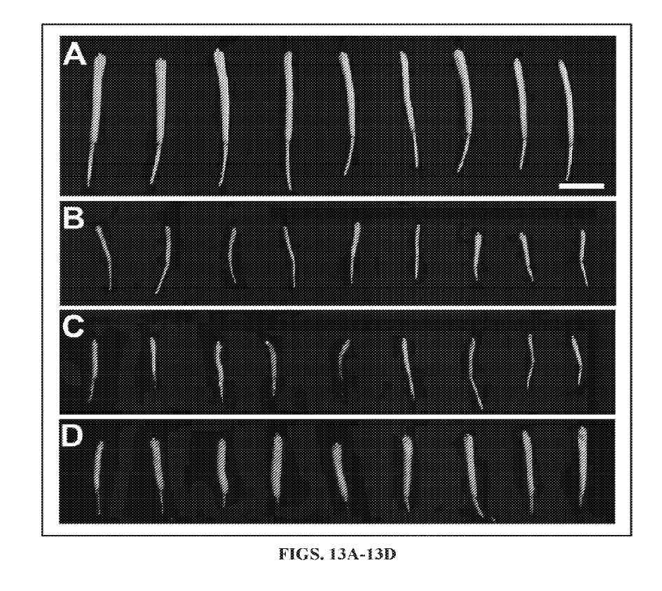

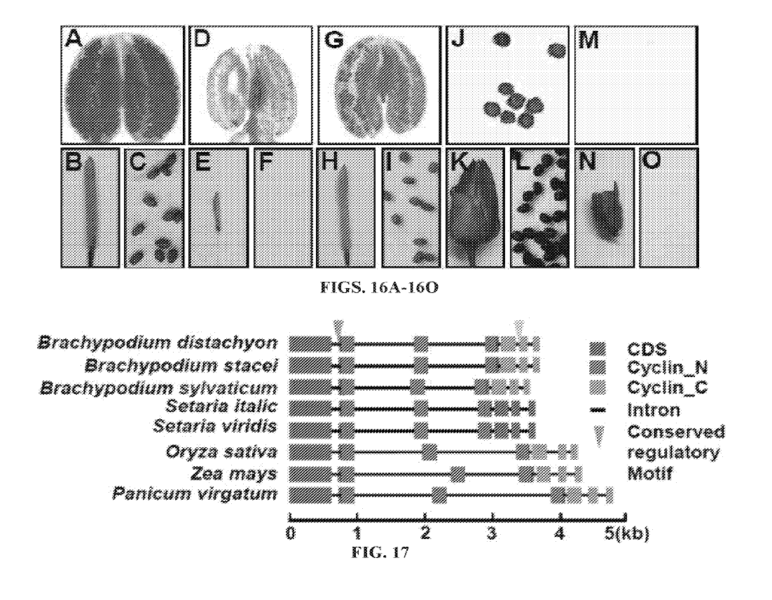

To restore fertility to SDS::SDS-BARNASE plants, we generated the ER::amiR-BARNASE construct to produce an artificial microRNA (Schwab et al., 2006, Plant Cell 18: 1121-1133) targeting the BARNASE gene under control of the estradiol inducible system (Zuo et al., 2000, Plant J 24: 265-273) (FIG. 11C). ER::amiR-BARNASE plants exhibit no differences from wild type, with or without estradiol treatment. SDS::SDSBARNASEER::amiR-BARNASE double transgenic plants showed the same sterile phenotype as SDS::SDS-BARNASE plants without estradiol treatment, while after the treatment with estradiol, the fertility of 40% (12/30) of examined SDS::SDS-BARNASE/ER::amiR-BARNASE plants was partially rescued, indicated by the formation of pollen grains in anthers (FIGS. 12C and 13F) and elongation of siliques (FIG. 12J; FIG. 13D). Real-time qRT-PCR showed that the accumulation of BARNASE transcripts was decreased after estradiol treatment (FIG. 12K). Offspring from recovered seeds are completely sterile without estradiol treatment (FIGS. 12L and 12M). Our results showed that male and female sterility of SDS::SDS-BARNASE can be restored by the inducible artificial microRNA approach. See also FIGS. 16A-16O.

### Example 7

### SDS::SDS-BARNASE Causes Male and Female Sterility in Tobacco

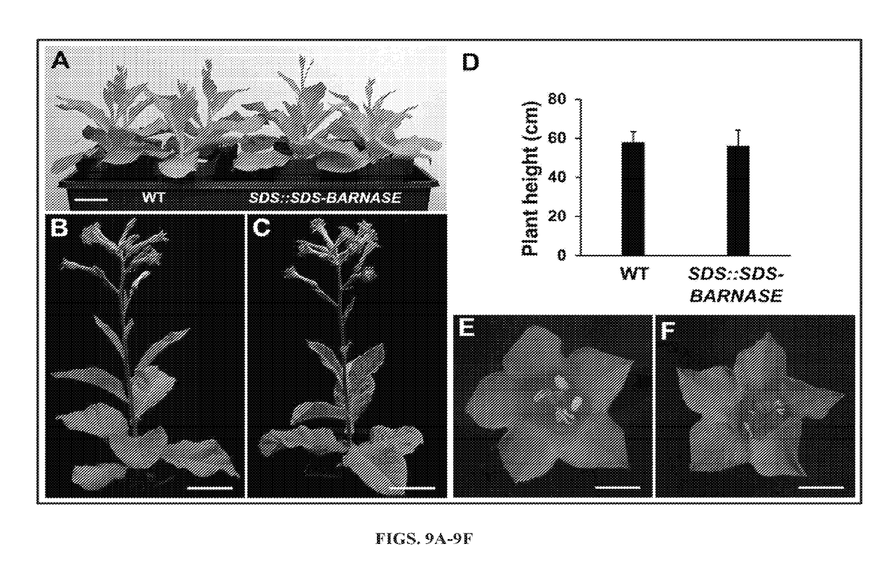

To test whether SDS::SDS-BARNASE can provide a general tool to create both male and female sterile plants, we transformed it into tobacco and generated SDS::SDS-BARNASE tobacco transgenic plants by tissue culture. Among 14 examined SDS::SDS-BARNASE tobacco transgenic lines, leaf shape and size (FIGS. 9A-9C), as well as the plant height (FIGS. 9B-9D) were the same as that of WT plants. In addition, the SDS::SDS-BARNASE tobacco flower had the same size, color, and structure as that of wild type (FIGS. 9E, 9F). Therefore, SDS::SDS-BARNASE did not affect growth or development in tobacco plants.

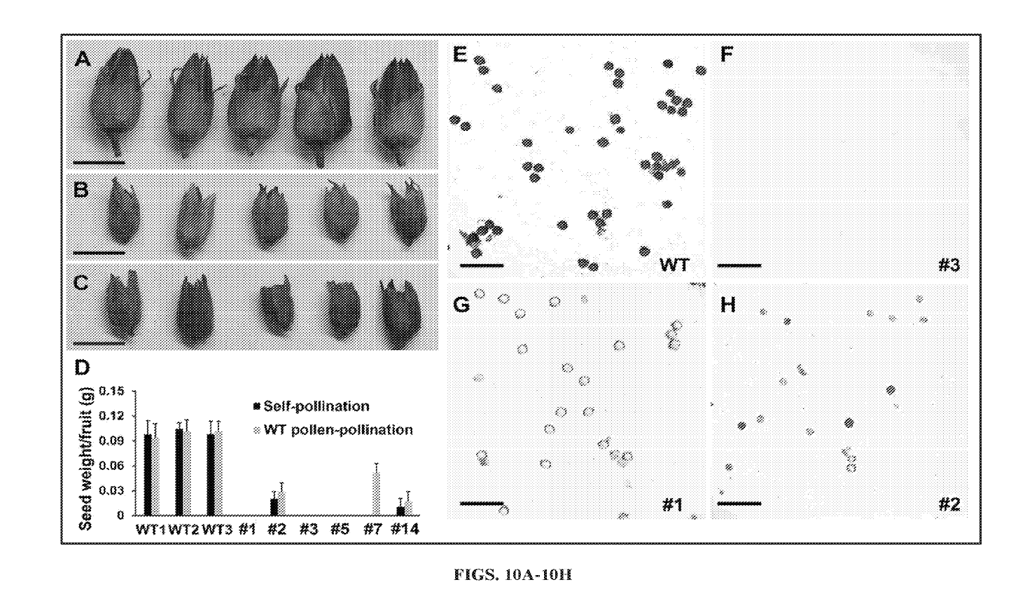

Ten examined SDS::SDS-BARNASE tobacco transgenic lines were completely sterile. WT tobacco plants produced large fruits and per fruit averagely contained 0.11 g of seeds (FIGS. 10A, 10D). Conversely, SDS::SDS-BARNASE plants produced small fruits and no seeds were found when self-pollinated (FIGS. 10B, 10D, e.g., plants #1, 3, 5, and 7). Further pollen viability analysis showed that WT tobacco anthers produced viable pollen, indicated by red color (FIG. 10E), whereas anthers from sterile tobacco plants either lacked pollen grains (FIG. 10F) or formed dead pollen grains (FIG. 10G). The four non-absolutely sterile lines produced a few seeds (FIG. 10D, e.g., plants #2, and 14) and only some functional pollen grains were found in anthers of those lines (FIG. 10H, e.g., plant #2). SDS::SDS-BARNASE may impair male fertility in tobacco.

The female fertility in sterile tobacco transgenic plants was examined. The fertility of manually male-sterilized WT flowers could be rescued by cross-pollination with WT pollen (FIG. 10D), but following cross-pollination with WT pollen, the fruit size of SDS::SDS-BARNASE sterile tobacco plants did not change (FIG. 10C) and no seeds were produced (FIG. 10D, e.g., plants #1, 3, and 5). Thus, SDS::SDS-BARNASE tobacco transgenic plants were also female sterile. Manual pollination partially rescued the fertility of line #7, indicating that the line #7 is a completely male but partially female sterile plant, while lines #2 and 14 were nearly male and female sterile plants (FIG. 10D). Collectively, a majority of SDS::SDS-BARNASE tobacco transgenic plants were completely male and female sterile, suggesting that SDS::SDS-BARNASE is functionally conserved, which can be used to create both male and female sterility in general.

### Example 8

### Completely Sterile Brachypodium

A Brachypodium regenerating system is established and a BdSDS::BdSDS-BARNASE construct is generated. The SDS::SDS-BARNASE construct is modified to generate the BdSDS::BdSDS-BARNASE construct. A 2-Kb upstream sequence and following genomic sequence of BdSDS containing 7 exons and 6 introns is used to replace the Arabidopsis SDS::SDS fragment. To achieve a high B. distachyon transformation efficiency, the ablation construct described above was modified using the HPT selectable gene (conferring resistance to hygromycin) under control of the maize ubiquitin promoter (FIG. 18B). Moreover, the 35S::BAR fragment used for transgenic plants selection in Arabidopsis is replaced by UBI::HPT which is suitable for transgenic Brachypodium selection. The Arabidopsis SDS::SDS genomic fragment is replaced with the BdSDS::BdSDS genomic fragment that contains a 2-Kb promoter sequence following a genomic fragment with 7 exons and 6 introns (FIGS. 18A and 18B). The resulting construct (named BdSDS::BdSDS:BARNASE) will be used to transform B. distachyon Bd21-3 via tissue culture. The Agrobacteria harboring the BdSDS::BdSDS-BARNASE construct is transfected into Brachypodium callus. The BdSDS::BdSDS-BARNASE plants are regenerated.

The following results are expected: (1) produce bisexually sterile BdSDS::BdSDS-BARNASE Brachypodium plants with normal growth and normal flower organs; (2) obtain male sterile Brachypodium from transgenic plants derived from one of mutated constructs; (3) restore the fertility of the sterile BdSDS::BdSDS-BARNASE Brachypodium plants by either sparing or watering with ethanol.

### Example 9

### Male Sterile Only Brachypodium Plants

The regulatory motif responsible for the SDS expression in male meiocytes is identified. A system that only ablates male reproductive cells for achieving male sterile only Brachypodium plants is developed. 4 novel putative regulatory motifs (M1, M2, M3, and M4) in the BdSDS promoter and introns were identified. BdSDS::BdSDS-BARNASEΔM1, BdSDS::BdSDS-BARNASEΔM2, BdSDS::BdSDS-BARNASEΔM3 and BdSDS::BdSDS-BARNASEΔM4 constructs are generated by deleting M1, M2, M3, and M4, respectively. Then transgenic plants are generated to test the male fertility.

### Example 10

### Restoring Fertility of Sterile Brachypodium

Maize ubiquitin promoter controlled ethanol-inducible system and amiR-BARNASE are used to rescue target plants fertility by inserting the inducible unit into the construct containing fertility ablation unit. Ethanol-inducible system has been successfully used in both dicots and monocots. Considering the price, availability and non-toxic in a moderate amount, ethanol is suitable for field application. The best concentration of ethanol will be tested by spraying on flowers or watering.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. An isolated polynucleotide construct comprising a first polynucleotide and a second polynucleotide, the first polynucleotide comprising a SOLO-DANCERS (SDS) gene or fragment thereof, the second polynucleotide comprising a Barnase gene or fragment thereof, wherein the SDS gene comprises the SDS promoter.

Clause 2. The isolated polynucleotide construct of clause 1, wherein the isolated polynucleotide construct is operably linked to the SDS promoter.

Clause 3. The isolated polynucleotide construct of clause 1 or 2, wherein the SDS gene comprises at least one regulatory intron.

Clause 4. The isolated polynucleotide construct of clause 3, wherein the at least one regulatory intron comprises a sequence of any one of SEQ ID NO: 22-26 or 47-51.

Clause 5. The isolated polynucleotide construct of any one of clauses 1-4, wherein the SDS gene comprises a polynucleotide sequence of any one of SEQ ID NO: 1-21 or 29-46.

Clause 6. The isolated polynucleotide construct of any one of clauses 1-5, wherein the Barnase gene comprises a polynucleotide sequence of any one of SEQ ID NO:27.

Clause 7. A vector comprising the isolated polynucleotide construct of any one of clauses 1-6.

Clause 8. A plant cell comprising the vector of clause 7.

Clause 9. A plant comprising the plant cell of clause 8.

Clause 10. The plant of clause 9, wherein the plant is completely male sterile and female sterile.

Clause 11. The plant of clause 10, wherein the plant is a gymnosperm or angiosperm.

Clause 12. The plant of clause 11, wherein the plant is a grass, tree, or ornamental plant.

Clause 13. The plant of clause 11, wherein the plant is Arabidopsis, tobacco, alfalfa, soybean, maize, rice, Brachypodium, switchgrass, Miscanthus, poplars, cherry, or Eucalyptus.

Clause 14. A composition for generating a complete male sterile and female sterile transgenic plant, the composition comprising the isolated polynucleotide construct of clause 1.

Clause 15. The composition of clause 14, further comprising a second isolated polynucleotide construct, wherein the second isolated polynucleotide construct comprises an inducible promoter operably linked to an artificial microRNA (amiRNA) targeted to the Barnase gene or fragment thereof, wherein the fertility of the plant is restored by inducing the expression of the amiRNA.

Clause 16. The composition of clause 15, wherein the amiRNA comprises a polynucleotide sequence of SEQ ID NO: 28.

Clause 17. The composition of clause 15 or 16, wherein the inducible promoter is an estradiol inducible promoter, an ethanol inducible promoter, a dexamethasone inducible promoter, a methoxyfenozide inducible promoter, or a temperature inducible promoter.

Clause 18. The composition of clause 17, wherein the temperature inducible promoter is a heat shock inducible promoter or a heat inducible promoter.

Clause 19. The composition of any one of clauses 14-17, wherein the isolated polynucleotide construction of clause 1 and the second isolated polynucleotide are encoded on the same vector.

Clause 20. The composition of any one of clauses 14-17, wherein the isolated polynucleotide construction of clause 1 and the second isolated polynucleotide are encoded on separate vectors.

Clause 21. A vector comprising the composition of any one of clauses 14-18.

Clause 22. A plant cell comprising the vector of clause 21 or the composition of clause 19 or 20.

Clause 23. A plant comprising the plant cell of clause 22.

Clause 24. The plant of clause 23, wherein the plant becomes male fertile and female fertile after the induction of amiRNA.

Clause 25. The plant of clause 24, wherein the plant is a gymnosperm or angiosperm.

Clause 26. The plant of clause 25, wherein the plant is a grass, tree, or ornamental plant.

Clause 27. The plant of clause 25, wherein the plant is Arabidopsis, tobacco, alfalfa, soybean, maize, rice, Brachypodium, switchgrass, Miscanthus, poplars, cherry, or Eucalyptus.

Clause 28. A method for generating a complete male sterile and female sterile plant, the method comprising introducing into a target plant an isolated polynucleotide construct of any one of clauses 1-6 to generate a transgenic plant.

Clause 29. A method for ablating microspore and megaspore mother cells in a plant, the method comprising introducing into a target plant an isolated polynucleotide construct of any one of clauses 1-6 to generate a transgenic plant, wherein the microspore and megaspore mother cells are ablated.

Clause 30. A method for restoring fertility in a male sterile and female sterile transgenic plant, the method comprising: (a) introducing into a target plant a composition of any one of clauses 14-20 to generate a transgenic plant; (b) introducing into the transgenic plant generated in (a) an isolated polynucleotide construct of any one of clauses 1-6 to generate a double transgenic plant; and (c) inducing the expression of the amiRNA, thereby restoring fertility in a complete male sterile and female sterile transgenic sterile plant.

Clause 31. A method for restoring fertility in a male sterile and female sterile transgenic plant, the method comprising: (a) introducing into a target plant a second isolated polynucleotide construct, wherein the second isolated polynucleotide construct comprises an inducible promoter operably linked to an artificial microRNA (amiRNA) targeted to the Barnase gene or fragment thereof to generate a transgenic plant; (b) introducing into the transgenic plant generated in (a) the isolated polynucleotide construct of claim 1 to generate a double transgenic plant; and (c) inducing the expression of the amiRNA, thereby restoring fertility in a complete male sterile and female sterile transgenic sterile plant.

Clause 32. The method of clause 30 or 31, wherein the isolated polynucleotide construct and the second polynucleotide construct are encoded on the same vector.

Clause 33. The method of clause 30 or 31, wherein the isolated polynucleotide construct and the second polynucleotide construct are encoded on different vectors.

Clause 34. The method of any one of clauses 30-33, wherein inducing the expression of the amiRNA comprises contacting the transgenic plant with estradiol, ethanol, dexamethasone, methoxyfenozide, or temperature.

Clause 35. The method of any one of clauses 30-34, wherein the target plant is a gymnosperm or angiosperm.

Clause 36. The method of clause 35, wherein the target plant is a grass, tree, or ornamental plant.

Clause 37. The method of clause 35, wherein the target plant is Arabidopsis, tobacco, alfalfa, soybean, maize, rice, Brachypodium, switchgrass, Miscanthus, poplars, cherry, or Eucalyptus.

Clause 38. The method of any one of clauses 28-37, wherein the SDS gene is an endogenous gene of target plant.

Clause 39. The method of any one of clauses 28-37, wherein the SDS gene is a transgene to the target plant.

Clause 40. The plant of any one of clauses 8-13 or 23-27, wherein the SDS gene is an endogenous gene of target plant.

Clause 41. The plant of any one of clauses 8-13 or 23-27, wherein the SDS gene is a transgene to the target plant.

Clause 42. A transgenic plant produced by the method of clause 28.

1-41. (canceled)

42. An isolated polynucleotide construct comprising a first polynucleotide and a second polynucleotide, the first polynucleotide comprising a SOLO-DANCERS (SDS) gene or fragment thereof, the second polynucleotide comprising a Barnase gene or fragment thereof, wherein the SDS gene comprises the SDS promoter.

43. The isolated polynucleotide construct of claim 42, wherein the isolated polynucleotide construct is operably linked to the SDS promoter.

44. The isolated polynucleotide construct of claim 42, wherein the SDS gene comprises at least one regulatory intron.

45. The isolated polynucleotide construct of claim 44, wherein the at least one regulatory intron comprises a sequence of any one of SEQ ID NO: 22-26 or 47-51.

46. The isolated polynucleotide construct of claim 42, wherein the SDS gene comprises a polynucleotide sequence of any one of SEQ ID NO: 1-21 or 29-46.

47. The isolated polynucleotide construct of claim 42, wherein the Barnase gene comprises a polynucleotide sequence of any one of SEQ ID NO:27.

48. A vector comprising the isolated polynucleotide construct of claim 42.

49. A plant cell comprising the vector of claim 48.

50. A plant comprising the plant cell of claim 49.

51. A composition for generating a complete male sterile and female sterile transgenic plant, the composition comprising the isolated polynucleotide construct of claim 42.

52. The composition of claim 51, further comprising a second isolated polynucleotide construct, wherein the second isolated polynucleotide construct comprises an inducible promoter operably linked to an artificial microRNA (amiRNA) targeted to the Barnase gene or fragment thereof, wherein the fertility of the plant is restored by inducing the expression of the amiRNA.

53. The composition of claim 52, wherein the amiRNA comprises a polynucleotide sequence of SEQ ID NO: 28.

54. The composition of claim 52, wherein the inducible promoter is an estradiol inducible promoter, an ethanol inducible promoter, a dexamethasone inducible promoter, a methoxyfenozide inducible promoter, or a temperature inducible promoter.

55. A vector comprising the composition of claim 51.

56. A plant cell comprising the vector of claim 55.

57. A plant comprising the plant cell of claim 56.

58. A method for generating a complete male sterile and female sterile plant, the method comprising introducing into a target plant an isolated polynucleotide construct of claim 42 to generate a transgenic plant.

59. A method for restoring fertility in a male sterile and female sterile transgenic plant, the method comprising:
introducing into a target plant a second isolated polynucleotide construct, wherein the second isolated polynucleotide construct comprises an inducible promoter operably linked to an artificial microRNA (amiRNA) targeted to the Barnase gene or fragment thereof to generate a transgenic plant;
introducing into the transgenic plant generated in (a) the isolated polynucleotide construct of claim 42 to generate a double transgenic plant; and
inducing the expression of the amiRNA, thereby restoring fertility in a complete male sterile and female sterile transgenic sterile plant.

60. The method of claim 59, wherein the isolated polynucleotide construct and the second polynucleotide construct are encoded on the same vector or on different vectors.

61. The method of claim 59, wherein inducing the expression of the amiRNA comprises contacting the transgenic plant with estradiol, ethanol, dexamethasone, methoxyfenozide, or temperature.


SEQUENCE LISTING (71 sequences)

SEQ ID NO: 1 - DNA, 3617 bp, Aquilegia coerulea
atgaatgagacggtgagttcaattcttcaacagagcaattcgatcagaaaacggaagttaccagttacatttactaagaaactccgatcaaagattcctcgtcggaaacgctttcagatctctccaattcttactggatcatctaattctctgttttctgacgattttaagccgggattatcagctttccaagctgcttcgagtacttcttcatgctttcctgacgaaatttcttctctatcaacctggatatcagccggatcggaggataagattaagaaacggagatcaactcgccagaagaagagttttgaagatgttcgaggttttgaagttgcagatgtgcgaatttctcttcaattggagaatcggccgataacgagaacatatttgaagctgatagaaagcaaaagagaattgaagttgaaaggttatgtagatctaggagttgagttatcggaaacttcttgtgttgaatcgtcttcaataacagagaatggagttctcatcaaatccggagagctttctgagaagaatttgaagtttaatgatagagctcatagactacttgagaaggaagagcaaatcgatgtaactgcaacttctgatgcaatttctctttcagaagtttcttgcattcaacacgaaaatgctcagattgtctacgaacaaggtgaattcgatgaaacatcgaagctgaaagttacagaacctacagaagatgaagagaatgaaggtgttagcgttttttcagatgagacgaacagagcacctgaattatcattttcagaaatctctggaaattgtttggaatcaaatgtgacgacgacgcttgatcagaagccgaaggaccttgatttggagtatagtcttgcttgttcagaaaaattctcgtatgaagagaattcagattactcaacaagccatgaattgatgctttctgagttcgaatcagagtttttccctaaaacttctagtttagaattctcagattacagtccgtctatgctaatcgattcacttgactattcatctcaagaatccaatataaactgtactcctccagtttacttcccgttcttcgttcaataccagaagcagtttaccaaattgaactcttctacagatatcaaagcctctaatcaagtgcaagaagagttctacgatgaatttacagtaaataaatcttaaactgttgttaaatttaaagcttattagtgttcattttattgtttgattttcagattcttatatttgcttttttgtgaatttttaattcagttgttgagattccataacgaagagtatgaagagagctacaggaagtttagatgcagagagaggaaattggttttacacgactatgctgaggactattcttctataactgaattcggtgatcttgtgctcaagcagcggttgttgatggttaattggatagtggaggtaagaagttctcgaccacgtttctattctgttattaacttttttgagtgttctttctgtttaaatcgtcttcaccacggagtaacatatgttaacttacgcctcaatgcaaagatgacacgtgctagtgaagcatgtttcttatgtacattgataaccattcaccacgtgatgcactattttggagaagtaaactttcttccgagagcatgcgatcttgtaaaaagtaccttcttaacttttttttagttttgttcacactttggtatcagcctaaagtcctaaaccgatgcatgtgggtccagtggtccacgtacaattgacagtggcggttctccacaactaaaatcactaaagcagttttattttctcgatcaattgactacaatgccccttttcttaagacacaattacgaacaacattggagtagtttgtggccatactttcctttcatctatctgatggcacaagtagcttctactatggaagtatggaacacattttatacatggatgatgggaacctttatcgttttgctagaatatagatgtgggttgcatctaacatttactgataaatagcactgtcaattggacttgatagttggattggttatatttaaatggttgaataggaatgtgtgttttaaagaatcaaatagttaccatgtacttttagccttaagcacaaattatgattgcccaaaatttctaccttggtgtacagtactactgttttttggtgacatatttccctctctgtcttaagactcataaaaacattcaaaatgcaatctcttttcattcttccttggcttcctgctccctgtaaattactgatttgttggaatttggtgtgcgcaaccctatgcagcaatcaaatgtactggagcttcactctgagacactgtttctaggggttagcctattggatcggtttctgagccgaggatttttcaaacacaaaaagaagctccaagtacttggaatagcgtgtctcacgcttgcaacaaggattgaagaaaaccagccttacaattggtaaacttttctcaccctaatcccagcagtataatcctcatactatttcagtaatttgatcaatatagttttctacttttcttctgtttgaatggaattatattcccattaagctataaattaccaaatctaggccttgttagttatcaccaaaacaaaatcaaggcgttaagataaaatcactttagttttttctggtccaagattatatttagtgaattaatcatagctttatatacttgatggaattgcagtgtgagccagagcacattctatgtaggaaaaaatgcatacagcaggtctgaagtaattggcatggaatggctagtgcaggaagtccttaacttccaatgttttttgcccaccatttataacttcttatggtacgtcactactcccatttcgcataacatactataaaatgacctgcccttccttttgtggcctggcgaccattcaactgagtttgtgcaccaagagattgataaagagttgcttataggttctacttgaaggcagcaaatgccaatgctgaggtagagaagaggactaactacttggcagtgctctcacttctcgaccaggaacgactgtgctactggccttcaactgttgcagctggcttggtcatactggcttctcttgcagccaatcaggagtcctcgtgtcaatgggtcatggaggtaattttcaaaatacaccatttatcaattagctcctcttgaagtttagattatgagcactatagtcattttagttaaacatgtaaaattcatcattagtacttcctacgttttactgtatttcacttattagtttccgcggcaagtgaacaactaaattagtatatacttagttttctgccatgaaatttgctcagtatggtatccttcaaatgtgaacatgatttcatggatcccaaatgaaggcccaacattctggccatgctgatttcatggtatgaaatctgattgttatacttgtggtgctgacatcaaagatcgtagagagtttcactgacctaatggccgccttaattttgtcatttgcagactcatgttagaaccaaaaatgacgatctgccagaatgcatacaaagcctggagtggttggtgaactatgtccgttag

SEQ ID NO: 2 - DNA, 3026 bp, Arabidopsis lyrata
actgcatcagcccactctctagtctctaactaacgaacttccaatttcaaattcgaaattctaaattctacttttaagcattcgtacggagaaaaaatgaaggagatcgcgatgaggaattcgaagcgcaagcctgagccgacgccgttcgccggaaagaagctccggtcgacgcgattacgccggaagagagctcagatctctcccgttcttgttcaatcacctctctggagcaagcaaatcggagtctctgctgcttctgttgattcctgctccgatttgctcttcgtagctgatgacaacgtctcctgtggttcgagcagagtcgagaagagctcgaatccgaagaagagtctaattgaagaggtagaagtttctaaacctggtaataatgtgaaggaggtgataggcgattccaaattccggaggattacgaggtcttactctaagctatacaaggagaaggaaggagatgagatcgaagtaagcgaatcgtcttgtgttgattcgaattctggtgctggattaaggagattgaatgtgaagggaaacgaaattaacgacaacgatgaaatctctttctcacgatccgatgtgactttcgccggacatgtctccaacagccggagtttgaatttcggatcggagaataaggagagcgacgtcgtttcagtcatatctggagttgagtgctgttccaagttcgggagcgttaccggaggagctgagaacgaagaaattgaaatctccaaaccaagcagcttcgtggaagctgattcctctcttggatcagccaaggaattgaagccggagcttgagatagtcggatgcgtctctgatctcgcttgcactgagaaattctctgacgaagaggtttcggattctttcgatgatgagttatctgagcagcgttccgagattttttcacagtattccgacttcgattactcggattacactccgtcgatttttttcgactctggtagcgaattctctgagaaatcttcctctgattctccaatttcacattctcgctctctgtatctcgagttcaaggaacagttctgtagatccacaattccgaacgatttgggatcttcttgccaggaagaaattcactctgaagtaagtggtataatgatttcatatctcttggaatagttgctagtagttagagattgaagatgtggttatgtgcttgaaattgttcactcgattactagtctgttttgatctgagacttgttctgctctgtgttcaagacttgactctgaaattttgttctggaatgaatcttaagtatacattttcgttttagttgctaaggtttgatgatgaggaggtggaagagagctatctaaggctgagggaaagagagagaagtcatgcatatatgcgggactgtgctaaggcttactgctccatgatggaccgtactgatctcatccctcgtttacgctctatcatggttcagtggattgtgaaggtgaatattatctttctgttcaaatgcatttgcttatatatatccattgatctctgaatgttgaagctcggatatatgtatcagtagcagaatattatgaagagaatgaatgtttggagatcttgtttctggttttaagaatgttttagcctaaggaaatctatagcttactttggaatcttttaaggtttatgtatcagtaagctatgatattcttaatttgttgctgattgtcttctccctgattacaagcagcaatgttctgacatggagcttcagcaagagacattgtttctaggagttggtctgttggatcgattcctgagcaaaggatcattcaaaagcgaaaggactctaatactagtcgggattgcgagtcttactctggccaccagaattgaagaaaatcaaccttacaacaggtactaaccatattccatcttcatgattctaacttccaatgttcattagataactgttctgaataggaaaaagattatgaccattacaagaaactgagtattacactcaaccactaataatggtcttatacacacccttatggtttctgtaagcaattaatgaattggcttttcatgtgtgtcaatattggattatgtttctcaaaaacactctttactgcaaaaaccttctacaacacagaggaagagttcaattttcttcagctgctcacctgatacttctctttttaaatagcatccggaaaaggaacttcaccattcagaaactaagatatagccggcatgaagtggtggcaatggagtggctggttcaagaagtcctcaacttcaaatgcttcacacccacaatcttcaacttcttgtggtaattaaaacctctcttactatatattttcatattccaagatatacacacacagaaagttacatataactatcatttatacatctcaggttctacttaaaagctgctcgagccaatccagaagttgaaaggaaagccaaatccttggctgttacctcgctatccgaccacactcaactctgtttttggccctcaactgtagcagctgcactcgtggttctcgcctgcatcgaacacaacaaaatctctgcgtaccaacgagtcataaaggtatcatcatcagtcccttcaataacactttaataccttttagtatcgagaacatacaagaatcttcacaatcccaaaacctctctttctctccaggtccatgttagaacaacagataacgagttgcctgaatgcgtcaaggtattttcagtaacactctcattatatacgaatctcatttttatccactaaacgtaaggtaagtgactgttttcaaatttgtgtttcctatacaacagagtctggactggttgcttgggcagtaagcaatcaaaaagaatcaaaagccctaaaacaaggacacagtatactccgatacgaacacacggttattattaccatttacaaaacaaacacaaggtaagtaataagaaattcctctacagatttatatacttaatcgagctggacttaattagctctt

SEQ ID NO: 3 - DNA, 3114 bp, Arabidopsis thaliana
actgcatcagcccactctctagtctctgactaacgaacttccattttcaaaattcgaatttctaatttctagtttcaagctttcgtacggagaaaaaatgaaggagatcgcgatgaggaattcaaagcgcaagcctgagccgacgccgttcgccgggaagaagctccggtcgacgcgattacgccggaagagagcacagatctctcccgttcttgttcaatcacctctctggagcaaacaaatcggagtctctgctgcttctgtcgattcctgctccgatttgctagctgatgacaacgtttcctgtggttcgagcagagtcgagaagagctcgaatccgaagaagactctaattgaagaggtagaagtttctaaacctggttataatgtgaaggagacgattggtgattcgaaatttcgaaggattacgaggtcttactctaagctacacaaggagaaggagggagatgagatcgaagtaagcgaatcgtcttgtgttgattcgaattctggtgctggattaaggagattgaatgtgaagggaaataaaattaacgacaacgatgagatctctttctcacgatccgatgtgaccttcgccggacatgtctccaacagccggagtttgaatttcgaatcggagaataaggagagcgacgtcgtttctgtcatatctggagttgagtactgttccaagttcgggagcgttaccggaggagctgataacgaagaaattgaaatctccaagccgagcagcttcgtggaagctgattcctctcttggatcggccaaggaattgaagccggagcttgagatagtcggatgcgtctctgatctcgcttgctctgagaaattctcggaagaggtttcggattctctcgatgatgagtcatctgagcaacgttcagagatatattcacagtattccgacttcgattactcggattacactccgtccatcttcttcgactctggcagcgaattctctgagaaatcttcctctgattctcctatttcacattctcgctctctgtacctccagttcaaggaacagttctgtagatccacgattcccaacgattttggatcttcttgcgaggaagaaattcactctgaagtaagtggtataatgatttcatatctcttggaataattgctagtggttagagattgaagatgtatgtggttatatggttgaaatttcattcgattactagtctatttttgatatgagacttgttctgctctgtgtttgattctgaaattttgttctggaatgaatcttaagtatacattttcgttttagttgctaaggtttgatgatgaggaggtggaagagagctatctaaggctgagggaaagagaaagaagtcatgcatatatgcgggactgtgctaaggcatactgctccaggatggacaatactggtctcatccctcgtctacgctccatcatggttcaatggattgtaaaggtgaattttaactttctgttcaaatgcatttagttacatatacattgatctctgaatgttgaagctcagaaatatgtatcagtagcagaagattatgaagtaaatgaatatttggagatcctgttcctggttttaagaatgttttagcctaaggaaatctatagcttactttggaatcttttaaggtttatgtatcagtcagctatgatattctttgttgctgattgtctgctccctgattacaagcagcaatgttctgacatggggcttcagcaagagacattgtttctaggagttggtctgttggatcgattcctgagcaaaggatcattcaaaagcgaaaggactctaatactagtcgggattgcgagtcttactctggccaccagaattgaagaaaatcaaccttacaacaggtaccaaccatattccatcttcatgattctgacttccaatgttcattagaaaagtgttctgagtaggaaaaagattaggaccattacaagaaactgagtattacgcttaaccaaatcaaggactaataatggtctaatacaaacccttatggttcaatgaattggcatttcatgtgggtatcgaatattggattatgtttctcaaaaacactctttactggaaagaaccttccacaatacacaggaatagttcaattttcttcaactgctcacctgatacttgctctttttaactagcatccggaaaaggaacttcaccattcagaacctaagatatagccggcatgaagtggtggcaatggagtggctggttcaagaagtcctcaacttcaaatgcttcacacccacaatcttcaacttcttgtggtaaaacctctctgactatatattttcatgttccaagacacattatccacacagaaagatacatatgactatcatttatacatgtcaggttctacttaaaagctgctcgagccaatccagaagttgaaaggaaagccaaatccttggctgttacctcactatccgaccaaactcaactctgtttttggccctcaactgtagcagctgcactcgtggttctcgcctgcatcgaacacaacaaaatctctgcataccaacgagtcataaaggtatcatcagtcccttcaataacactttaataccttttagtatcgagaatatacaagaatcttcacaatcccaaaacctctctttctctccaggtccatgttagaacaacagataacgagttgcctgaatgcgttaaggtgttttcagtaacactctcattatatacaaatctcatttttaccactaaacgtaaggtaagtgactgttttcacatttttgttccctatacaacagagtctggactggttgcttgggcagtaagcaatcaaaaagaacaaaaaccctaaaaccaggacacagtatactccgataccaacacacaggttatcattactatttacaaaaacaaacacaaggtaagtaataagaactcctctacagatttatatacttaatcgagctggacttaattagctcttagtataccaattattagtgccaccatttgtgtcgctcatacacatttatttcttattttccctaattcattagactctcatattcttaaaaagaatatttccttgtttg

SEQ ID NO: 4 - DNA, 4041 bp, Brachypodium distachyon
atgcctcccaccatgctcgcaccggtgcccacgaggccgcgctccaaccccttccgccggcggagaggggcggctgctccgctcccagcccagatcgtagcagcggcggcgccgaagcggcccgcggagtcgtccacatcggcatcctcctccttccgcagcgaggtaatctccaccacctcctccaccgccctcgccgcagcccagcgcccggacaagaggcctcggcttcaggacgcggacgaggcgcggcccgcagcctccgagtgttcagaggtcatcggcggcgcaagggcgcgcgccgcggaggtcgaggtctccgagtcatcctgcctcggctccgtcctcgagtccgaccttgcctgccccgagcagctcgccgacgatgcagaggcgactgagtactcttcggcccgcgatgacctgacgcagtcagacgccgaagaggaggttctcagtgctcccagcccttgctccgagtactccctgacccccctgatcgactcctcttcctcgtctgacgacgacgatgacgccgccccctctcccaccttctccctcttcctcgccttcgccgagcaattcgtcccctgcgcgcacaccaaagcgcacgccgtcgccgacgttccgatcccagaggtgagcgggttaatttctacacagttgttctaaatttgtttgaaattgggtctgtttgcaagtgtcggtgcggtgtttcatccgattaggtggcttggtgggaatgtttgtgacaggggaagcggtttgaggacttggacgacgaagagacctacgagcggttccggcgccgtgagcggcggggagtggtggcgtgtgactacaccgaagtgtacatctgcatgccaggcagctatggccgtgccgtcgtggagcagcgtgctgtcatggtgaactggatcatcgaggtcggtttaatactgcgattatcactctggctcgtttgatttttgtggtagaagcgtgccttcaggtttacagtaaaatacatgcatacaatggaagtcacgtagtactctaaattctgtgttgttttgtttgtctgagtccgagtgtccaatatgctctgaaagcacggtagttttgtgactgcgctaataagctgatctctggtgtagatgtttgtgctggcctagtgaggcagcagatttagctatgcgatttcgtgattagtgcagcggcaagttgtgtactatctaagaatttgttgtacaacattctgataagaagattgcgcaactgacattgttcgctgaacagaaggatccccattttttttttggaactgttgttgaccaggccatacttattgcagtactcaaaaggactctgatcaccaattttgactgttagaccatccaagtcaaagagatcagtgctaggatgttttagcaggtgtttgttttgacctttgacatttactatttgaaaaggaatggacaaatagatagttcagttatgctgagaagttattcagtgagccatttgacatgtcatccgcatgtggcctcgacgcctcgtgtgtctggaaagcatattataggagtagcaattaggatatctgcataatttctatgtacatatgcaattcatgagtacttcggtataatcacttatttagcctcctatgaaaaatcttagtttgtctatgcacttgatattgcattgagactggaaagaacttctgataatactgacaccactgtgtcttccacctgaagatttgggtgtcttccaccttgtactgtaatattcctgaaaagcattgtactatgattcctggagcaaagatttattttcagataaaatttcatctacagctgacgaaatactgactgtttcccatgaatttttctagcatgggcatgttaccgatctgcagccagagacagtgttcttggggattggactaatggatcgcttcttgacgcgtggatacgtaaagggcacaaggaatatgcaattgctgggcattgcctgcatcaccctggccacccgcattgaagagaaccaaccatacaattggtaatgagtgcccttgcattacatgttcattccgtttcagtaaaattggtgcatgctatattttctgaggataaagctcatggtatattttcatgtccccactttcagtatactactgtaggttgttggtatacagtagtcagattgtgtcatttgaagtgtgtaccctcttaactgatgcattgctaaatgaaataatgcttcaaagaagctcctcatctaaattcagatcttagttcaacgtagtttcctacttcctccgtccaaaaaagatgtctcaagtttgtcaaaatttgaatgtatctagacatgatttagtgtatagatgcattcaaatttagtcaaagttgagacatcatttgttggacggagggagtattacatatttacattgtgacatggttgtagtacataatactgttagttcctacctaagctattctctgtggtatttgcttttctgttgctaaagctcattgcagtatgatttaattgggaacttgatagaccttagcaagtatccttgggaagccttggtttgttggaactgtcatcgtctaatcacatgatggatctccatagaaacatgtgacaatagttcatacacggtgtttacttatctcattgcaggcttatcggctatcactgcatgctagtatttgcaaattgatcattaatcaacttccatttttatgggttgagcatttcagaaattgactttcttaattgatttacctgtggtcagctagcatcttcagtttagaacacaaaatccattcatatgttatccccactgaagggagttgaaccattgtacgagtgatcctaggtagcataaggtccaaactttttgattgtgcatacttacatgattgttcaagtgaaatcagagcctttttgtggttgttttaaagtttttgagcctgaattcaagtggatctttccttattattaacagcaggtctgaagataataaatcattatgtgtcacacagtagtacctccgttcctaaatacttgtcgctgttttagtgcaaacttgcactaaaacagtgacaagtatttaggaatggagggagtactatatatgcagaaacaatagagtacttaagattaacgtcaacaggagcactgcagcattattgttgaacttctgggtttattgtctatgggatcaacatttgtttcctcattaatgtttctgttcaaaaaatgtgtgatgaggaacctcactatattatctctttcagcatcctgcaaaaatctttcaaggtagggatcaacacttatggccaaagcgaggtcgttgccatggagtggctggttcaggaggtcctcgacttccaatgctttctcacgacagtccaccatttcctctggtactacgtgtttcctgcattcttatctgtgccaagccatacaaaacgaacgagaagctaacagctgactttgttctaatttggcaggttctatctgaaagctgcgaaagcggatgacaaagttgaggatatggcaaagcacctggccttgatctcacttctggaccataagcacctctcctactggccctcgaccgtcgcagcagcagtggtagcccttgcttgccttgccacagacaacgactcgtcctgccaactggtaatggaggtaaatacttaaatccttaaccaaaactatacatttctctatcttttctgtcgtttggaactttgataagacgtactgttcctaacaaagactagaatcaccgaactgtttgctctttgcataacgcagactcacatgaggacgaagaacgatgatctgcctgaatgtttaacggtttggcccctcactcgcattctgatacctggcatgaacttaactgtgttgttcactatttgcagtaactgatgtacggaggaagtacaattttgtggtgccacggcaactaacattcgttatgttatgcaacttctcgtgcagagtctcgagtggctgataaactatgcttcgtag

SEQ ID NO: 5 - DNA, 4477 bp, Carica papaya
atgaagttgaaaacatcgcgaaaagtgaagcgaaagatggaaccagtagcgaacatcgggaagaagctccggtcaaagcgacctcgacggagacgattacatatttctccggttttatgctcctctattctctgtaacaagagcggcttgtcttctctctttgccgattccagctctaacactcacctagatactgaagtttcctgcgattccaacagagtctccaagaatttcattgcgaagaagagagagtttactaaaactgaagaacctggatctagtattctgagaattgaaaggactggcgactggcagtttaggaggatcacaagatcgtactatagacagatggaaaatgagaaaagaataaatgagttggaagtgagtgaatcgtcttgtgtcgagtcgaattctggagttgattttggagtaaggaaccagaagttgaagaaagcaaggagaaatttgacaggaattgagagaagcgtgaacgatgcagtttcggtcatgcaatcagaaatttcttgtgttgagcaagtttccgattccaggaatctggaaacatcactggagaataaggaaaatgatgttgtttctaatatttcaggagtggaattttgctctgaagcggtctttagtattgcagtcaaaggaaacggaaacagagcaccacaagaaggtgaattgtccgagacatccaagccagacaatgccgctgaagccaatttatccatctcaaactcggagtcagttctggagcagaggcaaaagctgatgagctttggatttgattccgatctcgcttgcacagagcagttctcctacgatgacatttcagaatactcggagttgcaggacatttttcaggagaattcagacctcgatttctctgaatatactccatcaattttctacgactctggaagcgaattctcgcaaaaatcatacagtgattctcctccttctatgacttactctctgttgctggaattcagagaacaattctctagatcaactactcccctagatgttgcaattccttcaaaccttgaagaaaattatccaaatcaacctacagtaagattgctagaagtttttcttaatgttgtttttttgcttcatcgttttcgcggtttttttgcaaataaacagagttggcatgatattccaatatgattgatgaattcaatattttcattttcgagtggagatataaactagtttgcgtttgtaatacgtgcttcagtcactttaccccgtattgttggttaaatctggaattctggttcttttgtcagttggtgagatttgacgatgaggatgacgagcagagctatcagatgttcagggagagagaaagaagagaaaagtttttgcgcgactataccgaagagtaccgctgcacaacggagtacggcgatctcatcctccagcaacggtcggacatgatccactggatagttgaggtcagcaaattatccctggttaaatattacgttggttgtggccattttaagtttccaatttttcgttttttgttttttcttttttccccgaaattgccatggcacttgtacacatggtttcacgttcagttggagacatgcagaagcacaagtacagtcagtttggatgatgaagctcaaaaaatttatacgactattttcgtagtctctcttattctaatcatgtcgtgacgagtcatttaagtgctttccgttggtgacgttagcacttgtcaatgaacgtttaggggtgttttggtcatttcgttggtcgaacaaaactggacttcctgtttgactttacatctacacgtatatttgatatcatgaggtgatccatgggagcatgtaactttgtgcctcagaatgtaaccgaagcaaacttgatttaaactggaataccatcggggagccaatgacatcttgagtcagtataaactttggtgtcaaatgtcccctttccatacataacgaatttccgcatattgaaatacaagataactcaaatgagttctaccggacttcaaacaaacttttcttttcagaaattgaatatcacaagaccgtttaagcaaaaaaaaaaaataataataaaaaaaagaaacaggaccagaattctacgtgctggcaggctgaaataatttacgggccagatttttttccttttttagactattaatttgttatttggccatagatttgcaggtttatttggctgtattttcttttgcttttgtcccctttggttgcgttttaccgcatcaaaggagttaaaatatgtacgaaccaaaatgtgcctttctatttacctctgttaaatcctcaagggtagcgcttatatagtgagattatcaatcaattacctaggttacttcattatgtatcatctctgtttttttctctcctctaattagaacttctggtttttgtagcaatgcagtaaaaaggagcttcagcaggagacgatgtttctaggagttagcctgcttgaccgatttctgagcagaggattcttcaaaaacaaaagaagtcttcagattgtcggaattgcttgtcttgtattggccaccagaattgaagaaaaccagccttacaacaggtaaatcatctaccatatgcttgtccatacaatccctaatttcatgctataccaagttggaaggaaaggagtaaggcaaatgcattacgcagaaatggttgtaagccaaggcttattttacaaaactttaagcgtgataggctgtccacctcctttccttgaaataactgggtgaaggtacgtatttacaggttggggtggtctggtaagtcatcctgcagtagactagctatatgcctgacttcaaatagagaaaacaaagcagatacttcatgcatagtgtttctcactaaattgaaagctatagtccattcttatcataaatgaaaccatcttttggcgaaacgattaccaaacaatctcagatccactcataatcatcacgtccatcatccaagtacttagtttataagaaaattatgtctaaattagtaaatttcattcactttagagtttcttgttggcatgttagcgtgcggcaaaagagtttctgcattgggagcgacacgtatagcagaaatgaagtcgtagccatggaatggctggtgcaggaggttctcaacttccaatgtttcttgcccaccatctacaacttcttgtggtaaactctttccccagcatccccagagctctttccgttggaaaaagtagattgattcacctctttgcttgatttccattgctgttacctttagttggaagcttgtgttaaataccatctcgtgattgttcttgctaatatctaatttgaggttccgcaatacatctttaggttttacttgaaagctgctaaagctgatgcagagatggagaagaaggtgaagtaccttgctgtgttggctctgtccgaccatgagcagctgtgctattggccctcgacagtcgcagctggtctcgtcattctggcttccatggaaaacaatcaaaatacatcctgcaaacgtgtcatcgaggtataaattaataagtgtgtggaaacatattcttccctcttcccctcttctttaccgcgtgcttgtgcctccaggtgtcttgaaataagatacattgcatcgagccatttcggctgatctttagtgggtttcgaatagtttggatactgggtactattatgaaacctatggcattatgttctgctacaagatttaatatgactaccgtgattttgatttggtcaaggatcagccttaaattttggaaaagttacatgagaatgttacttctattggatgacgaaggacatgttcttcgtgttttttgccttttccaattttttgaacaaccttagcatctggttagctagaaatttttatgtggttctggactacaagaccctataaacttattgacccatgcagaccttttgtcgggccatatcagctttttcttacgcaaaatataaatgtaacactattaaaaagtgggtgtttcaagaatccaaatcacttgtagtgcaagagtatgcaataatttcccctgcttagcaccggagatgtagacataataggtgttatctaagtaattgattttcagatcttgatcaatctcagctgtttttgcagattcatgtcagaacaaaagatagtgatttacctgaatgcataaaggtatagaaaacagaatcacgttatcattgttttagtttacatccactattttgacataattcattcctatgcgagtagttgatgcaatatatatattttttttaattgcagttgttcattgtaatttctcatccgtatttaagctgaaattttttctttatcaatggtctctgatggccaattttgaaagattaaaaggaactgctttctgatagtctcgtttgtgttgacagagcctgcagtggttgctacagtttgtaagctag

SEQ ID NO: 6 - DNA, 4266 bp, Citrus clementine
atgatcaaatcaaagccgttcatcaagaagaagctccgatcaaagcgatttcgtcttccacgttcgaagatctctccgattgtgttcagtgaaaacaagaccaccaaaagcttctcggaattctccgtcgattcgagctcctgctctcatttcggcgacgaagtttcttcaaacatcaagaagaggcagttcgaagaagtaattgaacctaatgaaacgacgaagaagattcaaggcgacgagccgtttcggaggatcacgaggtcgtattacaagcgtcagatggagaacgaaaccaaagcttatgaagttgaagcaagtgagtcgtcttgtgttctatcaaattctggagctgcttttggagagataagttgtaagttcaagaaagttgaaccaaacgacaattcacgatctgatatttcgtctttcgaacgaaatccggtgtgcaaggacaacaacgacgtcgcttcaataagctctggtgttgaatcttgctccgtggcgaagctaagtgaaagcagagcagtagaagaaaaacttgaactctccgatatttcaaaaaacggtggcgttgattcgaatttcatcgtttcaaaatcggagtccgttgtcgagcaagagacgaggagctctaaatttgattcggatctcgcgtgtacggagcagttctcttatgagaacacatctcagtactcgtcaagtcacgagaatgcattttctgagctgcaattcgacatattcccggagaactctgacctcggtttctcggattacacgccgtcgattttctttgattctggaagcgaattttctgaacggtcgaccggcgataacagtcctccatcgcttacttattctctgttccatgagtttagcaagcagttctctaggtcctctgttcctctagattccagaaagtgttttctcgttcaagaagagaatcagccgtctacagtaagttaaattcttcatctctgtttgctttctttaattttttcttagtagcagcgtagctaaacataattaatccttcgtgttagtctcattttgttaaggttgtttttattttctcatttttatatttttaatcaatattttaatattcctattggtttctgtgtaaactgcataatcgatgaaacaaatacttttggttttattcagctaatttgactattctcttacattactgcttaatttttttgaaatttcgccgttatcaaacgtgcaaatgcctgattttcaaccgagttaattcttaattattctcatttttttactgtgaactttttaatcttcagtttgtgaggttcgaagatgaggaagacgaagagagctaccaaaggtttcgtgaaagagagcgaaggcaaacgtttttgtacgactacgccgaggaatacttttccggtacagagtacggcgacctcatacgcgaacaacggtctcagatggtccactggatcgtagaggtgattacatttgactcatgactagtgatttgcgtccttactgttacgtaattaatcgcctggtttaagttcaatttaatcgtattttcactgggcctggacagcctacccttcagaacactgttagcaagtagattctgttgcagggaacaaacaaaacatacggtgacggttttcacgaaaaaaaaattttgtgtccattcagtctgtggcacgtaatagaatgtagaggtggtgcacagtgtttgttataatttgcaaactaggctggctttatcctcctacttgagagcgcgaaaggcatggcaccgtgtccgtgttagggtatttttgtcaattcaatctttaccctgcttcatgagtgatggagaggtctgcgtgacaaagtagtagcacgtggtttggtgtatgtaggtgattttaaagcattttcctcctagctgagtggactccggggagagcgccaatggcatctagtccgagagtttggttgaaagtagactttttgttactcatcaaaattcttatagaactccattttaattgaaattatttaaacaactaaatacaatctaatttatttcttattttttaaattgaaatttaattaaatacttaaataaattatttgtttaactaacattttacattgactccatttcataatttatatctacactttagttctaataaaaaaatattttttgagaaaattataatggtttaaagttacgaaaataaaactataaaattggaagaaaatacttcaaggttttagattttatatgtattttataaataaaagtatacattttttaagtaaaaaaattaggggaagataatcaattttaatatattagttatattttataaattaaacttagggtataaggagtgttataatgatgttagagggatgttaaaagctccactattttgttaagtttgttgggcaagcctatattcttgtggactttttatatgttaagtggtctttaactactctcaaacccaaaaattagttcaaggaagatttttttcttgcatttataaactcatcacgagcccttccacaattaatgtgaaatagtttagttcacaaggtagtgtttgacatgtagaaatctcaacgtaaagaaaatagataaagtagaaaacaacgagattacatgaatttaaaatattcaatgtattttgctcgcaaaagtgattaattgctttgagcttttaaatcagattctttgtcttctttttttctcttgcgggtatattttgaagtgtttgactgtttattatctcgaatttacaaggccttggattggcatttagtatcaacaaagcgaaatatcagttgttcaattgattttctttcttcattaattatcagttgttgatcagaacatgttactttggattagcagcaatgtactgctaaggagttacaccaggagacaatgttccttggggttagccttctcgatcggtttctgagcaggggattcttcaaaatcaaaaggaatcttcaaattgttggagtagcttgtcttgcattggccaccagaattgaagaaaatcagccctacaacgggtgatgacatttttctctgccgttttttcctatagaatttttaagatcaaaagatatgaataatttgtgcctactttaatatgtcaaatatttggatggagatagctgtactgaaatttttttttatggctcagtgtgcggcaaaaaaatttctacatagggaacaacgtttatagcagatgtgaagtggtggccatggaatggctggtgcaggaggtcctcaacttccagtgcttcctccctactatctataatttcctatggtgtggatctttcctcccaagtatattcataggatgatggatgtttaactttagtttaccgttgagaatattcatagttgttttgattttccttcttagtctataaactatcctcagtcaaagagcttgatagagatccatttattttattcttagtccagttgggaaattgtcgaaaatgtaggttctacctgaaagccgccaaagctgatgcaggggtggataagaaggccaagtacctagcagtgttggctctctcagatcatgagcacctgagctattggccctcaactgttgcagctgcacttgtcattctggctttgctagaaagccatcaggatacatcctaccacagagtcattgaggtaaaaatactaacccgaaagatccagaaagaaaaaaataaaaaaagacattgaactttaaatctcatgaaatagaagatagttgaggccattatcattgattcaggggtgactggagaccatactatgaaactatttgttgtctacaaagcattcaaaatggctactcttatattgttgtttcaaaacgattcaattttcaatctatcgtttgtaaatgatgaaatattgtcctttttagtcaccagtattccattttttttctgctttaatatggaccaactggtccatccacacagtcactcttttacattctgattactcttatctaactttgcagattcatgtcagaacaaaagacaatgatctacctgattgcataaaggtatcaaaataatcccatattatgactttctcatgtgtgttgagagtattaattcggtaattgcagcaatataacaaaatcaccaagtccagatattttgtgattgctgttttttttttttttcaagttaatatgttcactgataatctgtaaactttatcagagcctggagtggttggtacagtatgtaagctag

SEQ ID NO: 7 - DNA, 4069 bp, Citrus sinensis
atgatcaaatcaaagacgttcatcaagaagaagctccgatcaaagcgatttcgtcttccacgttcgaagatctctccgattgtgttcagtgaaaacaagaccaccaaaagcttctcggaattctccgtcgattcgagctcctgctctcatttcggagacgaagtttcttcaaacatcaagaagaggcagttcgaagaagtaattgaacctaatgaaacgacgaagaagattcaaggcgacgagccgtttcggaggatcacgaggtcgtattacaagcgtcagatggagaacgaaaccaaagcttatgaagttgaagcaagtgagtcgtcttgtgttctatcaaattctggagctgcttttggagagataagttgtaagttcaagaaagttgaaccaaacgacaattcacgatctgatatttcgtctttcgaacgaaatccggtgtgcaaggacaacaacgacgtcgcttcaataagctctggtgttgaatcttgctccgtggcgaagctaagtgaaagcagagcagtagaagaaaaacttgaactctccgatatttcaaaaaacggtggcgttgattcgaatttcatcgtttcaaaatcggagtccgttgtcgagcaagagacgaggagctctaaatttgattcggatctcgcgtgtacggagcagttctcttatgagaacacatctcagtactcgtcaagtcacgagaatgcattttctgagctgcaattcgacatattcccggagaactctgacctcggtttctcggattacacgccgtcgattttctttgattctggaagcgaattttctgaacggtcgaccggcgataacagtcctccatcgcttacttattctctcttccatgagtttagcaagcagttctctaggtcctctgttcctctagattccagaaagtgttttctcgttcaagaagagaatcagccgtctacagtaagttaaattcttcatctctgtttgctttctttaattttttcttagtagcagcgtagctaaacataattaatccttcgtgttagtctcattttgttaaggttgtttttattttctcatttttatatttttaatcaatattttaatattcctattggtttctgtgtaaactgcataatcgatgaaacaaatacttttggttttattcagttaatttgactattctcttacattactgcttaattttttgaaatttcgccgttatcaaacgtgcaaatgcgtgattttcaaccgagttaattcttaattattctcatttttttactgtgaactttttaatcttcagtttgtgaggttcgaagatgaggaagacgaagagagctaccaaaggtttcgtgaaagagagcgaaggcaaacgtttttgtacgactacgccgaggaatacttttccggtacagagtacggcgacctcatacgcgaacaacggtctcagatggtccactggatcgtagaggtgattacatttgactcatgactagtgattcgcgtccttactgttacgtaattaatcgcctggtttaagttcaatttaatcgtattttcactgggcctggacagcctacccttcagaacactgttagcaagtagattctgttgcagggaacaaacaaaacatacggtgacggttttcacgaaaaaaaaattttgtgtccattcagtctgtggcacgtaatagaatgtagaggtggtgcacagtgtttgttataatttgcaaactaggctggctttatcctcctacttgagagcgcgaaaggcatggcaccgtgtccgtgttagggtatttttgtcaattcaatctttaccctgcttcatgagtgatggagaggtctgcgtgacaaagtagtagcacgtggtttggtgtatgtaggtgattttaaagcattttcctcctagctgagtggactccggggagagcgccaatggcatctagtccgagagtttggtggaaagtagactttttgttactcatcaaaattcttatagaactccattttaattgaaattatttaaacaactaaatacaatctaatttatttcttattttttaaattgaaatttaattaaatacttaaataaattatttgtttaactaacattttacattgactccatttcataatttatatctacactttagttctaataaaaaaatattttttgagaaaattataatggtttaaagttacgaaaataaaactataaaattggaagaaaatacttcaaggttttagattttatatgtattttataaataaaagtatacattttttaagtaaaaaaattaggggaagataatcaattttaatatattagttatattttataaattaaacttagggtataaggagtgttagaatgatgttagagggacgttaaaagctccactattttgttaagtttgttgggcaagcctatattcttgtggactttttatatattaagtggtctttaactactctcaaacccaaaaattagttcaaggaagatttttttcttgcatttataaactcatcacgagcccttccacaattaatgtgaaatagtttagttcacaaggtagtgtttgacatgtagaaatctcaacgtaaagaaaatagataaagtagaaaacaacgagattacatgaatttaaaatattcaatgtattttgctcgcaaaagtgattaattgctttgagcttttaaatcagattctttgtcttctttttttctcttgcgggtatattttgaagtgtttgactgtttattatctcgaatttacaaggccttggattggcatttagtatcaacaaagcgaaatatcagttgttcaattgattttctttcttcattaattatcagttgttgatcagaacatgttactttggattagcagcaatgtactgctaaggagttacaccaggagacaatgttccttggggttagccttctcgatcggtttctgagcaggggattcttcaaaatcaaaaggaatcttcaaattgttggagtagcttgtcttgcattggccaccagaattgaagaaaatcagccctacaacgggtgatgacatttttctctgccgttttttcctatagaatttttaagatcaaaagatatgaataatttgtgcctactttaatatgtcaaatatttggatggagatagctgtactgaaatttttttttatggctcagtgtgcggcaaaaaaagtttctacatagggaacaacgtttatagcagatgtgaagtggtggccatggaatggctggtgcaggaggtcctcaacttccagtgcttcctccctactatctataatttcctatggtgtggatctttcctcccaagtatattcataggatgatggatgtttaactttagtttaccgttgagaatatcatagttgttttgattttccttcttagtctataaactatgctcagtcaaaaagcttgatagagatccatttattttattcttagtccagttgggaaattgtcgaaaatgtaggttctacctgaaagccgccaaagctgatgcaggggtggataagaaggccaagtacctagcagtgttggctctctcagatcatgagcacctgagctattggccctcaactgttgcagctgcacttgtcattctggctttgctagaaagccatcaggatacatcctaccacagagtcattgaggtaaaaatactaacccgaaagatccagaaagaaaaaaataaaaaaagacattgaactttaaatctcatgaaatagaagatagttgaggccattatcattgattcaggggtgactggagaccatactatgaaactatttgttgtctacaaagcattcaaaatggctactcttatattgttgtttcaaaacgattcaattttcaatctatcgttggtaaatgatgaaatattgtcctttttagtcaccagtattccattttttttctgctttaatatggaccaactggtccatccacacagtcactcttttacattctgattactcttatctaactttgcagattcatgtcagaacaaaagaaaatgatctacctgattgcataaag

SEQ ID NO: 8 - DNA, 4573 bp, Cucumis sativus
atgaaatccaagaaacgaagaccaaaccccaaacctcaatccttctctcctcccaagaacaagaagctccgttctcagctcccacgccgcaaacgccctctgattttaccctttttctgctgttatttggattccgattccccccctccctccaccaccttttcttttgcttcttcttcttctttcactgccgcacaatccacctccacttccttcttcccaaccggacctgaggtctctagccacctcaaccccctgaattttaggaagacacgatttgattcgaacaaggaggttggagtagggagtaatgagcaagtgtctgaatcctcttgtgttgaatctaattctggactcgattttggtgtttccggaccaagcactacttccaagttgaagaataggagaactattcacggaaatgaagatccaattgatccagcggagaatggagttgatgcctcgtcgaagctttgtggaaagggagctgtggtactcacttcttgtgtagagtcttgtgctgaatctatctttcagagtgtttgttcgttcgaagagaaaggattagaggttgaagataacagactatgggaaattcagttacctgagctacagaaaaacgaaattaataaaactttcactgtttcgaagtcggattcgacgatagaacagtggcctggcagcttgaagattgaatcggaccttgcttgcacagagcaattctcttacgatgatgtttcggaatacttaagccagccgttgtcgcttcagtcaactattctattggagatgtctgatgactgctcagattacactccatcaattttcttggaatccggaagcgaattttcagagaaatcgaacgaagacgcagctcctacatcgacatttaccatgttgctgcagtacagacgcgaatttataagcttaaatttctctcacatcagaactagctcgtctattgaagaagaagaagtagatcaatctacggtaattcgctgttttcgtgcttccttttcgtttaacatctgcaacaaaacctggagctaatcaaacaacttattatatatatatatggtttatttaatcgattttaaattagattttgagatttgaagaattggacgatgaagaagcctatcgaatgttcagaaatagagaaagacgccaactgattatttgcgactacatagaggaatatcggtccacaacggattatggcgatttcattcttcagcaacggtcaaatatggtccaatggatagttgaagtaagtcctggatttcaaacctccatgtttctcttaaaaattcctgaattagcataagcaattccccctgtcttccattttcatcgttaatagctttggtattctgagacattagaactgtagagtgtataggcactgtctatcatattacaatttgtactgaattgccaatttgttcttagcatgtcgtaaaatgagtcccctgccttatttgatttggaactttatccaacaatgtgatttactgatgaaaattacaaagtcattactatgatcatactttttactatttaaggcaagcagttcatgattctgcacacatatacacctagatgttacaagcttcagtgcatcttgaattagccaagttcagctgatttttcttttcattttgtacttctacttagatacataatctgtttatttttaacttaataatagaatactgattcataacagcgagatttgtgctcattactgtgaatgttaggattttcttcgaaatactccaacgtagttgcattttcatcatcggttcatggaatacattctttataatctctttcaattccttttcatgcgttaaggcttgtcatcaatcagttggatcaaacttttttacattatatagctttaatttgttgaatgatggcagcgatctagagaaaagaaacttcatcaggagacgacatttttaggagttacccttctggaccagattctgagcaaaggattcttcaaagctgaaactcaccttcaaattctaggcatagcatgtctaactttggcgactagaattgaagaaaatcagtcatacagctggtgacttttgaatctctcttttgtcccatttgtcttcatctcagttttaattatataacaactgtggttcctatctactatgaccttaacttcgttagtctatattatgtatgatgaatattgataggaaaccaaactgtatgccaattggtcttcttgtttcaatccaagggtgtagaattgagtaaagttaggatcaaatggtaagtagtacactagaaataataatcagaaaaaactgtctaaaagtacttgaattcaatagtcttgaatgttttccttgagctcaaagtgccgggactgaaactttttccgttcatgaacaaaataacgttgtgtgattatatcgtagatcctcttataggaaactatgtaacagaaaatagccatacatgttacattagtgtcgatgcacacacctcccgtacggcactgcagtcgaatcctattgccttaacaatatcttaagttcgtaagttaacaactcgtgcacagatgatatccaagatccaccaagaaaacattatatggcaaaccacttcaatcacttgatcgggccatcagacaataaaattctgatcatatagagctccaagtcaagtcagatgtaaaacaattgtttaaaactgttcttctctctctctctctctcttcctcaaacttcctcttatctagtcttaatttatctttgactggtaattttcatgaaaagtgataaataatcatcgtctgtttcattaaatagagctttgagaactgaaagtatgatagtacttatttgtttttgggcaattcaggttacagcaaaggaatatccatgtagggagcaacacgtacagaagatcaaaagttgttggcatggaatggctcgttgaagaagttctaaagttccattgtttcttgccaactgtttacaatttcttgtggtaaatcttcctttcactaacttcaccatttgtgggaagggaaaaggttttccctttcgtgcatccattgctaataaactcctgtccattaacaattcgaatctactccaaataacatgctttatttaaatttcttttgttgaaaattatcttaggttctacctgaaagctgctggagctaactcagatttggagaatcgagctaagaacttcgcagtgctcgttcttgcagaaaaagtccagttttgttatttcccttcaacgattgcagctgcagttgtcatcttggcgtccttaggagaaaaacaagatgcaccaagtgaacgagtcattgaggtacaaaaatacaaatacctttaagagggaaaactcctttatctatatttatatacttatgatttatccaacagaacaaaaataaacaagtaagtctagaagaaaatttgaagttttacaaaaaaaaaaaaaaacagcataatctaagtccaattagattccaacacgtaaagtgcacatataaattccgtactcatacatatactaaaaggaagtgctaggttatagtgttagtttagattacatatcaaattcataatagtgaactttctactgttaatcaatataaatatgaaggtttgtttattataaatttataacagtaatttatgtatttatttagattactcttgcatatttcttactttatcttgaggaaggtttcctgtcttataaaaacccttccatgaccaaaatttcaaccttagactagtcccattgaatcaatggaaggatatatgtccatccttccaaaagaacaagaatcatcgatcttgttcttcaaaacgtagattttaccttttttttcttttcttttcgaaacaaaattgaaaggacattgaatccatgatcacataaacattaaaatatgccattaaagttgaatttgtgaggcaaacatgcaatgagttaaccctttttttttctaattactatctatttttaataagttatttctcttatacactttttgtagtttgaataaaggaactactactaataccttgcaatttctttcgaaatctacaataatagaaataacatgtatttagacatcttgttgaactaactcataacatcgattgtattgtggttttgcagatacacgtcagaacagaaaacgatgatctccctgaatgtatcgaggtatttatagtcaactataaaaaaatcaattgatcattgcataaatctctaattagataaaattttcaaggcttaaaaacacatttttaatagatacaactttttcaagcattaaaaaaggatcaatccaaacaaatccttattttttcagcaaaaaaaaaagtgagtaatacagttggaattttaacagagcttggagtggctattaaagtttctatgatggaagcatgaattcctaagacagcaagaaagaaagcaagcaatttatgttccattggtctttttgacacattcttccttcctgacctgaccacttagacatcatcttgaacacagctagtgaagctcaccc

SEQ ID NO: 9 - DNA, 2879 bp, Eucalyptus grandis
atgatgcagccgccgccgccgccgcacttgcggacacacaggatgctccggtcgaagcttcctcagaggaggcggtctcagttctctcctctcccttgcttctcgcgcaactccgccgtctccgcttccgtcgattccagctacttggacggcgaggtctccgtcgggacggcggacgcgaagaaggagaagaagagcgcgagcgcgagcactcggaagaggcgatccgatgctgtcggaggcggcgattgcgagtttaggagagcgacgaggtcgtcgtgcaagcgacgacgggaggaggaggaggaggacgaaggcggagttgccgaagttgttgcggaggtctcggtgtcggagtcgtgttccggttcgtctttcgaggttgattccaaagaggagagctccgagttgaaagacaccggaacctaccatggtttgaaggagttcagaggaaatgcggcttcggagtcgatcgcgcgatcggagatctcctgcgtccaccagcattcgccggcgaagtccagtgagctctccagcggattgtcgaggaacagaaatgctgaaaatgaggtcgtctctctcccgtcttttgttgactcgtgctccaaggcaactagcggtttccgaatttccgaattggaggacgagacgcttgaagtcgagtgccatgagaacagagcatcgtcggagattttctctgaaacaacaaaaggtcacgtcaccggttgtttactcgcttcaaagtcggaatcgactgtcgatcaaaagccaggctccatcaattacgacaccgacctcgcctgcacagagcaaatcgcgtacgacgacatctcggaccactcttccgatctcgattcattctccgagctacaaaaggagctattcgaggaaaactcggacgaatactcctccgagtacattctgtcgatttcctctgaatctggatttgagttctccgagaaatcagatgaagattcgaatccttccccaaccttctcttttctcctccagtacagggagcaatatttgagatcaagtacccgtgcagacaaccacaaagctcggagtctcagttctgaagtaaatgtaagctcttactgtacgaagctgttattgtacgtgcgtacgattttcgttgtttgaaatttgaatttgattaacttttatgaactcgaattttagcatggatgtggcgacgaggaggatgcggagagctatcagctgttgagaaacagagagaggagagaagtcttcttgcggaactacgttgaggagtacggtgagcccgtgctcaaggaaaggacgcgcatggttcactggattattgaggtgagtcacacgaagatagggtccgagttttcacccctcatctttaattagatgcaagatgtcgatggccaagacattgatccgttgaaatccatttataatttgcataaattgcttgtagaaaccacttttgcatggcatatagataactgcatacattcctcactgttagatccatgcctgtacataagagattgcttcaagctggacaaaacgtcatgcctctatatatttacatggatcctgtttatatggatctttctttccttctgtggtccttcctttgacctcttacaagtttgatattccatgttaacagcattctagtgcgagagagctccacaacgagactttgtttctaggggtgagcctcttcgacagattccttagtaaaggatacttcaaacagcgaaggaacttccaaattgctggaatagcctgtctcaccctagcgaccagaattgaggagaatcaaccttacaactggtaatcagattttatctccttcattttccttcatacagagggatgctaatcaatgccctcattagaaggcaataactgctttggacctttcggttagcaaatgtgaagatgcttagacctcgagctttacaaatcttctttttacttatgctcccagcaatcatttcaccataacttcagacgacaccacatttggaatgttcattggatcgagtaatttcagttacatgtggtagaccgttaggaaatacatcatgaacaacttcaaaggtttgccatcgcaaaggaattaccgatcactcaaccttcactatgtccgggtccgggttgccagtagtgacttacaatcactccactgcataattcatttatggatggtaaacacccaattggttcatgcttagtccctaacagatgtaaatggtactggtaataaccaattactcaaacttccctatgtccgggtccaggttgccagtagtgacttaaaatcactccactgcatgacttcttatggatggtagacacccagttggtttatgccaagtccctaacagatgtaaatggtactggtatttcagcgtaagacaaaagaacttccgtgtggggagagacacctacagcagatgcgaagtggtggcaatggagtggttggtacaagaggttctcaactttcagtgtacattgcctaccatacacaacttcttatggtacagtccatatcttgtctgcgcaacctcgatcggcagacttattttcttttctctcatattgtgtgttttcatgggttaagacttacttttcagacttagcaaattccgaccacttgactaatctgagtaaagaaagtgattctgctgaatattactgaaatatgcagaaatgattgcaggttctactcaagagctgcaagagctgatgctagagtgattaagaggaccataaacctggcagctttagctctgctagaccatgagcagctgtcctactggccttccacagttgcagctgcgcttgtcatcctagcatcagtggaagacgcatcctgcaagcgagtcatgcag

SEQ ID NO: 10 - DNA, 5222 bp, Glycine max
atggcatccagatcgagaaaatcgaagcgcaagctcgagccggagccacatccgctcgtcatcaccaagaagctccggcagaagctccctcgccggcgccgtcaaaacatctcgccagtgctcctcgtcggcatctccgcccagaatcctcgtttctccgtcgattccagctccgtctccgacttcgccgtaggcgaagcctcgtgcaactccagcagagcctccgtcgccggcaagggaaacataaactcgagaaacgaattctcgaccgattcaacgagaaatcggagattcgagaagcggaacgagaacgaagttgaggtgtcggagtcttcgtgcgttgactctgcttcgttcgcgagcgaacgtaacagaagcttgattctgaagtttaaaagagaagataagaatctaaacgaaaacgacgacgtttcggaagcgtgcacgaaatctgagattactactgttctgaagttcaaaagcggaagcgagactaagaatgcaaaagaagacgacgacgtttggtgcgcgaagtcagagattacttgtagcgaggaacagttcaattcaaactcaaagtcctccggtaacggtaacggaaacataaaagtctcttcggattcaaacgcaaacgacttcgtgtcgtttagttccggtgttcgcgcgtcgtcgtttcatgaggaagcgaacagaaacaaggaaaacactaaaaacagagcatcggaatctgaatactctgaagtttctagaagcctccacgtggaagagaattgcgctgatttaatagcgcaatcgatgacgaaggaggattcggatgtatacgacgtcgttgcggatctcgcttgctctgaggatctgcgtttctcgtactgcaacgacgacgacgacgacaacgaatcggagtactgttcgagtcagggaaccgtgttatccgaatttcattccgagcttttcggcgaatgctcgcagaatgagctttcggattactgtccgtcgtcgctgttcgtggattccggaagccagttttcggaaggatccgtcggagaaactccttcgccgacgcatttgttgttccttcagtacagcaaggagttcgcagagctagtttctgctcctccgttgaagaacgcttcaaacgttgaagatgttgtcaatgtaggttttgcttgcttcgatttcagaattcgaagttattatatttatattattattacttgttctgttagaaattattatctgtttagcctagctcttattatatgatagtttgtaattattaaatgttagtgtaatttattgtatgtggatttaagtttgtgagatttgaagatttggatgacgaagacagctaccagatgctgaggaagagggagaggaggcaaggctatgtgttgaattatggtgatggatatttctctaccactgaattcggagacaccgtgattgagcaacgtgcgcaaatggttcactggatcattgaggtaggtttgtctaaaacaaaatccaactcattatattattatatatgctgttactcttgtcgtttgattaatttcacttttatatagtttttgagtaaataagtacgccttaaaaaaaagagtaaataagtacagtatatgttatgaattgtactaaaatttagttacaatttatcatatataatttatttcttcatgatttttgattgattaatgaccgagtataaaaattaaacaagttgattaaggagagctcgtgctttgattattagttagttgttatttttatttatttattggtgtatagatagtcgctctttattgagtcaggttttggatggtgagtgagtgagcgagagagaacaacacgtttgaagagtgagtgagaatcaaacttgattcggttaaaaaggtaaacactttgtacaacaagttgttgggagtaatattgaatggcgtccaattgtctcgtgacacgtcagagtcatttggaagacgaaagcctctacgctagtatcgcggcggacttgagtaaatccctgtatcgtcatgctttttgatgatgcaatcatgcaacgcacttttctcgtattcgtgctcgtgcatgtgggtagttacttcacaagggatgatacattttgcttttcacttagaacccaaaacattgaggagcattggagtagggaaaaattatttccttctagttagtcgtcccccaaaaattacttacattcaatttttgcgagtctagctaatgttacacaagtatagatgctactgcggtacatacacatacactcacacacacacacacacacacatatatatatatatatatatatatatatatatatatatatatatatatatatatatatatatatatatatattacaccagtttcaccattattttaacatttcatatataggtaggatgcattcgacattttgtattggaatggcaaggactaaaggagcttgacagaagatttaattgaatggcaggccacggcctaaaaaaataaaataaacgctacttctaatgaatatgcaattgttatgtttctacaaaataggagtatttcttttttaaaattttaataaaattatttcataaattaaacaaatgaatataatattatttatattttttattatttgagcattgtacatagtgcgaaaatatttcggagcatcaatcaacaactcatgccgtttctcttattaattattaatttattattattattaatgtgttttttatttgaagaaattattaatgtggttattttattgttgttatcatctttcatgatcgaataaccataagaacattctttccttttgatgaagggaatccattttctttttccacctgtttcagacagcgacactaatcatgaatatgtcattttttattttttgtccatataaagcttatgtattttgctaacataccgtcacctaccaatttacattaaaaaaactattgtatgcatgcgaacctatttagatttggataacaatgtttttagattcaggtattgagtctgtcatttctaattgctgcatttgtggactttgaattgccagttgaatattttttaaaatgctttatgtttgaaacttaattggcagcaatcttgtcgaagacagcttcggcaagagactctgtttcttggagtcaacctacttgatcgtttcctaagcaaaggatacttcaaagccaaaagaaaccttctaattgttggaatagcttgcctcacgttagcgaccaggatcgaagaaaatcagcaatacaacaggttcatcatttaatctttacatttcttccccatcctccaagttatcttgggcaaaaatgtcgttttcaatcatccctctctttacttgtatttggttgatcgattgtgaagtgattgttagtgacggtcttgaattaaggaaaatggaaatagattatgatttaatttttctagagtttgagtttatatatcctcattaatcatgcattgtgccttaattaaaagaataacaaattttccctaatggttcaaacattatccaattttcacataaattttattttcttacacatgaactttcttgaagtttaggtaaaaaaaaaaaaccataaagtttgtaagttttaatatcccatgtgtaaagttagccaaagctgcttgtaaaaatttttactttcttcgtttacatattcttgattttcgtacaactagggaaagtgaaaattaacagaacttataattttttcttccaattaaactcatgaaagccatattcaatgaataattgaactcttttgtgggtactacgtacagagtggggcaaaaaaatttctacataggaagcaatgtgtacagtagaagcgaggtggtagctatggaatgggtggtgcaggaggtgctcaagtttcagtgctttctgcctaccatctacaatttcttgtggtataactttttattctttcagcacgaatatgacctgaaattctgcaaaaattaaaggttagctaataatcgttgttcaaggtctccattcatcattaaaaaaaaaatctgtttcttactgggaaacaacttgaattttaaaatgaacggttgaagcaaagacttgctatcatttctctaataagggatttctttttgccttaaaaactctgcataatttataaaattaaaacaaacttttgtaattgtgacttcacatagcctcaataagaaaactcataagcctttttgtttatgaattcagctaaataacactccactctataatttttcaggtattacctaaaagcagctaatgctgatgcagtcgttgagaagagggtcaagtatctggcagtgctggcactgtcaggtcatgagcaactgtgctactggccttcaacagttgctgcagcacttgtaatcctggcttgtcttgaattcaatcaaatttcatcccacaaagtcataggggtaattacattattgcgatatagaactaactctttatttgaatcttaagttaacagagtcagggaggttaatcaaatgacacgatttggcaagaacagatttgcttcatagatatcagtttaatttggtcattccttgatattttaataaatcattgttattttagtacacaaaatcaatcaaaatggaatacaatagtatttttatggtctagattggcactggagtttcaaaaagattggcactgaatgcagagttgtgcgtgcattcctttttcctgaatgctcaatgtttctattttatatctttattttctttagattggattatcctgtaatgtccatattagattgtgaatagaatgccgtatagataattaatcttgtgattttgcagattcacgttagatcaaaagatgagaatttgtacgaatgcatagaggtatgctagtcctatatatttattgtaatttgtcagtgtacatttttttgaggtttaaactaacaaaaagaatagagaaaaaaactgaaataccataatgaaaaagcgcctcacactacaagtattatgaaattaatcaaatcatttcatttattttgctgagaaaaaccccacaccacgattaggatcgatgaaatatatcattttcgttaattatcattcaatttctcctattatcagtattgttcatgtatttaacatgaaacttatattcacttcaacagagcctggagtgggtgttacgctttcctggatag

SEQ ID NO: 11 - DNA, 4302 bp, Manihot esculenta
atgaagctcagaacgactaaactcaagccaaaactcgatccacatcaatacatcacgaagaagcagcgaaccaaacgtcctcgccgaatccgctggcagatctctccgattcttatatctcgattgagatcagctcctccacaaaagagccaaggtttccatgctttctcggtaaattccatccccagtttgtactttggagatgaactgtcttgcgattcaagcagagttacattcaaatctagtgcaactaagagaaaattacgtgaaaccgaaggaattgagaagattgaagacgatccgtttcgaagaataacgagatcttactataagcagaaggagaacgagagaaaggaaaatgaagtagaagtcagtgagtcgtcttgtgtagagtcgaaatccagagttgattgtgttgctcccggaaagagaagaagttcgaagttgaagaagagagctgaagactcaaaagaaattcaaattagtgaaggttctacatcggttaccaaatcggagatctcctccttgcaccaaaatttaagtttcaatggtaaaagcttggagaacatatcgtcggagggtaaggacaacgacaccttctctattgtttccggtgtagagtcttgcttatcacatggaacgattgaaagagtcaaaagaacagaaacggagctctctgagatttccaaacacgatgccttctctatcgacgagtccgtagtggaacaaaagccaaagagcttgggagcagtcgaagcggatctcgcttgtgcggagcgcatctcttacgacgacgttgttacggaatactcttctagtcatgagacggcgttctcggagctgcaatctgaagtgttcctggggagttcgtccgacatcgaattctcagattacacgccgtcgatttttttcgattctggaagcgaattttccgaaaaatcggttgatgattctcctccttcgcaaacgtactcattgttgctcgagtttagacagaaattctcgcgatcaagcgttcccctagacatgattagatgcccgttcactgaagcagagtacctattacattccagcgtaagttctctccagttttttgaattaggcacatcgttttctgatttcttcaggaatttcccagtcatacaaacagggccgtttggctagtttcttatttaatgattttaaaatcaaacataatttctatagtagttttagtttaaattctcttttttagtattccaattagcttcgatctctatatgtctgctgccatttttaggaaaccgtagcttgccctcctttctaggcagccaaacacattgattttaatatgtaattgttcaattgccagtttgtgaaattcgaaattcaggacgacgaagagagctaccaaaggtttcgggaaagagaaagaaggcaattattcttgcacgactacgtcgagttgtattgctccacgacggagtacggtagtctcatcttagagcaacggctccagatggttcactggattgttgaggtgagttccttaccagcctgataagtggtaacttattcatcggctagctcatcggcgtatttaacatttacttgcgctcatgcggcgattagagacagtttacttagatgtcccaatatttcacgttcatagaacgtgtaagatgatagctggcaagttctttcggttataggagcaaatattctctacggtaacatttaccagagaacatcccatctacttgtgacacgtcacagaggaagggcatctggtgttgcatgtagagctttcatactgctcatcatgtgccctcttctcgcagagcttgacatactgttctagtatatgaaggacctgactggatcactttgcaatagaaaacttgattctcttcttctattggttatgcaagagaactattagaaatgtttctggagagatcgcttttgttctttggcaacttgaactgtttcgcgactgaactagttgcgaaatgctttctcgtttttcaacttagtgaaaaacacgtaggacgaaatttgtacctccatggtatacgttagcactattcctataacccgaaagacttgcaacggcattgttttttggcatgaccattaccaagcgatttcaacgagtaaaataaagaatttaagagtaactctaatttccccatggttgggagtaaaccataagcatttttccttcttttgggtgggatttgcggtatttttattattgttattgttgttgttgttattattattatattattattaatggattgtctattgcattatcagattacatatagtattcattcatttattgctgcatgttactttgggtaagcagcaatcaaccgcaaaggagtttcagctcgagacaacgttccttggagttagtctactggataggttcctaagcaaaggattcttcaagaacaaaagaagccttcagattgttggaatagcgtgccttacattggccaccagaattgaagaaaatcagccctacaataggtgattatcttaacttgcctcaatttttttaccactgctgtggttaataattaaggattgaaaataaaggtaccagctagcctttgttttgttttgttttatttttatcgacagctagatttgatcagaagcaagattttcttcacaattattaatatggatcatatcatgcagaaatcaaatatcacaaaattcaataggagaaaattgaatttcagcttataggtagattatgccaagacaaaaatattctttctgtgattgatcgacaatttttcgctcaatataatcattttatgcccttctcttaattccgaaaacacactattatttagaagacattaattatattagttttagacgtttttccgtgaacctgctataggaccgcggctgaaatgaactcgtaatccaaacttgactgatcaacatgaaacatcgtgtttatcatcctgattaatccgttccttcgaatgttctcctacttatttgatggccagatcacaagcgaatgtcatcacttaaagatcaaagataatattatagctaaatgaaaattttgtactggccaatatgggaatccatgaacttacttttactgcgggcatttcagtgtgaggcaaaagaatttccccatggagagcagtgcatacagcagatttgaggttgtagccatggagtggctggtgcaggaggtactcaactttcagtgcttcttgcccaccatccacaacttcatgtggtaatttcctttcccattttatcaattaactagtactagattagcatcttatatacagaagcaaacttgaattcctcttgtaatttcataaaggcaaattttgttggaagagtttaatatattacgttcgtattaacggatcttaggttctatctgaaagctgctagagctgatgcgaaggtggagaagaaggcaagatatctggcaaagttagctttatcggaccatgagcatcttcggtactggccctcaacagttgctgcgggacttgtgattttggcttctctagaaagcgatcaaattgaatcctaccaacgggtcattgaggtaaaaatacttaagcagagcaacaacgtatccttgaatttaggcccggtcagtaacttcaaaattaggttgatttaggttttctttttttttttttttaattcaatctgatacaggatgttaacacactaaggattcatcctaggcaactcattactgtttggactagcattactgatccattcttatattgtgaagaatacagtttgacattctaattaattagtaattttgcaggttcatgtgagaacaaaggaaaatgacttgcgtgaatgcatgaaggtaaaaaggatacagctatgacataaaattatatcaatttgtcgtctcctttttacctctcggtttggcataattcatttgtaaggaaagaatttaaatgaatcttgaaaaatcaaggcatagttttatttcttttcaaaatacaaaatttatcaattaaaaaaaaatttttcattcaaacaacaaaattgatagaaaagaaattgaattgaattcttgtaaaaaaaaaattattttaaacaaggagtacagcaatctatttaggattttgacaaaatttatatttatatagaaaggttttgatgtattctaactggtcacaattctcaaaagcatttccaaaacatgtttttacaggatcgttcttaacttttttaatcatatgttaattttggcagacaatggagtggctgctacagtatgtaaactag

SEQ ID NO: 12 - DNA, 3926 bp, Mimulus guttatus
atgaagcgcaagttagcagcacgagctggtagatcggcagccgaatcgccaccagccaaagagcgatcgctcgacgtcgctaggaagcagcgttcgcgtctctctcgccgccggcgacttcacatctctcccatcgttcaacaatgcgttgccagttcacatctcacggaattaagtgaagtctcgcacgaatctagcgtcgcctctgtcggcaaccagaatccgccggagttccggagagttgttaccagatcgtattacaggaagaaatttaagaacgtgagcagtaattgtgaagcggcgccggagttatcggaaaattcgtgcgtggagtcgtgctcgggagcaaacagagagttgaagcagaagagtgcggacgtggaggaattagaagttgaaggagaagaggttacaaaacctgaagtttctagcacttcgcgattttcattcgccggaaacgacgtgggcaaagtgacggaaaatgagagtgccgtcgaaattcctgacgttcagagcatttttcatgaagaaatcacgaattccaaagctgaagaggataattcatctgaggactgtactctatcacttcactcgaccgagagaacagctaaaaccagtgaaaacagagctgcctccggagatctgcaatttccaaagctcgtcgcgtttggcattaatttggcttgttcagagcagttctcgaacggaggcgttatcaatggcggtgaagaagacgatgaggtgcatagctcctcgtcatcagagatttaccatgtgatttccgattcggaattcacgagttcagactatacgccttccttttggagttatgcttctggaagtcaattttccgagaagtcagtcggagaagacaattcttcaccaacttatgaattgttccgggaattcaaacagcagttcttcagatcggattttgccttcaaagcttttgacgatcacaattctcatgaaatgaatgtgagtttctcataattccattatcgtataaaaatctgttcatatttgccgttgatttatttatcagtgctatattttttaattctatttatattattgataatcaggatttaggattagaagttataattatacctaaatcaacactaacaactgatgacgtaaaattttacaattagtagaggtttacacgagcaaatgaagctggaaactcaacttttccttccgtttgatttcgaaatagaattttactcctataataattattttctttgttgattgcgtttctgtattaaggtattggggctagaaaatgaagaagaagaagagagctacagaatgatgaggaaaagagagaggaggcaagagtatgtgcgtgactatgcagaggaatactgtaataatactgcttatggtgaacttgttatccagcaacgcttacagatggtccactggatcgttgaggtcagctcagtcaatctctactgtttctgtactttaatctttgtgtagtaattaccagaattgactatatataatctgatcgaaatcgaaattaatgtgaaatcggagaatattagtaaaacgtagacaaatttatggatcaaattggttgttgaaatggagtaagtagactgattagcatgctaatgttttgagctaccatagcgaccttgaacgtagattttttgttgtcgtatttaaaattaaaaaggatattaaaaaattggtcatggtacttcaaatcatatcgtacacttaagataaagatctggctttctaatttgttttgaatgttaactcaatataatgacaatgtaatggaataattagtagcagatgttcagtagtaaactgctacttgaagaagaggtaatctaaacattttaggttggagtttgtgtgatgcatgaggtaccgtcacaagagtgtgctcgttggagtagtaacttttacagatagtgaaaacatttcaattttgttacacaattcattcgaaaaaagaagaaataaaacgagtaaagaagaggaaattgttgctgaacaatggcattaaatgggagaataggttacacgtttaatcatacacccccactcgcttctcatacagaaaaatcatattatttattagatttattaatctgatcttgatatttgatcatgcagcaagctaccaagaaggagcttcagaaggaaaccatgttcttaggagttagccttcttgaccgatttctgagcaagggatacttcagaaacgtgagaaatcttcagattgctggcatttcgtgcctcactctagcaaccaggtttgaagaaaatcaaccatataactggtaatgatctcaataccttgaatcattctgttttcgttgagatgggaatgcactctttgaaaatcgttttatgttttagattattaagatgtccttgcaaattagttcgacatgagaattgttactgcatcaataacataaaccagaaaatgtgtatcacatgatgaaacaattgtggtacaagtggtaaaattctcacgagtaaaaccttttatgaaacaccattataatgcatgtctatattttatcccattgccgtgaataatgaacacgtggtgccagaaaaaaaaaagtgataataggaacatatggaaaatgcttgtgggctatcaaaataaatgctaatgttacaagatcttattgatttgattgtatgctgttttttcaatcgtctaggattttcctcggttattaagatgcttacttaccaagttaggtagttacttggatagttggaaaagatatccagtcataattttcacaatgccttttactttactagattgtatccgtgtttagtaaaagatagtatttatctctctccttataatccatgacacaggaactaacttcgataaacgtattctgtttactcccattgtctctgaacttttagcaagaaattattccacgaaactgctgttggataagcttcaattacatactgttaccatcaattattaatgcaaatgcttgattatttattggagtttacaatttcacaccagtgtacgtataaagacattcaacgtgggaggcactatgtacagccggtgcgaagtggtggctatggagtgggtggtgcaggaagtcctcaacttccaatgcttcctccccaccttgtacaacttcttatggtacgcctttttattctctcagtcatccatgtaaatagtatataacctctttgaatgcaaattattttcgtgttcttatggtacatgtttttttttcttcttttttttttagtatgaaaattgtttatgtgaatatgtaggttctaccttaaagcagcgagagctaacgagaacgtggagaagacaaccaagtacttagcaatactcacactcatgggccatcagcagctctgctattggccatcaaccgtcgcagctgggcttgttatcctcgcctcaattgcagcaaatgaagatgcatcctgccacctagttacagcggtatgcatgcatttataatatcaccgggtttttttcccctgtatattattttagccacttgaatatacaggtgattattccaaagaataaatatagtttctatcaatcaaaaaaaaattaaaaaattgaatttacagcataattggttggaattgatactgatgaatccaacaactcaattgtgcagattcatgcccgagaaaatgataaggatttacctgaatgcataaaggtaataatatatattctgcttcaaattttaattcgctactgcgtgctaaatcgaacgtgtaacagttgtaaatttgccgtttttcgtatcttatatcttttttttgcagttattcatcctgaactaaataaagctaaaaaaaatatctacattatttttttttctgttttacagagcctggaatggttggtgaagtatctctga

SEQ ID NO: 13 - DNA, 5639 bp, Oryza sativa
gtaggcccgtagcatcacaaattcacaattcacacacaaagcacacagcgccgcacgccaatgcctcctaccatgcttgcatcggtgccgacgaggccgcgctcccatcccttccgccggcggcgaggcgcggcggcggcggctcctcctctgctccctgatcagatcgcggcggcggcggcggcggcagccaagcgacccgcggagtcgtccacctcggcctcctcctgcttccacagcgaggtcatctccgccacctccaccacctgccccacctccctcgccgcagctcagcgcccggagaagcggcctcggtaccaggatgtggacgaggagcagcccgccgcctccgagtgctccgagatcatcggtggcgcgaggccgcgcgccgccgaggtcgaggtctccgagtcgtcgtgcctcgcctccgtcctcgagtcctacctcgcctgccccgagcagctcgccaacgacgccgagacgaccgcctactcctcagcccgcgaggacctcacgctgtcggagaccgaggaggaggaggaggaggaggaggtgcgcagtggtccctgcatctgcaccgattgctcgtttagcccccttcatgagtcgtcctcgtcgtccgacgacgacaacgccgtgccctcccccaccttctccctgttcctcgccttggcggagcagttcgtccccttcacgcatcccaaaacgcccaccgccacggatgtcgctctacaagcgggagaggtaagcaggttccacacagcttctctcaaatttgttggagtttggctgctctctcccgaatcagtgcgacatttgatgtaatcagatgggaattattgtatcaggggaagagatttgaggacttggacaatgaggtgagctacgagcggttccggcggcgcgagcggcgaggggtggtagcccgcgactacattgaggtgtactcctccatgctcggcagctacggccgcgccgtcgtggagcagcgcgttgtcatggtgaactggatcatggaggtcagtatgctctgcattgtacacgtatgctgcgaccatgactttgccttggtccaataattcatctagctcgagcgcaatttgttgtgtggtagctgcagatgtttgttgcacgggtattgtgctgataatgcacatgtatactttcggagtgctgtactgatctagattctatcagcttatctgtctgactgatgtgttctgcaagaacagtagttgcttgcattttgcactaacatgctactctgcagtgtaaatgctgtgctgggtgggagtgctgaggtagcagatccggttgtgatcttgcatattttgtgtaggggaagctatctgataccaatatacgcattgcatttgctgattcgtttatcacacagttagctgcctgtacattttgcagcattccccgtgttagtctgccagcagtgtggtggtaatcctagtgatttcaatgtaactctagtcattaacttttatgtcatcgcattcaagttctatagagcagtgataaagattttctaggtgcttattttggcatgcttgcatattttttttggatgtaatggcatgcttgtatgaaaagaaaagagatacataggatttttctaggtgtttattttcgcacctgaaagttgctctgtagtcatttgccatctgaaatcccatggcattgggcattggcatatggcctccagaaatgtctgcatcactttttacttctaaacctatatatgcaataagtgttagaaaaacatattgccagtgtattccctttttttgtgtatccaccatcatctgaatttaatcttttctaactctagttccatcagtttatatttgcattgaaacttcaggtagcctttgaaaatgaaatcgctgtgtgttttttgtttgaagtttttttgggcaactcagcgataagtacttaagtgatatgatcatttatacttgtaatttgatttttctgaaaataatgtactgtcatttgtggagaatatgtttgttcttacatagagttcatacataacttgcaaagaatgacttgttctctatgcatcgctagcattcacaagcgatgaagctgcagccagagaccgtgttcatggggatagggctgatggaccgcttcttgacacgtggatatgtaaagggctctaggaacctgcagttgctgggaatcgcctgcaccaccttggcaacccgcattgaagagaatcaaccctataattggtaatggctcccttaaatgaccagtttcagtatgaatacttctgcaagttgttggtgtctgcttgtctggtactcacctaaatagttcgtctcatttgaatatagcatgtattattaaatgggatagtgctcaattgcaatgctgcaaattccttagtattctttgatagtttgatttttctgaaagttgaacttaattctaacagacattagcatgagtttatgagtttaattatgttctataaataacttaataacaacaggaacatccgatggtaactcatatctcatctagtagctatttatcactttcaatactgagcactgcatgtgagaactcagaccattggaccattgttctacaaaaaggtttcacacgatgggtattttagaaatgaagggcttgtttggttctaagccattgtgggccataccaattttttggcaatggcaagatttagccactccaaaatcttggcaaaggacttgtttggtttgttgacaaactttttggcaagattgattagtttagtatttagtttgctaccaaggaaaaatattggtgttgccaaaatttggtgacaaaaagaaagctaacaaaattttaggcagaccaaaatattggtatggttttgattggctgagaaccaaacacaccagaagtttcttgattgtgttgactatgatcaagcatctctcaaaataatttaccgcgccagttggcatccttgagctattgttttttaagaaaaaaagaactaagctatcaattattgggagagatgcgattttgagaaattatcacttgcatagatctccttgatatagttttatttctctgtttttgatgttacaataaagctattacatcagcgctctggtgggcgagatattctctgtgttttcccttaagttttaaagactaaactcatatgaatcttcaacatttttgaaagtagaccttatttgtatccctgagaacttgctagtaaatgaatgttttatgtgccacttattactgcacagaagcatgtgaagtgaacccaggaactattcttctttgcctgtctactttgcacgaagtatcacttttgaacttatgagtttgcctctggatgcgcatttgtttcaagcgcctagtttctgtcaagggcaatgtgcaatatagaccttttctttagaacactctacaggaacctaataatacgatctctttcagcatccttcaaaaagctttcaaagtagggatcaatacttacagccgaagtgaggtcgtcgccatggagtggctggttcaggaggtccttgacttccagtgctttgtcacaacaacccatcatttcctctggtactatgcctgtgtgtttgcttcattttctgtgtcagctggacagaatgaataagaaacttacaattgtttggttcaactttgcaggttctatctgaaggctgcgaatgcagatgacagagtcgaggacctggcgaagtacttggccttgctctcacttctggaccacaaacatctctccttctggccctccactgtggcagccgcagtggtagcccttgcttgccttgccacaaacaatgagtcatcatgccatttggttatggaggtaaatactttaatcttccatcactggctatgctatttctcttatatctgcagtctgctattcgttcagaacttccttaaggaaaaagattctgaacttttgctcagttttgtatgttgctgctttcattttatctcgtagccagatgacaaatgccatgaatcacctaggtcttcacatgcttattccaattcacaacatactgatccttgcaaagaatacaaatgtctaaccacttgctcttcacataaatgcagactcacatgagaacgaagaatgatgatctgcctgaatgcctaatggtttgttttctcctcaaattatgattctggtgaaaagtgttgtacacagtttgcagcaatttgactattacccggatctttagcgtgccatggcatataacgtgcagtaatccttttatgcagagtctcgaatggttgaccaattatgcttcctgatttgaacaaacccaggtgatatgccgacccaatcttctgcccattcccagaaacacagtgtagtacgtacagagtcgcttaggaggaggtgtatatagtacacgtgcactgtagctaattcttcgtgcatagcattagtttgacagcagggtagaaaagagggcaaagaagccggctaaacgtggttgtgatggcaccagacttatagggagcatcgcaaccacgaaattttgctactactgccggcttcagtgactacgactaacctcctgttgtaagctgtcttaaaatcagccttcgttgtgttaagggcaaccaaaggttgagactttgttgttctaatcaaacatcctgtttacaaattgcaactggtgccagaatgaaatttgttctccgggtggtattgcactgcacgaggttcgattctgtgaccctgaaagacgcatactcagatactctgatgctcaggtttcagattctgggatgaataaaatgctactagtagtatcatcttcttctggatctgcaaacagatgttcagttagctgaagatccagagtaccacctcgtttgctcaggtacatgtcatcaaaacatctactactaatctctagttttattctctagacttctctattccaatctatctccaggatgcaacaggaagggtctgcatccaggtgggctacctgaagtggacgaagcggtcatcttcgtgccgctgctcgagtggcctagtggggaccccggctgcgcccgatgcctcggccactttaaaaactcggatcgcgagagttcctgcacgccataaaaatgaggaaaacgaggtggggagggcgtggcttcttttctctatgatgaagtgcgtggcatttgttgagccatcctaatcagagtgttgttcatgcatgtgccccgaatgttgggggcgtctgcagaaagaagggagggcgttcgcgtacacccccctttcagccccacttgcgcccgcgctctcgttgagccgtctgccttttggggttctagagtgaatagggtcggcacgttagcggcgagccggcgacgggtaatgtggtgtgccaacaactttcagctgctgctgaagtgaagagcaggtggcgatcaaccagtcccggcactgaactactgaacctgaagtggttactccattcatcgacagagatatttaattatttgtcacttttagatgtaatattttaccacataaacctacaaatcataaacatataagtgacaaattatttaacgccacatcgaaaaatggtaaatgcttatttgccccttcccactctctc

SEQ ID NO: 14 - DNA, 3512 bp, Populus trichocarpa
atgaagctcaaacagagcagcagcagcagcagcagagtgaataagaaaatcgagcgagaaccaccgtgcatcgtgaagaagcagcgaagcaagcgtcacttacaagtccgctctcagatctcaccaattttactctccactaatctctctctcaataagaacaaaggagtctctgtggattcgatctcctgctctcacttcggcgatgaagtttctagcgattcaagccgagttacagttgaatcaagtctgatcaggaagagaaagttcgaagactcaccgtttcggaggatcacgagatcatactacaagaacaaggagaaagagaggacatcattcgaagtagaagtgggtgaatcttcttgtgtggaggattcgaaaaatagtagttccaaaggaattcaagtcaattacacttctaccttatcctctcttcaccaaaatctcaattcgaggaaaatttcctcggagattaaagaagatgacgccgtgtctgtggcttcaggcgtggaatcctgcttatcacgtaaaacgacgacaaacagagagacagacctctctgagatttccagaaacaatgcagtttctgttaatgagtgtgttgtagagaagcagaagaagccaaacagcttgggaggaggaggaggagaaagtgatgacctggcttgtacagaggagttgtatgtggacgacggagtttcggattactcgtcttgtcaggagacgttgttctcggagctgcaatcggagatattccgggaaaagtattcatcggacgacctcgatttctctgatgattacacgccgtctattttcttcgaatctggaagcgatttttctgagaagtccgtaagtgattcgaatccttcgcagacttattccctgttgctccagtacagacagcaattctcgcgatctagtttacctctagaaactacaaaatcatcgtcactccttgaagcagagtatcaagagaatttcgccgtgagtttttgaatcaacaattacttttgttttcgtgttattttgctatgttttaatgctctcattgttttttaatttgtttaatttctgtgcttttcatttgtttttattgcttaagtttgcgagattggacgatgaggaagatgaggagagctacaagagattgagagaaagagagagaaggcaattgtttttgcacgactaccctgaattgtaccgtaacaacacggagttcggcgatctcatcctccagcaacggttgcagatggtacactggattatcgaggtaagttttttataattaatcgcggtagctaacaaatcaattgctcggatgcggcgattttgcgacagttggctatttttttcttgtcaatacacacacgtttgaagaagttgattccttcaattatggaagatgtcctctacactggagattctactactggaagtgttaagcaaatggggttgtgacacgtcatagtttgagattttgggttttggactttaaaactgacgggtctagcaattgtatttgtaccatttgtggtatggtcagtactcagtactgtccttgtactagattaaactagattttctctgtggctcattttttctttctatttactaagggtatttttatcatttaaacttgggctgtttgtagtggtttcaaggcactaaacactaatcaacaacagtggactttcaattgacaatctgtatgagcaatgtgtgccttatgttcttctaattaaatggtaatcacttatgttaaaaaatggtaatcacttatgttaaaaaaatggtaattgcatgttgaattcgtgtctgttgctttgcagcaagcaactgcgaaggagtttgacctggcgaccgtgtttcttggaattagccttctggaccggttcctagcaatagggttcttcaagaacaaaagtcaccttcaaattgttggtatagcttgtctttcattggccaccagaattgaagaaaaccagccctataactggtaaatatctctgccccgttcttttttgtggtcgtgatgtcctggattgcttaaggaataaaataaaacaaggtgccagtcttggatgcataattctttccttttagtcctttgcatactaatggcttgcatttacaacatagaattgtcaaaaaacgattaggatcgtgaataaacttgttgatcatcttaagaaacagatcacaagtgagtgttgtgtggttattttttatggacttactagaaaagaaaaattgctgtcaacttgttgccagatcaaggctcaaatgaacttgaacacactggatgaactatactttaatttcttaacgtttgcaagtactaagacattcctataccactctccagttcattctttttcatggttatataatctgcctcattttcatcaaagctagctgtccatcacttctcaaaactgaaggtaatatgatatctagctgcaagttctgctccggtcaatgagagctccgctgacagttttataatgggaatttcagtgttaggcagaagaatttcaacattgggaacaatgtgtacagcagaagtgaagtagtggccatggaatggctggtgcaggaggtccttaacttccgatgcttcttgcccaccatccacaatttcatgtggtaactccttttccctttccactttgctttatagtttatggtggtaagatttgtatcctgtcttggatatgaacactttagttctttctgtcattcatatgacctgtagtttaggaagtctaatacaaccttgtcctttttccatgctgaacattaggttctacctgaaagctatgaaagctggtgcagaggtggagaagagggccagatacttagcagtgctggcactgtcagaccttgagcaacttaggcattggccctcaacagttgcagccacgcttgtcatcctggcttctctagaaagcaatgaaattgcatcctatggacgagttatcgaggtataaatattaattggcaacacaagcatgccgtgctcgaataccaaaataaaacgatttgggctgttaatgggtggaggaggtgctgttgcttgtttcatttaatatttttggacaagatttgccgtatgatatgattcagtgaaatatctgaaaactgtcctaggacatactttttccccgggcctggccccggcccattcctaaagtgtactgcaattaacatgctcttaggttctgattagtcaatttgattttacaggttcatgtaagaacaaacgaaaatgacctccaccagtgcataaaggtatgcaaataaagatcttttctaaaacggtataattgtaccctcttttttatgcaattcttttctaggaaggcgctgctattgatgcatttacattcacattcacattctcgaaagctagtgccgctggtgttgattttgcctttattgttaatttgattcgaattatttgactacggtattaacagagcctagagtggttgctgcaatatatgagctag

SEQ ID NO: 15 - DNA, 4405 bp, Prunus persica
atgaagtttaaatcgatccaagcgatgcaaaacctccaaacggcgccgtacctcaagacgaagaagaagctccggtcggagctccctcgccggacgcgatctcagatctctccggttctgtactcgtctctcaagttcaatgcccccagtgagacctctggtttctcttccttctctgtgaactcgaactcctgctcgtacttcggcggtgaagtttcgtgcgagtcaagcagagtttccgttggatctgagagtgaggcgaggtcgagtttgcggaagagacacttcgaagaaactgagaaacaccgtaaaatgctgtttcctagagctactagatcgtattatggaggaaaggcggagaagaaagaggtcggtggagacggcgaaccagaggtttctgagtcgtcgtgtgtggagtccaattctggagctgattttgggggtttcgtagacagaaagttgaagctaaagagtaagagaggaaaaggaagtgaaattgtgaacgaaaatggaggaaacgaaggttctgaagcagttagtagaccagagatttccgagtccgataaggtctcattggaattcaaggaaaacaaagttacgtcattcaactctgtcatcgaattgtgctctgaatcgaaattaccagagaaaacggtcaaagacggagaaaacagagcaccggaatttgaattccctgaaatttcggaaaactacttcggtgaaaatttcgcgatttcgaactctgaatcgacgatagagcagaggccagacagcttgcaaatcggttctgaccttgcttgtaaggagcagttctcctacgacgacgtctcagagtactcttcgagtcagacattgtcggagctccaatcgaatattttcgacgaaaattccgagctcagtctctcagagtacactccgtctattttcttcaattctggaagcgaattctcggagcgctcggatgaagattcaactcattcacctacttttaccttgctccttcaatacagagaagaatttacgagatcgagcactgctttatatttcattgctgcttcctgcgttaaagaactgtataaggatgattatacagtaagttttatcaagttttgaattacgatcttaattttcccttgctttcctgcactttctcgccaaccaaacaaaaatagattgttgattttttatcttttttcattactccttctgtattgatttttgttattttaataatattattttagtttcttaagttcgaagacgaggaggacgaagcgagctatcagctgcttaggaacagagagaggatacaagtatttttgcgagactacacggaggagtactcttccacgacggaatgcggcgatcttatcctccagcaacggtggcaaatggtccgttggatcgtcgaggtgattggctttaccgaaattcacgtttctctgattaagttcaattaatcgtcgttttctaaatttaaataaggtcgaagttcaactaatcgtcgtttttatctatttaaatttggtcgtagttaaattaagcgtcgtaattagttctgtttggaaattggacctgcacacgtttgtggaagtacatgccgtcaagtagcacatactatctacattgacgatattctacaaccatttaacatccaatcaaatctgtgccacgtcattgaatggatgtgtttggcactgaaactgaatgcatttctagtttttatgttaggatcattcatgtactttttatcacaggcactggcacatcactagtttcgcttttctcctattggccaaagtaatacacatttgtaatgtatagggagattaatttgatacatttttgcgaaaaatgattttatgtaaatttgatacatgtacgtctccaaattcaattttacgtaatccttcttagcttaacaacttccacgttgccccacacttagaacgcagtagtagcacgtgcattcgcaccagcgatggtgcgtatataacatttgttgaggggtacttgttacttgttggacaagtctatacactccacgattttttgcgtaggtgcgattaatttcaagaatttcaatacaaagaacttgcatacagcatactgactagggtggatatccaaacggtcaattaagtaattggatcgatttggttccatttctataaaaaaaagaaaaagaaattaaattaattcataattagtttggtttagttcaattcattctctataagaacaagctaaatcgaactaaactgcatattattatttatttattttggcacaaggagttttatattggtaatgatcatttactattatgtttcttcccactatttatatcaacatttaatataattgtattgttagtttgttactttaatggaatgttgaatattgttagtgtactagagatttaaaaagtaaaatggtgaatgcaatgtttgctttacaagtgcttgaaattgttaagattgttaagttattttgactctcatgtatagaattcttgtgttgcacatgagtatattgggtggtggctatgtttatactttgttggtggatgttcgaatgccattgtcaatttgctttgcttgacatgcattgagatgtgaatttaagatattgatgctcttcatgctttttctgataaagtggtaataagagattgcattatagttaaaaatagtgttcaccctcatcattatagtggttaattttcacgtaaaactctaatattccgtttcctgtgggagagggtgcataggctagctgttatccgtatttcttataactaacgttttattatctcttgttattacgttaatgcggtgcttttgattggctttcaacaagcagcgatcgaatcaaatgaagctacagcaggaaacgaagtttctaggagttagcctccttgaccgattcttaagcaaaggatttttcaagagcaaaaggatccttcagattgttggaatagcctgtctaactctagccaccagaatagaagaaaatcagccctacaactggtatatctttatacttttttttatcattcatacatttttacctagtctgtttcctcataaacctggtgttgaggtgttttttagccttttttgtgtggcaattaaagcattacttatagatgaatacaaaaatcagaagttgaagcagtccaattccttctgcaagtgatttgtctggaaatgaatgtattagagaaactgtgaagttgcttagagctcaaacttaaagttaacccacatccccttttggtacttaaataacgatgtaattgagacatgtcaaagcaggacgcatcataattaccttctaaatagacctttctgatggccatttcagcgtgcggaaaagggatttccatgtagaaagcaatgtgtacagcagatgtgaagtggttgctatggaatggctagttcaggaggtcctcagcttccagtgctttctgcccaccatctataacttcttatggtaactctcagtctcccattttctccaaagtactatactagacaaatgcatgctgcttctcttgcattgacaacctggcaaactcaggctcctgatcttctttctctgcatatctagattatccttcaacttttattttctttttttctgggcaaagaaaatgtttctgatttgagaggttcatgccatcttcattccatgaactaattggataacataggttctacctgagagctgctagagctgatgcccaagtggagaagagagccaagtacttggcagtgctgcagatgtcggaccatgtgcaacttcgttactggccttcaacagttgcagctgcgcttgtcatccttgcttctttagaaggcaatcatgaggtgtcccgccaacgagtcatagaggtaactgccataatctttcttttaaactacattgaattataatgtagagggatgccattatgtcatcatgaagaacacatctggttcttgatatctcattgatgcgatctcatggctttgcagactcatgtgagaacagaaggtgatgatttacatgaatgcatagaggtaaggataaaatatgaggtatcataaagttcaataatttttcattttcatttctccaattatctcgccatctagcacagcatcatttcttttctgtttttcttaatgtttcgaatatattgtcattttaagatagttgataggtgctgatggtgtgctaactttaacagagcctagagtggttgttacattatgtgtgatttctgtttgctgactccctcatcaaaagagatggatggaaggtagatcaaggtaaagcctgatcaataggtaacaaaacaaatctgattttttcgtcaattaagacgaccgtgcagctacttgtaaacatttcatag

SEQ ID NO: 16 - DNA, 2788 bp, Ricinus communis
atggaaccagaggcacacatcacgaagaaaccgcgaagcaaacgctgttggcgattccgccataagatctctccgtcgaattcggtgtcttgcaattcaagcagagttacagtcgaatcaaatgctaagaagagaaaatcacttgaactagaaagcaacgtgccgttccggagaatcacgagatcttactataagcagcaaaacgagaagaagaaagcaaatgaattagctgaagtgagcgactcttcatgtgttgaatcaaattctggagtaattgattctgctgttttcgttaggaaagtgtccgatgacgtcttttctactgcttctaaagcaagtcaaaacaaatcaatcgttgaaacagagctctctgaaatttcaaaaaacgaagcagtttcagttaacgagtctttagtagaacaaaagtcaaagagcttagaatctgaaactgatcttgcttgcgctgagcatttctctttggacgatgtcgtttcagattactcttctagtcacgggacagctttctctgagcttcaatttgaagtattcccggagagttcatcggacggccgagagttttcagatgattatacgccgtcgattttccttgattccggaagtgagttctccgagaaatcatctgatgatgctccgccatcacgtacttattctttattgctcgagtttcgatgtcaattcttaagatcaagtgtttctccagacacgataagatcacttattggagcagaaagccatagcgtaagtaattaattttccagtgcatccaaacagtgcctaattagtttgtttacttcttttatatgttttaaatacttatcagtttatgaggttaaaaaatgaggacgacgaagagagctaccaattgttacggggaagagagagaaggcaactatttttgcatgactacgtcgagttgtatcgatccacgacggagtacggtgatctcatactccagcaacggctacaaatggtccattggatcgtcgaggtgaatttctagcgtttgattttaatttattatttattcatcgtcgccgtagctatcgtgaaacaggcaactcctttagttataatttatattaaaataggatatgaaataaatttgtgacacgtcagcgcagcaagatcttttggtctgtactttttgtatcatgtaacagtgttgtagacaagttctagtatatatggattgatcttttcttcttctattggctagaaaatggtctttcgtttttttagtcctttcgcgatgctttcgtttcacatttgcatgtgcattggctgaccttattcctcaatgctgtattggggtacgttacaaatccttttttaaaaaaaaaattcattcgtgatgtacgctttgaataaaacagcagtcaactgccatggagtttcaacatgagacattgttccttggagttagccttctggataggttctttagcaaaggatattttagcaacgtaaggaaccttcagattgttgggatagcctgtcttacattggccacgagaattgaagaaaatcagctctgcaatcggtaattctctttagttacctcattcggttaccagcactatctcgctgaatattaagggatgtaaaaaagatactgccttatttagttgcatgattcctgtacaattatcactataaactttaattgtcatatttcaattggagctggaaagggatattaagtacttcagatattgccaaactgttcattatataatgcactcatgattggccttagtttattggtttatatttacttgtgtgataatgagataaagctcaaatgaatttgataattccagctaaggcgtgacatcaggactaaacatcaatccatttctctgatgctctacttatttagccaaatcacgccacacactctcactttgaagaacacagacatagtttatgcaaggttaatgttactgggatatttcagtgtgaagcgaaggaacttccacatagagagcaatgtatacagcagatcagaagtggttgccatggagtggttggtgcaggaggttctcgacttccaatgctacttgcccaccatccacaacttcatgtggtaatttccattgctttttaatacatattatttagatttgcatcttgtattttacatagcatatttgtattgcacttttaagatggctctcatagtagttcattgtaactcacaggttctatctgaaggctgctagagctgatgcagcgatagagaagagggccagatacttggcaaggctagcactgtcagaccatgagcatcttcggcactggccctcaacagttgcagcagggcttgtcattatggcttctctgcaaagcgagcaaattgaatcttaccaacgggtcattgaggtaagcattcacattgccaaagcctttaaagccagtgatcagtagctttaaaacagagctgaaaaagcattacaaagaagacattattgtttggactagatcctcgcattagagtgtgctttaagatcttaattagtccagcgtttttgcaggttcatatcagaacaaaagaaaatgacttgcatgaatgcataaaggtaaaagacaaaaagccatgccattaatttatatcattctatcatctcctgaaaaagtttttggaaagttttacaacaattcctatagcatgtgcttgtagatgtaattgttaaattagttttgattagtctgttcacaatatttactttgacagacaatggagtggctgatacagtatgtaagctag

SEQ ID NO: 17 - DNA, 3923 bp, Setaria italica
atgcctcccaccatgctcgcgccggtgcccacgaggccccgctccaacccctaccgccggcggagaggggcggctccgctgctcctcgatcaggccgcgactgcggcagcggcggggaagcggcccgctgagtcgtccacctcggcctcctcctgcttctacagcgaggtgatctccgcctcctccacctctctcgccgcgtatcaacgcccggagaagaggtctcgccgccaggacgaggacgaggcgcgcccggccggctccgagtgctcggtggtgatcggcggcgcgagggcgctccccgccgaggtcgaggcctccgagtcgtcgtgcctcggctccgtgctcgagtccgacctcgcctgcccggagcagctcgccgacgacgccgaggcgaccgagtactcctcggcgtacgaggagctgaccccgtcggagcccgatgaggaggaggaggtgctcagcggtccttgcagctgcgccgagtactccctcagccccctgatcagctcccccttgaccgacaacgacgacgacactaccgcgccctccgcaaccttctccctcttcctcgacttcgccaagcagttcatcccctgcgtgcaccccgaagcgcgcgccgtcaacaatgccgctctcgatctcctgacggtgagcgggtttccacgcgatttctgtcaaattcgttgggaattgggtttggctgcattgatcagtgcgacgattgatctggtaaattggtaattgttgtggtgggaatgcttgtgtcaggggaggcggtttgaggacttggacgacgaggagagctacgagcggttccggcggcgcgagcggcgcgaggcggttgcacgcgactacactgaggtgtacggctccatgcccggcagcgacggccctctcgtcgtggagcaacgtgtcgtcatggtgaactggatcatcgaggtcagtgtatactacactcactctgttgtgtgcgtacggtgcgatcaacagttcacatcgggttatgcatcggatgtgaccagatttgttgccctggtaggagctacacaggcttatgcagtaatgtctgcattgtactggtggcatacaactctaggtcctttggtggtttgttgtcagattcaatggtctagaatttgtttaccagtgttcagcaagcaccgcaactgcataattgcataagctgttctctggtgtgaatactttttttagtaatgattattgtgctcaagcagcatgtctgattccctttcgtattttgtactggggaaactatctgctatctgaaagtttgttagtttaacactagttgatgggtggatttgaaaatggcaatgctattgacatatcaaggttcaggggtgtccttgtgtagctttctgtcagtgagtagtcatgcttgtactggctcagtttggtcactcattttgaggaccaaaccatcctggtcccagagatcagctatctctaacatgggctgtttgaaaaggaagagagaaacagcggattcagttgaataaaatgtttctccgcagtccccatttgacatctgaaacatgatttcattggaatgttgcttccatgtctgggattacatagtgtagcaattaggattcctgaatcttcgctctctaaatgctattgtgtctatctgtaactctgaatatgccctttattgcatatgcatccccgcaaatttaagcttttcgatgcactttctattcgtatgagattcagataactcctgaaaatattgttatcaccattttctatcagaaggtttcttggaattaagcatttcattgatgttttccttgcattgtactataattgtgtcactactcaaaagcatggcatgcacaatttatgctccttcattgttccaaaataaagagttcagttgcaaagaatgactttcccttgcaatgccagcattcgtatctgacgaagctgcagccagtgaccgttttcatggggattggactgatggaccgcttcttgacacaaggatacatgaagagtctgagaaatctgcagttgctgggcattgcctgcatcaccctggccacccgcatagaagagaaccaaccgtacaattggtaatgttctcccttgttatgtctgctgtaagagattctgttctgcatatttggtgaatcctaactagatcactagttccgttccatgctttgtttcagtataactactgacttaagtcacaccaaattagctccttcttttaatcatgcattgatcctgcatagtccctcagatgatagaatatatgctgcaaggtcataactatgtttcttttcccagttgcatccctacctcgctgaaatacgccttgggtgtttgaaaagctttaggaggcaatgagatggttcggttcagaagagctatttcactgtttaatcatgttatgaatctgaatcatattagcatttgacggtggttttcacattctcatctgtcatttgtcccttttgatactagcactctgtgcagcttgcatttagaagtgttcacagggtgatcatttcagaagttccttagtttcctgattggactgtgctgctgttacgtgttagtgatagtaaaagaattgacgatgctggttgccatttttggttgattgaatcatatattttgatatgtgacacgcatcgctgcactttgcattgcaagacaactagacgtatctttagctgaaattgcactgaagtgtatctatgagttttgtctcctgatcataacttgttttcgattatttatttgtgctaagcttgatgtgcaacattatctcattgcttgattcctttcagcgtccttcaaaagaccttcaaagttgggatcaatacttacagccggagtgaggttgttgccatggagtggctggttcaggaggtcctgaacttcaagtgtttcgtcacaacaactcaccatttcctctggtaccacaaacttcctgtcttatctgtatcagctgagcataaggacaggctaacaccattctgttcaaatttcgcaggttctatctgaaggctgcaaaggcagatgacagggtagcggacctggcaaaatacctgtccttgctctcacttctcaaccataagcagctctccttctggccctcaactgtggcagccgctgtggtagcccttgcttgccttgccacagacaaggagtcctcatgccatttagtcatggaggtgaacacctcggtcctccatttctagctataaattgtcattacacttgctattgtttagaactcgataaacataagcaaaatatgaactgtaactttcagaatctcctgtcagtcagcttctaaacatgcaccaactccaattttacatgttctgattctagctagactggaaatgcctaacgagtagctctttgcatgtatgtagactcacatgaggacgcaggatgacgatctgcctgaatgcctaatggtacatttttctcctctgattttttataagttactgggcacaaatcaccatgaaccatgctcttacagttccagcaataggcagaaatctacttttatcaccatctaacattcattaccccgtgcaatcttctttcatgcagagcctagagtggctgatcaactacgcttcgtgatacccgtgactcccaggtgacgaaattgatccacagttttgccgattcccagttacacagcacagttcaagcggtcagatagacatgaggatgtgtaggccatacgtgaatcttagcattaacagattattctgtacatgccattagttttccctgtaaggtagatataagataagccaaggcagcataaaacgtagcctgtgattatacgactttctggctaggagcaaggcaaggatcgagagtttggtattgagctgtcggcctttagggactactga

SEQ ID NO: 18 - DNA, 3712 bp, Sorghum bicolor
atgcctcccaccatgctcgcgccggtgcccaccaggccgcgctccaaccccttccgccggcgtagaggggctgctccgctgctccacgatcagactgcggcggctgcggcgaagcggcccgctgagtcgtccacctcggcctcctcctgcttctacagcgaggtgatctccaactcctccacatccctcgccgcgtatcagcacccggagaagaggcagcggcgccaggacgcggacgcggacgcgggcgaggcgcggccggctggctccgagtgctcggaggtgatcggcggcgcgagggtgcgcctcgccgaggtcgaggcctccgagtcgtcgtgccttggcgccgtgctcgagtccgacctcgcctgcccggagcagctcgccgacgacgctgagaggaccgactactcctccgcgtgcgatgagctgaccccgtcggagcccgatgaggaggaggtgcttagcggtcccagccgctccgctctgtactccctcagccccctgaccagctccccattgaccgaggatgacaacgacggcgcgccctccgcgaccttctccctcttcctcgacttcgccgagcagttcgtcccctgcgttcaccccaaagcgcgcgccgtcaccagtaccgctctcgatctcctgacggtgagcggtttctaagcgattttgttcttcaatttgttgggaggggttcgattgcattgatcagtgcggtgtttgatctggtaaattgataatgttttggtgggaatgcttgtgtcaggggaggcgatttgaggacttggacgacgaggagagctacgagcggttccggcggcgcgagcggcgcgaggctgttgcgcgcgactacactgaggtgtacagctccatacccggcagctacggccgtctcgtcgtggagcaacgtgtcgtcatggtgaactggatcattgaggtcagttcatactaaactctgtattctgcccgtacggtacgtcattcagatcaacaatttacctcaggttatgcatctgatatgaccgaatttatacggtgttaagggctgtacaggcttatgcgcgtgagttcagacttgcattgtgccggcgttgcaccggagcgtacgtctctagcaccatagctgtatgattgcagcaagatctgttgtctcatgtgaaggccttcgtgctgagtcagcagattagttagttctctttcttattttgcaccggggtagttatctactactgaacacgttgttaacactagttgatgagtagatttcacaatagctatgttatgcagatggatgagtgtacctttttgtctgtcagtacacagtagtcatgctagttctcattctatactttgatcactggttctggcaaccaacagaggtatgtttgaatagtaggtttacatggtctgtttgataagcaagggagaagcagctggttcagttactcaacaatgtttctccgaatttctcgtttcgcatctgaacccctatctcatcagcacactgcttgcatgtctggaatcatatcctgtaccaatcaggatgcttgaatcttcagtatctaaatatgcaactttctatctgtaactctttatataccctttatttcatatcgatcccaaatttaagctttctgacgtgcttggtattttatgagattccagagaacccctgaaaatactgtcacactatttttacatctgaaggtttgaattgcgatgaagcattacagttatatttcccttgtactctgctagaataatctcactgctcaaaattatgcgatgcaaatttatgttgattcattttttgcaaggaatgactctttattatgaaatgcagcattcacgtctgatgaaactccagccagttacaatgtttatggggattggattgatggaccgcttcttgacacaagggtatatgaagggtttgagcaaacttcagttgctgggcattgcctgcatcaccctggctacccgcattgaagagaaccagccatacaattggtaatgttctcccttgtattacatgtttcaacttttatgtctattttttagagattctgtttcttcacatttggtgagttatagctagaccttctagttctcttctatgctcaatttcagtgaaactactgatgttagtttgtgattccatggtgtcagattgcctaattagatatccacagaattaatgtttagcaccaactgatgtgtaatcagtagcactctgagtgagtgaactcctcctttaatttgagttagttctaacattgcctcataggatatatgctgatcataagctatgtttttatccatttgcatgactaccgctgaaatgtgccttgggtctttgacaggctttagcagggcagatgagatggtttgatttagtagaactattttaccattgaatcatattatgaatttgaaccgtatatgcatgtggaaatggtttccatgcatttccatctgtcatttgtttttgtttttgttttaagtgaaaccatctgccatttgttccttctgatacttggtatctgtgcagcttgcgtttacaagtgtttgtaaggtgaatatttcagaagtttcttttttccaggacataaatttgggtttcctgattgtgctgttatctatgataaaggcattgagcatactagttagcattttttttagttgacttgatatttctatatatttgatttgatatgtattactacaattcggatttggaagacatgtgaaagaagtatatttagctgaaattgcacttgagcatgtcctttgttctccagatcatttttcttttcctattccttttccttgtttctgttaagctcaatgcacaacattaatttcactgcttggtccctttcagcgtccttcaaaagacttttaaagttgggatcaatacctacagccagagtgaggttgttgccatggagtggctggttcaggaggtcctcaacttcaagtgctttgtcacaacaactcaacatttcctatggtaacacgaatttcctgctttcttgtctgttcagttgaaaaaaagtaatgggagactaacaccattctgttccaatgttgcaggttctatctgaaggctgcaaatgctgatgacagggtagcagacctggcaaactacctggccttcgtctcacttcgggaccataagaagctctccttctggccctcgactgtggcagccgcagtggtaacccttgcttgccttgccacaggcaaggggtcctcgtgtcatttggtgatggaggtgaacacccaagtcctccatttgtagctataaatttcgttacatctgttactattgtttagaacatgctaggcatatatataaatcaaagtatgaactgtaactaccagaagctcaagccagttagcttgaaaacagataccaaactccaaatttacgtgctcagattctagttagactggaaatgcctaatgagtagctctttacatatatgcagactcacatgaggacgcaggacgatgacctgccagaatgcctaatggtacgcttctcctcttcttctttacctcttttgtttttggaaagacactgggtaccatgaatcatgattcatgcatgctacagtttgcagggctgctaaccttacattcgttatcctatgcaatctgtttcatgcagtgcctcgagtggctgctcaactacgtcccgtga

SEQ ID NO: 19 - DNA, 4919 bp, Vitis vinifera
atgaggcccaaacgcaatcgaaagaagcgaaacttcgaatcacttccatacaacaagaaactccggtcgaagctacctcgccggagacgatctcagatctctccaattttgtacacgacttcgaactcgaatgctgtccgtacgaaatctggtttacatgcttttccattgcgttcaagttcttcatcttacaattcgagtagaatttcaagttttctgaggaagagagagtttgaggagataggagcttctggagcagtaactcgaggaaacgagaaggtccggagaattactagatcttattaccgacagaagcagaatgagagaaagttggagattggagacggtggtgtagaagcgtctgaatcgtcctgtgtggagtcctgttctggagcagatgttcgagtctctgcagaggcaagttctaagttcaagagaaaaaatgctgaaaatgccaaaataattggaggaaacgaaaatccggaagcagttctgcgatccgagatttcatcgattcaacaaatcgccggtgaaaatttgaagtccgatgccagaaacatcaagaaatcttcggaaaggaaagacaacgacgtaactacttcagttacctctggcgtggaattgtcttcggaaatgaagtttcagaatgcgagttcacctcttggaaacagagctttagaatctgagatctcgcgaagttcacgaaattacgtcgatgcgaacttcactatttcgaattctggttcaaattcggaacagatgccaaagggcttagtttttgactgtgatctttgctgctcggagtatctgtcatacgatgaggtttcagattattcgtccagtcacgagatgctaatttccgagatgcaaacggatgttctcccggaaaatcccgaactagatttctccgattacactccgtctctcttcttcgagtctggaagcgaattctctgagagatcagagggagattcgactcggtcacccactttctcgttgtttgttcagtataatcagcaattttccagattagcttctcgtcttgatgcacgagtttcttcatcactcgttcaaaacgagtatcgagatgaatttacagtaagtcatcgtttctgtgctttgaaatttagttttctcatttagtctgaccagtaagaaaaggcaagacaagaaaaagaaaaataaaagaaaatccgttcaattcttttatttcttcactgtttccacggtctcatgaaataatcggagtctatctcttgagtttaaaatttatttggtttagttgttgaggttcgaagacgaggacgacgaagagagctaccagaggttcagaagcagagagagaaaagcgtgcctacatgactatggcaaagaatactgctccaagacggagtacggcggtctcgtcgccgagcagcggttattgatggtacactggatacttgaggtcagttttctccgaccaagtttccttctacaaaatcgctgtggttataagtaatcattcgtcaattcatcgccggtggcgatctttttctgatctaaaacagtcgcacaactttttcgtggttttttgggctgttccacaaatcctctggttaaatacagcatgcatagttataatcatattcttttattatttgttgatcatcgatcgatctgtcaactaaattactgagttaaaaataaaaaataaaagtgactaaaggtcataaaaatataaatttatcaaataatttacttttaaacattaaattgttcatgttatataattttgtttataaatttaataatattcatgcaccatacaagaatttatttgaaatacgaggctctggtatagaaggctctaaagataaagctgcgaaatcaatcttttcctcaccaaaccaggagtatttttcttttccacgcttcaagtagttcatgagcgtgcgttcaatttgattgatggggaaaatatttcatataactatattttgctgcttaatgtgatgtctattggaactgctgatttactgcatcacgcatctagtctctagtattggcgacaccgccagtgatctctttactttgcttctgttggctacttctcttctcatgtatggcaaaggtactaggtcatggaacgtgaacatctagttcaaatattcccattttgttttttctggtcactttatgttggttacttgtactgtatgggaatggaacattgcaccggcaattccatggagttttcatgcaacttattgagtagcatacaacgtgtttagactgctctgacgtttcatgtaatattgcgcggtagcatgcaattttgtgagtttgtaagttgtgtaatgctctttttacccgactgaatcgaatgttgaaggcaccaatgacatctggagtttgttgaaccaaatctcggggcaccagttttccaacacataagaagtgcgctgggaatgattcagtttgacgggtgccattgcaaatgattgcctcttaatggtttgacatttttttcaccaagtcttgcttttaggtttaccattttaacaaaagaaataaaaaagtaattttttacatctaattgaaacttctaataatattttaagaagtacctgagatcagaaactacaatcattgaattatttttagttttttgtctacgaaaaaaaaatggagatttgactttttaatacaataaggagccgtgggaagatctgaaatcccatggaattacgatacgcaacaactttcgaccatctgaattagagttagaatatcaaatcatattgacatgacgttttaattttattttctgaatagtagtttactttaatagggagcttagggactttgcagttggtggtaaatcctcaccagtactggattttaaattttgcttgtctggtaggagtaatcttcctttcctatgatcatgcagaggcggtccggcttattgagtgattgatatattggttatgtcgatatcaatatatagtattgttcttttttttcattaatcactggttgactcatccatgttccctgaattagcagcaatctgccgcaaaggaactccagaaagagaccctgtttctaggagtaaacctccttgaccgattcctcagtaaaggattcttcaagaacaaaaggagccttcaaattgtcggaatagcatgtcttacactagccacgaggatagaagaaaaccagccttacaacaggtatggttaaactttttctgttgtttcattttcatctctttttagataaaattagttttcttttgatttatcaaaaatgggagaaatggaaagaaattaaagttctgattattaggttatttctgtttggctcttgagaaaacaaaatttctactcagctaagtttaacttcatgcgtagctgggctgattttttcattaaaaacaccaaagaacataagattaaaattttaatttgttctcctatggctttttcttatttttgtcaatcaaatgaaacacaggaggaaaagcataagttctcaattagcttactgccatttccgtaagaaaagacctgtttggatactatggggtcttgggtaggaagtttccacagcataaatttgataatgattttacagtaaagaaattccccccaactctatggcatatcctgatttaaacgtcagtagacatgtttaaagcccaccgtgccatgaattgttaatgtggatactaatgacaaactgttacttatggcatgaaaacagtatgtaaagtacagcttatttgtggatttcttcatatggatacgtaagttgcagttcccataaattttcttatatacatgtgattttcagcctgcggcagaagacattttgcataggaaacaatgtgtttaacaggcgtgaagtgatagcaatggaatggctggtgcaggaagttctcaatttccagtgcttcatgcccaccacctacaactttttatggtgtgccatttctcctcatcaaattcttgtgatgaatgcaatctgggttccatgaacacatgccaacatgctctttttgtccataaatttttttaactgtttcctgtacatgggtgcatgaaaaaaatccataggttctacctgaaagctgcaagggcgagtgcggaggtggagaggatggccaagtacctggcagtgctagcgctgctggaccatgagcagctgtgctactggcgctcaactgttgcggcagggttggtcatccttgcttctctagcagccaatcaagatgcatcctgccaacgggtcatggaggtattcatagagcgaagcattgtctttctttcagctgtttagttcagtgagttgagtgctttctcagtgcatctaatttgcacattatttccataggcttaggagtatatgcacattccctgaaaatgcaaccactgcattatgttttacatagaattaaatctgtcctgtgatttgggtgaatcatatttgagattttaagagatttggcggccttcgcaaattttgctctattattggtgaggtctccttgtgtgcttgtgtctttgcaaattaaggttcagtttatttacttgtcttcattttcatttatttccattggatatccagactttcttgacatgtcctaaataaacttcctatggaattgtcacgaagaaaaaataaattttgattgcagacccacgtaagaacgaaagatgatgatctacctgagtgcataaaggtaaggaataaaaccaaatgatagtgtcctgtcattcagtcacccttttttctacaattgtttctccaaaatgaaattatgaatgcggatgtcatggtcccatttttaagataacgttggttgacaagatccctgatactgtatataactgcaacagagtatggagtggttggtaaagtacgtgtcctag

SEQ ID NO: 20 - DNA, 4494 bp, Volvox carteri
atggaaagctgctcgccttgtggcgcctcgaccgtaagcagagcaatatccggcgggtcctcgagttcactaagcttggaggattatgatcaaggaatcttcaattacgatggcatgcctccacgagaagcaccgcacgatgaggaacggattgttggcatcaaaactttcgtggactccaccggggccaaggcgcatcgtatggaacccaactcaatactaaggtgaggcttacgggtatagtatgtacagtccgtcgtggacttaggtagattttccgatacgggtacgtcacggagaccgcatttaagacgtgaccttcctgatgtatccaagcagctataaaatgaatatgcctacctttctatgtagctgagggacttttcctatgcggaaggcttctaggctgcacctgtcgattgttgtcaggagcgctcgatgtttaacacagcatctgtctacagggcactgctaattttgccaaagcagtatggcaggtttcagcccttttaggttgttagtcgctgcgcggctcgaacttcgtcttggagggttaacgtgcatattctaattcatagtcacatttccatacgcagtttacggctccaaatattcgaatcattatagcttacgtgcgcatatgcacagttggtacaggcagtaagatggtagctggtttcgactggttgcgatgacaatcctactgcttgcgccggcaatcctattagtatgaagccggttgcacacccatacggttaattaaagcaactcgttgtctaggagtgtaaatgtctttattgtgcatggcgggcacatttaatcagtcccccgttttgggaaaaagtattactcacaaaagggtaaattcacaatcactgcacgatacaggatgttttaaccgttttgatacgttatgcatcattgcgcatgtaaaaggcggatttgcgtaaccccaacgcgtgctaaaaggtccgcccgtgtcaaaagactttcgcactggcgtcatcacgggcttctatctcagcagcaggcccgacccccatgcattaagaggagcaaatggcttaagtgttcttggcccgttgtccaacatgtgcggacggcaacccaaaggccacagtatgcactgcctatcttggtcctgtctctcctccacacgacgtcgtggcatcgaaacttacaagtgtacaaacagtcagtcccacacacagccgttttgtgagccgtgtcccgacgcgcgggactaaatccacctgtcgatttttggcggccaaggttattaccctggattttgtgagacgtgcacttctgctgacttaccgcgtgcacttccatccgtagttccatcctccttcctcttccccggcccccctggcaccagggacgacctccgcaaacagcaatgcatgcgcagctctttagcggctcggtgttcacgacagtacggcagcggtggtgttggtttgccaccatgccctctgcttcctgcctcgtaccgtgcgcgtatggttggctggatgcgggaggtgtctgtggccctgggcctgcagctgtccacgcttttcacggccacatccgtactggaccggtttatagctgcatctgaggtgcggatgtacggcagggaggtgtgtatatatgcatgtgtgtgggcacacagatactagcttgttttgccagcttctgcacctcgagtcttgagatttgcacaaccttccccgtcaagcgcaatgatttcgcattttgagcggccgtgcatacatacatacagtgtaatctaagtcatacatacatacatacatacatacatacatacatacatacatacatacatacatacatacatacatacatacatacatacatacatacatacatacatacatacatacatacatacatacatacatgcatacatgcatacatgcatacatacatgcatacatacatgcatacatacatgcatacatacatacatacatacatacatacatacatacatacatacatacatacatgcatacatacatacatgcatacatacatacatcacccctacgccgtaaggcgaaggtgcactgctgtggcacgtgaatggcctccgtgcggtactgtcgtactgattgttcgtggtttcttcttttccacaggttcttccccccgaggggttgctgcagctggtcacgctggccagtatgtcagtggcagtcaagtacgacgaggtgcatatgcaatgcgccgtactgcgtgtggatgagcacggggacaatccggcaactcatccatgagctgcccttccatgtacactttttagatcgctgctttccgccatgttttttccttgctgtgaatgcaatggtttgacagcatcggacatagatagacatgcgcctgtgttaccacgctggtactggtcttctggcccctaagctcatcaggacgcgctatctgagccgttccagctccctcacggagtgtaattttttaatgtatgagggaaactcgtgggattagtacctttaacagaagtacttcaaaatccaaggcatgaattcaaatgcttaggcgtataactccaaaattgcggaagggtgcaagaaacatcaagaaagtgcaagcgactcgtgacatccaattattaaaaaccggtaaccaactccaatttgtgcgcagcacagacttttaagcgcacttcaatgttcgtctccgtgagttagagttttttttgcttcgtcctccttagctccgaaacccaggaggctcctgtccccaatccggcgggaacttttactgcccccttgcttcgccatcgcagacgtcacaagccgtgtcacaagccgtgtggctgagcttggccgtcaatcccgatggcaagcaactctactcggtaggcaggtcaggggcggggatacgcgctttatgcatacgactagtggtgggtgtgggctcctcctctttccttacactgtttttcttgccaaaatcattaatatcattcacacgggaagaggtaagggcgagaatcccgggagaatctccctgccacccgggactcatggaccagggtacccaaggattgtgtttactgtgctcctgggccccgtaaatcaatatccctgttcgcgcacagggctccccccctgatatcagtattgacatgtggaacacggttaaggatggcatgtggctgttaagtgatggtttcggagcgctaacgtttcagcaggagtggtgtcgatgagttgttcgtaatgggttggcagaccggagggttggtgccgccagggccccaaattgcgagttttgacatcactttatcaaatgccaccttccccccccccgcccttctcccttcccctctccatgggggcccctcccccaccccctcccccaccccacccatcaggcccgagatctgcagcggtgcgagttcacgttgctgcaaaccatcaactggcgcctgcaccaacccaacacctacaccttcctggaacacttccttacatgcctatcgccgtcagacgacgacgacgacgagtgcattgatcggcacgctgctgctgccacttcagctgctgctgctgctgctgctgctgacggcgatcccacagtcaccgccgctgccagcctcgaccatctcatggatattgaaggcgccatgccaaacgacccgtacgccttccccggatcgccgatgccatacaactccctggcggcggctgccggaccaatcacggccctcacccccggctccactgcggcatccacgcagcagttgcagctgcagctgccgaagggatcggcttgtattacgtcgaggagtctatcccgggagtggaggacgttcctggggactgctgtcggattagcggtgaggcgctggtggttggttcgggggaagcctggggacgcgggggggattgaggaaactgcctccgcgggataggcagggcccaccactaccccacctactgtcattaccaacaagggtgctgcttgctgacgtgtagcgttcgctgtaaaatgtatatatatgtatatatgtgtgtatatacatatatattacatgtatataatgaatacgtcatgtggcataattttgttcacgtgcaggaggcatctcttcttgatggggctcttctgtcgtacgatcacagcgcagtcgcgatggcgtgcattgtactggcggagcgcgtcacgttcagcgacagcaagggcggcagcagcagcagcagcagcgcctgctccggtggcggcggcggcggcggatcgcctgtacggatgcccgcatcgggctctcatgtggctccgaaggggacggctaccggcttgggtggtgactgcgccgctaccttagccgccgtaaccaccgccgctggtcttccattgcctgttctggctccttgtttagtgagctgcatgacggccctggagggttactaccagcagctcaaggacgcagcagccgcggaggcggcggaggcggcggcttag

SEQ ID NO: 21 - DNA, 4743 bp, Zea mays
  misc_feature (4181)..(4300): n is a, c, g, or t
gccggtgccactgccagtgcgacacggatcacgcaatgcctcccaccatgctcgcgccggtgcccaccaggccgcgctccaaccccttccgccggcgcagaggagctgctccgctgcttctcgatcagacttcggcgaagcggcccgctgagtcgtccacctcagcctcatcctgcttctacagtgaggtgatctccaactcctccacatccctcgccccgtatcagcgcctggagaagaggcatcgacgccaggacacagacgcgggcgaggcgcggccggctggctccgagtgctcggaggtgatcggcggcgcgagggtgcgccccgccgaggtcgaggtctccgaatcgtcctgccctgtctccgtgctcgagtccgacctcgcctgcccgaagcagctcgccgacgacgctgaggcgatcgagaaatcctccgcgtgcgatgagctgaccccgtcgtcggagcccgatgaggaggaggtgctcagtgatcccagccactcggggtactcccccagtcccctgatcagctccccattgaccgaagatgacagcgacgacgcgccctctgcgaccttctccctctttctcgacttcgccaagcagttcgtcccctgcttgcaccccaaaccgtgcaccgtcaccagttccactctcgatctcctgacggtgagcagttcctacgcgattttttcttcaatttgttgggaggggttcgattgcattgatcagtgcggtatttggtctggtcaattggttgtctaatgttttggtgggaatgcttgtgtcaggggaggcggtttgaggacttggacgacgaggagagctacgagcgcttccggcggcgcgagcgacgcgaggcagttgcgcgcgacttcactgaggtgtgcagctccacctccatacccgacagctaccgccctctcgtcgtggagcaacgtgtcatcatggtgaactggatcatccaggtcagtgagtctgtgtcagactgtcagtgcaccataaactctatagtctgcccgtacggtacgctcatcccgagttcccgaccaacaatttacctcagattatgcatggaatgtgaccgaatttatacggtgtaggggctgtacaggcttatgcgagtgagttcagatttgcattctgccggcgttgcaccagtagcatacgactctagcaccatggctgcaaattagtagatttcgcaatagctatgttatgccaatgaatgttgtctcgtgtgaatgccttcgtgctgaggcagcagattagttctctttcttattttgcactggggtagatatctactactgaacattttgttgttaacaccagttgattagtagatttcacaatagctatgttatgctgatggatgtcgtagtgtatctttttgtttgtcagtagtcatgctagttattgtatactttgatcactggttttggcagccaacagagttaggagtatgtttcaatagcaagtactcatgctttttttggaaatggaaacattgtttcgcccttttgcatttgcatgcatacaaccttataactcaattattacatcaacctgcaacaatttgtagttcaaacaaccttcaaccaaataatgatatggagtaaaataaaacacgagcactaagcctctttaattcttgcatggaaccgccacctgtgactcgcaaagaactccatggccacatcctccaaagactggcacacgacgaggatttgctgctgtgtttcttctttctgcaatagtctccaaaatctgaaccagtgtgtgtccctgaagatagtctgtataattgacggtatagatttatgattaaacaccacatcattacggcaaagccaaatcgaccaaaacatcgccgcaacgccagtaagaagcaagtttttatgcgtgcaacctttgttggatttccaatcccctataatatgattaatattaatggtacatggcctgtttaataagcaagggagaagcagctggttcagttactcaacaatgtttctcccaatttcttgtttcgcatctgaacccctatctcatcggcacagtgctggtatgtctggctggaatcatatcttgtagcaatcaggtgcttgaatattcagtatctaaatatgcaagtttctatctgtaactctgtatatacccttcatttcatatttattcccaatttgagctttctgatgtgcttggtatttttatgagatttaagagaactcctgaaaacaccatcatcaccatttttccatctgaaggtttgaattgtgattaagcacaacagttatatttcccctcgtactctgctacaatgatctcaccactcaaaatcacgtgatgcaaatttgaaatttatgtgtattcatttttttataaatttgttaaaaaaattagagttcagttgcaaggaatgactctttattattcaaaatgcagcattcacgtctgatgaagctccagccagttacaatgttcatgggaattggattgatggaccgcttcttgacacaagggtatatgaagggtttgagaaaccttcagttgttgggcattgcctgcatcaccttggctacacgcatcgaagagaaccagtcatacaattggtaatgttctccattgtattacatgtttcaacttttatgtctgtttttaagagattatgtttcttcatatttgttgagtgatggctagaccatctagttccattatatgttcaatttcagtaaaactactgatataagttggtgattccatggtgtcatattgcctaattagatatcgacgggattaatattcagcagcaactggtgcctaatcagtagcatctgagtctgtgtgagctcctcctttaatttatgttggttccataagctatatttttatccatttgcatcactaaagctgcaatatgccttgggtctttgacaacctttagcggggcaaatgagatggttttgatttagtaaaactattttaccatttaatcatattatgaatatgaaacatatctgcatgtggcaatgctttccatggtatttccatttgtaatctttttttgagcaaaaccatctgtcatttgttcctttcaatacttagtatctgtgcaatttgcgtttagaagtgttcacaaggttaacatttcagaagtttattttttccaggacataaatttgggtttcctgattgtgctgttatctatgataaaggcatttgaccctactagttagcattgttttagttgacttgatgcttttatctatttgatttgatatatattactacaattcacatttggaagacatgtaagagaagtatatttagctgaaactgcactggagcatgacctttgttcttcagataattttttcttttcatattccttttccttgtttctgttaagctcaatgtacaacattaatttcactgcttgatccctttcagcgttcttcgaaagacttttcaagttgggatcaatatctacagccagagtgaggttgttgccatggagtggctggttcaggaggtcctcaacttcaagtgttttgtcacaacaacccatcatttcctatggtaccacgaacttcctgccttttgtctattcagctgaacaaaagtaatgagagactaacaccattttgtttcaatgttgcaggttctatctgaaggctgcaaatgctgatgacagggtagcggacctggcaaactacctggccttcatctcacttcggaaccatgagcagctctccttctggccctcgactgtggcagctgcagtggtagttcttgcttgccttgccacaaacaaggagtcctcatgccatttggtgacggaggtgaacacccaggtcctccatttctagctataatttttgttacatttgttattgtcagaacttggtaggcatacctaagtcaaagtatgaactataacttccatcaaaacagataccatactccaaatttacatgctcagattctagctagactggaaacgcctattgagtagctctttacatatttgtagactcacatcaggacgcaggatgatgatctaccagaatgcctaatggtacacattctcttatttttctcttcttttttgggaatacactggtgggcatgaatcatgattcatgcatgctacagtttgcaaggctgttaactttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacaatctgtttcatgcagagcctcgagtggctgatcaagtacgtctcgtgatacccagagctcccaggtgatagcagtgttttcacttttttctgtatggggacgtgaaatcttagcattgacaaatagtctgcctgtagtgtagataagatagccatgcggcatgaaacgtagcttgtggattttgattttgcagctttctgattaggagcacgacaaggacgaggaatttggtattgagcttggcctttaggaataactgaacttctgtatcgggggatgtctatctttacatcggttagtcgctctttttagaaggacggctaaggctgggcgttgttgtactcgttgatctatttgtttaaccaatgtattgctgatggatgatataccactgaaatctgttgttctggtgtgacaagcggcatgatcttttgatccc

SEQ ID NO: 22 - DNA, 51 bp, Artificial sequence
  Syntheic
tgggttcgttgcattgcattgatcagtgcggtgtttgatctggttaattgg

SEQ ID NO: 23 - DNA, 50 bp, Artificial sequence
  Synthetic
ctagttagactggaaatgcctaacgagtagctctttacatatatgtaggt

SEQ ID NO: 24 - DNA, 27 bp, Artificial sequence
  Synthetic
ggtgggaatgcttgtgtcaggtcagtg

SEQ ID NO: 25 - DNA, 46 bp, Artificial sequence
  Synthetic
cataatctcactgcttgatccctttcaggtaccacgaatttcctgc

SEQ ID NO: 26 - DNA, 15 bp, Artificial sequence
  Synthetic
ctgattttgcaggta

SEQ ID NO: 27 - DNA, 336 bp, Bacillus amyloliquefaciens
atggcacaggttatcaacacgtttgacggggttgcggattatcttcagacatatcataagctacctgataattacattacaaaatcagaagcacaagccctcggctgggtggcatcaaaagggaaccttgcagacgtcgctccggggaaaagcatcggcggagacatcttctcaaacagggaaggcaaactcccgggcaaaagcggacgaacatggcgtgaagcggatattaactatacatcaggcttcagaaattcagaccggattctttactcaagcgactggctgatttacaaaacaacggaccattatcagacctttacaaaaatcagataa

SEQ ID NO: 28 - DNA, 699 bp, Bacillus amyloliquefaciens
ctgcaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatgaattcctgcagccccaaacacacgctcggacgcatattacacatgttcatacacttaatactcgctgttttgaattgatgttttaggaatatatatgtagaggtggagacatcttctcaaattcacaggtcgtgatatgattcaattagcttccgactcattcatccaaataccgagtcgccaaaattcaaactagactcgttaaatgaatgaatgatgcggtagacaaattggatcattgattctctttgattttgagaagatgtctccgcctctctcttttgtattccaattttcttgattaatctttcctgcacaaaaacatgcttgatccactaagtgacatatatgctgccttcgtatatatagttctggtaaaattaacattttgggtttatctttatttaaggcatcgccatggggggatccactagttctagagcggccgccaccgcggtggagctccagcttttgttccctttagtgagggttaattccgagcttggcgtaatcatggtcatagctgtttcctgtgtgaaattgttatccgc

SEQ ID NO: 29 - DNA, 5114 bp, Arabidopsis thaliana
acatgaacaactgttcggtgctactatgtcaatgcattttgccaaattactactcagtctactcacgatttattgtactgcgtttacgtaacgcgtttgtatgatcgtttattggtaaccgtaatttatggcatgccctcctgcttttttatttaagaaaaataaaactaattatattgtaaatattgcattgatcatttagtcacactctttagaaaacaacagtaaaatttaaatataaaaacaacactagcttccatgattatttttcataaccatttataattgcgtcatcttgtaagttgtaacgcattgcctttcttactatgtaacggttgttgcatatttttgtgtacataaatttatacacaaagataaaaagtgactaagcttaaaatatccttgaaaaagcctttgggtcattaacatggtgtaagactacaggcgcattcagcaattggagttccgattctattacagtaagagggaacagaaccgtaataatcgcgacacatttgttcgcatttgttagcatcgcatggaaccattggccagaaaacggggcaagtttgttccatcattctcgtctctctcgcacctttaaacaaacatcagaaaatttgtgacattaattaacaggatttggcttcttataaagataagattaaaactactatttaaaagataatctgtacctgaggctgaaacgatgaagatggtcatgataagaacagcgaaatttatgaggtttctcatggttttatgtttttttttttcttaacaaagacgtaaacttgaatcgttttatatgcgaaattgacagagaaaaccggaaaagataggatctccttttctttctttcttttagtgaaatagatgataaacttgtttctgctaaaagaggtgtttattttggaaattatgaattttctggtcaatgtgatcttagaattttaaataggctggattttgtgacctgattccgtgtcttatatctgtatttactatatttagatgattctctgataactgatgttttaaaaagaagataattttgataaagaagtgattacgaactttccaacattaaaagtttagagtttatttgattttatatctaatcttggtttatatgtttttgatggggtttactaattatattataccattcaagttgaaatatatacaagttttttttgttttatccctaaattctctaatgtgatatatataatatataatttggatcggattcaaccaaaccatgaacgagatttacattttgccgttttccgaaatgttttgggcttcgtaaagaactaaaggtgatatttagatattgggtatactatttgttgtattgggcttaaaagtttacttttttggcccaaaattaatcaactaaaataagatcaccaatggaaaaagaaacaaaaaaaccagtaaaacatatgcagaaaatgtaaatttacagggcctaatataatctgcttgaccatgccattgcgacataacaaatgttacacaagtagtgtacctataaagtagtgtacctataatatattaacagtgatcaatttcagtgtataaaaaaagtcttcttaaatcatcttttaattccaacaatatgacattcacaaacttatctatgatttttttaaaaaaaaattcacacgtgtgctcaatttatgtttcttttagttcttccacgtgatttgatgcaagaaaaatgattagactgtatgttaaaaagcatactagagaaattaattataaaacatcaatcagttgaagtaattatcaaaaccgcatgcttttttagctaaatctgtgattgtactgacgcagatgcataaattcaaacgcaaacgctgatctctacattagccaaacaagaatagcgtccaaatttacgactggtttcacgtgcaccaaaccgtagggtataatatctctctctcactctccaacatccccactcttcccaagaaacttctataactgcatcagcccactctctagtctctgactaacgaacttccattttcaaaattcgaatttctaatttctagtttcaagctttcgtacggagaaaaaatgaaggagatcgcgatgaggaattcaaagcgcaagcctgagccgacgccgttcgccgggaagaagctccggtcgacgcgattacgccggaagagagcacagatctctcccgttcttgttcaatcacctctctggagcaaacaaatcggagtctctgctgcttctgtcgattcctgctccgatttgctagctgatgacaacgtttcctgtggttcgagcagagtcgagaagagctcgaatccgaagaagactctaattgaagaggtagaagtttctaaacctggttataatgtgaaggagacgattggtgattcgaaatttcgaaggattacgaggtcttactctaagctacacaaggagaaggagggagatgagatcgaagtaagcgaatcgtcttgtgttgattcgaattctggtgctggattaaggagattgaatgtgaagggaaataaaattaacgacaacgatgagatctctttctcacgatccgatgtgaccttcgccggacatgtctccaacagccggagtttgaatttcgaatcggagaataaggagagcgacgtcgtttctgtcatatctggagttgagtactgttccaagttcgggagcgttaccggaggagctgataacgaagaaattgaaatctccaagccgagcagcttcgtggaagctgattcctctcttggatcggccaaggaattgaagccggagcttgagatagtcggatgcgtctctgatctcgcttgctctgagaaattctcggaagaggtttcggattctctcgatgatgagtcatctgagcaacgttcagagatatattcacagtattccgacttcgattactcggattacactccgtccatcttcttcgactctggcagcgaattctctgagaaatcttcctctgattctcctatttcacattctcgctctctgtacctccagttcaaggaacagttctgtagatccacgattcccaacgattttggatcttcttgcgaggaagaaattcactctgaagtaagtggtataatgatttcatatctcttggaataattgctagtggttagagattgaagatgtatgtggttatatggttgaaatttcattcgattactagtctatttttgatatgagacttgttctgctctgtgtttgattctgaaattttgttctggaatgaatcttaagtatacattttcgttttagttgctaaggtttgatgatgaggaggtggaagagagctatctaaggctgagggaaagagaaagaagtcatgcatatatgcgggactgtgctaaggcatactgctccaggatggacaatactggtctcatccctcgtctacgctccatcatggttcaatggattgtaaaggtgaattttaactttctgttcaaatgcatttagttacatatacattgatctctgaatgttgaagctcagaaatatgtatcagtagcagaagattatgaagtaaatgaatatttggagatcctgttcctggttttaagaatgttttagcctaaggaaatctatagcttactttggaatcttttaaggtttatgtatcagtcagctatgatattctttgttgctgattgtctgctccctgattacaagcagcaatgttctgacatggggcttcagcaagagacattgtttctaggagttggtctgttggatcgattcctgagcaaaggatcattcaaaagcgaaaggactctaatactagtcgggattgcgagtcttactctggccaccagaattgaagaaaatcaaccttacaacaggtaccaaccatattccatcttcatgattctgacttccaatgttcattagaaaagtgttctgagtaggaaaaagattaggaccattacaagaaactgagtattacgcttaaccaaatcaaggactaataatggtctaatacaaacccttatggttcaatgaattggcatttcatgtgggtatcgaatattggattatgtttctcaaaaacactctttactggaaagaaccttccacaatacacaggaatagttcaattttcttcaactgctcacctgatacttgctctttttaactagcatccggaaaaggaacttcaccattcagaacctaagatatagccggcatgaagtggtggcaatggagtggctggttcaagaagtcctcaacttcaaatgcttcacacccacaatcttcaacttcttgtggtaaaacctctctgactatatattttcatgttccaagacacattatccacacagaaagatacatatgactatcatttatacatgtcaggttctacttaaaagctgctcgagccaatccagaagttgaaaggaaagccaaatccttggctgttacctcactatccgaccaaactcaactctgtttttggccctcaactgtagcagctgcactcgtggttctcgcctgcatcgaacacaacaaaatctctgcataccaacgagtcataaaggtatcatcagtcccttcaataacactttaataccttttagtatcgagaatatacaagaatcttcacaatcccaaaacctctctttctctccaggtccatgttagaacaacagataacgagttgcctgaatgcgttaaggtgttttcagtaacactctcattatatacaaatctcatttttaccactaaacgtaaggtaagtgactgttttcacatttttgttccctatacaacagagtctggactggttgcttgggcagtaagcaatcaaaaagaacaaaaaccctaaaaccaggacacagtatactccgataccaacacacaggttatcattactatttacaaaaacaaacacaaggtaagtaataagaactcctctacagatttatatacttaatcgagctggacttaattagctcttagtataccaattattagtgccaccatttgtgtcgctcatacacatttatttcttattttccctaattcattagactctcatattcttaaaaagaatatttccttgtttg

SEQ ID NO: 30 - DNA, 7639 bp, Oryza sativa
ggatgcttgctactggataggagtcatggaagagaacggggtgctctgtgacactgatgtctacaatggtttgttgcttaggctgtgtgtggaagggcatgttggtgaggccttggcgttggctaagaaggttgctgagagggggattctcatagaggcttcttgtgctgatcgtttgatggatttgctaaagcaatatggtgatgaggagctagcaccaaaaatatcagaactgaggaggtgctctgaagtgctgtcacattaaccaatgtgtgatccgaaccctcctacaagtatcatgcttggttgatttcaaatcaagaaaaatgcttccgtgctgcatgattacagcaagaaaaggctttgagggtttgttacgctgaaatagattggtggggatagggtgcagcacagagtgatttgtgtgagcaaaatgtggatgagttacttcatttacttgcccatttcctgtagtttttctgaactctgttcagatcctccagtccaagggatgcttcaggacatgtgaactatgattgcgatggaattctcaggttcctcattagtatgctcccaaacagatatgtttgtttaagtggtgatcaatcaaatgttttacatttttaaagaacacatatgctgacactgtaacttgtagtagttcttcgacctccgttgtatagcggccaactctaatcaagatcaggttaccgatttacagctagaatgttccaacttgcatcctttgatgcaagtgttttagttcactgactttagtagtgaatgttgttttacgggaactcttgtgtttccccagggtgatgcacaagggaaccaaggttttcggtactctgttcagaattcagattcagaggagacgtttctgaagtctgcggcaaatgacggtcttcagaagtgtgtatcagactatcaatcagtccatcagggtcccatctacatgcatacactttccttttctttcatttcctctttaccgagctatttgctccaaaccttatccaagccgtttcaagggccctttgaatcgtaggaatgaaaaaacagaggaataggaaaaacacaggattctgacaggaatacaattgtaaaatagaggattgcaaaacacaggaatggccatttgattggatcacaggaaaaacacaggaatcagatgagagagatagactcagaggaaatgttccaagaggatagacctattgctaactttcctccaaaatgtgcataggattatccattccataggaattttaaaggattggataagattcaatcctttgtttcaaatgccttcataggatttttttttcataggattgaaatcctccaaaattccttcatttttcctacaaatcaaaggagctatgtgtaacttgaaataccaccggaataccagcagattcaaacaatcgagcttcaactgtacttccttaaaaacgtagtgatccgagtatgcagtgacccaattggagacaacctggttgggattgggtaattcttccccgatcccaactgggttaaccggatcgttcgatcgagattgttcgggtaaggaattaatcagggtcaggatagcattttgacaacctggccgccgtccgccaatcccgcatatgcgctggccccagcccgacattccccactggaagcaaacgcctcgatttgcctgggaggctgggacacgggaggcaaggccgttattccggcggatcgctcgtgcaccagatgcatctgggacccacagggatacgccggcgaatctacacggactggagtacacaggccatccaatggagcgcgagcggcgcatgtgcatcgtcgcagcacagtcgtggcttgaggatttttggaagttcaaaaaacacttcaccgatcaggcgatgcggcgaacagccgaaggttgctgaacactcctctcccccttcctctccacgcctcaagtcacggctatataactagcaagccaaacgaacatagccgtaggcccgtagcatcacaaattcacaattcacacacaaagcacacagcgccgcacgccaatgcctcctaccatgcttgcatcggtgccgacgaggccgcgctcccatcccttccgccggcggcgaggcgcggcggcggcggctcctcctctgctccctgatcagatcgcggcggcggcggcggcggcagccaagcgacccgcggagtcgtccacctcggcctcctcctgcttccacagcgaggtcatctccgccacctccaccacctgccccacctccctcgccgcagctcagcgcccggagaagcggcctcggtaccaggatgtggacgaggagcagcccgccgcctccgagtgctccgagatcatcggtggcgcgaggccgcgcgccgccgaggtcgaggtctccgagtcgtcgtgcctcgcctccgtcctcgagtcctacctcgcctgccccgagcagctcgccaacgacgccgagacgaccgcctactcctcagcccgcgaggacctcacgctgtcggagaccgaggaggaggaggaggaggaggaggtgcgcagtggtccctgcatctgcaccgattgctcgtttagcccccttcatgagtcgtcctcgtcgtccgacgacgacaacgccgtgccctcccccaccttctccctgttcctcgccttggcggagcagttcgtccccttcacgcatcccaaaacgcccaccgccacggatgtcgctctacaagcgggagaggtaagcaggttccacacagcttctctcaaatttgttggagtttggctgctctctcccgaatcagtgcgacatttgatgtaatcagatgggaattattgtatcaggggaagagatttgaggacttggacaatgaggtgagctacgagcggttccggcggcgcgagcggcgaggggtggtagcccgcgactacattgaggtgtactcctccatgctcggcagctacggccgcgccgtcgtggagcagcgcgttgtcatggtgaactggatcatggaggtcagtatgctctgcattgtacacgtatgctgcgaccatgactttgccttggtccaataattcatctagctcgagcgcaatttgttgtgtggtagctgcagatgtttgttgcacgggtattgtgctgataatgcacatgtatactttcggagtgctgtactgatctagattctatcagcttatctgtctgactgatgtgttctgcaagaacagtagttgcttgcattttgcactaacatgctactctgcagtgtaaatgctgtgctgggtgggagtgctgaggtagcagatccggttgtgatcttgcatattttgtgtaggggaagctatctgataccaatatacgcattgcatttgctgattcgtttatcacacagttagctgcctgtacattttgcagcattccccgtgttagtctgccagcagtgtggtggtaatcctagtgatttcaatgtaactctagtcattaacttttatgtcatcgcattcaagttctatagagcagtgataaagattttctaggtgcttattttggcatgcttgcatattttttttggatgtaatggcatgcttgtatgaaaagaaaagagatacataggatttttctaggtgtttattttcgcacctgaaagttgctctgtagtcatttgccatctgaaatcccatggcattgggcattggcatatggcctccagaaatgtctgcatcactttttacttctaaacctatatatgcaataagtgttagaaaaacatattgccagtgtattccctttttttgtgtatccaccatcatctgaatttaatcttttctaactctagttccatcagtttatatttgcattgaaacttcaggtagcctttgaaaatgaaatcgctgtgtgttttttgtttgaagtttttttgggcaactcagcgataagtacttaagtgatatgatcatttatacttgtaatttgatttttctgaaaataatgtactgtcatttgtggagaatatgtttgttcttacatagagttcatacataacttgcaaagaatgacttgttctctatgcatcgctagcattcacaagcgatgaagctgcagccagagaccgtgttcatggggatagggctgatggaccgcttcttgacacgtggatatgtaaagggctctaggaacctgcagttgctgggaatcgcctgcaccaccttggcaacccgcattgaagagaatcaaccctataattggtaatggctcccttaaatgaccagtttcagtatgaatacttctgcaagttgttggtgtctgcttgtctggtactcacctaaatagttcgtctcatttgaatatagcatgtattattaaatgggatagtgctcaattgcaatgctgcaaattccttagtattctttgatagtttgatttttctgaaagttgaacttaattctaacagacattagcatgagtttatgagtttaattatgttctataaataacttaataacaacaggaacatccgatggtaactcatatctcatctagtagctatttatcactttcaatactgagcactgcatgtgagaactcagaccattggaccattgttctacaaaaaggtttcacacgatgggtattttagaaatgaagggcttgtttggttctaagccattgtgggccataccaattttttggcaatggcaagatttagccactccaaaatcttggcaaaggacttgtttggtttgttgacaaactttttggcaagattgattagtttagtatttagtttgctaccaaggaaaaatattggtgttgccaaaatttggtgacaaaaagaaagctaacaaaattttaggcagaccaaaatattggtatggttttgattggctgagaaccaaacacaccagaagtttcttgattgtgttgactatgatcaagcatctctcaaaataatttaccgcgccagttggcatccttgagctattgttttttaagaaaaaaagaactaagctatcaattattgggagagatgcgattttgagaaattatcacttgcatagatctccttgatatagttttatttctctgtttttgatgttacaataaagctattacatcagcgctctggtgggcgagatattctctgtgttttcccttaagttttaaagactaaactcatatgaatcttcaacatttttgaaagtagaccttatttgtatccctgagaacttgctagtaaatgaatgttttatgtgccacttattactgcacagaagcatgtgaagtgaacccaggaactattcttctttgcctgtctactttgcacgaagtatcacttttgaacttatgagtttgcctctggatgcgcatttgtttcaagcgcctagtttctgtcaagggcaatgtgcaatatagaccttttctttagaacactctacaggaacctaataatacgatctctttcagcatccttcaaaaagctttcaaagtagggatcaatacttacagccgaagtgaggtcgtcgccatggagtggctggttcaggaggtccttgacttccagtgctttgtcacaacaacccatcatttcctctggtactatgcctgtgtgtttgcttcattttctgtgtcagctggacagaatgaataagaaacttacaattgtttggttcaactttgcaggttctatctgaaggctgcgaatgcagatgacagagtcgaggacctggcgaagtacttggccttgctctcacttctggaccacaaacatctctccttctggccctccactgtggcagccgcagtggtagcccttgcttgccttgccacaaacaatgagtcatcatgccatttggttatggaggtaaatactttaatcttccatcactggctatgctatttctcttatatctgcagtctgctattcgttcagaacttccttaaggaaaaagattctgaacttttgctcagttttgtatgttgctgctttcattttatctcgtagccagatgacaaatgccatgaatcacctaggtcttcacatgcttattccaattcacaacatactgatccttgcaaagaatacaaatgtctaaccacttgctcttcacataaatgcagactcacatgagaacgaagaatgatgatctgcctgaatgcctaatggtttgttttctcctcaaattatgattctggtgaaaagtgttgtacacagtttgcagcaatttgactattacccggatctttagcgtgccatggcatataacgtgcagtaatccttttatgcagagtctcgaatggttgaccaattatgcttcctgatttgaacaaacccaggtgatatgccgacccaatcttctgcccattcccagaaacacagtgtagtacgtacagagtcgcttaggaggaggtgtatatagtacacgtgcactgtagctaattcttcgtgcatagcattagtttgacagcagggtagaaaagagggcaaagaagccggctaaacgtggttgtgatggcaccagacttatagggagcatcgcaaccacgaaattttgctactactgccggcttcagtgactacgactaacctcctgttgtaagctgtcttaaaatcagccttcgttgtgttaagggcaaccaaaggttgagactttgttgttctaatcaaacatcctgtttacaaattgcaactggtgccagaatgaaatttgttctccgggtggtattgcactgcacgaggttcgattctgtgaccctgaaagacgcatactcagatactctgatgctcaggtttcagattctgggatgaataaaatgctactagtagtatcatcttcttctggatctgcaaacagatgttcagttagctgaagatccagagtaccacctcgtttgctcaggtacatgtcatcaaaacatctactactaatctctagttttattctctagacttctctattccaatctatctccaggatgcaacaggaagggtctgcatccaggtgggctacctgaagtggacgaagcggtcatcttcgtgccgctgctcgagtggcctagtggggaccccggctgcgcccgatgcctcggccactttaaaaactcggatcgcgagagttcctgcacgccataaaaatgaggaaaacgaggtggggagggcgtggcttcttttctctatgatgaagtgcgtggcatttgttgagccatcctaatcagagtgttgttcatgcatgtgccccgaatgttgggggcgtctgcagaaagaagggagggcgttcgcgtacacccccctttcagccccacttgcgcccgcgctctcgttgagccgtctgccttttggggttctagagtgaatagggtcggcacgttagcggcgagccggcgacgggtaatgtggtgtgccaacaactttcagctgctgctgaagtgaagagcaggtggcgatcaaccagtcccggcactgaactactgaacctgaagtggttactccattcatcgacagagatatttaattatttgtcacttttagatgtaatattttaccacataaacctacaaatcataaacatataagtgacaaattatttaacgccacatcgaaaaatggtaaatgcttatttgccccttcccactctctc

SEQ ID NO: 31 - DNA, 4774 bp, Brassica rapa
cccgctggtgattcccgaagtgaatcccgaggcgatgaaagggattaaagtcggaacggggaaaggggcgttgattgcgaaccctaattgctctacaattatctgcttgatggctgttacgcctcttcatcatcacgctaaggttcgattttttttttttgcaatgccaacgtcttcgcgttttgtgctatgagtaacgttttgattttggttataacaggtgaagaggatggtggttagtacttatcaagcagctagtggtgcgggtgctgcagcgatggaggagcttgtgcagcagactcgcgaggttttgcttctttttttaaccattccattgactttgattaacgataatgctgagagtttggattggtgtttgcttaggttttagccggtaagccgccgacttgtaacatcttcagccagcaggtgaatagtcaattttgcttatagtttaattttcaaatggtggtttagtgttctgattctgaattacttttttgattgatttgttgtcttcgatagtatgcatttaacttgttttcgcacaatgctcccatcactgagaatggttacaacgaagaggaaatgaaacttgtgaaagagacaaggaagatttgggtgagtggttacttgaagaactgttttgtagtaatatcactctaatattttgttcataacgctggttatgttaagaggctatttaccttttcctgcttttcgcagaatgacacagaggtcaaagtaacagcgacgtgcatacgtgttccggttatgcgtgctcatgcagagagtgtgaatctccagtttgagaaccccctcgatgaggtaataataatacacttcaaactcgtattctactaagtttgttattacttattagtagtttctgaagcatggttcatagtgaatttcaatttgaatcatgggtaaaacggcattctataggcatttttaacttcttttccaaggacctgtgatcagcgttagtaagcttggatagttcttgaggaaccttgcagagttaaatcaccttagaattgttatttggacttgttctgctagcatctttagagagcttgtgattcttcttacgcgacaaaaaataatcttatgatcaagttctttgttatcttaacagaacacagcaagggagctattgaggaaagcacctggagtttacattatagacgaccgtgcctctaacaccttccctactccacttgatgtctctaacaaagacgatgtagcggttggtaggatcaggcgagacgtgtcccaagatggcaatttcgggttaagtctcactctcttttctactaaatttaagatcatatgagttctttccattaagttaaaaggctataataactttgtgaactttcagactggacatattcgtttgtggagatcaaatacgcaaaggagctgctctaaacgctgttcagatcgctgagatgcttctctgatttggagtcccctcactcacttggcttctcctgattcttgacatgatcagatttgagccaagaacttgtctcaatttttttgtttccctatttgaccagttttgttacttttcattattcatgaagttctctctgggatctaaatcatccacaactctggaaccttgccaatttccggttcgaaccgataccggcttggttaatgagtctttgcatgtgatattatccaagaaaaattattagaccgctaataaacgcgcgaagttaatttttatatataccaagaagttgaagtaattaacaaaccgcatgtttaagctattgtaatttcgatttgtgatacaaagcacttaaagccaaacgctaacgctgatcttagattgactagcgtccaaggttgcgatttgggaccacagggacgctcacatggacctttccgcaggatattaaaacctttctcactctccaccatcctcttcaacttccataataactgcatcacactctctagttctaaccaacagaaacgaactcccattttcgaatttgaaaaatatcttcggagaagttttcgtgtcaaaggaaggatgaaggagatcgcgacgaggatttcaaagcgcaaggccgagccgacgccgttccccgggaagaagcttcggtcgtttccttcacgccggaagagagctcagatctctccagtttccgtcgagccacctctctatcacaaggaaacaggagtatccgctgcttccgtcgattcctgctccaatctgctctctgcagtcgacgacaacgtttcgtgcggttctagcagagtcgagaagagctcgaagaagaatcgaattgacgaggaagaagaagtctctagacctggcaaagacgtgaagaaggagacgatcgctgatccgaagtttcggaggatcactagatcatactctaagctaaccaaggagaagatcatagatgaggctgaagtaagcgaatcgtcattcacacgatccgacgtgacattcgccgagagtaaggagagcgacgtcgtttcattcgtttcggctgtggagtcttgctcgaagctcggaggcgataacgaagaaaccgaaatctccaaaccgagcggattcgtggaagaggtcaaggaaacgaagccggaggttgagacagtcggatgcgtatccgatctcgcttgcacggagacgttttccggcgaagatgtttcggatgattacgaggatgagttatcggagcagcgttccgagatgttttcactatcctccgacctcgattcatcggattacactccgtcgatgttcttcgattccggaagccaattctctgagaaatctagctttgattctccaatttcacatactcgctctctgtaccttcagtacaaggaacagttctgtagatccacgattgcgaacggtttcgaatcttctcgccatgatcaaaaccgtgtaattcactctgaagtaagtgctatttagattacagattgaaggtgtggttaattacttgatgtttcactcgatttgctagtctaatttgatctgagatttgttctaaaatatacttagcatttaaatccgatattctgttatggaatgaatcttgaatatacgttttcgtttagctggtaaggtttgaagataaagaggtggaagagagctatcaaatgctgagggaaagagagagaagtcatgcgtatttgcgtgactgtgctaaggcttactgctccaggatggaccacgctgatttcatccctcgtctacgcttgatcatggttcaatggattgtggaggtaagcactatcattctgttcttatatgcatctgaatgttcaatctcagaaatatatacatcagtaggagacgattgtaaatggaaatgcaatgttttgttgctgattgtctgctctctgactatatgcagcaatgttctgacatggggtttcagcaagagaccttgtttctaggagttagtctgctggatcgattcttgagcaaaggatccttcaacagcgagaggactctagtactagtcgggattgcgagtcttactctggccaccaggattgaagaaaaccaactttacaataggtactaaccattatccatattctgaccttttctgttcattagataaatgttttgaataagaataagatgaggaccatttcaagaaactgattattactcattagattaataggttaagagtcatgtgtgtcaaatattgaattatgtttctagaaagctctttaactggagaaccctttcaacacacacgtagcaatagttcagtttgcttcagctgttcacctgatacttcctccatttatgtagcatccggaaaaggaacttctacattgagaacctaaagtatagccgtcatgaagtggtggcaatggagtggctgattctagaagtccttaacttcaaatgctgctcacccacaatctttaacttcttatggtaaaaacctctattactatatattttctgttccaagacacaaacacacactgaaatatacatagtaacctatcatttatatttctcaggttttacctaaaagctgctcgagccaatcgagaagttgaaagggtagccaaatcctttgccgttgcctcactgtccgatcacactcaactctgtttttggccctcgagtgtagcagccggactcgtagttcttgcctgcatcaaacacaacaaaacctcagcctaccaacgagtcgtaaaggtaccagtctcttcaacactactttaaatactttttgatttgaagaatatacagaataattacaatcccaaacctcttttttctcgccttctgcaggttcatgttagaacaaaagataacgacctgcatgaatgcgtcaaggtatattttaaacatcactctcatactaatcagaccacttattctccactaagagggttagcgaaggagttttatattagtgtttctatatacagagcctggaatggttccttgggcagtaagcaatcaacaacaacaaaatacccccccaaaccagaacacagtatactcccatacgaactcacaggttattattaccatttatgaaacaaaagtatataattggtataaactaagcaatgcctctacagatttatatacttaatctctggactttttagctgttgtattggcatactaatacccaattatttgtgtcgctaccaacatttattgcttattttccccaatacactacactcccattttattaaaaatcattttattgttcagtatctcctcttcaataaggtctggaag

SEQ ID NO: 32 - DNA, 5694 bp, Medicago truncatula
  misc_feature (853)..(853): n is a, c, g, or t
aacctaccaatatcataggttcacttctatcacccaacttctttctctttgcatcatgaacatgcctggagcacaaggaaccaaacactctcaaatattttgcagacttactttcttattagcaatgaaaattgtgaggttacaagtttatatacaacatcatatgtgagttagttgctatacaattaataaaccaagacttactaatttctaacaaagtagacaacaaactaacacattgttttaactacttttattattgcaactaacttgaactaaaaactcacgattagtagcagaagaatatttcttcatcacattttacaaatacataacaaacattgttttgttgattttgtttttagttacagtcgtaacatttgggaaaaaaatatttatattagagttaactcacgcgtaaggtcgtagattaaactttcatcgtcgatgtgaacacacctttattgattgatctataaatggtgaggcctagattaccctctcttgtttatagctgaaaagatggtttattaaaattgaagtgtttggtaaaattagttgatgaagtggctgataagtaaaaaatgacataaaaggacatgtttatatatatatagacatttttctaatgtatttgttttttaatattttaatttatggttaactatgttttggatccctataaatattcaaacttttggttttagtctccaataaaatttcaccgacaattttgatctctgcttattttatttttttgtacaaaattgagcaaaagttcattgatctcgacttttatgaatcccaaaaataagaggaaagtggaaaaaaaatataagcaagaatataaaangtggaaaaaaatgcaagcaagaatatataaaattttacaacgtaccgtagactagttatagttaaatataaagcatttcttttaagaaatatatataaagcattcattaaaaaataaaataaagcatgacagttttttttttaaaggagaaaacgtgacagttgttttattaaaaaataagctatgaacttggccgttatttttaagccatgaacatgttgttttattaaaaaataattaaattaaattaatatggttaaaattggaagaaattataaaaaaaaaaaaactaccagctataagctcaaaagctacttgaaatagtttctaaaaaacatttatgctagtgaaaaaaactttttaccaaacacatcttattatatcaaaacgagcttataagctagtccaacaagtcataagctagcttattcgtgttaccaaacacagtcatgatgggttgaatgtgatgtgaaattttaattgttacaaaccctatagtgtaaattaattatgatttatacctaaatataattaaataaaaattaaaagttaccttattaaaattgattttttttatagaaaaattgactcatttatttgagatttaatgttgcatttgtatattacattttgattggttgatatcttgaaggtgtaaataccttctaaataagaaagtgtaatgtagaaaaaacctctattaaatacatttataaacatttgttaaacatcgagatatgttccgacaatgatgagtctagagtccaactaacaaaaacttttttttttatataaaaaagatattatgttaaaaaaaattgataaatattattattaccgctactgcattatataatttgtatatatatatatatatatatatatatatatatatcaaatgcacttttcaaaaaattaaaaatatcaaaacactttattacagttaactttcgtcatgatgtatgttgtgatggacgtggggcacggaaaatgcactacgtgggtccacctcatataaaaaccctcctccctcgtttttccttcaatttcataaccatcccttcgaacactctttccttcactcatctcaactaaacttaactccaacaaaccaaattcaattttcactgcattttctcacttcacaatgataataatcaaatctagaaattccaaacgcaagcttcaacacgaaccttcaccgttacacgtcatcagcaagaagctccggtcgaagattcctcgccggaaacgacgtcagatctcaccggtgctacttgtttctccgagattcaaagcttctcgtgagaatcgcggtttttctgttggttcagttgattcgagttctggttcggatttcgccggaggtgaagtttcgtgtgattcgagtagaatctctgctgttaaaggaagaacgaactcgagaagtgaaatttcgagtggtgttgaatgtgttcgtagatttgagaagaggaatgagaatgaagttgaagtttcggagacttcgtgtgtggattctagttctggagttcgtagaaacttgattttgaagtttgaaaatggaaaagagaacgatgaagtttctgaagtttgtacgaaatcggaacttacttttgctgagaattcaaaatcgagtaacggaaattcgaatttgaatttgaatttgaatatctcgtcggagataacacgaaacgatgttgtttccgttaacagagcatcggaatctgaattttctcaaatttcgagaaatcgtaatgctgatgagaattgcgttatcgcgcaatcgattatgaagaattattcggataattcaggttacgattccgatctagcttgttctgagaaactgcaattctcttactacgacgatgatgaatcggaggagtattgttcaagtcagggaactacattctctgatcttcactcggatattttcagtgaaggttcagattattctccgtcgcagttcattgattctggtagcgagttttcacaaggatccgttggtgaaactccttctcatacttattcgttgcttcttcgataccgcgacgaattcgcaaagctaggttctccggttaaagctacttcaattgttgtagaagatttttctcttcataacaatgtacgttttgtttcatcaatttcttagttcaaaatgttaataatatgctaattattctagtttaacttttatttattttatttttgttttagttttcgaggtttgaagatattgatgatgaagagagttaccagatgctgagaaagagagaacgaaggcaagctttcatgtggaattatggagaaagatatttctctgcaacggattttgcggaagtatttcagcaacgttcacgaatggttcattggattgttgaggtagtttactctataactacattcaattaagctataatttattattatgattaattaattaattagttaattgttataaaagtatgtattattatgcttaatcattgtttaattgctttgatatggttttgttaattaagaaattgaaacagtgacatggttggaaatggagaattgaaacagagttaggctttgcgtttgaatgtagtagttatttttatttatttgttattacttaattaaaagctagtagttcgttattaagtagtgttgcatgtgagagagcacgcttgaagagtgtgatgagataaagattgttggagaatcagttagtacttagtagcttccacttgtcttgtgagacacgcttttgaaggttagaaaataagaacaagaacgagagtgagaaatgtgatcaatcaaatatttatcaaatgtgaaataaaatgaagtgtgtgggttaaattcacattttagtacatggaatatgtgcacttttctttttactcgtgcatgcggttagttacttcaggttttccaacgagagattttaccttttacttgcaagttgtaaatgtagtgtaaatttctttttaaaaattgtaccagctttaaataaaatatctataagattatagaaattattataatattaacattttggcatgcctgtaaccataggctagcaaaacaacttatacaacaatgatctaaaaatttaagaatttaatttgtatataacgatactgtaaaacttttttacacgcgcataaaaataaattttaaaatttaatttggttgttttattgttacattacatgattgtcttcttctcatccttattttaattaagagacaaaactatcataaaggaatgagcaaataatttttattgctaccatttttattttatgctatctataatcctcatctatgttcacattgaatttttggagtttcaatttctagtttacttatttaaaattgcttagttttgaaatttgctttgaatatgcagcactcttatcgaaaacagcttcgaccagagacaatgtttcttgcaatcaatctacttgaccgtttcctgagcaagggatacttcaaagcagaaagaaaccttcaaattgttggaatagcgtgtcttacattggcaacaagaatcgaagaaaaccagcaatataacaggttcatattttacaaatttagatttttgttttggtgaaactacagattgagatttttatacttattaattatgcactgtcagataactaaaaaatttagttcatacgataattatatgtgtaaatttaaagaactcttaaatattcaatttctttttataggtataaatttattttacaatatattaactagctcagttgaagtttgagttaagaaaacaataaagtcaattaatgatggataatcaaggttctcatgatttcaataatgatatatgtactacctttcttcgttttaagttaatagtaacggagtcttttgttggcattatagagtgaaccagaaaaatttctacatagaaaaaagtgtgtacagtagatgcgaagtggtggctatggaatggatggtgcaggaggtgctagagttcgagtgttttcatccaaccatctacaatttcttgtggtataagtttttctgtacattcttatagcatctaaaattttgcagtttcagtaaacataccattataattgcatcttacatatatagcaccaatgaataaaaacttgtaacgccttaacaaagtgtatttagatctttcatgaatgaaacaaagttttactcgcccattaaccccttgtaaaattttgcagtttctaccttaaagctgctaatgctgatgctgttgtggagaaaagggtcacgtgccttgcattactggctctgtcaggtcctgatcagctgtgttactggccctcaactattgctgctgcaatagtaatcctggccagtctagagctcaatcagaaagcatcccacaaagtcataggggtaattactgttgcgatacagaactaagccttttttgttgtacaagaaacaagcctttgaatcttaaattcatcaagcatatcatgttggttaccattatatgacacggtatgtttagttgtaaattctttaggcaagggccacatatgcttcaggggtttaagttttattggtcctttgatattttgatttaccaatgtagtttaagtaatcaaaattaatcaaaatgaagtaaaaggatcatgtttgtatggtcttgatttgccctaaagtttgctaaacacttgatgatatatacagttgtgtatgctttctttgaatattataagtttcacttttcctcattagatcaaaggattttaatgttcataattaatcgtgtgattttgcagatccacattagatcaaaggaagagaatttgcatgaatgcatggaggtatga

SEQ ID NO: 33 - DNA, 7437 bp, Glycine max
tgaacctatattattattttaatatttaataaaataatattgtactgcagtaaatacattttttattgaatattattagtaattaattaaactattaattggactgaccaagcagttctattgcttttggttgaccataatatgtcaaacatcaaagataaaaaaactaaacatatattagcatagttagtcaattaaaattaaagtttggataaaaaattgagaggggtaagttttttctaatttataaatttaatgatgacttttatgaatatgactccatgtgacatttctcattttattgtaggattcttcctatttgtaggtaatttttggcacgcataaaacctccggaagttgtcgcaggatttgaaaaatgaattgattcagattttgaacttgttctttccctgatttctctcatttgagacatgaaatcctatgaggaagtaataatcaattttattttattataaatatactttaaatattaggttgaggtgaactagttgtgttgaaataatacttcttttttcctttcttttcttagtttctatataaaaaagattccctggaataaaatttgatacttgttatgcattcttgccaattctaaacgagtaaattgttgatacaacaaagttcaaatacatcaaatgtacaattaataaagacacaatatttatattgtatttaaaagaaacatttttaaccaacaagtcatttcttcgttttataaaagaaaaaagtaattaaaaagaaaattttccctaaaaatgataaactaaatttcttaacaaagattaattattaataaaattaataaatatttcagattaatttcatgacttaaaaaaaaaagaagaaacaacttcaaactacacattttatctctccatgaatacaagtataaatagagaaaataaaacatctaatgttggttactactttgaaaccgcattttctactctagccatcattattttttttgggtcatattttaagtgcatttattgcagcagtacaaaaaatattgctcaggcattactgttatttagtgcaaccatctttattttattagttttgtaaaaagaaaatctttattttattaatataaatatatgaagaagatagttgtatttttttttctttatttgaaaatgtgatgtatttttcttttttttatagaaaagtgcatgaggaggctgaaaaaatctaaattaaaaaatacttgaaaaaaacacctgggaagtgaacatggtggttatgcttatcttgcatgcgttattctaatcagtgaaaaacttggcaaaatggtcatgaactagacataaacatgtgttctgaaataaatcaggatacatcggtttgggctttcactttttcatctctttgcttatttttcctaatcaaaacaagtataaattgtaaagttttcctttacacaatatatttcgtttaaacattttttttactgtggtacgtatcacttcaacgatacctaccccttttcataatcgtcctacaccacaaatctctttaaaaaccaaatgtttacccaaaatatatttttgtttcaacaactcaattaatgaaactaatcacaattcacagaacctcttgaaaccctcgaagccatacacatcatttcaattttctgtgtcgcattaattgatctcgagcacaacaaccttcttccacaattcggttctagctaggtcttgcaaatgcgcaaagatcatttacatttcataatttgagattaatatcacaaatcccaaattatttactctaaaatattgttaattcccagtcaaaaaaatacttgtaaatccaaaatactagtatttgtcaacttaaataattaattaatgagtttccaaaaacaatttttacaatcaacaagcgtggacgtggtgcacggaaaacgcattacgtgggaccgcaccgtataaatacccctccaacctcgttttttcttccttccctcaactcccgtaaccgttcaagcacacttcccacacactctctcttcattcaatcataacaacaacaacgattttcaattttctctctgcgtttcttcgttaaccgaactccaatggcatccagatcgagaaaatcgaagcgcaagctcgagccggagccacatccgctcgtcatcaccaagaagctccggcagaagctccctcgccggcgccgtcaaaacatctcgccagtgctcctcgtcggcatctccgcccagaatcctcgtttctccgtcgattccagctccgtctccgacttcgccgtaggcgaagcctcgtgcaactccagcagagcctccgtcgccggcaagggaaacataaactcgagaaacgaattctcgaccgattcaacgagaaatcggagattcgagaagcggaacgagaacgaagttgaggtgtcggagtcttcgtgcgttgactctgcttcgttcgcgagcgaacgtaacagaagcttgattctgaagtttaaaagagaagataagaatctaaacgaaaacgacgacgtttcggaagcgtgcacgaaatctgagattactactgttctgaagttcaaaagcggaagcgagactaagaatgcaaaagaagacgacgacgtttggtgcgcgaagtcagagattacttgtagcgaggaacagttcaattcaaactcaaagtcctccggtaacggtaacggaaacataaaagtctcttcggattcaaacgcaaacgacttcgtgtcgtttagttccggtgttcgcgcgtcgtcgtttcatgaggaagcgaacagaaacaaggaaaacactaaaaacagagcatcggaatctgaatactctgaagtttctagaagcctccacgtggaagagaattgcgctgatttaatagcgcaatcgatgacgaaggaggattcggatgtatacgacgtcgttgcggatctcgcttgctctgaggatctgcgtttctcgtactgcaacgacgacgacgacgacaacgaatcggagtactgttcgagtcagggaaccgtgttatccgaatttcattccgagcttttcggcgaatgctcgcagaatgagctttcggattactgtccgtcgtcgctgttcgtggattccggaagccagttttcggaaggatccgtcggagaaactccttcgccgacgcatttgttgttccttcagtacagcaaggagttcgcagagctagtttctgctcctccgttgaagaacgcttcaaacgttgaagatgttgtcaatgtaggttttgcttgcttcgatttcagaattcgaagttattatatttatattattattacttgttctgttagaaattattatctgtttagcctagctcttattatatgatagtttgtaattattaaatgttagtgtaatttattgtatgtggatttaagtttgtgagatttgaagatttggatgacgaagacagctaccagatgctgaggaagagggagaggaggcaaggctatgtgttgaattatggtgatggatatttctctaccactgaattcggagacaccgtgattgagcaacgtgcgcaaatggttcactggatcattgaggtaggtttgtctaaaacaaaatccaactcattatattattatatatgctgttactcttgtcgtttgattaatttcacttttatatagtttttgagtaaataagtacgccttaaaaaaaagagtaaataagtacagtatatgttatgaattgtactaaaatttagttacaatttatcatatataatttatttcttcatgatttttgattgattaatgaccgagtataaaaattaaacaagttgattaaggagagctcgtgctttgattattagttagttgttatttttatttatttattggtgtatagatagtcgctctttattgagtcaggttttggatggtgagtgagtgagcgagagagaacaacacgtttgaagagtgagtgagaatcaaacttgattcggttaaaaaggtaaacactttgtacaacaagttgttgggagtaatattgaatggcgtccaattgtctcgtgacacgtcagagtcatttggaagacgaaagcctctacgctagtatcgcggcggacttgagtaaatccctgtatcgtcatgctttttgatgatgcaatcatgcaacgcacttttctcgtattcgtgctcgtgcatgtgggtagttacttcacaagggatgatacattttgcttttcacttagaacccaaaacattgaggagcattggagtagggaaaaattatttccttctagttagtcgtcccccaaaaattacttacattcaatttttgcgagtctagctaatgttacacaagtatagatgctactgcggtacatacacatacactcacacacacacacacacacacatatatatatatatatatatatatatatatatatatatatatatatatatatatatatatatatatatatattacaccagtttcaccattattttaacatttcatatataggtaggatgcattcgacattttgtattggaatggcaaggactaaaggagcttgacagaagatttaattgaatggcaggccacggcctaaaaaaataaaataaacgctacttctaatgaatatgcaattgttatgtttctacaaaataggagtatttcttttttaaaattttaataaaattatttcataaattaaacaaatgaatataatattatttatattttttattatttgagcattgtacatagtgcgaaaatatttcggagcatcaatcaacaactcatgccgtttctcttattaattattaatttattattattattaatgtgttttttatttgaagaaattattaatgtggttattttattgttgttatcatctttcatgatcgaataaccataagaacattctttccttttgatgaagggaatccattttctttttccacctgtttcagacagcgacactaatcatgaatatgtcattttttattttttgtccatataaagcttatgtattttgctaacataccgtcacctaccaatttacattaaaaaaactattgtatgcatgcgaacctatttagatttggataacaatgtttttagattcaggtattgagtctgtcatttctaattgctgcatttgtggactttgaattgccagttgaatattttttaaaatgctttatgtttgaaacttaattggcagcaatcttgtcgaagacagcttcggcaagagactctgtttcttggagtcaacctacttgatcgtttcctaagcaaaggatacttcaaagccaaaagaaaccttctaattgttggaatagcttgcctcacgttagcgaccaggatcgaagaaaatcagcaatacaacaggttcatcatttaatctttacatttcttccccatcctccaagttatcttgggcaaaaatgtcgttttcaatcatccctctctttacttgtatttggttgatcgattgtgaagtgattgttagtgacggtcttgaattaaggaaaatggaaatagattatgatttaatttttctagagtttgagtttatatatcctcattaatcatgcattgtgccttaattaaaagaataacaaattttccctaatggttcaaacattatccaattttcacataaattttattttcttacacatgaactttcttgaagtttaggtaaaaaaaaaaaaccataaagtttgtaagttttaatatcccatgtgtaaagttagccaaagctgcttgtaaaaatttttactttcttcgtttacatattcttgattttcgtacaactagggaaagtgaaaattaacagaacttataattttttcttccaattaaactcatgaaagccatattcaatgaataattgaactcttttgtgggtactacgtacagagtggggcaaaaaaatttctacataggaagcaatgtgtacagtagaagcgaggtggtagctatggaatgggtggtgcaggaggtgctcaagtttcagtgctttctgcctaccatctacaatttcttgtggtataactttttattctttcagcacgaatatgacctgaaattctgcaaaaattaaaggttagctaataatcgttgttcaaggtctccattcatcattaaaaaaaaaatctgtttcttactgggaaacaacttgaattttaaaatgaacggttgaagcaaagacttgctatcatttctctaataagggatttctttttgccttaaaaactctgcataatttataaaattaaaacaaacttttgtaattgtgacttcacatagcctcaataagaaaactcataagcctttttgtttatgaattcagctaaataacactccactctataatttttcaggtattacctaaaagcagctaatgctgatgcagtcgttgagaagagggtcaagtatctggcagtgctggcactgtcaggtcatgagcaactgtgctactggccttcaacagttgctgcagcacttgtaatcctggcttgtcttgaattcaatcaaatttcatcccacaaagtcataggggtaattacattattgcgatatagaactaactctttatttgaatcttaagttaacagagtcagggaggttaatcaaatgacacgatttggcaagaacagatttgcttcatagatatcagtttaatttggtcattccttgatattttaataaatcattgttattttagtacacaaaatcaatcaaaatggaatacaatagtatttttatggtctagattggcactggagtttcaaaaagattggcactgaatgcagagttgtgcgtgcattcctttttcctgaatgctcaatgtttctattttatatctttattttctttagattggattatcctgtaatgtccatattagattgtgaatagaatgccgtatagataattaatcttgtgattttgcagattcacgttagatcaaaagatgagaatttgtacgaatgcatagaggtatgctagtcctatatatttattgtaatttgtcagtgtacatttttttgaggtttaaactaacaaaaagaatagagaaaaaaactgaaataccataatgaaaaagcgcctcacactacaagtattatgaaattaatcaaatcatttcatttattttgctgagaaaaaccccacaccacgattaggatcgatgaaatatatcattttcgttaattatcattcaatttctcctattatcagtattgttcatgtatttaacatgaaacttatattcacttcaacagagcctggagtgggtgttacgctttcctggatagcatactagttggcgtggaggcattagttatacaattcagtttggtgacgatggttcctaaccatcagaagacagaatggctttcttatgcctggttgattcttcattaatacag

SEQ ID NO: 34 - DNA, 6573 bp, Cucumis sativus
atcttgtacctagcctaaacgatcttgtactcttgtacttaatctaaatgacattttacctagtctaaatgatctattagtgatagtggtttatctatgtctatgtagaatagacaactgatcgtttagatattggtatactatcatttagatcttgtactaagaagaaaaaaaaagagatgaagaagaaggaagaaaatctagaaaaggaaatgaaaaaatcgcaaacaaaaagaagtgtgaatatgaggaaagagaagtaataatatgaagtagagaataataaatagcaaagaaaaaaaaagtgaagtgaagacacaatattaaaaaaagaaatggcaaatctgaaatttatgaaaaaacaaggagactttatagattttaattttttttttgttaaacgatccataaatatttttgggatttgttaaactatccaaaaattgattgataaatgaatattaaagtatcatttttaataatactagaaaattttagcatgcattgcatgtgaggaccttgttaacaatataatatatattgttgctaaaatgaacaatagcatgtagtggttaaatgagtcattctttatatacaatatttttattcatacattttcaacattcatacattttcaacatttgaatttttataataatttcatttctcttgaactttctctataacaatcttagaaattataatgctaaaagtgaataataaaaataaatttaagaaagcctaagtagaagtgaacgtttgaatgttgaatgtttgcacaaaaatagttgaagggagttgtttatattgttaataaaaatagaaagttacaaaatattttaaacttatatgaattattctaaaaatttaattattaaaataaagagagattttcatgttttaacctaagaagggttgtgagaatgtaattaaaagataataaataataattatttttaataataatagtaattaaatcattttaacttcggtgtgtgataagtgatgttttcttattttatttttaataattaaataattttagccttaatatgtcacaagtaatgttttttatttgtttttaaaaaatactcattgcattgtgtacccgagtgttttttaagttaccaacctaccctaaaatattcaataacaatatttttcatgtttaaaagactttttaatcaataagaatataaaatattaattacataagctagaatgaaacaaaaaatataaacaaaaattagagaattatcatttggacaaaatggttcaaaatggttcaaaaatgattgtaaatgttagattgtaagataatataagatagatatctagataagttggtacaaaagtaatactaaaaatgcatgtgttttgtaataattttttttacaaataaaaacttttatatacaacaaattcatatcataaaactaaccaaataacgttgatgaaggtagatttggataaggataaaagatattttttattaaatatttttattttttaaaatctttttacaatacgagtaaaatatgaagatgtaaagagaaaaaaaatatattttcatcttgtaaactaaaagaaatgaatagttttaagtgatttagtaaaagcaaggagttgatgaaggtagaagaagaagatgaattagatgagttaacaatttaagttgtgaaagcgatagataattgatatgaggagggtatgttggtatgttatagcaaaagatgagattaagtaatttcacttcatcccccaaactcaaaactaaaataaattatatatttgagaaaataaatattgattaattttatttacaaaaaggaaattagggggtgggggtgggggtgggacccatacccactacccacaacgaaaagaaagctcacttccaattctcatttctcttttcgcttcttaacttccataaccgctctttcttcatcttcatcttcatcttcatcttcctctttctcttcctccaaacaaacaccatgaaatccaagaaacgaagaccaaaccccaaacctcaatccttctctcctcccaagaacaagaagctccgttctcagctcccacgccgcaaacgccctctgattttaccctttttctgctgttatttggattccgattccccccctccctccaccaccttttcttttgcttcttcttcttctttcactgccgcacaatccacctccacttccttcttcccaaccggacctgaggtctctagccacctcaaccccctgaattttaggaagacacgatttgattcgaacaaggaggttggagtagggagtaatgagcaagtgtctgaatcctcttgtgttgaatctaattctggactcgattttggtgtttccggaccaagcactacttccaagttgaagaataggagaactattcacggaaatgaagatccaattgatccagcggagaatggagttgatgcctcgtcgaagctttgtggaaagggagctgtggtactcacttcttgtgtagagtcttgtgctgaatctatctttcagagtgtttgttcgttcgaagagaaaggattagaggttgaagataacagactatgggaaattcagttacctgagctacagaaaaacgaaattaataaaactttcactgtttcgaagtcggattcgacgatagaacagtggcctggcagcttgaagattgaatcggaccttgcttgcacagagcaattctcttacgatgatgtttcggaatacttaagccagccgttgtcgcttcagtcaactattctattggagatgtctgatgactgctcagattacactccatcaattttcttggaatccggaagcgaattttcagagaaatcgaacgaagacgcagctcctacatcgacatttaccatgttgctgcagtacagacgcgaatttataagcttaaatttctctcacatcagaactagctcgtctattgaagaagaagaagtagatcaatctacggtaattcgctgttttcgtgcttccttttcgtttaacatctgcaacaaaacctggagctaatcaaacaacttattatatatatatatggtttatttaatcgattttaaattagattttgagatttgaagaattggacgatgaagaagcctatcgaatgttcagaaatagagaaagacgccaactgattatttgcgactacatagaggaatatcggtccacaacggattatggcgatttcattcttcagcaacggtcaaatatggtccaatggatagttgaagtaagtcctggatttcaaacctccatgtttctcttaaaaattcctgaattagcataagcaattccccctgtcttccattttcatcgttaatagctttggtattctgagacattagaactgtagagtgtataggcactgtctatcatattacaatttgtactgaattgccaatttgttcttagcatgtcgtaaaatgagtcccctgccttatttgatttggaactttatccaacaatgtgatttactgatgaaaattacaaagtcattactatgatcatactttttactatttaaggcaagcagttcatgattctgcacacatatacacctagatgttacaagcttcagtgcatcttgaattagccaagttcagctgatttttcttttcattttgtacttctacttagatacataatctgtttatttttaacttaataatagaatactgattcataacagcgagatttgtgctcattactgtgaatgttaggattttcttcgaaatactccaacgtagttgcattttcatcatcggttcatggaatacattctttataatctctttcaattccttttcatgcgttaaggcttgtcatcaatcagttggatcaaacttttttacattatatagctttaatttgttgaatgatggcagcgatctagagaaaagaaacttcatcaggagacgacatttttaggagttacccttctggaccagattctgagcaaaggattcttcaaagctgaaactcaccttcaaattctaggcatagcatgtctaactttggcgactagaattgaagaaaatcagtcatacagctggtgacttttgaatctctcttttgtcccatttgtcttcatctcagttttaattatataacaactgtggttcctatctactatgaccttaacttcgttagtctatattatgtatgatgaatattgataggaaaccaaactgtatgccaattggtcttcttgtttcaatccaagggtgtagaattgagtaaagttaggatcaaatggtaagtagtacactagaaataataatcagaaaaaactgtctaaaagtacttgaattcaatagtcttgaatgttttccttgagctcaaagtgccgggactgaaactttttccgttcatgaacaaaataacgttgtgtgattatatcgtagatcctcttataggaaactatgtaacagaaaatagccatacatgttacattagtgtcgatgcacacacctcccgtacggcactgcagtcgaatcctattgccttaacaatatcttaagttcgtaagttaacaactcgtgcacagatgatatccaagatccaccaagaaaacattatatggcaaaccacttcaatcacttgatcgggccatcagacaataaaattctgatcatatagagctccaagtcaagtcagatgtaaaacaattgtttaaaactgttcttctctctctctctctctcttcctcaaacttcctcttatctagtcttaatttatctttgactggtaattttcatgaaaagtgataaataatcatcgtctgtttcattaaatagagctttgagaactgaaagtatgatagtacttatttgtttttgggcaattcaggttacagcaaaggaatatccatgtagggagcaacacgtacagaagatcaaaagttgttggcatggaatggctcgttgaagaagttctaaagttccattgtttcttgccaactgtttacaatttcttgtggtaaatcttcctttcactaacttcaccatttgtgggaagggaaaaggttttccctttcgtgcatccattgctaataaactcctgtccattaacaattcgaatctactccaaataacatgctttatttaaatttcttttgttgaaaattatcttaggttctacctgaaagctgctggagctaactcagatttggagaatcgagctaagaacttcgcagtgctcgttcttgcagaaaaagtccagttttgttatttcccttcaacgattgcagctgcagttgtcatcttggcgtccttaggagaaaaacaagatgcaccaagtgaacgagtcattgaggtacaaaaatacaaatacctttaagagggaaaactcctttatctatatttatatacttatgatttatccaacagaacaaaaataaacaagtaagtctagaagaaaatttgaagttttacaaaaaaaaaaaaaaacagcataatctaagtccaattagattccaacacgtaaagtgcacatataaattccgtactcatacatatactaaaaggaagtgctaggttatagtgttagtttagattacatatcaaattcataatagtgaactttctactgttaatcaatataaatatgaaggtttgtttattataaatttataacagtaatttatgtatttatttagattactcttgcatatttcttactttatcttgaggaaggtttcctgtcttataaaaacccttccatgaccaaaatttcaaccttagactagtcccattgaatcaatggaaggatatatgtccatccttccaaaagaacaagaatcatcgatcttgttcttcaaaacgtagattttaccttttttttcttttcttttcgaaacaaaattgaaaggacattgaatccatgatcacataaacattaaaatatgccattaaagttgaatttgtgaggcaaacatgcaatgagttaaccctttttttttctaattactatctatttttaataagttatttctcttatacactttttgtagtttgaataaaggaactactactaataccttgcaatttctttcgaaatctacaataatagaaataacatgtatttagacatcttgttgaactaactcataacatcgattgtattgtggttttgcagatacacgtcagaacagaaaacgatgatctccctgaatgtatcgaggtatttatagtcaactataaaaaaatcaattgatcattgcataaatctctaattagataaaattttcaaggcttaaaaacacatttttaatagatacaactttttcaagcattaaaaaaggatcaatccaaacaaatccttattttttcagcaaaaaaaaaagtgagtaatacagttggaattttaacagagcttggagtggctattaaagtttctatgatggaagcatgaattcctaagacagcaagaaagaaagcaagcaatttatgttccattggtctttttgacacattcttccttcctgacctgaccacttagacatcatcttgaacacagctagtgaagctcaccc

SEQ ID NO: 35 - DNA, 5683 bp, Solanum lycopersicum
caattattttttatttttttcaaaaagctaaaagtaggtcatagtggcccatattattaaacaataaagtagattttgcacaaatcttttagtgttataagttaagttagagagaaaatcacctatttgattgaacatgctgccaattaaaaatttacctctcttattatagtgtgtgtttttgggggatgcgcatgcaggaaggaagtttgagtctgatgtaaatgactaaaatatcatcataagatctattttattaacaagatttatcaatattttagcgatttttgtgacttcgcaagttactcggaattctaggttattgaattttctcatgttagcattatacaatggtcaaactagatcactttcatatttgggaccttttactttttttcatcaggcggaagttagaattaattcttgtttatctacatttggccatgtggggaggatagaaacgtttgtattcagctacaaaatttactttgtagtgaggcgttttgtttttatcttaatgtgttttgagttctgttttattcaaaagcctgcatctagcaccagtggtctagtggtagaatagtaccctgccacggtacagacccgtgttcgattcccggttggtgcaattaatatgtttgcggggatagctcagttgggagagcgtctaaatatctactttcaagctatctaagtgtgaacaccttcaacaccactgaaaagtgtagcatagtggtcgttggagttcattaattagcagtcgtgtgtttgattctccctaacatcatatttttttggagagattgaaatattttttattttagctatattttaaaaattacataacattttagattcaatattcatgcttacgtacaaaatatgatgttagagaggatcaaacatacgactgctaattaatgaaccccaaagttcactgggctacacttttcagtggtgttgagggtattcatttaaagttataaacttataatattcaaatctaatatcgtatatactgtgtaatttttcgatcaaaagagttcgggtgaaccccttacctcacacttagatccgctcgtcgaagtaggaaacattagcattcttatacatgaagtaatttgaagaaagtgaaatgatttatgaagtacttatttttgcatctaacttatggctttcaattaattgaatcgtactaaattttggataagggtccatcgatatcttatatgatttcttattgaattttgcatagagatccacagacatcaaaacacatcttttgaaattatttttatttgttaagttttgaatattacttttactctttatatattttcaattaagaaataaatattaatagaaagtaattcgtcaacaataaaaatattattccttgtatataagatttgtttgagcaacattgtataatgaacgtgttattcattgaagatattaatttatacaagtcaattagtttggagtattttattgaaatcagaagcaaattatgcaaaaacttgtaatgctgtgagctacaattctcactctcaaaacgaaaatatccacatttaaattaatactagtagatttatcttattcagaattaaataatcggctgacttcttttataagaaataaaataatttaaactatttgtattttttaaaattttaaaaatatatatatatacacactctattttattttatgtgatatttttttataaatttttcatcaaatttaaattatttttgaaaaagaaaatattacctagagaaaaaaataaaagaaaaagaaaatgtgaaagaaaaatacacaacaacgtgacatcaacgtggtcccactcgaccacagcgtatataagctctcacactccccattttcctcattttctctccgagcaaacaaacgccattaacggctttctctcactgacgcacacaacttgaacacactcagtttgagaaaattcacacgttctaagcaaagtacaagcaatgaagcgaaagttacatgcagaagcagttcaaccggcggttcagcaaccgaaggaaatcctaccggcagtgaagaggcagctccggtcgaaattacctcgccggaagcgatcacatatatctccgattctgcgttcattctcaattgctgcttcgtcgtacttgacaagtgaagtctcgcgtcaatcgagcaaaggttctgtgaataaggaagtgaagaagcgtgaaattgaaggagaggaatttcggagaattactagagcttatttcaggaagaaattacttgtggatcagaagaaggattctgaagtcgaattatcggagtgctcttgtgttgattcgtgttctgaagttatcggaaaaatcataaaaattgaagatccagttgatatctcacgcgatattgtttcaaagcggaatagaaatgcaaaagtaattgaaggaactgaggattctgaagtaatttcgagatttctgaaagcttctggtggtttttgcggtgaaagctcaaaatctggagaggatgctgttgcaagaagcaggaatgctgcgaaaatcatccatgaagatgtcgtttcattcaattccgtcttacagtcgccttcggagtcaaaatgtggaaatttatcagttcaatcaatcaaatgtagtgaaaacagagcagcggaagaggtcgagtctgaagtttcacgagtctgtccagaggtagaattatctgctgtagaacaagctcatgagaaactcgttgaagcagaattggatctggaatgttctgaaaatttctcaattgttgatgtctctgatgactattcatcagcttattctgaactccaatcggaaatattcccggagagttccgatatagatatctccgactatagtccgtcgtattggtacgactccggaagccagttctctgagaaatcgaatgcagacgctagtccttcacctactttcacgttgtttcttcggttcggtcaacagttctgcagatcaaccgctgctttgcaatccactcccataaactcgtcggaagatcaaatttctactgaattcactgtaagctatttatttaacttcctttaatctgtactgaaattacatttcacaaaatctattgatctctctctaaattcatcgaatttatagttcatgaatactgagtaatggagtgaaacaaatatagttgtaccagagtaagagaggattttaattagcggattgaactggttttagattgagatagtaattattgaagttttttaaaaaaattatgttgtatactattagttagccttaacatcatcatgattctgtttatttccttaatttatggagggattggaagatgaagaggatgaagagagctataggatgataagaaacagagagaggaggcaattgtatctacacgactacgccgaggaatactgttccactacggactacggcgatctaatcgtgcagcaacggttacagatggttcattggattcttgaggttagttagtgatgaacgtgtttactccccgcgttcctttctagttgatctgaaatgcagttaaaaatttctctggaagttgatgctattagcgaatcaaatcagcttttcgtgccatatagatttacggcttatagtgcatgtggaagttatattttttaacaactggtagaaagacaatagaatccaccttgcacgtgatcctataatactgtccatattgcttgtgtgtgcttaaatattagtagtacaattttactgaaataaagtattggtccatagagtataataattgaagattagtttgatatatattactgtaaaatattaaaattttaaattcatgagatcttagaaagttgtgtagaataatgtcgtcaaacattttggcgagttgattacggacaggatgccatgtaagttggaaagaagtaagagtattataactcgatactatttcccttttattcatatagtaactcattcacaggatgctgttaggaccttgtaatgaaaattaaagaatttggatccactggcatttcaagcgtttaccacaagatggccctaaaacaccaggtcctgggagtagttcaagggatctaagattcccatttttttctgcagagaactgcaatacaaaagcaatacaaaggaactaaaaattgtttcaacggcctatgctttggactctttacatacacatactaatgtcactagtactttggcacttttccttgtctaatgatggacatgtttcttttatagcaagccacgaggaaggaccttcagaaggagacgatgttcctaagtgttaacctctttgacagatttctaagtaaagggtacttcaaaaccaaaagatgccttcaaattgctggcattgcctgccttactctggcagtcaggatcgaagaaaaccagcctttcaacaggtaacattccttgctctgatgttaaatccaggcttgttcccttgagagcaactagagtatatagtatcaaaatgaaatgtcgaaaggaagtgcacataagcagagcctcaaaatcaattgtaaaatctgagggaactgctcagcattgcaaattaccttgcgtttattttttcggatgttacttgctgggaaatacagtaagaaattctcaaaggaagttcaaattgaccaagctaaggacattacagtttaaaagtcttgatatatacattccttatcattagtaaagcatactagtcttcttaagtctcatttgctgaaaagttattaagttgacaagttcacttctttcatttcagcattcgccagaagacattctctgttgcaggcactacatatagctgttctgaagtggtggccatggagtggctggtgcaggaggtcctcaacttccaatgctttcttcccacaatctacaacttcttatggtacaatggttaaccatgctatacaattgcttaaatttgatacttgatgacttagccttaacatacactaactgaagaagtaaagagtcatccatatattttatttggcgtttgtcacaactgcacaggttctatcttaaagctgctacagctaccgaatatatggagaagacagctaaatacctggcagtcctagctttgttgggtcatgaacacttgtgctacagaccatcaactgtagcttctgcactggtgattctcgctttatcagctgccaatctttatgcctcatgccatttggtcaccaaggtaacttctgaaagacataaatattaatccaagtgaaatgtgtttgtgactttcttttcacttggaaatgtatcgtcaaggatgaatccaagtatattcttaagcactattttatctaaaattgtgctttgcatttgcacattttttataagtgtagagagtcatgatcagtaatgcatttgaaaactctaattcacatgctttttcatctcttcattcacagactcatgccaaaatagaagacgaagatttacctgaatgcatcaaggtactatatccctacctagcaatatttagttcatcttgtttctctgctgaaaaacaggcagagtcagagttttgtatcatcaaagctatgatattagtaaatgaagttactgcttttagcttatcaaagaagttattttctaatggcatattcattcatgacagagcttggaatggctggtga

SEQ ID NO: 36 - DNA, 6727 bp, Zea mays
ctcgccgcttgactgtctcgctttctacaacaacattattgccaagaacatttttgatgatgtactgcattccatagcagcattattgtccgtagcagatgatggcaattcgctgctagcactagctgaagcttccttaacttgagcgggaatatcaatcttaactgaaggtctgattgctcgaattggagctgccagtgattcctttgggccttgggtttgcgcatccttcatctaaactggtatggcaactgttagtgatccacaagaggctagcgttatgtatcatcatttactaggacagaaatcacaggagaagttgagctcttccttggttggtattgtacatcctttacctgaaaagggatcacagatggaactgcttccttccttgattgatcttgcatgcccgtgtgcgccgtggagggcaggccggagtcttggacgatgctggccgtggcagccgtcgacatggaaggtgctgtgtctggccgatctctgctgttcttcttttaatcgattttttgttttattaaataggcaaaagacattttaacgtatgcgtcttttttgaaaatagcatataagcgaagtcttttaaaataatggtaaattggcgaaactcacttgccataatggcaaaattctaaatttctcctcacgcaagtcttcggcgccaggtcgcaaccgtcacgtcacctcgccgcctccccccgcccgccctccagaaatagggtcagagcagaaaatatctctcttccctggtcgggtgatgagttatgctgattcggtagcagaaagagcgcgtagggtagaggtggagtggaggagcgagaggagtgggctgcccgccggccctggtggcgtggtgcgacgctggagggaaggtgtgtgtgagagggaggaggccgtggggaatcgagcgcagacaaggcagcgcaaagcgggcggagaagatgacctcccgctcccgcctgcgtgggtagcgtcatggcgcagcaacagaggcagatgggtggtgtgcgccgcgtgcgtggacgaacggatggcatgtgcgcgtaattagcgatcgggcgcgggggcggggcgggaagagcacggacggtgggtgctggtgccgctgccttcggcctcacccaatgccggcagaaggggggtgcggggctggccagaccatacggtgggacttgtgagcgaccgggttgcccgccccggagccggtcggatcgagacgaacgactgcgacagcgagcgatcgcgcatcgcgggcacctggcacgcgtacgagcccaccaccactcgcgctcgtacccgcggtaggcagtccagtccactccagtgcggctcccctggtcaggctagggcctgatggacctgacaggctggcacacacgcgctggcgatgtgtgtgcgcctactttgctcctctgttttaccgtacggcgagcggggcagctggcccagcatggcttcattcccacctgttcaactgtttgatgtaccaatttttttatatatctctacttctctactatctattaaggcaattgtgtagaccatctctgtgccccccgcctccgcgaaacctcagcgacatccacgccgactccgcgcacacggagctctgactccacgaaacccccgcccacctgcatcggatagccaacctccgcacgcgacctccgcaacctcagtaaactgctattgtagctccatgacatttgcgccgacgactccgcgacctcctccaccacgatggctctaccacaccgtgcgcctcatcgcaaaaacctacgcaatgtgtcgtgcggctcccacccgccgtgcccctgcctcccctccgcatacaccatgcgtgtcgtttcgtatatatattttatactattattttacttccgtgacaacgcacgggcacatatctagtagtattgattaaaatgctgtaaaaaataccatagtttaaaatactttggtgcccacgccgcagcccgtaggcagttgccggtgccactgccagtgcgacacggatcacgcaatgcctcccaccatgctcgcgccggtgcccaccaggccgcgctccaaccccttccgccggcgcagaggagctgctccgctgcttctcgatcagacttcggcgaagcggcccgctgagtcgtccacctcagcctcatcctgcttctacagtgaggtgatctccaactcctccacatccctcgccccgtatcagcgcctggagaagaggcatcgacgccaggacacagacgcgggcgaggcgcggccggctggctccgagtgctcggaggtgatcggcggcgcgagggtgcgccccgccgaggtcgaggtctccgaatcgtcctgccctgtctccgtgctcgagtccgacctcgcctgcccgaagcagctcgccgacgacgctgaggcgatcgagaaatcctccgcgtgcgatgagctgaccccgtcgtcggagcccgatgaggaggaggtgctcagtgatcccagccactcggggtactcccccagtcccctgatcagctccccattgaccgaagatgacagcgacgacgcgccctctgcgaccttctccctctttctcgacttcgccaagcagttcgtcccctgcttgcaccccaaaccgtgcaccgtcaccagttccactctcgatctcctgacggtgagcagttcctacgcgattttttcttcaatttgttgggaggggttcgattgcattgatcagtgcggtatttggtctggtcaattggttgtctaatgttttggtgggaatgcttgtgtcaggggaggcggtttgaggacttggacgacgaggagagctacgagcgcttccggcggcgcgagcgacgcgaggcagttgcgcgcgacttcactgaggtgtgcagctccacctccatacccgacagctaccgccctctcgtcgtggagcaacgtgtcatcatggtgaactggatcatccaggtcagtgagtctgtgtcagactgtcagtgcaccataaactctatagtctgcccgtacggtacgctcatcccgagttcccgaccaacaatttacctcagattatgcatggaatgtgaccgaatttatacggtgtaggggctgtacaggcttatgcgagtgagttcagatttgcattctgccggcgttgcaccagtagcatacgactctagcaccatggctgcaaattagtagatttcgcaatagctatgttatgccaatgaatgttgtctcgtgtgaatgccttcgtgctgaggcagcagattagttctctttcttattttgcactggggtagatatctactactgaacattttgttgttaacaccagttgattagtagatttcacaatagctatgttatgctgatggatgtcgtagtgtatctttttgtttgtcagtagtcatgctagttattgtatactttgatcactggttttggcagccaacagagttaggagtatgtttcaatagcaagtactcatgctttttttggaaatggaaacattgtttcgcccttttgcatttgcatgcatacaaccttataactcaattattacatcaacctgcaacaatttgtagttcaaacaaccttcaaccaaataatgatatggagtaaaataaaacacgagcactaagcctctttaattcttgcatggaaccgccacctgtgactcgcaaagaactccatggccacatcctccaaagactggcacacgacgaggatttgctgctgtgtttcttctttctgcaatagtctccaaaatctgaaccagtgtgtgtccctgaagatagtctgtataattgacggtatagatttatgattaaacaccacatcattacggcaaagccaaatcgaccaaaacatcgccgcaacgccagtaagaagcaagtttttatgcgtgcaacctttgttggatttccaatcccctataatatgattaatattaatggtacatggcctgtttaataagcaagggagaagcagctggttcagttactcaacaatgtttctcccaatttcttgtttcgcatctgaacccctatctcatcggcacagtgctggtatgtctggctggaatcatatcttgtagcaatcaggtgcttgaatattcagtatctaaatatgcaagtttctatctgtaactctgtatatacccttcatttcatatttattcccaatttgagctttctgatgtgcttggtatttttatgagatttaagagaactcctgaaaacaccatcatcaccatttttccatctgaaggtttgaattgtgattaagcacaacagttatatttcccctcgtactctgctacaatgatctcaccactcaaaatcacgtgatgcaaatttgaaatttatgtgtattcatttttttataaatttgttaaaaaaattagagttcagttgcaaggaatgactctttattattcaaaatgcagcattcacgtctgatgaagctccagccagttacaatgttcatgggaattggattgatggaccgcttcttgacacaagggtatatgaagggtttgagaaaccttcagttgttgggcattgcctgcatcaccttggctacacgcatcgaagagaaccagtcatacaattggtaatgttctccattgtattacatgtttcaacttttatgtctgtttttaagagattatgtttcttcatatttgttgagtgatggctagaccatctagttccattatatgttcaatttcagtaaaactactgatataagttggtgattccatggtgtcatattgcctaattagatatcgacgggattaatattcagcagcaactggtgcctaatcagtagcatctgagtctgtgtgagctcctcctttaatttatgttggttccataagctatatttttatccatttgcatcactaaagctgcaatatgccttgggtctttgacaacctttagcggggcaaatgagatggttttgatttagtaaaactattttaccatttaatcatattatgaatatgaaacatatctgcatgtggcaatgctttccatggtatttccatttgtaatctttttttgagcaaaaccatctgtcatttgttcctttcaatacttagtatctgtgcaatttgcgtttagaagtgttcacaaggttaacatttcagaagtttattttttccaggacataaatttgggtttcctgattgtgctgttatctatgataaaggcatttgaccctactagttagcattgttttagttgacttgatgcttttatctatttgatttgatatatattactacaattcacatttggaagacatgtaagagaagtatatttagctgaaactgcactggagcatgacctttgttcttcagataattttttcttttcatattccttttccttgtttctgttaagctcaatgtacaacattaatttcactgcttgatccctttcagcgttcttcgaaagacttttcaagttgggatcaatatctacagccagagtgaggttgttgccatggagtggctggttcaggaggtcctcaacttcaagtgttttgtcacaacaacccatcatttcctatggtaccacgaacttcctgccttttgtctattcagctgaacaaaagtaatgagagactaacaccattttgtttcaatgttgcaggttctatctgaaggctgcaaatgctgatgacagggtagcggacctggcaaactacctggccttcatctcacttcggaaccatgagcagctctccttctggccctcgactgtggcagctgcagtggtagttcttgcttgccttgccacaaacaaggagtcctcatgccatttggtgacggaggtgaacacccaggtcctccatttctagctataatttttgttacatttgttattgtcagaacttggtaggcatacctaagtcaaagtatgaactataacttccatcaaaacagataccatactccaaatttacatgctcagattctagctagactggaaacgcctattgagtagctctttacatatttgtagactcacatcaggacgcaggatgatgatctaccagaatgcctaatggtacacattctcttatttttctcttcttttttgggaatacactggtgggcatgaatcatgattcatgcatgctacagtttgcaaggctgttaactttacatttaggaggtgtttgaatgcactagagctaatatttagtggctaagattagtactagcaaatttttagccaaccaactattagctctagtgcattcaaacactcctttattctcctacacaatctgtttcatgcagagcctcgagtggctgatcaagtacgtctcgtgatacccagagctcccaggtgatagcagtgttttcacttttttctgtatggggacgtgaaatcttagcattgacaaatagtctgcctgtagtgtagataagatagccatgcggcatgaaacgtagcttgtggattttgattttgcagctttctgattaggagcacgacaaggacgaggaatttggtattgagcttggcctttaggaataactgaacttctgtatcgggggatgtctatctttacatcggttagtcgctctttttagaaggacggctaaggctgggcgttgttgtactcgttgatctatttgtttaaccaatgtattgctgatggatgatataccactgaaatctgttgttctggtgtgacaagcggc

SEQ ID NO: 37 - DNA, 4113 bp, Panicum hallii
cctatacatgtttggtgaaatgcctctttgggaaggggagggtggcagaggcgcttggcgtgctggataggatggcaggtagaggggtgacgccaaaccgggtttttgtgcagacactcctcgaaggtgtctgcacggagcagagggtggccgatacatataatgtggtcgagcgtgtggttggtgatcggggcatgtcgagtgagcagtgctacaatgttctacttatttgcttgtggagggttggcatgacagctgaagctgaaggattggcgcagaggatgatgaagaaaggggtgcagttgtccccgcttgctggcagttcgatggtgagggagctctgtgtaaggaagaggtcgttggatgcttaccactggttgggaatgatggaggagaacggtgtgctgtgtgactccaatgtgtatggaactctgttgcttggtctgtgtgaggaagggcatctccatgaggcatcagcattggggaggaaggttgtcgagagagagatccacatagaagcatcttgtgctgaacgtttagtggagttactgaagcaatatggtgatgaggagctagcatctcatttattaggattgaaacagtgccctggagggttgtcattttaagcaatgcgcgattctgcacaaccctcgtgcatgaagcacgtcgtggttagtcatggggtgtgccaagaatagtgcttcaccgctttgttgggaatttgcctgagaactgatttagccaaatggcttagtgcagtcaaaagtttactgttgttgaataaagcatggaacagaattcaaccgaagtgccactgaactacttgcttcttttgtataaatttgctgaagaacatgatgcagatccagaagacacttggcgtcatgtaaactaccattttgatcacttctcaggtacatcaccttgtctcccaggctgatgacatgcttggacaagtgccgtgcctgtcagtcgaacattttagatatgtttcatgtgctgtaatcctaggaagttatgtacaacggtgctgaagtcattttacatgatacgtgcccataagcacctactctgacatgctgtaacgttttcgagttactctcagtttttgttgtcccctcatctgaaggaactgaaaagagaatttactttctcattttcttccaatttgtttgtattcaacctgcacctgcaaacaaggtttgcccacattgctttttaggaacatttagttgaaaattttggtgtccgtcaaatctgacattctgctcttgtcggtgtgaaagaaatccaactaagaaggacaagcaaacaaaaccgcggtcaaatctgacattgcatttgcaggtgggtgggcgctggaggcagcggtcgagtgagattgttttcacataaccctaatgcagactgcagacactagcattcttcaagttcaggaatcagggaccattctgatttgcaaccgaaatctgactagttgctgggatttgctgctgggaccgcagtgagccattgaactctgaaaatggagttcaggagaacttcgacagcagctgagagaaaagtcgcgtacctcttgccaccccgaatcaagcagcagatcacacatcgcagcaaagtaaatcacggcatgacagtgacagtccgagacaactggcgtttgctcagtctgcaacagccccggacattcccaacggaggctgacacggccgttgttctggcaatcgcaagtcgccggcacgctgtcaatctactctggctgcaggtgggaccagtgaagcacacccgtccatcaccgttcaggatttaaattcgaattgcttttcgggctgggcgttcatcgttgatctccccttccccttccccaagtctcagtggtctccacacaggcagcggcaggtcggagctatataatcaaggcaaacacggcaacatctagccgtagcaagtagcacaacacacgcacaaccccgggggcacgcgcacacacggatcacacaatgcctctcaccctgctcgtgccggtgcccacgaggccccgctccaaccccttccgccggcggagaggggcggctccgctgctcctcgatcaggccgcgactgcggcggcggcggggaagtggcccaccgagtcgtccacctcggcgtcctcctgcttctacagcgaggtgatctccgcctcctccacctccctcgccgagtaccagcgcccggagaagaggcctcggcaccaggatgcggacgaggcgcggccggccggctcggagtgctcggaggtgatcggcggcgcgagggcgtgccccgccgaggtcgaggcctccgagtcgtcgtgcctggactccgtgctcgagtccgacctcgcctgcccggagcagctcgccgacaccgccgaggcgacagagtactcctcggcgttcgaggagctgaccccgtcggagcccgaggaggatgaggaggtgctcagcggaccttgccgctgcgccgagtactccctcagccccctgatcagcttccccttgaccgaagacgacggcggcgacgccgcgccctccgcgaccttctccctcttcctcgacttcgccaagcagttcgtcccctgtgtgcaccccgaagcgcgcgccgtcaacaatgccgctctcgatctcctgacggtgagcgggtttccacgcgatttctgtcaaaatttgttgggaattggatttgactgcattgttcagtgctgtaatcgatctgttaagttggtaattgttgtgaattgtgatgggaatgcttgtgtcaggggaggcggtttgaggacttggacgatgaggagagctacgagcggttccggcggcgcgagcggcgcgaggcggttgcgcgcgactacactgaggtgtacggctccatgcccggcagcgacggccttctggtcgtggagcaacgtgtcgtcatggtgaactggatcatcgaggtcagtgtatactacactctgctgtgcgcgtacggtgcgatcaacagtacacctcaggttaagcatcggatgtgaccagatttgttgcactggtaggagctatacaggcttatgactgcattgtattggtggcatacgactctagatcctctgttggttaattggtttgttgtcagactcaatggtctagaatttgtttccagtgttcagcaagcaccgtaactgcataattgcgtaaggagctgttctctggtgtgaacgtttttttaattaatgattattgtgctgaggcagcacatctggttccctttcgtattttgtgctggcgaaattatctactatctaaaagtttgttagtttagcactagttgatgagtggatttgaaaatggcgacactattgagatatcagggttcagtggtgtccttgtagctttttatcagtgagtagtcatgattgtactgacgcagttgatcactcatttttgcaaccaaatcgtcctggtcccagagatcagctatgtctaacatgggctgtttgaagaggaagagagaaacaactggttgagttgcacaaaaaaattctctgcagttcccatttggcatctggaacgccatttcattggcatattgcttccatgtctgggattacatattgtagcaattaggatagctgaacctacgctctctaaatgcaattgtctatctgtaactctgaatatgccctttattgcatatgcgtccccacaaatttgaacattttttatgcatttggtatttgtttgagattcggagaactcctgaaaacattgccatcaccatttcccatctgaaggtttcttgaaattaatcattacagatgttttccatgcactctactactgtgtcactactcaaaaacatgacatgaacatttcatgctccttcatttcttagtttgttccaaaattgaagttcagttgtgaagaatgactctttcccttgtaatggcagcattcgcatctcatgaagttgcagccagtgaccgtgttcatgggcattggactgatggaccgcttcttgacacaaggatacatgaagggtctgagaaatctgcagttgctgggtattgcctgcatcaccctggccacccgcatagaagagaaccagccgtacaattggtaa

SEQ ID NO: 38 - DNA, 6723 bp, Setaria italic
cgcgtcgcccctcctcctccgccgccgcctctccacctgcccgccccaccgcgaccaccccaaactcgccgcgctgctggacgtcctcacgtcgacgtcgacgtcccccacgccgctcccacacgcgctctcccgcgccttcccgtccccctccgacgccttccctctccgcacgctgccccgcctcctcccgctgctcccctccccgcttctctcccttcgtttcctcctatggcgcctgaccccctcctcgccgctcccctccccgcatgctctctcctcactcgccacctctctccccgacctctcctcctccgtaccgctcctcctctcctcctccgcacagcccctcccactcccgcactacgccctcctactcaacatctccgcgcacgccggcctcttccccgcctccctcgccgccctgcgccacatgcggtccttcggcctcgtccccgacgccgccttcttccactacgccctccgcgcggcgggctctgcctccgatgtctccgccgtgcttgagatcatggccgggtccggcgcctctccgaccgtgccggtgatcgtgaccgcggtgcataagcaggcgtccgctgggaactttgagagcgcccgccggctgatcgataaaatgccggagttcgggtgcgtgcccaatgctgtggtttacaccgcattgctcgatgggatgtgcagtttagggaacgtggatggcgcgctgaggttgatcgaggagatggagagcagcggtttggatgcaaattgtgcacccaacgtggtgacctatacatgtttggtgaaatgcctctgtgggaatgggagggtggcggaggcgcttggcgtgctggataggatggcagagagaggggtgatgccaaaccaggtttttgtgcggacactggtcgaaggggtttgcacagagcggagggtggctgacgcatatgatgtggtcgagcgtgtgatcggtgatgggggcgtgtcgagcgggcagtgctacaatgttttactcatttgcttgtggagggttgacatgacacctgaagctgaaggactggcgcagaggatgatgaagaaaggggtgcagttgaccccgcttgctggcagttcaatggtgagggagctctgtgtgaggaagaggtcgctggatgcttgccactggttgagaatgatggaggagagtggcgtgctgtgtgactctgacgtgtacggaactctgttgcttggtctgtgtgaggaagggcatgtccatgaggcatcagcattggggaggaaggttgtggagagggacatccacgtagaagcatcttgtactgaacgtttagtggagttgctgaagcaatatggtgatgaggagctagcatctcatttattaggattgaaacagtgcgctggagggttgtcattgtcattgtaagcaatgtgcattcttcccaaccctcatgcgtgagaacgccaagaacagtgcttcacagtcttgttgggaatttgcctgagaacagcttcaacaaattggattggtgcagtcatgatgctacgtttagactgttgcttgatacagcatggaacagaattcaaacaaagtgctgctgaattacttgcttcttttgaatgaaattgctgaagaacatcatgtagatccagaggacgcttggcgtcttgtaaactaccattttgatcacttctcaggtacatcaccttgtctcccaggctgatgacatgcttgggcaagtgctgtgcctgtcagtcaaacatgttagatctgttttatgaacttgcaaccctaggaagttttgtacaatggtgctaaagttattttacatgatacgtgctcaagcacctactctggtatgttgtaactttttcgaattactctcagtttttgctgtccccttttctgaaggaacagaaaagagagcttgctttatcaatttcttgcaacctgtttgtattattaagcacagcctgcacataaattttgcccacattgcttttcaggaacatttagttgaagtgagactgagagttgcatgtacataaatgttcgactggaagcagcggtcaagtgggagtgctttcacataaaccgtactgcagaggctgcattcactagcagtattcaagttcaggaccattctgacttgcaagttgcaaccggaatatgacctgttgctgggattttgctgacgggaagacagtgaggcattaaactctgaagaaaattggcgtttcaggagaactttgacatctgctgagataaaaagtcacgtacctcttgccacgaaccaagctgatcacagcaaagtgaatcacggcgtgtcagtgacagtctgagacaacctggcgtttgctcagactgcaacagccgcggacattcccaacggaagctgacacggccgttgttctggcaatcccacgcggccacgccgggaccgggcctcgcacaccccgctgtgaatctactctggctgcaggtggggaccagtgaagttcacctgtccaccacggctcaggatttaaattcgaagagcttttcgggccgggcattcatcgttggtctctccttcccttccccttgcccttccccttccccttccccaagtctcagtgacctcctcacagacagcagcagcagcagcagctggtcggagctacaccccgcaggcacgcgcacgcacggatcacgcaatgcctcccaccatgctcgcgccggtgcccacgaggccccgctccaacccctaccgccggcggagaggggcggctccgctgctcctcgatcaggccgcgactgcggcagcggcggggaagcggcccgctgagtcgtccacctcggcctcctcctgcttctacagcgaggtgatctccgcctcctccacctctctcgccgcgtatcaacgcccggagaagaggtctcgccgccaggacgaggacgaggcgcgcccggccggctccgagtgctcggtggtgatcggcggcgcgagggcgctccccgccgaggtcgaggcctccgagtcgtcgtgcctcggctccgtgctcgagtccgacctcgcctgcccggagcagctcgccgacgacgccgaggcgaccgagtactcctcggcgtacgaggagctgaccccgtcggagcccgatgaggaggaggaggtgctcagcggtccttgcagctgcgccgagtactccctcagccccctgatcagctcccccttgaccgacaacgacgacgacactaccgcgccctccgcaaccttctccctcttcctcgacttcgccaagcagttcatcccctgcgtgcaccccgaagcgcgcgccgtcaacaatgccgctctcgatctcctgacggtgagcgggtttccacgcgatttctgtcaaattcgttgggaattgggtttggctgcattgatcagtgcgacgattgatctggtaaattggtaattgttgtggtgggaatgcttgtgtcaggggaggcggtttgaggacttggacgacgaggagagctacgagcggttccggcggcgcgagcggcgcgaggcggttgcacgcgactacactgaggtgtacggctccatgcccggcagcgacggccctctcgtcgtggagcaacgtgtcgtcatggtgaactggatcatcgaggtcagtgtatactacactcactctgttgtgtgcgtacggtgcgatcaacagttcacatcgggttatgcatcggatgtgaccagatttgttgccctggtaggagctacacaggcttatgcagtaatgtctgcattgtactggtggcatacaactctaggtcctttggtggtttgttgtcagattcaatggtctagaatttgtttaccagtgttcagcaagcaccgcaactgcataattgcataagctgttctctggtgtgaatactttttttagtaatgattattgtgctcaagcagcatgtctgattccctttcgtattttgtactggggaaactatctgctatctgaaagtttgttagtttaacactagttgatgggtggatttgaaaatggcaatgctattgacatatcaaggttcaggggtgtccttgtgtagctttctgtcagtgagtagtcatgcttgtactggctcagtttggtcactcattttgaggaccaaaccatcctggtcccagagatcagctatctctaacatgggctgtttgaaaaggaagagagaaacagcggattcagttgaataaaatgtttctccgcagtccccatttgacatctgaaacatgatttcattggaatgttgcttccatgtctgggattacatagtgtagcaattaggattcctgaatcttcgctctctaaatgctattgtgtctatctgtaactctgaatatgccctttattgcatatgcatccccgcaaatttaagcttttcgatgcactttctattcgtatgagattcagataactcctgaaaatattgttatcaccattttctatcagaaggtttcttggaattaagcatttcattgatgttttccttgcattgtactataattgtgtcactactcaaaagcatggcatgcacaatttatgctccttcattgttccaaaataaagagttcagttgcaaagaatgactttcccttgcaatgccagcattcgtatctgacgaagctgcagccagtgaccgttttcatggggattggactgatggaccgcttcttgacacaaggatacatgaagagtctgagaaatctgcagttgctgggcattgcctgcatcaccctggccacccgcatagaagagaaccaaccgtacaattggtaatgttctcccttgttatgtctgctgtaagagattctgttctgcatatttggtgaatcctaactagatcactagttccgttccatgctttgtttcagtataactactgacttaagtcacaccaaattagctccttcttttaatcatgcattgatcctgcatagtccctcagatgatagaatatatgctgcaaggtcataactatgtttcttttcccagttgcatccctacctcgctgaaatacgccttgggtgtttgaaaagctttaggaggcaatgagatggttcggttcagaagagctatttcactgtttaatcatgttatgaatctgaatcatattagcatttgacggtggttttcacattctcatctgtcatttgtcccttttgatactagcactctgtgcagcttgcatttagaagtgttcacagggtgatcatttcagaagttccttagtttcctgattggactgtgctgctgttacgtgttagtgatagtaaaagaattgacgatgctggttgccatttttggttgattgaatcatatattttgatatgtgacacgcatcgctgcactttgcattgcaagacaactagacgtatctttagctgaaattgcactgaagtgtatctatgagttttgtctcctgatcataacttgttttcgattatttatttgtgctaagcttgatgtgcaacattatctcattgcttgattcctttcagcgtccttcaaaagaccttcaaagttgggatcaatacttacagccggagtgaggttgttgccatggagtggctggttcaggaggtcctgaacttcaagtgtttcgtcacaacaactcaccatttcctctggtaccacaaacttcctgtcttatctgtatcagctgagcataaggacaggctaacaccattctgttcaaatttcgcaggttctatctgaaggctgcaaaggcagatgacagggtagcggacctggcaaaatacctgtccttgctctcacttctcaaccataagcagctctccttctggccctcaactgtggcagccgctgtggtagcccttgcttgccttgccacagacaaggagtcctcatgccatttagtcatggaggtgaacacctcggtcctccatttctagctataaattgtcattacacttgctattgtttagaactcgataaacataagcaaaatatgaactgtaactttcagaatctcctgtcagtcagcttctaaacatgcaccaactccaattttacatgttctgattctagctagactggaaatgcctaacgagtagctctttgcatgtatgtagactcacatgaggacgcaggatgacgatctgcctgaatgcctaatggtacatttttctcctctgattttttataagttactgggcacaaatcaccatgaaccatgctcttacagttccagcaataggcagaaatctacttttatcaccatctaacattcattaccccgtgcaatcttctttcatgcagagcctagagtggctgatcaactacgcttcgtgatacccgtgactcccaggtgacgaaattgatccacagttttgccgattcccagttacacagcacagttcaagcggtcagatagacatgaggatgtgtaggccatacgtgaatcttagcattaacagattattctgtacatgccattagttttccctgtaaggtagatataagataagccaaggcagcataaaacgtagcctgtgattatacgactttctggctaggagcaaggcaaggatcgagagtttggtattgagctgtcggcctttagggactactgagtgccctattgggctgtctattatctttacatcatttcttcgctgtcttagggacaactaagggctgggctctgttatactcctattcaaccgatatatttgtttaaacggt

SEQ ID NO: 39 - DNA, 6152 bp, Sorghum bicolor
tgcagttttgggaacgtggatgcggcgttgaggctgatggaggcgatggagggcagcgagtttggtgcaaactgtgcacccaccgtggtgacctatacgtgtttggtgaaatgcctctgtgggaaggggagggtggccgaggctcttgctgtgctggataggatggcagagagaggggtgatgccaaaccgtgtttttatgcggacgctggtcgaaggattttgcactgagcagagggttgtcgaggcatatgatgtggtggagcgtgtaattggtgatgggagtgtttcaagtacacagtgctacaatgttctactcgtttccttgtggaaagttggcatggaagaagaagctgaaggactggcacagaggatgatgaagaaaggggtgcagctgaccccactcgctggcagttctatggtgagggagctgtgtggaaggaagaggtcgttggatgcttgctactggctgggattgatggaggagaacggggtgttgtgtgactctgatgtgtatggtagcttgttgcttgggctgtgtgaggacggccacattcatgaggcatcaacattgggaaggaaggttgtcgatagggggatcctcatagaagtatcttgtgctgaccgtttagtgaagttgctgaagcaatatggtgatgaggagcttgcatcacatatattgagattgagaaggcgctctgaagggttgtcattttaagcaatttgcgattctgctccatccttgtggatgaagaacatcttgattagtcatgggatgtgccaagaatagtgtttcaccaccttgttcggaatttgctcgtgaactgatttagcaaaatggcttaggccttgtttagtttccaaaaagtttcaagattccccgtcacatcgaatcttgtaacacatgcatgaaatattaaatgtagacaaaaacaaatactaattacacagtttatctgtaattcgcgaaatgaatcttttgagtctagttagtctatgattagacaatatttgtcacaaacaaacgaaagtgctacagtagcaaaaaccaaattttttcccaaactgaacaaggccttagtgcagtcaaaatgcttggagaagtgatgtgactgtttgtcgaacatcttagacctgtttcatgtacttgtaatcctaggcagctttgtacactgtctataaaaagtcatttactacattcccataagcacctagcctggtatagtggtatgcatgacgttttctagttatcctcaggttttgtcgtccccttttgcaaaggaatagaacagagattaatttctcgattccataaaatctgacgttctgcaatttttggtgtgaaagaagtatcgaggcgggccagctgatgccggtggaagcaaggatggcgctgattgagtaggcgcagccgcttgttgcatttgcaggtggctgcggcgcggggcgcttgaggcaggccctgaacatgggctgatgggcggtgtatcaatcttgtgtgaccagcaccggcagtgtgattgctttcacataaccgtagtgcaggctgcagatgctagcaatattcagtttcaggaccattctgacttgcaactggagtatgacttgttgctgtgattttgctgacgggaacacaatgggccatgacaatggctttccttatttccgcagctgctgctgacattctctacggaggctgacacctgacagtgaatctactcttgctgcaggtgggaccagactaccagagaagcgcacccgtagcgtctccatcacggttccggttggttcaggatttaaattcgaagagcttctcgggctaggcctccatcgttctgatgatcacccctcccttccccttccccaagtcgtagcggccagctgccagcaccgcagcaggcaggagctatataatcaaaggcaaacagccaaacacgcacacacacctagccgtagcattgtagcaacacgcgcactcgccgctgccgcacggatcacgcaatgcctcccaccatgctcgcgccggtgcccaccaggccgcgctccaaccccttccgccggcgtagaggggctgctccgctgctccacgatcagactgcggcggctgcggcgaagcggcccgctgagtcgtccacctcggcctcctcctgcttctacagcgaggtgatctccaactcctccacatccctcgccgcgtatcagcacccggagaagaggcagcggcgccaggacgcggacgcggacgcgggcgaggcgcggccggctggctccgagtgctcggaggtgatcggcggcgcgagggtgcgcctcgccgaggtcgaggcctccgagtcgtcgtgccttggcgccgtgctcgagtccgacctcgcctgcccggagcagctcgccgacgacgctgagaggaccgactactcctccgcgtgcgatgagctgaccccgtcggagcccgatgaggaggaggtgcttagcggtcccagccgctccgctctgtactccctcagccccctgaccagctccccattgaccgaggatgacaacgacggcgcgccctccgcgaccttctccctcttcctcgacttcgccgagcagttcgtcccctgcgttcaccccaaagcgcgcgccgtcaccagtaccgctctcgatctcctgacggtgagcggtttctaagcgattttgttcttcaatttgttgggaggggttcgattgcattgatcagtgcggtgtttgatctggtaaattgataatgttttggtgggaatgcttgtgtcaggggaggcgatttgaggacttggacgacgaggagagctacgagcggttccggcggcgcgagcggcgcgaggctgttgcgcgcgactacactgaggtgtacagctccatacccggcagctacggccgtctcgtcgtggagcaacgtgtcgtcatggtgaactggatcattgaggtcagttcatactaaactctgtattctgcccgtacggtacgtcattcagatcaacaatttacctcaggttatgcatctgatatgaccgaatttatacggtgttaagggctgtacaggcttatgcgcgtgagttcagacttgcattgtgccggcgttgcaccggagcgtacgtctctagcaccatagctgtatgattgcagcaagatctgttgtctcatgtgaaggccttcgtgctgagtcagcagattagttagttctctttcttattttgcaccggggtagttatctactactgaacacgttgttaacactagttgatgagtagatttcacaatagctatgttatgcagatggatgagtgtacctttttgtctgtcagtacacagtagtcatgctagttctcattctatactttgatcactggttctggcaaccaacagaggtatgtttgaatagtaggtttacatggtctgtttgataagcaagggagaagcagctggttcagttactcaacaatgtttctccgaatttctcgtttcgcatctgaacccctatctcatcagcacactgcttgcatgtctggaatcatatcctgtaccaatcaggatgcttgaatcttcagtatctaaatatgcaactttctatctgtaactctttatataccctttatttcatatcgatcccaaatttaagctttctgacgtgcttggtattttatgagattccagagaacccctgaaaatactgtcacactatttttacatctgaaggtttgaattgcgatgaagcattacagttatatttcccttgtactctgctagaataatctcactgctcaaaattatgcgatgcaaatttatgttgattcattttttgcaaggaatgactctttattatgaaatgcagcattcacgtctgatgaaactccagccagttacaatgtttatggggattggattgatggaccgcttcttgacacaagggtatatgaagggtttgagcaaacttcagttgctgggcattgcctgcatcaccctggctacccgcattgaagagaaccagccatacaattggtaatgttctcccttgtattacatgtttcaacttttatgtctattttttagagattctgtttcttcacatttggtgagttatagctagaccttctagttctcttctatgctcaatttcagtgaaactactgatgttagtttgtgattccatggtgtcagattgcctaattagatatccacagaattaatgtttagcaccaactgatgtgtaatcagtagcactctgagtgagtgaactcctcctttaatttgagttagttctaacattgcctcataggatatatgctgatcataagctatgtttttatccatttgcatgactaccgctgaaatgtgccttgggtctttgacaggctttagcagggcagatgagatggtttgatttagtagaactattttaccattgaatcatattatgaatttgaaccgtatatgcatgtggaaatggtttccatgcatttccatctgtcatttgtttttgtttttgttttaagtgaaaccatctgccatttgttccttctgatacttggtatctgtgcagcttgcgtttacaagtgtttgtaaggtgaatatttcagaagtttcttttttccaggacataaatttgggtttcctgattgtgctgttatctatgataaaggcattgagcatactagttagcattttttttagttgacttgatatttctatatatttgatttgatatgtattactacaattcggatttggaagacatgtgaaagaagtatatttagctgaaattgcacttgagcatgtcctttgttctccagatcatttttcttttcctattccttttccttgtttctgttaagctcaatgcacaacattaatttcactgcttggtccctttcagcgtccttcaaaagacttttaaagttgggatcaatacctacagccagagtgaggttgttgccatggagtggctggttcaggaggtcctcaacttcaagtgctttgtcacaacaactcaacatttcctatggtaacacgaatttcctgctttcttgtctgttcagttgaaaaaaagtaatgggagactaacaccattctgttccaatgttgcaggttctatctgaaggctgcaaatgctgatgacagggtagcagacctggcaaactacctggccttcgtctcacttcgggaccataagaagctctccttctggccctcgactgtggcagccgcagtggtaacccttgcttgccttgccacaggcaaggggtcctcgtgtcatttggtgatggaggtgaacacccaagtcctccatttgtagctataaatttcgttacatctgttactattgtttagaacatgctaggcatatataaatcaaagtatgaactgtaactaccagaagctcaagccagttagcttgaaaacagataccaaactccaaatttacgtgctcagattctagttagactggaaatgcctaatgagtagctctttacatatatgcagactcacatgaggacgcaggacgatgacctgccagaatgcctaatggtacgcttctcctcttcttctttacctcttttgtttttggaaagacactgggtaccatgaatcatgattcatgcatgctacagtttgcagggctgctaaccttacattcgttatcctatgcaatctgtttcatgcagtgcctcgagtggctgctcaactacgtcccgtgatacccagagctcccaggtgatagcagtgtttcacattttttctgtaaatggggacatgaactgacaaattgctctgtacatggcattagtctgccctgtagtttagataagataatagccaaggcagcacgaaacgtaggttgtgggttttgattatgcagctttctgattaggagcacgacaagggcgagattttggtcgtgagctgtcggcctttacggactactgaactgctgtattgggggatgtctatctttacatcgtgtattcgctctttttaggctgggcgttgttgtactcgttggtctatttgtttaaccaatgtattgctgatggatgatataccactgaaatctgttgttctggtgtgacatgaggcatgatcttgtgatcctggaagatgctcacgtatcaggtttcagtgagcagaatactatcctca

SEQ ID NO: 40 - DNA, 12762 bp, Brachypodium distachyon
atgaaaccacagaaaaaatttcaactcaaaactgggtcaaattaatgcaaaacttagttcggaattcaaatttggaggttcactcttgagatcgttcattttgtgaagtatgtaccattttaattttgtgtatttacttgtaaattttatccttgtgttgtataccaataacatggtaccatgccaaaaattctgaattttttataaatgtttaatattgtttcatttttccctattaaacgtatatagaaaatgataaataattatttttacataaaaagttagtattttaattcacattactcgtcatcaatgtcaaatgaaagtacagtaaaagtttcaactcaaaaagtggtggtttacatcataatttgaaacaagagaggaaagggatacaaaagaaaaaatattgagaaactctttgccggcggccggccttcggcaaagaatcccgtccgttttctccccgttaggcgccggtcaagtccacgtgggaccccttcctttgtcgtaggcatttctttgccgaaggcctttttgttctttgccggaggcttttctttaccggaggccttttttattctttgccggaggcctcttcttttttgccgtcagcaaagtcttagcctccggcaaaggcccaggccgccggcaaagaatgtttttcccgtagtgtactccctccgttcgttttgaaattttgctttgaccatcaattagaccaataataagtgaattatgtattataaaagtataccattggaaacctcttccaaatatgaatctagtggtataatttttatagcatattattttaattttattagtgtaattgatggtcaaagttagacatcaaaatacgtgggtacgttatattatagaacggagggagtacctcaatttgcctctcggacacaaggtccaaatgtcatcgccgatgaggctcgaagccgagaaatttctgtgaagattgtcgttgctggcccgaacactgcctcagcggaagtcatggaattcatcacttcaagtctaactcaacataattctgctgcatccgatattatcaaccaatattaagcccattccgaagaccaagagcttcagccaccagcggaggtagaaattcaaatattttgacgtcgaggggagccgactaaaagagtcaaacgaaatttttgtttttttcatgtagttaacaagtaaatgccacgtgttaggcgggcccttggccctgttggtcaccctccaccataggctcagctattgccgcactgccaatgtggaggatgaacgaggttgtcgtcgccatgaaaaacctctgatcatcactttatatgtctgattttttttgttacaataatagggtgtgactattttaaaaacaaggattaaatctcagttgtatcacaaccgttggaattatttggatgtcatgtcatcccggtcttctctttccatcgttcgttctgaatcttacagaaaaatcaaatctaatggttgagaaatcaaatcactgataactaaaaatcaggcaacttagatatggtaaaaccataatgaatttttgtaaaactttaaaaactcttgccaaaagatttgttctcctcgcaaaagaaaaagagacatagaagacgtgagaagaaactctgatcagcgaaattaccagaacctgactcctcaaaaccacgctcgcggtgtgataccgacttttattatcgtgtgcagtgatcgcatgtgcgcttcctaatcctgcagcagccgtcttccgtgttccgtctcgtttggaaacggggaacaccgaggctgttgacctgtcgttaccgtcaccgtcggtccatcgttccggtagatcgctcgtacaccggtgtttcctgggccgtgggatccgcacccactgcaagcggggtcctatggagcggtgtacgagcacccggcgcagctgggagctcgtgatttcctggccaagctcatgaatttaaaattcaaaaagtgctggtcgggctgtgagttcatcatggcaggaccgcaggagtcgtcctcccattcccccttcctccccagtctcgacggcctcgcaggcgcgattatataagcaaggcaattcacctagccgtagcccgtagcgacacacaagcacacacgcacacgaacacacactatgcctcccaccatgctcgcaccggtgcccacgaggccgcgctccaaccccttccgccggcggagaggggcggctgctccgctcccagcccagatcgtagcagcggcggcgccgaagcggcccgcggagtcgtccacatcggcatcctcctccttccgcagcgaggtaatctccaccacctcctccaccgccctcgccgcagcccagcgcccggacaagaggcctcggcttcaggacgcggacgaggcgcggcccgcagcctccgagtgttcagaggtcatcggcggcgcaagggcgcgcgccgcggaggtcgaggtctccgagtcatcctgcctcggctccgtcctcgagtccgaccttgcctgccccgagcagctcgccgacgatgcagaggcgactgagtactcttcggcccgcgatgacctgacgcagtcagacgccgaagaggaggttctcagtgctcccagcccttgctccgagtactccctgacccccctgatcgactcctcttcctcgtctgacgacgacgatgacgccgccccctctcccaccttctccctcttcctcgccttcgccgagcaattcgtcccctgcgcgcacaccaaagcgcacgccgtcgccgacgttccgatcccagaggtgagcgggttaatttctacacagttgttctaaatttgtttgaaattgggtctgtttgcaagtgtcggtgcggtgtttcatccgattaggtggcttggtgggaatgtttgtgacaggggaagcggtttgaggacttggacgacgaagagacctacgagcggttccggcgccgtgagcggcggggagtggtggcgtgtgactacaccgaagtgtacatctgcatgccaggcagctatggccgtgccgtcgtggagcagcgtgctgtcatggtgaactggatcatcgaggtcggtttaatactgcgattatcactctggctcgtttgatttttgtggtagaagcgtgccttcaggtttacagtaaaatacatgcatacaatggaagtcacgtagtactctaaattctgtgttgttttgtttgtctgagtccgagtgtccaatatgctctgaaagcacggtagttttgtgactgcgctaataagctgatctctggtgtagatgtttgtgctggcctagtgaggcagcagatttagctatgcgatttcgtgattagtgcagcggcaagttgtgtactatctaagaatttgttgtacaacattctgataagaagattgcgcaactgacattgttcgctgaacagaaggatccccattttttttttggaactgttgttgaccaggccatacttattgcagtactcaaaaggactctgatcaccaattttgactgttagaccatccaagtcaaagagatcagtgctaggatgttttagcaggtgtttgttttgacctttgacatttactatttgaaaaggaatggacaaatagatagttcagttatgctgagaagttattcagtgagccatttgacatgtcatccgcatgtggcctcgacgcctcgtgtgtctggaaagcatattataggagtagcaattaggatatctgcataatttctatgtacatatgcaattcatgagtacttcggtataatcacttatttagcctcctatgaaaaatcttagtttgtctatgcacttgatattgcattgagactggaaagaacttctgataatactgacaccactgtgtcttccacctgaagatttgggtgtcttccaccttgtactgtaatattcctgaaaagcattgtactatgattcctggagcaaagatttattttcagataaaatttcatctacagctgacgaaatactgactgtttcccatgaatttttctagcatgggcatgttaccgatctgcagccagagacagtgttcttggggattggactaatggatcgcttcttgacgcgtggatacgtaaagggcacaaggaatatgcaattgctgggcattgcctgcatcaccctggccacccgcattgaagagaaccaaccatacaattggtaatgagtgcccttgcattacatgttcattccgtttcagtaaaattggtgcatgctatattttctgaggataaagctcatggtatattttcatgtccccactttcagtatactactgtaggttgttggtatacagtagtcagattgtgtcatttgaagtgtgtaccctcttaactgatgcattgctaaatgaaataatgcttcaaagaagctcctcatctaaattcagatcttagttcaacgtagtttcctacttcctccgtccaaaaaagatgtctcaagtttgtcaaaatttgaatgtatctagacatgatttagtgtatagatgcattcaaatttagtcaaagttgagacatcatttgttggacggagggagtattacatatttacattgtgacatggttgtagtacataatactgttagttcctacctaagctattctctgtggtatttgcttttctgttgctaaagctcattgcagtatgatttaattgggaacttgatagaccttagcaagtatccttgggaagccttggtttgttggaactgtcatcgtctaatcacatgatggatctccatagaaacatgtgacaatagttcatacacggtgtttacttatctcattgcaggcttatcggctatcactgcatgctagtatttgcaaattgatcattaatcaacttccatttttatgggttgagcatttcagaaattgactttcttaattgatttacctgtggtcagctagcatcttcagtttagaacacaaaatccattcatatgttatccccactgaagggagttgaaccattgtacgagtgatcctaggtagcataaggtccaaactttttgattgtgcatacttacatgattgttcaagtgaaatcagagcctttttgtggttgttttaaagtttttgagcctgaattcaagtggatctttccttattattaacagcaggtctgaagataataaatcattatgtgtcacacagtagtacctccgttcctaaatacttgtcgctgttttagtgcaaacttgcactaaaacagtgacaagtatttaggaatggagggagtactatatatgcagaaacaatagagtacttaagattaacgtcaacaggagcactgcagcattattgttgaacttctgggtttattgtctatgggatcaacatttgtttcctcattaatgtttctgttcaaaaaatgtgtgatgaggaacctcactatattatctctttcagcatcctgcaaaaatctttcaaggtagggatcaacacttatggccaaagcgaggtcgttgccatggagtggctggttcaggaggtcctcgacttccaatgctttctcacgacagtccaccatttcctctggtactacgtgtttcctgcattcttatctgtgccaagccatacaaaacgaacgagaagctaacagctgactttgttctaatttggcaggttctatctgaaagctgcgaaagcggatgacaaagttgaggatatggcaaagcacctggccttgatctcacttctggaccataagcacctctcctactggccctcgaccgtcgcagcagcagtggtagcccttgcttgccttgccacagacaacgactcgtcctgccaactggtaatggaggtaaatacttaaatccttaaccaaaactatacatttctctatcttttctgtcgtttggaactttgataagacgtactgttcctaacaaagactagaatcaccgaactgtttgctctttgcataacgcagactcacatgaggacgaagaacgatgatctgcctgaatgtttaacggtttggcccctcactcgcattctgatacctggcatgaacttaactgtgttgttcactatttgcagtaactgatgtacggaggaagtacaattttgtggtgccacggcaactaacattcgttatgttatgcaacttctcgtgcagagtctcgagtggctgataaactatgcttcgtagtaccctgggcccccagaattgagcattcgatctaaccttcgctgatcagcacagcatagcagtcgtttagcaacaacaaaagagcgtacatgccatctggttgcacagcaggataactaaaaaggacaaggcagcaggtttatgactgtagggccaaccgttgtggtcgtctgtctttgcatcagcagctagctctttaggaacaattaaggatttaaggttggatgctgtagtattcctcaatgtcttttttagatcaacggtcttgtttaatgagcctgctaatgttagtgtatgattgctatttttcgccgggttactatagctctttaggaacagtcaaggttggatgctgttgtgttcctcaacttccatttttcaatgatcaacggtcttgtttataaggacttgtttagtgttagtgtacgattgtgatttgtcgcccggttacttctgatcatgacccaatcttgtcttcttttttctttctttttttagggagttacacggtcttgtctgccactactcttttcgttcgtcggcccaaccctcccaggttcagctcgcagctgtgccaagcagatacgttaacttagacaactcctcagtttcaaaaaaaaaaaaacttcgacaactccttccgaagcaacaatagctgaagatttttggagcgaaacaatagcggaagatggttgagtctacacctgcaggggaatgcgttttttctccttcggcaccagaccagagtagtaccagaccaccagaccagagaggcagagaccatcacctccgtagtccgtagtggacgccaccaccagatgcctgcgtgcgcgtccctcgtccgccgcctctccacccgccgcgatcccaacctcgccactctcctcgccgtcctccgctcgccgcagcccccatccacgccgctcccgcacgccctctcccgcgccttcccgtccccatcagacgcgttccccctccgcaccctccccggcctcctcccgctcctcccgtccccgctcctctcgctccagttcctcctctggcgcatgcccccttccccgccgctcccctccccgcacatcctctcctcgctcgccgcctcgctccccgacctccccaccgccgcgcccctcctcctctcctcctcccctcacccgctacccctcccgcactacgccctcctcctcggcatctccgcccatgccggcctctttcccgcctccgtcgcggtcctccgccacatgcgatcctcccgcctgacgcccgacgccgccagcttccactccgccctccgcgcagcgcgctcgcctggtgatgtctccgtcgttctggacatcatgtccggtgccggcgtcgaccccaccgtccccctggtcgtgacagcggtgcataagctggcatccgcgggcgagttcgaggacgcccgccgtctgatcgacaaaatgcctgagttcgggtgcgtggccaatgtggtggtttacaccgccgtgctcgacgggatgcgcgctttcggggacgtcgatgccgtggtggggcttttgaaagagatggaggacggcgggctgggtgcttggtgtgtgcccaatgtcgtgtcgtacacgtgtttggtgaaatgcctgtgcgagaaggggagagtggcggaggctctgagcgtgctggataggatgatagctagaggggtgatgccgaaccgagttttcctgcggacactgatcgatgggttttgcgcggacaggagggttggcttggttgccaaggcatatgatgtggtggagcgtgttgtcggtgacgggactttgtcgagcgagcaatgctataatgttcttctggttggcttgtgtggggcggggatgtcaggggaagctgaaggacttgcacacaggatgatgaagaaagaggtgcagctcagcccgctcgcggcaagtgcaatggtgagggagctttgcaggaggaagaggtggttggatgcttgccacttgttgggaatgatggagaagaacggtgtgctgtgtgactctgatgtctttgctggtttgttgctggggctgtgcgaggacgggcatgtccttgaggcctcagcattggggaggaaggtcatcgagagggggatacacatggaggcttcttgggctgattgtttggtgcagttattgaagcaacatggcaatgaggagctagcatcatatgtattaggattaaggactcgtgagtgatgtcactttgagcaatgtgtggtccttttccccaatccttgctttgctgcaacatggtaatgaagaagaaaaaaggtttgttttagttgaagcaaggaccatgtttggctccgaatgatacagctaggaaggatatctcttgtcaagttgcttttgctgcaacagataatcggtggatgcagcacagaaagactagtgtgatcaaattttgggtgccgcacagaaagacttgcttcgctgcaacagaagtacttacgtacttctacttgatgcttttgccaaagaacttgctttagatccagtggaaacttaggccatgtaattaccattacgaaggcctctcaggactcaggtgatcatcaccatgcctcccagatagatgtgcttgcaacactgctaatcaattgtaggagtggtgccataagatgcagacttcagtttaattgcttcaggcagttcaccacgatttagtggctattctttttgctaagtaaacctaccgtgtcaacctttttggtttcatatggttacttctgcaagagaaatcagggacttttttagtggttgtgttaaggtttttgagcctgaattcaagtggttcttatcatgcttactttttaacacttaaaagtttaaacaaaagtatattcattacgtggcactgtgtattgttcagaatcagtccacacttaagatatgagatgttcttttttaccagtcaactcctttgcattgcagaagcacaccgcggtatcacggcagaactcatttttttttttagtttgttctatttgcttctgttcaggacaatgggctcatttttttttagatgctcccaatgggctcattatgttatttctttcagcatattcatgttccttgtttctgttcaggacaatgtgctcattatgttatctctttcagtatcctgcaggtatcttccaagttggtatcgttacttacagccgaattgaggttggcgctatggattggcttgttcaggaggttctcagctttcagtgctacttcttttctacatctttgtccgtgccaaacttaacaaatgaacggtttgcttttttctgattttgcaggtcctgtctgaacatattcaggctccaaaatcagatgacaaagtcaaggacctggcaaaatacctggccttgctctcaattctatgccataaagcacctctctttctgaccctcacccgtggtagcccttgcttgctatgccacagaaaaagagccctcctgccatttggtaatagatcaaggtaaacactgacctccatcgcttgcctacatatattcttattttcactggttcttttcagaacttagataaggctataagccgcaatgatcctaaaaagaactggaaacgcacgcctaacagcttgctcttgtataaacgcaactcacttgaggacgtataatgacaatctgcctgaatacttaatggttcctcacgctctgattgacatcatgaactgatgtcctttttgtgctttaatcgatttacagcaacagatgtgctgatttctgtggtgccacggcaactcatgtgcaaccttttcatgcacgttgttgaatggctgatcaattatgcatgcatcgtactagtcttcagctgaatccaagtaaagcagggtagcagtcgtttggcaaattcttttgtacacgtaaaaaaagggcaaaggcagggcagcaggttctaccaaattttggcatcgcgctgtcgcatttagggacaactgttggggtcggctgtttgtacaacttggttcgctgtgtagcgacagtttaggttggatgtgcattcctcaacttctttttcttcaaccaatgatcttgttgataattagtgtacgtctggcatttgcttatgatcatgcaagaccttcaaatcgaatgtgaccgcccatccgccatccggtaatggaaagatcgcagagaacgaatatgtttggccaactgaatatccagagaagcaccaatcctgcttgttcgatcgcatacggattccttcacggaccaaacctctattcttcccttgtcttttccaaactgaaaggatggaacgatcagttggtttcacgagcctttacgcgcgagggcgaccccggccgcaccaggtgcttcagccacttttttttagggaagatgcttcagccacttgggatggaattcctgcacacaataaacgaggaaaatgcttgccaattgagccatccttgtgcgtatgttcacgtgcatgcgccagccattagcagttttaggatatgtttggtttgcgtctacgtatgtactatcaaaaatttggcagactgaagatttctggtgtgtttggtttgggcctttggggcttagccaaccgtaccaaaatttggcttgccaatggttgctcttgtttttgtcaatgttggcaagaaaaataaactactcactccgacccatattacttgtctcaaatttgctcaaatatggatgtatatatgtaatatttcggagggagtagcattaattaagaaaccatgtagtacataccaaaatattggcgaccatttaaacaagagccaaaattttggtcatgaccaaaataatcgcatgtctgatgttggtcaccatccaaacacggtctttgcttgcctggttgcgaaatattgcctatgtagtcagtccctgtatgtagggttgctaatccacctcacaatcccaatcttgtcaaattttggggctaagaatttttttgataaaatttccgtgaggaccttataaacctgaatagagggaatagcatgcatctctccttcagcctgcttgcacttgccataattttcactggcctgtcggggagtcgtcttccttttgagctcaggtcggagaaattgcaaaacgggggtaaggatgatatatgtatttttacccacaaaaggacaatgtacattggtagagaatgttgtaaatctttgtttatgtgttccacatcatttaacaaaacaaaacacacacacacacacatggcaggttgttttttaatatcatatcttgcaaccaatgctcccttgaataacattaatttgaaaacgtatcatacagagatgaaacctagtataatggaaaaggctatcttatcttcttctttttacaaagtgatcatcttttagttaagagaaatcaacaacctttattttatttcaattccaagttagtaattatcctacgtggtatgtgccctagtagtgtatacaattggggtgttagtctttttttaacgaaatcacacatgataaccggtggtgtggaaggaagagagatgagtctcatagtcacatgtttctaagttagattttaatcacttcatgatttagtggcatgtaaacattattgttagatactccctccctaggtctatgtttgtattgcagtgggttaggatgatctcatattttatttaaaattggtcttttctattttacatatttggttaaacaattttgttctatattccgtgactgattttttcaggaaccacaattccagccaagtcactacggtttgatacaatagaccacatacaaacctacgtgagattttcctagtcagtagtcacacaagtaaaaaacaaacgagccaacatgtagatggattttaagatgtgcatagcagtatttcgcggcatctcactaaatcacagaaatggacgtagtaacattaatcagacgatctgtttccgattacgctcagggctccaatcctgcaaccgttgtgcacatttccagatgccagccgggacggacaatcacacacagtaaactccaggtgtaaggctgcaaggaagccgggaggaacttactggcaatgctaaatgggaggcagaggaagcatccacttgctaataaatgggaggcagaagaagcatcgactccgccaccccgcttgtctcgtgcttacgaaggtgcaaaattccgtcacgtgttacctcaccttggttacttcccaaggtacgtttttatattcctctgcaagcagcaaaaggcaagggaaaagttgattgagatggagtggacagatgggtacaagtgacaagttgggcgccaacttgcgcctaaaatagacagggggcggagggtgagtcatagttatcttgtcacaattaatgtgtgcgtcattctgattttgttaatgccgttaggaaagggagatgagactgacactggtctagtgtcgctaataatgaagtgatttacttgtgtgactggtatggaggttatgattcagttgttatgcggagaaaatttaaagatgtgacatactcatgttcgtattgcgtacgcaaaaaggaaaa

SEQ ID NO: 41 - DNA, 3743 bp, Setaria viridis
cagtccaattattatgaagagactgggggtcggatggaaaaaggaggaaagagagggggataaaggggaaagtttttctctttctttgaaacatacagccaaagcagattgcgcatgggcggcggccggagtgtggcgtcgcgacctccgctcccgtctccgcctcccccctccctccatcgcgcctgctccatctcggcctcggcgcccccgcgcgggaggcggcggccgccgcgccgtgactccggcggttcgagccgacccggcctcgatctgcgccagtgcctggcggccgcgcccctatctccggggggcacgtgttctggcggacggagaaggacgaggacgagaggggcctcgaggcaacggaggccgccgtgcgcgtggtcgccacgtccgactgcatcgaggaggacaacacggcgaccgcatccacggcggtgtccctggcacgcgtgatgcgccgagccacagctcgcggagctggttcgcctcatcggctccgccgacttgaagagcgggcttgactggggcaccagggcgacggggctgtcgtgcctcgtggccgtcgcggcgacgcggcggggccccgactccggccttccccatcgatccatggagagggtgcacatcggcgacctcgtcttctacttcctgcagggacacctcgagcaggtaccctcccccgtttccactccctcctctctgcttccattggagttagctccgcatttgatatttccatggacgatgccctcaatatgttcgatgaaatgggtacacagtacaattttcttcttttgccgttcaattttgcttgctgttttgtacagtccccatctctatcagttgtgcttgtatattcaggttcaaaatttagagttcaatagagagattgacatgagctctatgagttcagcggaactgcaattgttcagtgcaattgattcaatttgtggtgtagagatgtagttgtttgaacttgtgcagagagcttccacatcatctcactctaatgtacgctattctgtcttttctgggttcaatcttctgtaggtgtatgtgtatcccatcaatacttgcgacatctttttttcttttctgcacgtaacagaaattatcatttgatgtgctcatgaaagcatatgacatagccttgatggatcaagattttctttcacaaattccctggcactatgcaattattgatgaggcccaacgtctaaacaatccatccagtgtaagtggccattttatttatgtagttcttttgaattcaaggtttgactgcttccttaccgttactgctgcttttgccaccaagcacctcttggtactactgctaagctacacaggatgtacctcccctcgtatttgtgcattatgtgatgttttcagttttgattcagtagcacggaagctcagactttcagtactgtttgggaaaagaaacatcttcactttcgagcgstgtattggtagagcataatgacagaggtaaattgggctctgtgactaagagaaaactaaagaatagctatgacttggtggcaccaagtggaaatattcatatttcactagcagtttgtggacttcaggttcagctgattagttacaacctgtgttgagcttgagagggctgcaaccataaaattgctcaaaaaacaaccccatcaatttccattacttgtaacgagaatattagagtttagttacattgccagataaaggaaacagaggagagagcagacaccccctagtagttctaacctgattcaagctaaaatcctaggtgcaattctgcatttttattggttcgttgagcttgtaatccaagcattagtgttaaaaggaattagattaataagtgtgcatgtgtcaaacactcaaatgctatatgttacacccttttccttttctccttttggttacgatcaattaaaatacacatatattttgtaacaaaccttccaagtttaactcagaaagttctttgccaggtactgtataatgtccttgagcaacgcttcatcatgccaagacgtctactactaacaggcactcctatccagaacaacctttctgaattatgggcgttgatgcacttttgtttgccttcaatatttggaaagctagatgagttcctttccaccttcaaggaagcaggggactcattaacgggtattactttcaaattctagaggggcgcattttagcatatgtcaattgccaggtaccttttgtgctgaacaacagtgcatttgtgcactcttgctatgaacactatattctccatgtagcttatgcactggtttattttccttatgcagtctgttctattgctaaaattttgcacagtttattcatgattcgtgttgtatcacatttgtataggtgaattgctgttggatgcttaatgtttttacttcttgattcctttcggcgtccttcaaaagacattcaaagttgagatcaatacttacagccggagtgaggttgttgccatggagtggctggttcaggaggccctgaacttcaagtgtttcgtcacaacaactcaccatttcctctggtaccacaaacttcctgctttcttatctgtatcagctgagcataacgacaggctaacaccattgtgttcaaatttcgcaggttctatctgaaggctgcaaaggcagatgacagggtagtggacctggcaaaatacctgtccttgctctcacttctcaaacataagcagctctccttctggccctaaactgtggcagccgctgtggtagcccttgcttgccttgccacagacaaggagtcctaatgccatttagtcatggaggtgaacacctcagtcctccatttctagctataaattgtcattacacttgctattgtttagaactcgataaacataagcaaaatatgaactgtaacatttagaatctcttgtcagtcagcttctaaacatacaccaactccaattttacatgttctgattctagctagactggaaatgcctaacgagtagctctttgcatgtatgtagactcacatgaggacgcaggatgacgatctgcctaaatgtataatggtacatttttctcctctgattttttataagttactgagcacaaaacaccatgaaccatgctcttacagttccagcaataggcagaaatctacttttatcacaatacattcattaccccgtgcaatcttctttcatgcagagcctacagtggctgatcaactacgcttcgtgatacccgtgactcccaggtgacgaaattgatccacagtttcctgattccaagttacgcagcacagttcaagcggtcagacgggcatgaggatgtgtaggccatacgtgaatcttagcattaacagattattctgtacatgccataagccaaggcagcataaaacgaagcctgtgattaaacgactttctggctaggagcaaggcaaggatcgagagtttggtattgagatgtcggcctttaggaactactgaatgacctattgcgctgtctattatctttgggtaggcttggttgtcgccacgtcggggaaaaaatggggaaagcagacggtggtcggtggatggatgactgtgcttcactgcacttgtgccatgccacgacttctactgtagtttactgcagttgatgttaaggcattcagtattactgatgatgtctctgttgtttcatatgtagtatttgttgatatcttcctataagtttcat

SEQ ID NO: 42 - DNA, 6178 bp, Brachypodium stacei
tatattttaccagtatttaaccataactagtcaaacatgttttgtttgtttttatggttttctgagcgagtatgctcttattttgcgtgatctgctgcctcctcatgtgcatctaatcatatcatgattgttcactagtaacgcgaaacctactacctgtatttaccaggtacaatagatgcgataataaagatgattcgctacgagggattgcatggattctacaaaggaatgggtacaaagattgtacagagtgtttttgccgcctcggtcctttttatggtgaaggaggagcttgttaagtttgtagttcttctagttgccaggagtaggactgtgcttcttacaagatataaaaaacaataggtcttgtttcatgataaaattatttaattgtctctacgcgtaatatcctgttcgaaattgctctttcaattctttattagttatgaaatatctcataatgctgctggtgctctttttgttggtgcccatctcttctactgcctccataaatccatgttcgagaaaaatattcatgtgtttcataaatccataatccaagtccgtcttaaaagagaacaggcttagcgtggcgtttgcatggtgccaccagacatacagcctggcggttgacttgggttgtcagacatgcatcaagaagtggctggccggtcacttgattggaagcagtaaattgtaccgattttggtactccctactaaatcagggatatttattacttattacgtatcggagtgaatagctgataatcgctatatattgattggtttgtttttttctttttcaagggtagggcgactttattcctgttacaatcaagttttgaataaagctagggggattatcgctccaagctgacaaggatttacatataaatgcctatctagccaagctatgagctacctcgtttgcctgtgggacgcaatgtcaaaatgacatgctcccgatgcggctcgcaagctgggaagtggagattgtggttgctacctgagctcacttcaaataaatctgactcaacacaattcagctgcatccgagattagcaaccaatgttatgcccattcaaaagagcaagagcaggcagcaagtcaaatattttggtgtcgaggggagccaactaaaagagtcatacacaatttctgggttttttcatgtatttaacaagtaaatgcctcgtgttaggcgggaccttggcccccattggcccctccttcctccgccattggcttcagctattactgcgctgccaatatggaggatgaacgaggttgtcacggccatgaaaacctctagtcatcgcgttatataaaagagacagcgcacaagcggggacacgcgtcgacctgcatgcatcgtctgcgttctctagtttgttccatccaccggccaacaacccatccggataagggaaccacaggcgtgctgtggaacaatgagcatgcgaagaaaccacccggctatgacgtttgtatcttacttgtatgttgatatttttcttataaattttgtcaaactctataaactcttgccaaaagatctgttctcctcgcaaaagaaaaagaggcgcaaaaggggagagaagaaactcaaggtcagcgaaactgccactgaaattcttcgcaggaaagagcttgactcctggaaagtggaaaccactcgcggtgtgacaccgacttaccgtgtgcagtcatagcagaagacaacatggcatcgtgcctgtccttccgtctcgcttggaaacggggaacaccgaggctgttgttaccgtcaccgtcggtccgttgttccggcagatcgctcacatacaccggtgtttcctcctgggacgtgggatccgcatccactgcaagtggggtcctacggagcggtgtacgtgcacctggcgcagctgggagctcgcgatttcctgaccaagctaaggaatttaaaattcaaaaagagctggtcgggctgtgagttcatcacggcaggacccgcaggagtcgtcctcccattccccttcctccccggtctcgacggcctcgcaggcgcggctatataagcaagacaattcacctagccgtagcccgcagcgacatacaccgatacacggccacgcgcacacactatgcctcccaccatgctcgcaccggtgcccacgaggccgcgctccaaccccttccgccggcggagaggggcggctgctccgctcccagcccagatcgcagcggcggcggcgccgaatcggcccgcggattcgtccacatcggcatcctcctccttccgcagcgaggtcatctccaccacctcctccaccgccctcgccgcagcgcagcgcccggagaagaggcctcggcgtcaggacgcggacgaggcgcggcctgcagcctctgagtgctcagaggtcatcggcggcgcaagggcgcgcgtcgcggaggtcgaggtctccgagtcatcatgcctcggctccgttctcgagtccgaccttgcctaccccgagctgctcgccgacgatgcagaggcgactgagtactcttcggcccgcgatgacctgacgcagtcagacgccgaagaggaggttctcagtgctcccagcccctgctccgagtactccctgacccccctgatcgactcctcctcctcgtccgacgacgacgatgacgccaccccctcccccaccttctccctcttcctcgccttcgccgagcaattcgtcccctgcgcgcacaccaaagcgcacgccgtcgccgacgttgcgattccagaggtgagcgggtatttccacacaattgctctaaatttgtgtgaaattgggtctgtttgcaagtgtcggtgcggtgtttcatccgattaggtggcttggtgggaatgtttgtgtcaggggatgcgttttgaggacttgaacgacgaagagagctacgagcggttccggcgccgtgagcggcggggagtggtggcgtgtgactacaccgagctgtacaactgcatgccagacagctatggccgtgccgtcgtggagcagcgtactgtcatggtgaactggatcatcgaggtccgtttaatactgcggttatcactctggcccatttgatttttgtggtagaagcgtgccttacaggattacagtaaaatgcatgcgtacaatggaagtcacgtagtactctaaattctgtgttgttttatttgtcagagtctgagtgtccaataaacctctggtgtagtggtgtaaatgttttgtgctgggcctgctgaggcagcagatttagctacccaatttcgtggttagtgcagcggaaagttgtgttatcgaagaatttgttgtacaacattctgatgagaaggttgcgcaattgacattgttcgctgaacagaaggatcctttttttcggaactgttgttgaaataccgtgcttattgcattactcagtaggatcccttatagtaggattctgatcaccaattttgactgtcagcccttccaagtcaaagagatctgagaggagtgttaggatgtttaagcaggtgttgttttgaccttcgacattcactgtttgaaaaggaatggacaaatagatcgttcagttatgctgatatcagtgccattttacatgtcatctgcatgtggcctgtgtcagggaaacatattaggatatctgcatcatttctatctagatatgcaattcatgagtactttatcggtataatcccttatatagactccaatgaaaatgtaagtttgtctatgcacttgatattgcattgattctggagagaatctctgagagaatactgacaccagcgtttcttccagttccaccttaagatttcggtatgcagtttaggtatttaaaagatgagaaaacttgtacagtaatattcctgaaaagcattgtactatgactcctggagcaaagatttattctcagataaaatttcatctacaactgacgaaatactgactgtttcccatgaattctagcatgggcatgttaccgatctgcagccagagacagtgttcttggggattggactgatggatcgcttcttgacgcgtggatacgtaaagggcactaagaaaatgcaattgctgggcattgcctgcatcacccttgccacccgcattgaagagaaccaaccatacaattggtatgagtgcccttgcattacatgttcattctgtttcagtaaaatcggtgcatggtagtcagattgcctcctttgaagtgtgtagcctcttaactgatgcattgctgaattaaataatgcttcaaagaatgtcctcatctaaattcaaatcttagttcagtgtagtttcttattatattgtcacatggttgtagtatagtactgttagatcctacctaagctattctatgcggtatttgcttttctgtttgataaagctcatcgcagcatgacttaatttagcatttgatagaccttagcaagtatggttgggatgccttggtcctgattgatttacctgcggtcaggtagctctcctctcaccaatcactagagcatagtacagagctagcatcttcagtttagaacataaaatctattactatgttatccccactgaagggaactgaaccattctacgagtgatccaaggtagcataagatccaactttagtttgattacatcaggcaattcatcatgatttagtgcccattttgacttgggtagaccgttcattccagagttcaatctattttttgccaagtaaacctgttgcatcaactttttggtcgtgcatacttacatgattgcacaagtgaaatcagagcggtttgtggttatgttattgaccgcatgtctggagataataaatcattgtgtgtcacccagtactatatatacagaaccaatagagtacttaatatttaacatcaacaggagcagtggagcagtgcagaattattgtcgaactctgggttcattgtctatggggtcaacatttgtttcctgattaatgtttatgttcagaaatgtgcaatggcgaacctcactatattatctctttcagcatcctgcaaaaatctttcaaggtagggatcaacacttacagccaaagcgaggtcgttgccatggagtggctggttcaggaggtcctcgacttccaatgctttgtcacgacagtccaccatttcctctggtactatgtgtttttttagatgatctctggtactatgtgtttcctgcattcttgtctgttccaagccatacaaaatgaacgagacgctaacaggttactctgttctaatttggcaggttctatctgaaggctgcgaaagcagatgaaaaagttgaggatacggcaaagcacctggccttgatctcacttctggaccataagcacctctcctactggccctcaaccgtcgcagccgcagtggtagcccttgcttgccttgccaccgacaacgactcttcctgcgaactggtgatggaggtaagtactgaaatctttaaccaaggatatacatttctgtcgttctgaactttgataaaacgtattgttcctaacaaagactggaatcacctaactgtttgctcgtcgcataatacagactcacatgaggacgaagaacgacgatctgcctgaatgtttaacggtttggtccctcactcgcattctgatacctggcatgaacttaactgtattgttcaccgtttgcagtagctgatgtaagtaaatttttgtggtgccgcagcaactaagattcgttatgttatgcaactttttgtgcagagtctcgagtggctgataaactatgcttcgtagtatccaggccccccagaatcgagcagcatagcagtcatttaacatcaacaaaaagagcgtacatgccatttggttgcacaacaggataaataaaaaggacaaggcagcaggtttatgactgtagggacaaccgttgtggtcgtctgtctttgcatcatcagttagctctttaggaacaattaaggagttaaggttggattctgttgtattcctcaacttctgtttttccttggatcaacggtcttgtttaatgggcttgtttaatgttagtgtatgcttggtatttttcgcccggtttactagtagctctttaggaacagttaaggttggatgctgttgtgttcctcaacttccatttttctatggatcaacggtcttgttta

SEQ ID NO: 43 - DNA, 8430 bp, Panicum virgatum
tcgacaaaatgccggagttcggctgcgtgcccaatgctgtggtttacaccgcgatgctcgatgggatgtacaatttcgggaacttggatggtgcggtgaggttgatcgaggagatggagggcagtgggttgggtgcaaattgtgcaccgaacgtggtgacctatacatgtttggtgaaatgcctctttgggaaggggaggttggcagaggcgcttggtgtgctggataggatggtaggtagaggggtgatgccaaaccgggtttttgtgatgacactcctcgaaggtgtctgcacggagcggagggtggccgatacatataatgtggtcgagcgtgtggttggtgatcggggcatgtcgagtcagcagtgctacaatgttctacttatttgcttgtggagggttggcatgacagctgaagctgaaggattggcacaaaggatgatgaagaaaggggtgcagttgtccccgcttgctggcagtttgatggtgagggagctctgtacaaggaagaggtcgctggatgcttaccactggttgggaatgatggaggagaacggtgtgctgtgtgactctgacgtgtatggaactctgttgcttggtctgtgtgaggaagggcatgtccatgaggcatcagcattggggaggaaggttgtcgagagagagatccacatagaagcatcttgtgctgaacgtttagcggagtttctgaagcaatatggtgatgaggagctagcatctcatttattaggattgaaacagtgccctggagggctgtcattttaagcaatgcgcgattctgcccaaccctctgcatgaagcatgtcatggttagtcatggggtgtgccaagaatagtggggaatttgcctgagaacagatttagccaaatggcttagtgcagtcaaaagtttacttttgttgaataaaacatgaaacataattcaaccgaagtgctgctgaactacttgcttcttttgtacaaatttgctgaagaacatgatgcagatccagaggacacttggcgtcaagtaaactaccattttgatcacttctcaggtagatgacatgcttggacaagtgctgtgcctgtcagtcgaacgttttagatatgtttcatgtactgtaatccgaggaagttatgtacaacgttgctcgagtcatttaacatgatacgtgcccataaacacctaccctgacatgctgtaacgttttcctgttactcagttttttgctgcccccttatccaaagaactgaaaatagaatttactttctcattttcttccaatttgtttgtatgattgagcacaacattttcttccaatttgtttatatgattgagcacaacctgcacctgcaccacattgctttttaggaacgtttacttgcaaattttggtgcccgtcaaatctgagtctgacattctgctcttgtcggtgtgaaagaaatctaagggcaagcaaacaaaaccaggcggtccagctgatgctgatggaagcaaggcggccgctgcgcttgcatagttgtatttgcattgcatttgcaggtggctgggcgctggaggcagcggtcgagtgagaatgttttcacataactgtagtgcagacggcattcttcaagttcaggaatcagggatcattttgatttgcaaccggaatatgactagttgctgggattttgctgctgggaacgcagtgagcgattgaactctgaaagaaaagtcacgaacctattgccactccgaatcaagctattcagcagatcacacacatcgcagcaaaagtgaatcacggcaatacggcatgacagtgacagtctgcaacagccccggacattcccaacggaggctgacgcggccgttgttctggcatcccacgccgcgagcggggctcgcaagtcgcacagcacgccgtcaatctactctggctgcgggtgggaccagtgaagcgcacccgtccatcaccgttcaggatttaaattcgaattgcttttcgggcctgggcgttcattgttgttgatctctccttccccaagtctcagtggtctccacacaggcagcggcaggtcggagctatataatcaaggcaaacacggcatctagccgtagcaagtagcacagcaaatcacacacacacccccggggggcacgcgcacgcacggatcacacaatgcctcccgccctgctcgtgccggtgcccacgaggccccgctccaaccccttccgccgacggagaggggcggctccgctgctcctggatcaggccgcgactgcggcggcggcggggaatcggcccgccgattcgtccacctcggcgtcctcctgcttctacagcgaggtgatctccgcctcctccacctccctcgccgagtaccagcgcccggagaagaggcctcggcaccaggacgcggacgaggcgcggccggccggctccgagtgctcagaggtgatcggcggcgcgagggcgtgccccgccgaggtcgaggcctccgagtcgtcgtgcctgggctccgtgctcgagtccgacctcacctgcccggagcagctcgccgacaccgccgaggcgacagagtactcctcggcgttcgaggagctgaccccgtcggagcccgaggaggatgaggaggtgctcagcgggacttgccgctgcgccgagtactccctcagccccctgatcagctcccccttgaccgaagacggcggcggcgacgccgcgccctccgcgaccttctccctcttcctcgacctcgccaagcagttcgtcccctgcgtgcaccccgaagcgcgcaccgtcaacaatgccgctctcgatctcctgacggtgagcgggtttccacgcgatttctgacaaatttgttgggaattggatttgactgcattgatcagtgctgtaatcgatctgttaaattggtagctgatgtgatgcgaatgcttgtgtcaggggaggcggtttgaggacttggacgacgaggagagctacgagcggttccggcggcgcgagcggcgcgaggcggttgcacgcgactacactgaggtgtacggctccatgtccggcagctacggccctctcgtcgttgagcaacgtgtcgtcatggtgaactggatcatcgaggtcagtgtatactacattctgttgtgcgcgtacggtgcgatcaacagttcacctcaggttaagcatccgatgtgaccgatttgttgcatttgtaggagctgtataggcttatgactgcattgtactggtggcatacgactctagatcctccgttggttaattgttttattgtcagactcaatggtctagaatttgtttgcagtgttcagcaagcacggtatctgcataattgcataagaagctgttctctggtgtaaatgtttttttaattgatgactatctggttccctttagtattttgtgctggcgaaattatctaccatctaaaagtttgttagtttgccattagttgatgagtggatttggaaatggcgacactattgtgagatatcagggttcaggggtgtccttgtagcctgtcattgagtagtcatgcttgtactgactcagtttgagcactcagttttttcaaccaaattgtccttgtcccagagatcagctatgtctaacatgggctgtttgaagaggaagagagaaataactggttgggtcgcacaaaaagaaaatctctgtagttcccatttggcatttgaaacttcatttcattggcatgttgcttccatgtctgggattacatattgtagcaattaggatacctgaaccttcgctctctaaatgcaattgtctacctgtaactctgaatgtgcccttcattgcatatgcatccccacaaatttgagctgttttgatgcacttggtatttgtttgagattcagagaaatcctgaaagcattgccatcaccattttccatcagaaggtttcttgaaattatgcattacgaatgtttttgaatatttacgttgcactttactgctgtgtcattacaaaagcttgacctgaacaatttatgctccttcatttctgctgttccaaaaccaaagttcacttgcaaagaatgactctttcccttgcaatggcagcattcgcatctcacgaagttgcagccagtgaccatgttcatggggattggactgatggaccgcttcttgacacaaggatacatgaagggtctgagaaatctgcagttgctgggtattgcctgcatcaccctggccacccgcatagaagagaaccagccgtacaattggtaatgttctcccttgtgttacatgttttaattttcatgtctggttttaaggtacgctattcttcatatttggtgaatcctatctggaccgctagctccctttcatgctctatttcagtataactaccgactatagttggtgatcctgtgttgtcagattgcctaattgatatatacaccattgacattcagcagcaattgatgcataattaactacattaacattcagaagcaactgatgcataattaattggcttagcactccaaattaactcctcctttaaccatgcattggtgctggacattctctcagattgtcaaatatatgctgcaaggtataactgtgtttcttttaccaactgcatcactacctcacagaaatatgggttgggtttgagtcagtagaactatttcacttttaaattatgttatgaatctgaactatgttagcgtgtgacagtggttgtcatgttattttcattttttatttgtccctttttatactagcactctgtagcttgcatttagaagtgtgcacaaggtgagcatgtcagaagtgttagagtatattaggcaactccggatatggttagtttaggattgattgtaatcccgggataatctttcttatctctaggaaatgctacttgccctccaagccatgtactcatatatataccgcccaaggggctcaatgcaatacatcgatcacattatacacatcctactttcttacatggcatcagacgcctaggttttagatcctgacctagccgccgccgcttccgctgccgtcgcgccgcccccggggagatcgatctccgccgggggtagcgccctcctaggatgcgccgcggatccctatgatccgcaccgccgttgttgtcaacacacagcagaaggacctacctcccatggtgatgcgatctcgttggctccctcattgccccttccacgccgactccccccgtgcgcgcggccccgtgctggccctctctcctcctgcgccgtcgctgccctgtgcgcaggctgcggaggaggcacgaggtgcgccaggctgctgcgccatcggagccgcgagattggggcgggttaccgtgccgctggtcgattccggcaccgcccgacgagatccgccgctgggacgacctgcagcggggccaccccgtttcgccccggcctccatgctccaccacctcgctcctccgccatcgccggcgggccgcaccatcgtgggggctccaccccatctcggcttccccagctcctcgccgccgtcgcctcccgacccgtcgagaccggcctcgtccgcgccttccgcggcggcgccgctcccatagtcccgatccgcgccttcctggagcggatccacttgctagaggcggaggcccgccgctacgccaccgccgcgccggaatcgagaaggcagcgccggcggcccactccctccttttcttctctgcccgtgttggccacggggggaacagataagggagggggaggggcaccgctaggggtaccctgagaatgtatccatggttcgtttgctgcagctgattttctttttttttccgatctaagatcggtttgcgtcgccttgccattcgtcgccgttcatcgacactcccgaaggacgaggttgttgctgcgccttccaggttgcagcgacagcgacgacgttcgtgatcaagccaccctaccggtgtcgtcgccttttcaggcggtggcgccactcgccgccggtcttcgtcaagcaggcgctcgatccgtctccgcgcctccagccgttctcgtgcagacatcgccgccgatgttcctgaaggaagacgtcgtcgccacccctgatctgagcgcgacacttgctgcaacctgcgccgtcgccacccctgtcctgagcgtgacctaagtcgcaaaccgcgtcgtctaccatcgtcattcgccgcaccgtcctgctgctgtcttcggcaagaagctgctgagtttgtgtactcgagcacatcaacgatgcttcgacccgcgccccctctacggcttcgaccacgtccacctcaacttcggctactacggcactaaagggctatcatttgcatgagtctctagtcaaagctttcgcaccggcattccgactgcagggggatatgtctccattgttctccagtctaaccgttcgtgttgctaccgctacgactgcggggggatgttagagtatattagtcaactccggatatgtttagtttaagattgattgtaatcccgggataacctttcttatctttaggaaaggctacttgccctccaagccatgtactcatatatataccgcccaaggggctcaatgcaatacatcgaccacattatacgcatcatacctttcctacaagaagttccttagtttcctgattgtgctgtcctgctgttatgtgttaacagtggtagaagaattgagcatactaattggcattttttgttgattgataatatcttttgctatatggttttcattcctgcattttgcatttgtaagacaattcacagacatatctttagttgaaattgcgctgaaacgtatcctatgagtttgtctcctgatcataacctgtttccaattatttatttctgctaagcttgatgtgcaacagttatgtggtttcttgattcttttcagcgtccttcaaaagacatttaaagttgggatcaatatttacagccggagtgatgttgttgccatggagtggctggttctggaggtcctcaactttaagtgttttgtcacaacaactaaccatttcctctggtaccacgaacttcctgctttcttgtctatatcagctgaacaaaaaggagaggctaacaccattctgttcaaaatttgcaggttctatctgaaggctgcaaaagcagatgacagggtagcagacctggcgaactacctgtccttgctctcacttctgaaccataagcagctctccttctggccctcaactgtggcagccgccgtggtagcccttgcttgccttgccacagacaaggagtcctcgtgccatttagtcatggaggtgaacacccgaatcctccatttctagttataaactgccgttgcagttgttattctttagaactcagtaaccataaaagccaagtatgaactgtaactttctgaacctctcgtctgataacttctaaatatatatcgactccaaaattacatgttctgattctagctagactggaaatgcaaatgcctaacgagttgctcacatgtatgcagactcacgtgaggacgcacgacgacgatctgcatgaatgcctaatggtacatttttcctctttttctatataaaaaaattaccgggcaccaagaaacatgttaattacagtttgtagcactaggcaaatatcgacttttatcactctatgacacctaattgtcattagcctgttcaatgttctttcatgcagagcctagagtggctgatcaagtacgcttcgtgatacccatgactccaaggtgatgacattgatccacattttttgctgattcccagttacctatcacagtacaaccggtcgggtatggggatgtataggccatacgtgaatcttagcattgacagattattctgcacatgccattagtttccctgtaggtagataagataaaagaaaggcagcataaggtagcgtctgattatgagagaatggctaggagcaagacaatgacgagaatttggtatttagctgtcggcctttagggactataccgaactgccgtattgggctatatatctttgcatcatttcttcgctgttttatggacaattaaagctgctctgttgtactcaccactgatgcaatttttttaaacggtgtcttgttcataacttttgctttagaaaagggtcttgttcatacctgatgtaagactgaaatttgttgcctgcgtgtggatcggggcgtgatcttgtgatcctggaagatgcttccgtatcccggtatccggttttatggggtgaacaaaacgactgtcctcagatctgtcagcttgtctgctagactggagatcgcaaacgaataaagtttcagttaactgcagatcctgcaagttcggatcgcaacacaagcctgcacactcggatccacatcgtatcactgattagtttcactttcacgtgt

SEQ ID NO: 44 - DNA, 3222 bp, Populus trichocarpa
tgcagtttctgttaatgagtgtgttgtagagaagcagaagaagccaaacagcttgggaggaggaggaggagaaagtgatgacctggcttgtacagaggagttgtatgtggacgacggagtttcggattactcgtcttgtcaggagacgttgttctcggagctgcaatcggagatattccgggaaaagtattcatcggacgacctcgatttctctgatgattacacgccgtctattttcttcgaatctggaagcgatttttctgagaagtccgtaagtgattcgaatccttcgcagacttattccctgttgctccagtacagacagcaattctcgcgatctagtttacctctagaaactacaaaatcatcgtcactccttgaagcagagtatcaagagaatttcgccgtgagtttttgaatcaacaattacttttgttttcgtgttattttgctatgttttaatgctctcattgttttttaatttgtttaatttctgtgcttttcatttgtttttattgcttaagtttgcgagattggacgatgaggaagatgaggagagctacaagagattgagagaaagagagagaaggcaattgtttttgcacgactaccctgaattgtaccgtaacaacacggagttcggcgatctcatcctccagcaacggttgcagatggtacactggattatcgaggtaagttttttataattaatcgcggtagctaacaaatcaattgctcggatgcggcgattttgcgacagttggctatttttttcttgtcaatacacacacgtttgaagaagttgattccttcaattatggaagatgtcctctacactggagattctacactggagattctactactggaagtgttaagcaaatggggttgtgacacgtcatagtttgagattttgggttttggactttaaaactgtatttgtaccatttgtggtatggtcagtactcagtactgtccttgtactagattaaactagattttctctgtggctcattttttctttctatttactaaagggtatttttatcatttaaacttgggctgtttgtagtggtttcaaggcactaaacactaatcaacaacagttgttaatcacttgtttatcatttttggactttcaattgacaatctgtatgagcaatgtgtgccttatgttcttctaattaaatggtaatcacttatgttaaaaaaatggtaattgcatgttgaattcgtgtctgtcgctttgattatgcagcaagcaactgcgaaggagtttgaaccgtgtttcttggaattagccttctggaccggttcctagcaatagggttcttcaagaacaaaagtcaccttcaaattgttggtatagcttgtctttcattggccaccagaattgaagaaaaccagccctataactggtaaatatctctgccccgttcttttttgtggtcgtggtgtcctggattgcttaaggaataaaataaaacaaggtgccagtcttggatgcataattctttccttttagtcctttgcatactaatggcttgcatttacaacatagaattgtcaaaaaacgattaggatcgtgaataaacttgttgatcatcttaagaaacagatcacaagtgagtgttgtgtggttattttttatggacttactagaaaagaaaaattgctgtcaacttgttgccagatcaaggctcaaatgaacttgaacacactggatgaactatactttaatttcttaacgtttgcaagtactaagacattcctataccgctctccagttcattctttttcatggttatataatctgcctcattttcatcaaagctagctgtccatcacttctcaaaactgaaggtaatatgatatctagctgcaagttctgctccggtcaatgagagctccgctgacagttttataatgggaatttcagtgttaggcagaagaatttcaacattgggaacaatgtgtacagcagaagtgaagtagtggccatggaatggctggtgcaggaggtccttaacttccgatgcttcttgcccaccatccacaatttcatgtggtaactccttttccctttccactttgctttatagtttatggtggtaagatttgtatcctgtcttggatatgaacactttagttctttctgtcattcatatgacctgtagtttaggaagtctaatacaaccttgtcctttttccatgctgaacattaggttctacctgaaagctatgaaagctggtgcagaggtggagaagagggccagatacttagcagtgctggcactgtcagaccttgagcaacttaggcattggccctcaacagttgcagccacgcttgtcatcctggcttctctagaaagcaatgaaattgcatcctatggacgagttatcgaggtataaatattaattggcaacacaagcttgccgtgctcgaataccaaaataaaacgatttgggctgttaatgggtggaggaggtgctgttgcttgtttcatttaatatttttggacaagatttgccgtatgatatgattcagtgaaatatctgaaaactgtcctaggacatactttttccccgggcctggccccggcccattcctaaagtgtactgcaattaacatgctcttaggttctgattagtcaatttgattttacaggttcatgtaagaacaaacgaaaatgacctccaccagtgcataaaggtatgcaaataaagatcttttctaaaacggtataattgtaccctcttttttatgcaattcttttctaggaaggtgctgctattgatgcatttacattcacattcacattctcgaaagctagtgccgctggtgttgattttgcctttattgttaatttgattcgaattatttgacttcggtattaacagagcctagagtggttgctgcaatatatgagctagcagtctagcaggagaaggaatgaaagatcataagatcgcctcttgtacactcgcattcttttctcacacgcatcactggttactgtagatgaagataattgtaaagcctgataataacaggtaacacaaatctatttcttttacggtacatgcttttaccgaactgttcataatatagaatgacaggatctgtattcaaggagctggctcatgtaaattcaaagacataatattccaagtattcttttgtatgttctatgcaaaacgatgatgggattatgcagacag

SEQ ID NO: 45 - DNA, 4879 bp, Eucalyptus grandis
atttttcaaaatatgactatttatattgtttataaaaataaatggacgaaaaatatttttatattaaatttatttcgataaattatttcaagcgatataatttaaaagctcatcaaactaatggattaggtccggtcattaatgtttcatctaaattgatcaatttaacacgtttttacttatttttacgtaatacaaatctaataacttatacttgacccaacccgactcgacacatatctcaatcgttcccaacctaatccaacccatttccaactctatccatattgcaaagtaagtatgagatcaagtgaaagcgagagagattaaagatagaattataattataaaagtgaattagatctaagtaaattgtaaatccatttatgacccatttactcaatataattaatctatttataactcatgtaatatccatatggattaagaaattggtttatgaccattttaacgggtctagatacaagtggttgttttcaattttctttctaaatcattaattttttacaaaacaaacaaagtctaattataattttttttctctcattagaatgttttgatgttacaaaaacctaaaaattcgccttatgtttatcctatattctttattcacgattgcattgatattttaaattttttttttttgataactgaggatccgctggatccgcctttcacttatgctaattggcaaccatggcccgtacaatgcacggtgcaccttagaccaagcatcaatacctcaggaaagttagcacagaactccaccaccataatccctctgcttaagatgtgttagcaactacgaagtttcaattttgggacctctgtagtaaagtgctcaaagcccaactaactcaactacccatcggtgggtgatgtattttaatttggtactgtaaaaaaaaaaagatatagaaaagctttagcatgattgccaatttctcgattgatcatttgaaatgtcaaagcatcaagtaaaaattgattttaccctctttgatagattacgtgtttattcttttagctaaagctagattatatgttttgaatgaatttattggattcttgaggctaaagggtcgaatcccgaattattccctcccttctttcttttcttttttatttagccctttaaaggctcaagtctaagaggcaacaagaaattaacatgttttgattcggttcattgatttgatgaattgaatcaagtcaatatcaaatatgatcaaaactttcgaccattccaattaaagtcaactaattaaaccgattagtccaaccctaattttaaataacattagagttgtcgctcacccctcttccatcaccactgtggctctgtgccgcctagtgttccgacgaaaaggcgaggccgaccatctcacatgcaaatcttttgcccttcacgttggcggaaattgcagcactgccaccgtgagcattctgaccggacaaaagagactttttcgccacttcaaaaaactcgataactgcccggtaaacgtaaaatatatacatatatatagagaccaaaaaactaacctattttccaaaaaaaggaagggaaaccagcccataaatactctctcggttcctgaacaattctcattatctgtgtggggcccatggccgagccagtgcgggccgcgcgatgatcaagtggatcacatcaacggctatgaccgatccgcagttcagtagcaggagatgcctcatgcggagcgatacactacatcgtgtccccttcacgacgtggttaaacccacgtaaaaccacactccccaaactccccataaccgccgcctcctcttctccttcttctccttcttctgctcctcctcgccacactgcgatcacttcacgaaattcctccgtcatcaaactcacaacggcaccgtctccaagcttcgaactagttcgacgaagttcgattcggacgacggtgggagccggaaccggataggaaccgccggaaaatgatgcagccgccgccgccgccgcacttgcggacacacaggatgctccggtcgaagcttcctcagaggaggcggtctcagttctctcctctcccttgcttctcgcgcaactccgccgtctccgcttccgtcgattccagctacttggacggcgaggtctccgtcgggacggcggacgcgaagaaggagaagaagagcgcgagcgcgagcactcggaagaggcgatccgatgctgtcggaggcggcgattgcgagtttaggagagcgacgaggtcgtcgtgcaagcgacgacgggaggaggaggaggaggacgaaggcggagttgccgaagttgttgcggaggtctcggtgtcggagtcgtgttccggttcgtctttcgaggttgattccaaagaggagagctccgagttgaaagacaccggaacctaccatggtttgaaggagttcagaggaaatgcggcttcggagtcgatcgcgcgatcggagatctcctgcgtccaccagcattcgccggcgaagtccagtgagctctccagcggattgtcgaggaacagaaatgctgaaaatgaggtcgtctctctcccgtcttttgttgactcgtgctccaaggcaactagcggtttccgaatttccgaattggaggacgagacgcttgaagtcgagtgccatgagaacagagcatcgtcggagattttctctgaaacaacaaaaggtcacgtcaccggttgtttactcgcttcaaagtcggaatcgactgtcgatcaaaagccaggctccatcaattacgacaccgacctcgcctgcacagagcaaatcgcgtacgacgacatctcggaccactcttccgatctcgattcattctccgagctacaaaaggagctattcgaggaaaactcggacgaatactcctccgagtacattctgtcgatttcctctgaatctggatttgagttctccgagaaatcagatgaagattcgaatccttccccaaccttctcttttctcctccagtacagggagcaatatttgagatcaagtacccgtgcagacaaccacaaagctcggagtctcagttctgaagtaaatgtaagctcttactgtacgaagctgttattgtacgtgcgtacgattttcgttgtttgaaatttgaatttgattaacttttatgaactcgaattttagcatggatgtggcgacgaggaggatgcggagagctatcagctgttgagaaacagagagaggagagaagtcttcttgcggaactacgttgaggagtacggtgagcccgtgctcaaggaaaggacgcgcatggttcactggattattgaggtgagtcacacgaagatagggtccgagttttcacccctcatctttaattagatgcaagatgtcgatggccaagacattgatccgttgaaatccatttataatttgcataaattgcttgtagaaaccacttttgcatggcatatagataactgcatacattcctcactgttagatccatgcctgtacataagagattgcttcaagctggacaaaacgtcatgcctctatatatttacatggatcctgtttatatggatctttctttccttctgtggtccttcctttgacctcttacaagtttgatattccatgttaacagcattctagtgcgagagagctccacaacgagactttgtttctaggggtgagcctcttcgacagattccttagtaaaggatacttcaaacagcgaaggaacttccaaattgctggaatagcctgtctcaccctagcgaccagaattgaggagaatcaaccttacaactggtaatcagattttatctccttcattttccttcatacagagggatgctaatcaatgccctcattagaaggcaataactgctttggacctttcggttagcaaatgtgaagatgcttagacctcgagctttacaaatcttctttttacttatgctcccagcaatcatttcaccataacttcagacgacaccacatttggaatgttcattggatcgagtaatttcagttacatgtggtagaccgttaggaaatacatcatgaacaacttcaaaggtttgccatcgcaaaggaattaccgatcactcaaccttcactatgtccgggtccgggttgccagtagtgacttacaatcactccactgcataattcatttatggatggtaaacacccaattggttcatgcttagtccctaacagatgtaaatggtactggtaataaccaattactcaaacttccctatgtccgggtccaggttgccagtagtgacttaaaatcactccactgcatgacttcttatggatggtagacacccagttggtttatgccaagtccctaacagatgtaaatggtactggtatttcagcgtaagacaaaagaacttccgtgtggggagagacacctacagcagatgcgaagtggtggcaatggagtggttggtacaagaggttctcaactttcagtgtacattgcctaccatacacaacttcttatggtacagtccatatcttgtctgcgcaacctcgatcggcagacttattttcttttctctcatattgtgtgttttcatgggttaagacttacttttcagacttagcaaattccgaccacttgactaatctgagtaaagaaagtgattctgctgaatattactgaaatatgcagaaatgattgcaggttctactcaagagctgcaagagctgatgctagagtgattaagaggaccataaacctggcagctttagctctgctagaccatgagcagctgtcctactggccttccacagttgcagctgcgcttgtcatcctagcatcagtggaagacgcatcctgcaagcgagtcatgcag

SEQ ID NO: 46 - DNA, 6769 bp, Prunus persica
atccaattattaaaataaaagattcaagaacctcaagaatctctccctccctaccagaagaagaagaagaagaagaagaatcaattaatttcagagataaagaattcgttgaagaagaaataatggctgaatgcagttaccagaacattaagaacccacaaccagaaccagaagatgaagaagaagaatacacagcggtggttggcaaacacttgtccatgctccgccttgacaacagcagcagcagcagcagcagcttcaaatctcccaattccagtcccaagcccaggagaacattgaaaaggcgatccccgtcccaatccccaccaacatcccaacccaaccccaagaaagagaagcttgatctccctcctgatcctcttcttcgccgctgcagttccgaacgcttcaacccaacttctcctcctcctcccccattttattcttttaattctcatcacaatcagctgcagtgccccaacgcagcctctcctgcctctgcctcaacagataaagcctctggcgcggctgctctctcttcctatgcctccacactccgccgctccgtttccaatcccaagccttcttcgtgttcgcctgctctcaaaaccttctcccgtcaatcctcctcctcctctggtgacgaagacgacaacgacgacgccactcccaattctaaggttttcttccttcacctccatctttcattctcttgactttgaatttcatccgtatctttcatatgaacgtatggttcttgaagatgaatttctattatttttgcagaggcttagaaggataaaatatcgcgtcagagagatgagcctgtggttccaacaagtcatgcttgaaaatgaagatgatgacgaggaagaagaagaagaactggaactggaacctcctcaagaacaacatcatcaacaaaatggagacactactgaggttggtaactctaactcatcatattttaatcatatctttacaacaattggtttcgttggttcccaagaattagaaattacaaatcctttttcacaagtattgggttcggacttagaaattccgttgatattatcatcatcatcatctctttctttccgttgatgaatttgatgttgcagttgcaggtcgatagtgacataaattttgcagaatctgtgagcgtggagaggatgggggatggcttagtcattcatttcaggtgccactgtggcgtcccctatcagttccttcttgctgggggcaactgctactacaagctcatgtagatttgtatttcacaacccccttttaccactagactacccacaaaaaccactttttattctgcctttctttttaccttttgctcaagtacaagttcatatgtgcaaacgatggcattaatttgttgattcttctggctatatggttattttctttctgtattgctgtatttcacttactcttggcaaaaagaaatgttctgcttttttattctacttactaatcacaatgttatcagccttaccacctgcagtaactagaagggcttagttagtaccatctccaaatagttgtgcgagtgtcatacataatctattttaaggatattttgtcattcgaggtatgaatattactttttttttaaaatcacatagtcgcacattgcaaaaaaaaatttaagtgacagtaccatattttttatttttgaagagagaccttgcggggagggttaaagttgaaatggcattggtataatatttatcaataatattcactttgcaaaacagaagagttgcgccttgcgtatcaagtaatgctaaagagggcggcccatatgccacatcaaaacccatcattaacgaaaaaagaacgcacacagcctatactaaaagaccaaagaggacctcgtcgcacaatgtagtagaatgttcggattcacgggaaaaattgacatgagtcgtggggccacaaaaacgaaaggacgaaagaggacccccagtccccaacttccgtaaccgcttacttgaacacactctcctcccttccctctcttccatttctcactctccactaaaaccaaccaaacagccctcacactcactgcaaataaattcagaaattcgaatttcaaagctaattccaaaatgaagtttaaatcgatccaagcgatgcaaaacctccaaacggcgccgtacctcaagacgaagaagaagctccggtcggagctccctcgccggacgcgatctcagatctctccggttctgtactcgtctctcaagttcaatgcccccagtgagacctctggtttctcttccttctctgtgaactcgaactcctgctcgtacttcggcggtgaagtttcgtgcgagtcaagcagagtttccgttggatctgagagtgaggcgaggtcgagtttgcggaagagacacttcgaagaaactgagaaacaccgtaaaatgctgtttcctagagctactagatcgtattatggaggaaaggcggagaagaaagaggtcggtggagacggcgaaccagaggtttctgagtcgtcgtgtgtggagtccaattctggagctgattttgggggtttcgtagacagaaagttgaagctaaagagtaagagaggaaaaggaagtgaaattgtgaacgaaaatggaggaaacgaaggttctgaagcagttagtagaccagagatttccgagtccgataaggtctcattggaattcaaggaaaacaaagttacgtcattcaactctgtcatcgaattgtgctctgaatcgaaattaccagagaaaacggtcaaagacggagaaaacagagcaccggaatttgaattccctgaaatttcggaaaactacttcggtgaaaatttcgcgatttcgaactctgaatcgacgatagagcagaggccagacagcttgcaaatcggttctgaccttgcttgtaaggagcagttctcctacgacgacgtctcagagtactcttcgagtcagacattgtcggagctccaatcgaatattttcgacgaaaattccgagctcagtctctcagagtacactccgtctattttcttcaattctggaagcgaattctcggagcgctcggatgaagattcaactcattcacctacttttaccttgctccttcaatacagagaagaatttacgagatcgagcactgctttatatttcattgctgcttcctgcgttaaagaactgtataaggatgattatacagtaagttttatcaagttttgaattacgatcttaattttcccttgctttcctgcactttctcgccaaccaaacaaaaatagattgttgattttttatcttttttcattactccttctgtattgatttttgttattttaataatattattttagtttcttaagttcgaagacgaggaggacgaagcgagctatcagctgcttaggaacagagagaggatacaagtatttttgcgagactacacggaggagtactcttccacgacggaatgcggcgatcttatcctccagcaacggtggcaaatggtccgttggatcgtcgaggtgattggctttaccgaaattcacgtttctctgattaagttcaattaatcgtcgttttctaaatttaaataaggtcgaagttcaactaatcgtcgtttttatctatttaaatttggtcgtagttaaattaagcgtcgtaattagttctgtttggaaattggacctgcacacgtttgtggaagtacatgccgtcaagtagcacatactatctacattgacgatattctacaaccatttaacatccaatcaaatctgtgccacgtcattgaatggatgtgtttggcactgaaactgaatgcatttctagtttttatgttaggatcattcatgtactttttatcacaggcactggcacatcactagtttcgcttttctcctattggccaaagtaatacacatttgtaatgtatagggagattaatttgatacatttttgcgaaaaatgattttatgtaaatttgatacatgtacgtctccaaattcaattttacgtaatccttcttagcttaacaacttccacgttgccccacacttagaacgcagtagtagcacgtgcattcgcaccagcgatggtgcgtatataacatttgttgaggggtacttgttacttgttggacaagtctatacactccacgattttttgcgtaggtgcgattaatttcaagaatttcaatacaaagaacttgcatacagcatactgactagggtggatatccaaacggtcaattaagtaattggatcgatttggttccatttctataaaaaaaagaaaaagaaattaaattaattcataattagtttggtttagttcaattcattctctataagaacaagctaaatcgaactaaactgcatattattatttatttattttggcacaaggagttttatattggtaatgatcatttactattatgtttcttcccactatttatatcaacatttaatataattgtattgttagtttgttactttaatggaatgttgaatattgttagtgtactagagatttaaaaagtaaaatggtgaatgcaatgtttgctttacaagtgcttgaaattgttaagattgttaagttattttgactctcatgtatagaattcttgtgttgcacatgagtatattgggtggtggctatgtttatactttgttggtggatgttcgaatgccattgtcaatttgctttgcttgacatgcattgagatgtgaatttaagatattgatgctcttcatgctttttctgataaagtggtaataagagattgcattatagttaaaaatagtgttcaccctcatcattatagtggttaattttcacgtaaaactctaatattccgtttcctgtgggagagggtgcataggctagctgttatccgtatttcttataactaacgttttattatctcttgttattacgttaatgcggtgcttttgattggctttcaacaagcagcgatcgaatcaaatgaagctacagcaggaaacgaagtttctaggagttagcctccttgaccgattcttaagcaaaggatttttcaagagcaaaaggatccttcagattgttggaatagcctgtctaactctagccaccagaatagaagaaaatcagccctacaactggtatatctttatacttttttttatcattcatacatttttacctagtctgtttcctcataaacctggtgttgaggtgttttttagccttttttgtgtggcaattaaagcattacttatagatgaatacaaaaatcagaagttgaagcagtccaattccttctgcaagtgatttgtctggaaatgaatgtattagagaaactgtgaagttgcttagagctcaaacttaaagttaacccacatccccttttggtacttaaataacgatgtaattgagacatgtcaaagcaggacgcatcataattaccttctaaatagacctttctgatggccatttcagcgtgcggaaaagggatttccatgtagaaagcaatgtgtacagcagatgtgaagtggttgctatggaatggctagttcaggaggtcctcagcttccagtgctttctgcccaccatctataacttcttatggtaactctcagtctcccattttctccaaagtactatactagacaaatgcatgctgcttctcttgcattgacaacctggcaaactcaggctcctgatcttctttctctgcatatctagattatccttcaacttttattttctttttttctgggcaaagaaaatgtttctgatttgagaggttcatgccatcttcattccatgaactaattggataacataggttctacctgagagctgctagagctgatgcccaagtggagaagagagccaagtacttggcagtgctgcagatgtcggaccatgtgcaacttcgttactggccttcaacagttgcagctgcgcttgtcatccttgcttctttagaaggcaatcatgaggtgtcccgccaacgagtcatagaggtaactgccataatctttcttttaaactacattgaattataatgtagagggatgccattatgtcatcatgaagaacacatctggttcttgatatctcattgatgcgatctcatggctttgcagactcatgtgagaacagaaggtgatgatttacatgaatgcatagaggtaaggataaaatatgaggtatcataaagttcaataatttttcattttcatttctccaattatctcgccatctagcacagcatcatttcttttctgtttttcttaatgtttcgaatatattgtcattttaagatagttgataggtgctgatggtgtgctaactttaacagagcctagagtggttgttacattatgtgtgatttctgtttgctgactccctcatcaaaagagatggatggaaggtagatcaaggtaaagcctgatcaataggtaacaaaacaaatctgattttttcgtcaattaagacgaccgtgcagctacttgtaaacatttcatagaagtacagaatctgtaataatatctgatggtctccaaggaccaaaagtaaactttatgaacttatgtttgaaaagtacttcactactcaccatgaatgttttacctgctttgtttctagcatgcgtcattatccatacaatggctgctttgctaagaatttaagcatcatgataccttgaatgtctatgcagactatttttgagggcgttgacatgccccctaaagtttgaaggt

SEQ ID NO: 47 - DNA, 37 bp, Artificial sequence
  Synthetic
tgttttggtgggaatgcttgtgtcaggtcaggtcagt

SEQ ID NO: 48 - DNA, 50 bp, Artificial sequence
  Synthetic
ctagctagactggaaatgcctaacgagtagctctttacatatatgtaggt

SEQ ID NO: 49 - DNA, 27 bp, Artificial sequence
  Synthetic
ggtgggaatgcttgtgtcaggtcagtg

SEQ ID NO: 50 - DNA, 47 bp, Artificial sequence
  Synthetic
cagtaatctcactgcttgatccctttcaggtaccacgaatttcctgc

SEQ ID NO: 51 - DNA, 14 bp, Artificial sequence
  Synthetic
tgattttgcaggta

SEQ ID NO: 52 - DNA, 34 bp, Artificial sequence
  Synthetic
caccggtaccccatcattctcgtctctctcgcac

SEQ ID NO: 53 - DNA, 35 bp, Artificial sequence
  Synthetic
cagtgtacatttttctccgtacgaaagcttgaaac

SEQ ID NO: 54 - DNA, 26 bp, Artificial sequence
  Synthetic
ccgctcgaggcaggctttatgaagac

SEQ ID NO: 55 - DNA, 28 bp, Artificial sequence
  Synthetic
gctctagagcggccgccgatctagtaac

SEQ ID NO: 56 - DNA, 24 bp, Artificial sequence
  Synthetic
ccaatgcattggcgtataacatag

SEQ ID NO: 57 - DNA, 24 bp, Artificial sequence
  Synthetic
ccaatgcatatggcagcgctggca

SEQ ID NO: 58 - DNA, 21 bp, Artificial sequence
  Synthetic
gaagatctggatccggcttac

SEQ ID NO: 59 - DNA, 24 bp, Artificial sequence
  Synthetic
gctctagactcgagctgttccacc

SEQ ID NO: 60 - DNA, 28 bp, Artificial sequence
  Synthetic
ccgctcgagtacgctgtgaggatctgtg

SEQ ID NO: 61 - DNA, 29 bp, Artificial sequence
  Synthetic
gctctagaaggatatcctgatccgttgac

SEQ ID NO: 62 - DNA, 20 bp, Artificial sequence
  Synthetic
ggaggaagacatgggatggc

SEQ ID NO: 63 - DNA, 23 bp, Artificial sequence
  Synthetic
cccttgttcaccaccttcacttc

SEQ ID NO: 64 - DNA, 19 bp, Artificial sequence
  Synthetic
ggagaactcgcagaccgcc

SEQ ID NO: 65 - DNA, 20 bp, Artificial sequence
  Synthetic
ccacctgggtcagctatgac

SEQ ID NO: 66 - DNA, 22 bp, Artificial sequence
  Synthetic
atggtatctctaaagtcccttg

SEQ ID NO: 67 - DNA, 21 bp, Artificial sequence
  Synthetic
ccaaatcctcggaactgaatg

SEQ ID NO: 68 - DNA, 20 bp, Artificial sequence
  Synthetic
cgtctccaggatcgaggaat

SEQ ID NO: 69 - DNA, 20 bp, Artificial sequence
  Synthetic
ggagatgggaaagctgagag

SEQ ID NO: 70 - DNA, 20 bp, Artificial sequence
  Synthetic
gttgggatgaaccagaagga

SEQ ID NO: 71 - DNA, 20 bp, Artificial sequence
  Synthetic
gaggagcctcggtaagaaga